(19) United States
(12) Patent Application Publication
(10) Pub. No.: US 2008/0261978 A1
(43) Pub. Date: Oct. 23, 2008

(54) Chemokine receptor modulators

(76) Inventors: Michael P. Clark, Duluth, GA (US); Mark A. Lockwood, Alpharett, GA (US); Florence F. Wagner, Duluth, GA (US); Michael G. Natchus, Alpharetta, GA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/073,648

(22) Filed: Mar. 7, 2008

Related U.S. Application Data
(60) Provisional application No. 60/905,580, filed on Mar. 8, 2007.

(51) Int. Cl. A61K 31/5377; C07D 211/70; A61K 31/44; A61K 31/17; C07D 265/30; A61K 31/5375; A61K 31/135; C07D 235/04; A61K 31/495; C07D 403/02; A61P 35/02; A61P 35/00; A61K 31/18; C07C 311/01; A61K 31/496; C07D 241/02; C07D 241/04; A61K 31/4184; C07C 211/00; C07D 413/02; C07C 275/00; C07D 239/02; A61K 31/505

(52) U.S. Cl. 514/235.8; 546/332; 514/357; 514/597; 564/51; 544/332; 514/275; 514/237.8; 544/168; 544/139; 564/305; 514/646; 514/394; 548/309.7; 544/398; 514/252.12; 544/390; 514/255.01; 564/86; 514/603; 544/363; 514/253.05; 514/254.08; 544/373; 544/122

(57) ABSTRACT

The invention provides compounds of Formula (I)

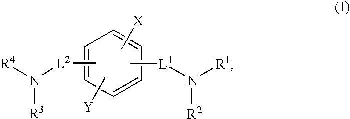

and pharmaceutical compositions comprising compounds of Formula (I). These compounds are useful treating or preventing HIV infections, and in treating proliferative disorders such as inhibiting the metastasis of various cancers

### CROSS-REFERENCE TO RELATED APPLICATIONS

The present application claims priority to U.S. Provisional Application No. 60/905,580 filed Mar. 8, 2007, the disclosure of which is herein incorporated by reference in its entirety for all purposes.

### FIELD OF THE INVENTION

The invention provides compounds, pharmaceutical compositions and methods of use of certain compounds that are antagonists of the chemokine receptor.

The compounds are useful to modulate a medical condition that is modulated by chemokine receptor activity or signaling, and in particular in the treatment or prevention of human immunodeficiency virus infections (HIV) or the diagnosis, prevention, and treatment of cancer.

### BACKGROUND

Cancer is currently the second leading cause of death in developed nations. In 2004, the American Cancer Society estimated that approximately 1.37 million new cases were diagnosed in the U.S. alone, and approximately 550,000 deaths occurred due to cancer (American Cancer Society, Cancer Facts & Figures. 2004, see URL: http://www.cancer.org/docroot/STT/stt_{—}0.asp).

Metastasis, the spread and growth of tumor cells to distant organs, is the most devastating attribute of cancer. Most morbidity and mortality associated with certain types of cancer, such as breast cancer, is associated with disease caused by metastatic cells rather than by the primary tumor. Therapy for metastasis currently relies on a combination of early diagnosis and aggressive treatment of the primary tumor.

The establishment and growth of metastases at distant sites is thought to depend on interactions between tumor cells and the host environment. Metastasis is the result of several sequential steps and represents a highly organized, non-random and organ-selective process. Although a number of mediators have been implicated in the metastasis of breast cancer, the precise mechanisms determining the directional migration and invasion of tumor cells into specific organs remain to be established. An incomplete understanding of the molecular and cellular mechanisms underlying metastasis has hindered the development of effective therapies that would eliminate or ameliorate this condition.

Several strategies have been developed to reduce metastatic invasion of malignant cells by regulating adhesion of endothelial cells with antibodies or adhesion molecules (see for example, PCT Publication No. WO 97100956, U.S. Pat. Nos. 5,993,817; 6,433,149; 6,475,488; and 6,358,915). However no commercial strategy has provided an effective treatment to prevent metastasis.

Chemokines are considered to be principal mediators in the initiation and maintenance of inflammation. They have also been found to play an important role in the regulation of endothelial cell function, including proliferation, migration and differentiation during angiogenesis and re-endothelialization after injury (Gupta et al. (1998) J Biol Chem, 7:4282-4287). Two specific chemokines have also been implicated in the etiology of infection by human immunodeficiency virus (HIV).

As of the end of 2004, an estimated 39.4 million people worldwide were living with HIV/AIDS, and the Centers for Disease Control and Prevention (CDC) estimate that 850,000 to 950,000 U.S. residents are living with HIV infection (UNAIDS/WHO AIDS epidemic update, December 2004; Fleming, P. L. et al. HIV Prevalence in the United States, 2000. 9^{th} Conference on Retroviruses and Opportunistic Infections, Seattle, Wash., Feb. 24-28, 2002. Abstract 11). Although new infections have decreased in recent years, an estimated 4.9 million new HIV infections occurred worldwide during 2004 and approximately 40,000 new HIV infections occur each year in the United States.

HIV entry within the target cells involves a series of molecular events. The three main steps of virus entry within the cell are: (i) attachment of the virus to the host cells; (ii) interaction of the virus with the co-receptors; (iii) fusion of the virus and host cell membranes. Considering the complexity of the molecular events involved in viral infection, all three of these steps have been considered for the drug design of HIV entry inhibitors. The T-lymphocyte cell surface protein CD4 is the primary receptor involved in the interaction with the viral glycoprotein gp120, but a cellular co-receptor is also needed for the successful entry of the virus within the cell. At least two types of such co-receptors have been identified so far, both of which are chemokine receptors. These chemokine receptors are therefore gateways for HIV entry, determinants of viral tropism and sensitivity.

Chemokines are a superfamily of small, secreted cytokines that induce, through their interaction with G-protein-coupled receptors, cytoskeletal rearrangements and directional migration of several cell types (Butcher, et al. (1999) Adv Immunol 72: 209-253; Campbell and Butcher (2000) Curr Opin Immunol 12: 336-341; Zlotnik and Yoshie (2000) Immunity 12: 121-127). The chemokine receptor, CXCR4, is known in viral research as a major coreceptor for the entry of T cell line-tropic HIV (Feng, et al. (1996) Science 272: 872-877; Davis, et al. (1997) J Exp Med 186: 1793-1798; Zaitseva, et al. (1997) Nat Med 3: 1369-1375; Sanchez, et al. (1997) Biol Chem 272: 27529-27531). T Stromal cell derived factor 1 (SDF-1) is a chemokine that interacts specifically with CXCR4. When SDF-1 binds to CXCR4, CXCR4 activates Gα₁-protein-mediated signaling (pertussis toxin-sensitive) (Chen, et al. (1998) Mol Pharmacol 53: 177-181), including downstream kinase pathways such as Ras/MAP Kinases and phosphatidylinositol 3-kinase (PI3K)/Akt in lymphocyte, megakaryocytes, and hematopoietic stem cells (Bleul, et al. (1996) Nature 382: 829-833; Deng, et al. (1997) Nature 388: 296-300; Kijowski, et al. (2001) Stem Cells 19: 453-466; Majka, et al. (2001) Folia. Histochem. Cytobiol. 39: 235-244; Sotsios, et al. (1999) J. Immunol. 163: 5954-5963; Vlahakis, et al. (2002) J. Immunol. 169: 5546-5554).

Compounds targeting CXCR4 have been developed primarily for treatment of HIV because CXCR4 is a major coreceptor for T-tropic HIV infection. For example, U.S. Pat. No. 6,429,308 to Hisamitsu Pharmaceutical Co., Inc. discloses an antisense oligonucleotide to CXCR4 to inhibit the expression of the CXCR4 protein for use as an anti-HIV agent. PCT Publication No. WO 01156591 to Thomas Jefferson University describes peptide fragments of viral macrophage inflammatory protein II which are described as selectively preventing CXCR4 signal transduction and coreceptor function in mediating entry of HIV-1.

Peptide antagonists of CXCR4 receptors have also been disclosed. Tamamura et al (Tamamura, et al. (2000) Bioorg. Med. Chem. Lett. 10: 2633-2637; Tamamura, et al. (2001) Bioorg. Med. Chem. Lett. 11: 1897-1902) reported the identification of a specific peptide-based CXCR4 inhibitor, T140. T140 is a 14-residue peptide that possessed high levels of anti-HIV activity and antagonism of T cell line-tropic HIV-1 entry among all antagonists of CXCR4 (Tamamura, et al. (1998) Biochem. Biophys. Res. Commun. 253: 877-882). The compound has been altered to increase its efficacy and bioavailability by, for example, amidating the C-terminal of T-140 and reducing the total positive charges by substituting basic residues with nonbasic polar amino acids to generate TN14003, which is less cytotoxic and more stable in serum compared to T140. The concentration of TN14003 required for 50% protection of HIV-induced cytopathogenicity in MT-4 cells is 0.6 nM in contrast to 410 mM leading to 50% toxicity. U.S. Pat. No. 6,344,545 to Progenics Pharmaceuticals, Inc. describes methods for preventing HIV-1 infection of CD4+ cells with peptide fragments. U.S. Pat. No. 6,534,626 to the U.S. Department of Health & Human Services describes certain peptide chemokine variants for treating HIV infections. PCT Publication No. WO 041087068 to Emory University describes CXCR4 peptide antagonists, particularly TN14003, and methods of their use to treat metastasis.

Other peptide-based antagonists have also been disclosed. For example, European Patent Publication Nos. 1 286 684 and 1 061 944 to the University Of British Columbia cover methods of treatment of diseases, including metastasis, using modified peptide CXCR4 antagonists derived from the native SDF-1 ligand. PCT Publication No. WO 041020462 to Takeda Chemical Industries, Ltd. provides peptide CXCR4 antagonists for treatment and prevention of breast cancer and chronic rheumatoid arthritis. U.S. Patent Application No. 200410132642 to the U.S. Dept. of Health & Human Services in part covers methods of inhibiting metastasis or growth of a tumor cell with a polypeptide CXCR4 inhibitor.

In mice transplanted with human lymph nodes, SDF-1 induces CXCR4-positive cell migration into the transplanted lymph node (Blades et al. (2002) J. Immunol. 168: 4308-4317). These results imply that the interaction between SDF-1 and CXCR4 directs cells to the organ sites with high levels of SDF-1.

Recently, studies have shown that CXCR4 interactions may regulate the migration of metastatic cells. Hypoxia, a reduction in partial oxygen pressure, is a microenvironmental change that occurs in most solid tumors and is a major inducer of tumor angiogenesis and therapeutic resistance. Hypoxia increases CXCR4 levels (Staller, et al. (2003) Nature 425: 307-311). Microarray analysis on a sub-population of cells from a bone metastatic model with elevated metastatic activity showed that one of the genes increased in the metastatic phenotype was CXCR4. Furthermore, overexpression of CXCR4 in isolated cells significantly increased the metastatic activity (Kang, et al. (2003) Cancer Cell 3: 537-549). In samples collected from various breast cancer patients, Muller et al. (Muller, et al. (2001) Nature 410: 50-56) found that CXCR4 expression level is higher in primary tumors relative to normal mammary gland or epithelial cells. These results suggest that the expression of CXCR4 on cancer cell surfaces may direct the cancer cells to sites that express high levels of SDF-1. Consistent with this hypothesis, SDF-1 is highly expressed in the most common destinations of breast cancer metastasis including lymph nodes, lung, liver, and bone marrow. Moreover, CXCR4 antibody treatment has been shown to inhibit metastasis to regional lymph nodes when compared to control isotypes that all metastasized to lymph nodes and lungs (Muller, et al. (2001)).

In addition to regulating migration of cancer cells, CXCR4-SDF-1 interactions may regulate vascularization necessary for metastasis. Blocking either CXCR4/SDF-1 interaction or the major G-protein of CXCR4/SDF-1 signaling pathway (Gα₁) inhibits VEGF-dependent neovascularization. These results indicate that SDF-1/CXCR4 controls VEGF signaling systems that are regulators of endothelial cell morphogenesis and angiogenesis. Numerous studies have shown that VEGF and MMPs actively contribute to cancer progression and metastasis.

Several groups have identified chemokines including CXCR4 as a target for treatment of metastatic cancers. For example, PCT Publication Nos. WO 01138352 to Schering Corporation, WO 041059285 to Protein Design Labs, Inc., and WO 041024178 to Burger generally describe methods of treating diseases and specifically inhibiting metastasis by blocking chemokine receptor signaling.

Although advances have been made, inadequate absorption, distribution, metabolism, excretion or toxicity properties of peptide inhibitors have limited their clinical use. Small non-peptide drugs remain a major goal of medicinal chemistry programs in this area.

At the present time, the metal-chelating cyclams and bicyclams represent one of the few reported non-peptide molecules to effectively block CXCR4 (Onuffer and Horuk (2002) Trends Pharmacol Sci 23: 459-467.36). One of these non-peptide molecules is AMD3100, which entered clinical trials as an anti-HIV drug that blocks CXCR4-mediated viral entry (Donzella, et al. (1998) Nat Med 4: 72-77; Hatse, et al. (2002) FEBS Lett 527: 255-262; Fujii, et al. (2003) Expert Opin Investig Drugs 12: 185-195; Schols, et al. (1997) Antiviral Res 35: 147-156).

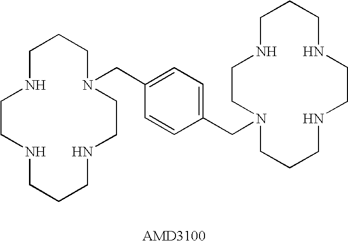

However, a clinical study showed cardiac-related side effect of AMD3100 (Scozzafava, et al. (2002) J Enzyme Inhib Med Chem 17: 69-7641). In fact, AMD3100, was recently withdrawn from the clinical trials due in part to a cardiac-related side effect (Hendrix, et al. (2004) Journal of Acquired Immune Deficiency Syndromes 37(2)). The latter was not a result of the compound's ability to block CXCR4 function, but due to its presumed structural capacity for encapsulating metals.

Other nitrogen containing bicyclic molecules have also been developed as CXCR4 antagonists. European Patent Publication No. 1 431 290 and PCT Publication No. WO 02/094261 to Kureha Chemical Industry Co., Ltd cover CXCR4 inhibitors that are potentially useful in treating various diseases including HIV infection.

U.S. Patent Publication No. 2004/0254221 to Yamamazi, et al. also provides compounds and use thereof to treat various diseases including HIV infections that are CXCR4 antagonists. The compounds are of the general formula:

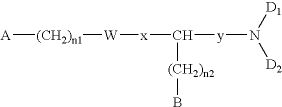

in which A is A₁-G₁-N(R₁)—; A₁ is hydrogen or an optionally substituted, mono- or polycyclic, heteroaromatic or aromatic ring; G₁ is a single bond or —C(R₂)(R₃)—; R₁, R₂, and R₃ can be optionally substituted hydrocarbon groups; W is an optionally substituted hydrocarbon or heterocyclic ring; x is —C(.O)NH—; y is —C(.O)—; and D₁ is hydrogen atom, alkyl with a polycyclic aromatic ring, or amine.

PCT Publication No. WO 00/56729 and U.S. Pat. No. 6,750,348 to AnorMED describe certain heterocyclic small molecule CXCR4 binding compounds, teaching that these are useful for the protection against HIV infection. The compounds are of the general formula:

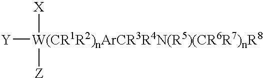

in which W can be a nitrogen or carbon atom; Y is absent or is hydrogen; R¹ to R⁷ can be hydrogen or straight, branched or cyclic C₁₋₆ alkyl; R⁸ is a substituted heterocyclic or aromatic group; Ar is an aromatic or heteroaromatic ring; and X is specified ring structure.

PCT Publication No. WO 2004/091518 to AnorMED also describes certain substituted nitrogen containing compounds that bind to CXCR4 receptors. The compounds are described as having the effect of increasing progenitor cells and/or stem cells, enhancing production of white blood cells, and exhibiting antiviral properties. PCT Publication No. WO 2004/093817 to AnorMED also discloses substituted heterocyclic CXCR4 antagonists which are described as useful to alleviate inflammatory conditions and elevate progenitor cells, as well as white blood cell counts. Similarly, PCT Publication No. WO 2004/106493 to AnorMED describes heterocyclic compounds that bind to CXCR4 and CCR5 receptors consisting of a core nitrogen atom surrounded by three pendant groups, wherein two of the three pendant groups are preferably benzimidazolyl methyl and tetrahydroquinolyl, and the third pendant group contains nitrogen and optionally contains additional rings. The compounds demonstrate protective effects against infections of target cells by a human immunodeficiency virus (HIV).

PCT Patent Application PCT/US06/000604, filed Jan. 9, 2006, describes certain compounds for the treatment of medical disorders mediated by CXCR4. These compounds include two nitrogen linked cyclic substituents off a central aromatic or cyclic alkyl or heteroalkyl.

In light of the fact that chemokine receptors are implicated in metastatic signaling as well as a number of other pathogenic conditions, it is important to identify new effective chemokine receptor modulators.

It is therefore an object of the invention to provide new compounds, methods and compositions that modulate chemokine receptors.

It is another object of the invention to provide compounds, methods and compositions that bind to chemokine receptors and interfere with their binding to their native ligands.

It is an object of the invention to provide new compounds, methods and compositions for the treatment of viral infection, such as HIV.

It is also an object of the invention to provide compounds, methods, and compositions for treatment of proliferative disorders, such as for the inhibition of cancer metastases.

### SUMMARY

In one embodiment, the compounds of the present invention are compounds of formula (I), or a pharmaceutically acceptable salt, solvate, prodrug, tautomer, or ester thereof:

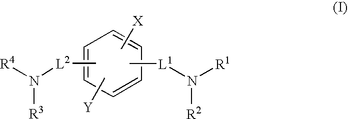

wherein

L¹ is —C(O)—, —S(O)—, —S(O)₂—, —N(R⁵)—C(O)—, —N(R⁵)—S(O)—, —N(R⁵)—S(O)₂—, -alkylene-N(R⁵)—C(O)—, -alkylene-N(R⁵)—S(O)—, or -alkylene-N(R⁵)—S(O)₂—;

L² is alkylene, —C(O)—, —S(O)—, —S(O)₂—, or a covalent bond;

R¹, R², R¹, R⁴ and R⁵ are each independently hydrogen, substituted or unsubstituted alkyl, substituted or unsubstituted alkenyl, substituted or unsubstituted alkynyl, alkoxy, alkoxyalkyl, alkoxyacyl, haloalkyl, cyanoalkyl, hydroxyalkyl, thioalkyl, alkylthioalkyl, substituted or unsubstituted aminoalkyl, substituted or unsubstituted aminoacylalkyl, substituted or unsubstituted amino, substituted or unsubstituted arylamino, substituted or unsubstituted heteroarylamino, substituted or unsubstituted arylacyl, substituted or unsubstituted heteroarylacyl, substituted or unsubstituted aminoacyl, substituted or unsubstituted arylalkyl, substituted or unsubstituted heteroarylalkyl, substituted or unsubstituted heterocyclylalkyl, substituted or unsubstituted aryl, substituted or unsubstituted heteroaryl, substituted or unsubstituted cycloalkyl, substituted or unsubstituted cycloalkylalkyl, substituted or unsubstituted heterocyclyl, or —S(O)₂—R^{z}, wherein R^{z} is selected from the group consisting of substituted or unsubstituted alkyl, substituted or unsubstituted arylalkyl, substituted or unsubstituted heteroarylalkyl, substituted or unsubstituted aryl, or substituted or unsubstituted heteroaryl; or

R¹ and R², taken together with the nitrogen atom to which they are both shown attached, form a substituted or unsubstituted 5- to 18-membered saturated heterocyclic ring containing at least one nitrogen atom; or

R³ and R⁴, taken together with the nitrogen atom to which they are both shown attached, form a substituted or unsubstituted 5- to 18-membered saturated heterocyclic ring containing at least one nitrogen atom;

X and Y are independently hydrogen, halogen, —CN, —OR^{x}, —N(R^{x}R^{y}), —SR^{x}, acyl, alkyl, alkoxyalkyl, haloalkyl, cyanoalkyl, hydroxyalkyl, aminoalkyl, thioalkyl, N-alkylaminoalkyl, N,N-dialkylaminoalkyl, alkylthioalkyl, —S(O)—R^{x}, —S(O)₂—R^{x}, —S(O)₂—N(R^{x}R^{y}), N-acylamino, —C(O)—R^{x}, —C(O)₂—R^{x}, and —C(O)₂—N(R^{x}R^{y}); wherein R^{x} and R^{y} are independently hydrogen, substituted or unsubstituted alkyl, substituted or unsubstituted arylalkyl, substituted or unsubstituted heteroarylalkyl, substituted or unsubstituted aryl, or substituted or unsubstituted heteroaryl;

with the following provisos:

(i) R¹ and R² are not both hydrogen;

(ii) when R³ and R⁴ are both hydrogen; then neither R¹ nor R² is hydrogen;

(iii) L¹ and L² are not both —C(O)—;

(iv) when L² is a covalent bond, then L¹ is —C(O)—, —S(O)—, or —S(O)₂—; and

(v) at least one of R¹, R², R³ and R⁴ is substituted or unsubstituted arylalkyl, substituted or unsubstituted heteroarylalkyl, substituted or unsubstituted aryl, or substituted or unsubstituted heteroaryl.

In another embodiment, the compounds of the present invention, or a pharmaceutically acceptable salt, solvate, prodrug, tautomer, or ester thereof have the structure of formula (I), wherein L¹ is —N(R⁵)—C(O)—, —N(R⁵)—S(O)—, —N(R⁵)—S(O)₂—, -alkylene-N(R⁵)—C(O)—, -alkylene-N(R⁵)—S(O)—, or -alkylene-N(R⁵)—S(O)₂—; and R³ and R⁴ are not both hydrogen.

In another embodiment, the compounds of the present invention, or a pharmaceutically acceptable salt, solvate, prodrug, tautomer, or ester thereof have the structure of formula (I), wherein L¹ is —C(O)—.

In another embodiment, the compounds of the present invention, or a pharmaceutically acceptable salt, solvate, prodrug, tautomer, or ester thereof have the structure of formula (I), wherein L¹ is —S(O)—, or —S(O)₂—.

In yet another embodiment, the present invention is directed to a pharmaceutical composition comprising at least one compound of formula (I), or a pharmaceutically acceptable salt, solvate, prodrug, tautomer, or ester thereof, and a pharmaceutically acceptable excipient.

In yet another embodiment, the present invention is directed to a pharmaceutical composition comprising at least one compound of formula (I), or a pharmaceutically acceptable salt, solvate, prodrug, tautomer, or ester thereof, and a pharmaceutically acceptable excipient, and at least one additional pharmaceutically active compound.

In still another embodiment, the present invention is directed to a method of treating a disorder, symptom or disease in a patient in need of such treatment, comprising administering to the patient an effective amount of at least one compound of formula (I).

In still another embodiment, the present invention is directed to treatment or prophylated of a disorder, symptom or disease that is modulated by chemokine receptor activity or signaling.

### DETAILED DESCRIPTION OF THE INVENTION

The compounds, methods, and compositions of the present invention modulate the effect of chemokine receptors. These compounds can be used to treat or prevent HIV infection, reduce viral load, or alleviate progression towards, or the symptoms of AIDS in a host in need thereof. In addition, these compounds can be used to treat tumor metastasis or any other disease, particularly hyperproliferative diseases involving chemokine receptors.

Compounds described herein have the capacity to interact with chemokine receptors and potentially inhibit receptor signaling. The compounds of the present invention have increased bioavailability and efficacy in inhibiting chemokine receptors.

Although not bound by theory, these compounds may inhibit metastasis through their capacity to inhibit SDF-1-chemokine receptor interactions, which can decrease cell targeting, and may also reduce VEGF-dependent endothelial cell morphogenesis and angiogenesis. This endothelial cell growth is a key event in metastases of tumors.

In one embodiment, the compounds of the present invention, or pharmaceutically acceptable salts, solvates, prodrugs, tautomers, or esters thereof, have the structure of formula (I) as described herein.

In another embodiment, the compounds of the present invention, or pharmaceutically acceptable salts, solvates, prodrugs, tautomers, or esters thereof, have the structure of formula (IA):

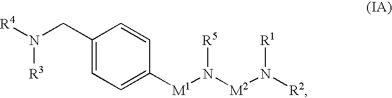

wherein L¹ is M¹-N(R⁵)-M²; M¹ is alkylene; M² is —C(O)— or —S(O)—, or —S(O)₂—; and R⁵ is selected from the group consisting of H, substituted or unsubstituted alkyl, substituted or unsubstituted aminoalkyl, substituted or unsubstituted arylalkyl, substituted or unsubstituted heterocyclyl, and substituted or unsubstituted heteroarylalkyl.

In another embodiment, the compounds of the present invention, or pharmaceutically acceptable salts, solvates, prodrugs, tautomers, or esters thereof, have the structure of formula (IA), wherein M¹ is —CH₂— or —CH₂CH₂—; M²-C(O)— or —S(O)₂—; and R⁵ is selected from the group consisting of H, substituted or unsubstituted hydroxypropyl, substituted or unsubstituted amino-CH₂CH₂CH₂—, substituted or unsubstituted amino-CH₂CH₂CH₂CH₂—, substituted or unsubstituted morpholinopropyl, substituted or unsubstituted imidazolylpropyl, substituted or unsubstituted pyrrolidinylpropyl, substituted or unsubstituted benzyl, and substituted or unsubstituted pyridylmethyl.

In another embodiment, the compounds of the present invention, or pharmaceutically acceptable salts, solvates, prodrugs, tautomers, or esters thereof, have the structure of formula (IB):

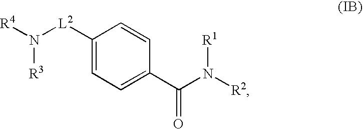

wherein R¹, R², R³ and R⁴ are as defined above.

In another embodiment, the compounds of the present invention, or pharmaceutically acceptable salts, solvates, prodrugs, tautomers, or esters thereof, have the structure of formula (IB), wherein R¹ is selected from the group consisting of H, substituted or unsubstituted arylalkyl, substituted or unsubstituted heteroarylalkyl, alkyl, and substituted or unsubstituted aminoalkyl; R² is selected from the group consisting of substituted or unsubstituted arylalkyl, and substituted or unsubstituted heteroarylalkyl; R³ and R⁴ are each independently selected from the group consisting of H, substituted or unsubstituted arylalkyl, substituted or unsubstituted heteroarylalkyl, alkoxyalkyl, hydroxyalkyl, substituted or unsubstituted aryl, substituted or unsubstituted heteroaryl, substituted or unsubstituted aminoalkyl, substituted or unsubstituted aminoacylalkyl, substituted or unsubstituted aminoalkyl, and substituted or unsubstituted heterocyclylalkyl; or R³ and R⁴, together with the nitrogen atom to which they are both shown attached, form a substituted or unsubstituted 5- to 18-membered saturated heterocyclic ring containing at least two nitrogen atoms; and L² is alkylene.

In another embodiment, the compounds of the present invention, or pharmaceutically acceptable salts, solvates, prodrugs, tautomers, or esters thereof, have the structure of formula (IC):

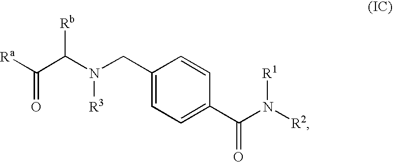

wherein R^{a} is selected from the group consisting of substituted or unsubstituted amino and substituted or unsubstituted heterocyclyl; and R^{b} is selected from the group consisting of H, substituted or unsubstituted arylalkyl, substituted or unsubstituted heteroarylalkyl, and aminoalkyl.

In another embodiment, the compounds of the present invention, or pharmaceutically acceptable salts, solvates, prodrugs, tautomers, or esters thereof, have the structure of formula (IC), wherein R¹ and R² are each independently selected from the group consisting of substituted or unsubstituted arylalkyl, and substituted or unsubstituted heteroaryl alkyl; R³ is H or alkyl; R^{a} is selected from the group consisting of bis(alkoxyalkyl)amino, substituted or unsubstituted piperazinyl, substituted or unsubstituted piperidinyl, substituted or unsubstituted (pyridylmethyl)amino, and substituted or unsubstituted (benzyl)amino; and R^{b} is selected from the group consisting of H, benzyl, aminopropyl, and substituted or unsubstituted heteroarylaminopropyl.

In another embodiment, the compounds of the present invention, or pharmaceutically acceptable salts, solvates, prodrugs, tautomers, or esters thereof, have the structure of formula (ID):

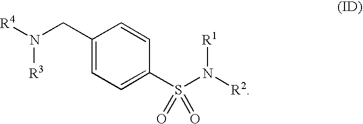

In another embodiment, the compounds of the present invention, or pharmaceutically acceptable salts, solvates, prodrugs, tautomers, or esters thereof, have the structure of formula (ID), wherein R¹ is selected from the group consisting of H, substituted or unsubstituted alkyl, alkoxyalkyl, substituted or unsubstituted arylalkyl, substituted or unsubstituted heteroarylalkyl, substituted or unsubstituted cycloalkyl, substituted or unsubstituted aryl, substituted or unsubstituted heteroaryl, and substituted or unsubstituted aminoalkyl; R² is selected from the group consisting of H, substituted or unsubstituted alkyl, alkoxyalkyl, substituted or unsubstituted aminoalkyl, substituted or unsubstituted arylalkyl, substituted or unsubstituted heteroarylalkyl, substituted or unsubstituted aryl, substituted or unsubstituted heteroaryl, and substituted or unsubstituted cycloalkyl; or R¹ and R², taken together with the nitrogen atom to which they are both shown attached, form a substituted or unsubstituted 5- to 18-membered saturated heterocyclic ring containing at least one nitrogen atom; R³ and R⁴ are each independently selected from the group consisting of H, substituted or unsubstituted arylalkyl, substituted or unsubstituted heteroarylalkyl, alkoxyalkyl, hydroxyalkyl, alkyl, substituted or unsubstituted aminoacyl, substituted or unsubstituted aminoacylalkyl, substituted or unsubstituted aminoalkyl, substituted or unsubstituted cycloalkyl, substituted or unsubstituted aryl, substituted or unsubstituted heteroaryl, substituted or unsubstituted arylacyl, substituted or unsubstituted heteroarylacyl, and heterocyclylalkyl; or

R³ and R⁴, taken together with the nitrogen atom to which they are both shown attached, form a substituted or unsubstituted 5- to 18-membered saturated heterocyclic ring containing at least one nitrogen atom.

In another embodiment, the compounds of the present invention, or pharmaceutically acceptable salts, solvates, prodrugs, tautomers, or esters thereof, have the structure of formula (IE)

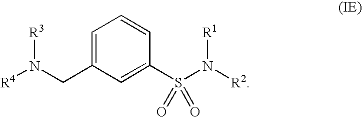

In another embodiment, the compounds of the present invention, or pharmaceutically acceptable salts, solvates, prodrugs, tautomers, or esters thereof, have the structure of formula (IE), wherein R¹ and R² are each independently selected from the group consisting of H, substituted or unsubstituted alkyl, substituted or unsubstituted arylalkyl, substituted or unsubstituted heteroarylalkyl, substituted or unsubstituted cycloalkyl, substituted or unsubstituted aryl, substituted or unsubstituted heteroaryl, and substituted or unsubstituted aminoalkyl; or R¹ and R², taken together with the nitrogen atom to which they are both shown attached, form a substituted or unsubstituted 5- to 18-membered saturated heterocyclic ring containing at least one nitrogen atom; and R³ and R⁴ are each independently selected from the group consisting of H, acyl, substituted or unsubstituted arylalkyl, substituted or unsubstituted heteroarylalkyl, substituted or unsubstituted heteroaryl, substituted or unsubstituted alkyl, amino, substituted or unsubstituted aminoalkyl, substituted or unsubstituted cycloalkyl; or R³ and R⁴, taken together with the nitrogen atom to which they are both shown attached, form a substituted or unsubstituted 5- to 18-membered saturated heterocyclic ring containing at least one nitrogen atom.

In another embodiment, the compounds of the present invention, or pharmaceutically acceptable salts, solvates, prodrugs, tautomers, or esters thereof, have the structure of formula (IF):

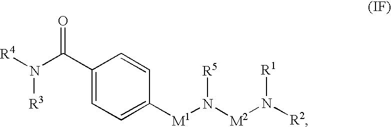

wherein L¹ is M¹-N(R⁵)-M²; M¹ is alkylene; M² is C(O); R¹ and R² are each independently selected from the group consisting of H, substituted or unsubstituted alkyl, substituted or unsubstituted arylalkyl, and substituted or unsubstituted heteroarylalkyl; R⁵ is selected from the group consisting of substituted or unsubstituted alkyl, substituted or unsubstituted aminoalkyl, substituted or unsubstituted cycloalkyl, substituted or unsubstituted heterocycloalkyl; and R³ and R⁴ are each independently selected from the group consisting of H, alkoxy, and substituted or unsubstituted amino.

In another embodiment, the compounds of the present invention, or pharmaceutically acceptable salts, solvates, prodrugs, tautomers, or esters thereof, have the structure of formula (IF), wherein M¹ is —CH₂—; R¹ and R² are each independently selected from the group consisting of H and substituted or unsubstituted naphthylalkyl; R⁵ is substituted or unsubstituted morpholinoalkyl; and R³ and R⁴ are each independently selected from the group consisting of H, methoxy, substituted or unsubstituted phenylamino, amino, and urethanyl.

In another embodiment, the compounds of the present invention, or pharmaceutically acceptable salts, solvates, prodrugs, tautomers, or esters thereof, have the structure of formula (IG):

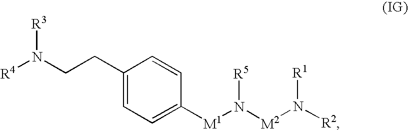

wherein L¹ is ML-N(R⁵)-M²; M¹ is alkylene; M² is C(O); R¹ and R² are each independently selected from the group consisting of H, substituted or unsubstituted alkyl, substituted or unsubstituted arylalkyl, and substituted or unsubstituted heteroarylalkyl; R⁵ is selected from the group consisting of substituted or unsubstituted alkyl, substituted or unsubstituted aminoalkyl, substituted or unsubstituted cycloalkyl, substituted or unsubstituted heterocycloalkyl; and R³ and R⁴ are each independently selected from the group consisting of H, alkoxycarbonyl, substituted or unsubstituted aryl-S(O)₂—, substituted or unsubstituted arylalkyl, substituted or unsubstituted heteroarylalkyl, substituted or unsubstituted aryl, and substituted or unsubstituted heteroaryl.

In another embodiment, the compounds of the present invention, or pharmaceutically acceptable salts, solvates, prodrugs, tautomers, or esters thereof, have the structure of formula (IG), wherein M¹ is CH₂; M² is C(O); R¹ and R² are each independently selected from the group consisting of H and substituted or unsubstituted naphthylalkyl, R⁵ is morpholinoalkyl; and R³ and R⁴ are each independently selected from the group consisting of H, butoxycarbonyl, substituted or unsubstituted phenyl-S(O)₂—, substituted or unsubstituted benzyl, substituted or unsubstituted imidazolylalkyl, and substituted or unsubstituted pyrimidyl.

In another embodiment, the compounds of the present invention, or pharmaceutically acceptable salts, solvates, prodrugs, tautomers, or esters thereof, have the structure of formula (I), wherein L¹ is selected from the group consisting of —(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; and R³ and R⁴ are not both hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, and —N(R⁵)—S(O)₂—; and R³ and R⁴ are not both hydrogen.

In another embodiment, the compounds of the present invention, or pharmaceutically acceptable salts, solvates, prodrugs, tautomers, or esters thereof, have the structure of formula (I), wherein L¹ is selected from the group consisting of -alkylene-N(R⁵)—C(O)—, -alkylene-N(R⁵)—S(O)—, and -alkylene-N(R⁵)—S(O)₂—; and R³ and R⁴ are not both hydrogen.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; and R³ and R⁴ are not both hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is alkylene, and R³ and R⁴ are not both hydrogen.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is alkylene; and R³ and R⁴ are not both hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is —CH₂—, and R³ and R⁴ are not both hydrogen.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is —CH₂—; and R³ and R⁴ are not both hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; R⁵ is hydrogen, substituted or unsubstituted arylalkyl, substituted or unsubstituted heteroarylalkyl, or substituted or unsubstituted aminoalkyl; and R³ and R⁴ are not both hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; wherein at least one of R¹, R²R³, and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; wherein at least two of R¹, R², R³, and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; wherein one of R¹ and R² is hydrogen, and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; at least one of R¹ and R² is substituted or unsubstituted arylalkyl, substituted or unsubstituted heteroarylalkyl, substituted or unsubstituted aryl, or substituted or unsubstituted heteroaryl; and R³ and R⁴ are not both hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; one of R¹ and R² is substituted or unsubstituted arylalkyl, substituted or unsubstituted heteroarylalkyl, substituted or unsubstituted aryl, or substituted or unsubstituted heteroaryl; and one of R¹ and R² is hydrogen; and R³ and R⁴ are not both hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; R¹ and R² are independently substituted or unsubstituted arylalkyl, substituted or unsubstituted heteroarylalkyl, substituted or unsubstituted aryl, or substituted or unsubstituted heteroaryl; and R³ and R⁴ are not both hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; R³ and R⁴ are independently alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl; and one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; R⁵ is hydrogen, substituted or unsubstituted arylalkyl, substituted or unsubstituted heteroarylalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; wherein at least one of R¹, R²R³, and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; wherein at least two of R¹, R², R³, and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; wherein one of R¹ and R² is hydrogen, and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; at least one of R¹ and R² is substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; one of R¹ and R² is substituted or unsubstituted arylalkyl, substituted or unsubstituted heteroarylalkyl, substituted or unsubstituted aryl, or substituted or unsubstituted heteroaryl; and one of R¹ and R² is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; R¹ and R² are independently substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; R³ and R⁴ are independently alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl; and one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is alkylene; R³ and R⁴ are not both hydrogen; R⁵ is hydrogen, substituted or unsubstituted arylalkyl, substituted or unsubstituted heteroarylalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is —CH₂—; R³ and R⁴ are not both hydrogen; R⁵ is hydrogen, substituted or unsubstituted arylalkyl, substituted or unsubstituted heteroarylalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is alkylene; at least one of R¹, R², R³, and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is —CH₂—; at least one of R¹, R², R³, and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is alkylene; at least two of R¹, R², R³, and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is —CH₂—; at least two of R¹, R², R³, and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is alkylene; one of R¹ and R² is hydrogen, and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is alkylene; at least one of R¹ and R² is substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is alkylene; one of R¹ and R² is substituted or unsubstituted arylalkyl, substituted or unsubstituted heteroarylalkyl, substituted or unsubstituted aryl, or substituted or unsubstituted heteroaryl; and one of R¹ and R² is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is alkylene; R¹ and R² are independently substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is alkylene; R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is alkylene; at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is alkylene; one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is alkylene; R³ and R⁴ are independently alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is alkylene; one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl; and one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is —CH₂—; one of R¹ and R² is hydrogen, and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is —CH₂—; at least one of R¹ and R² is substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is —CH₂—; one of R¹ and R² is substituted or unsubstituted arylalkyl, substituted or unsubstituted heteroarylalkyl, substituted or unsubstituted aryl, or substituted or unsubstituted heteroaryl; and one of R¹ and R² is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is —CH₂—; R¹ and R² are independently substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is —CH₂—; R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is —CH₂—; at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is —CH₂—; one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is —CH₂—; R³ and R⁴ are independently alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is —CH₂—; one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl; and one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; at least one of R¹ and R² is substituted or unsubstituted arylalkyl, substituted or unsubstituted heteroarylalkyl, substituted or unsubstituted aryl, or substituted or unsubstituted heteroaryl; and at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; at least one of R¹ and R² is substituted or unsubstituted arylalkyl, substituted or unsubstituted heteroarylalkyl, substituted or unsubstituted aryl, or substituted or unsubstituted heteroaryl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is alkylene; at least one of R¹ and R² is substituted or unsubstituted arylalkyl, substituted or unsubstituted heteroarylalkyl, substituted or unsubstituted aryl, or substituted or unsubstituted heteroaryl; and at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is alkylene; at least one of R¹ and R² is substituted or unsubstituted arylalkyl, substituted or unsubstituted heteroarylalkyl, substituted or unsubstituted aryl, or substituted or unsubstituted heteroaryl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is —CH₂—; at least one of R¹ and R² is substituted or unsubstituted arylalkyl, substituted or unsubstituted heteroarylalkyl, substituted or unsubstituted aryl, or substituted or unsubstituted heteroaryl; and at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is —CH₂—; at least one of R¹ and R² is substituted or unsubstituted arylalkyl, substituted or unsubstituted heteroarylalkyl, substituted or unsubstituted aryl, or substituted or unsubstituted heteroaryl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; R⁵ is hydrogen, substituted or unsubstituted arylalkyl, substituted or unsubstituted heteroarylalkyl, or substituted or unsubstituted aminoalkyl; at least one of R¹ and R² is substituted or unsubstituted arylalkyl, substituted or unsubstituted heteroarylalkyl, substituted or unsubstituted aryl, or substituted or unsubstituted heteroaryl; and at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; R⁵ is hydrogen, substituted or unsubstituted arylalkyl, substituted or unsubstituted heteroarylalkyl, or substituted or unsubstituted aminoalkyl; at least one of R¹ and R² is substituted or unsubstituted arylalkyl, substituted or unsubstituted heteroarylalkyl, substituted or unsubstituted aryl, or substituted or unsubstituted heteroaryl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is alkylene; R⁵ is hydrogen, substituted or unsubstituted arylalkyl, substituted or unsubstituted heteroarylalkyl, or substituted or unsubstituted aminoalkyl; at least one of R¹ and R² is substituted or unsubstituted arylalkyl, substituted or unsubstituted heteroarylalkyl, substituted or unsubstituted aryl, or substituted or unsubstituted heteroaryl; and at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is alkylene; R⁵ is hydrogen, substituted or unsubstituted arylalkyl, substituted or unsubstituted heteroarylalkyl, or substituted or unsubstituted aminoalkyl; at least one of R¹ and R² is substituted or unsubstituted arylalkyl, substituted or unsubstituted heteroarylalkyl, substituted or unsubstituted aryl, or substituted or unsubstituted heteroaryl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is —CH₂—; R⁵ is hydrogen, substituted or unsubstituted arylalkyl, substituted or unsubstituted heteroarylalkyl, or substituted or unsubstituted aminoalkyl; at least one of R¹ and R² is substituted or unsubstituted arylalkyl, substituted or unsubstituted heteroarylalkyl, substituted or unsubstituted aryl, or substituted or unsubstituted heteroaryl; and at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —N(R⁵)—C(O)—, —N(R⁵)—S(O)—, —N(R⁵)—S(O)₂—, -alkylene-N(R⁵)—C(O)—, -alkylene-N(R⁵)—S(O)—, and -alkylene-N(R⁵)—S(O)₂—; L² is —CH₂—; R⁵ is hydrogen, substituted or unsubstituted arylalkyl, substituted or unsubstituted heteroarylalkyl, or substituted or unsubstituted aminoalkyl; at least one of R¹ and R² is substituted or unsubstituted arylalkyl, substituted or unsubstituted heteroarylalkyl, substituted or unsubstituted aryl, or substituted or unsubstituted heteroaryl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is alkylene; one of R¹ and R² is hydrogen, and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is alkylene; at least one of R¹ and R² is substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is alkylene; one of R¹ and R² is substituted or unsubstituted arylalkyl, substituted or unsubstituted heteroarylalkyl, substituted or unsubstituted aryl, or substituted or unsubstituted heteroaryl; and one of R¹ and R² is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is alkylene; R¹ and R² are independently substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is alkylene; R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is alkylene; at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is alkylene; one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is alkylene; R³ and R⁴ are independently alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is alkylene; one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl; and one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is —CH₂—; one of R¹ and R² is hydrogen, and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is —CH₂—; at least one of R¹ and R² is substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is —CH₂—; one of R¹ and R² is substituted or unsubstituted arylalkyl, substituted or unsubstituted heteroarylalkyl, substituted or unsubstituted aryl, or substituted or unsubstituted heteroaryl; and one of R¹ and R² is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is —CH₂—; R¹ and R² are independently substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is —CH₂—; R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is —CH₂—; at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is —CH₂—; one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is —CH₂—; R³ and R⁴ are independently alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is —CH₂—; one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl; and one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; at least one of R¹ and R² is substituted or unsubstituted arylalkyl, substituted or unsubstituted heteroarylalkyl, substituted or unsubstituted aryl, or substituted or unsubstituted heteroaryl; and at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; at least one of R¹ and R² is substituted or unsubstituted arylalkyl, substituted or unsubstituted heteroarylalkyl, substituted or unsubstituted aryl, or substituted or unsubstituted heteroaryl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is alkylene; at least one of R¹ and R² is substituted or unsubstituted arylalkyl, substituted or unsubstituted heteroarylalkyl, substituted or unsubstituted aryl, or substituted or unsubstituted heteroaryl; and at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is alkylene; at least one of R¹ and R² is substituted or unsubstituted arylalkyl, substituted or unsubstituted heteroarylalkyl, substituted or unsubstituted aryl, or substituted or unsubstituted heteroaryl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is —CH₂—; at least one of R¹ and R² is substituted or unsubstituted arylalkyl, substituted or unsubstituted heteroarylalkyl, substituted or unsubstituted aryl, or substituted or unsubstituted heteroaryl; and at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is —CH₂—; at least one of R¹ and R² is substituted or unsubstituted arylalkyl, substituted or unsubstituted heteroarylalkyl, substituted or unsubstituted aryl, or substituted or unsubstituted heteroaryl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; R⁵ is hydrogen, substituted or unsubstituted arylalkyl, substituted or unsubstituted heteroarylalkyl, or substituted or unsubstituted aminoalkyl; at least one of R¹ and R² is substituted or unsubstituted arylalkyl, substituted or unsubstituted heteroarylalkyl, substituted or unsubstituted aryl, or substituted or unsubstituted heteroaryl; and at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; R⁵ is hydrogen, substituted or unsubstituted arylalkyl, substituted or unsubstituted heteroarylalkyl, or substituted or unsubstituted aminoalkyl; at least one of R¹ and R² is substituted or unsubstituted arylalkyl, substituted or unsubstituted heteroarylalkyl, substituted or unsubstituted aryl, or substituted or unsubstituted heteroaryl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is alkylene; R⁵ is hydrogen, substituted or unsubstituted arylalkyl, substituted or unsubstituted heteroarylalkyl, or substituted or unsubstituted aminoalkyl; at least one of R¹ and R² is substituted or unsubstituted arylalkyl, substituted or unsubstituted heteroarylalkyl, substituted or unsubstituted aryl, or substituted or unsubstituted heteroaryl; and at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is alkylene; R⁵ is hydrogen, substituted or unsubstituted arylalkyl, substituted or unsubstituted heteroarylalkyl, or substituted or unsubstituted aminoalkyl; at least one of R¹ and R² is substituted or unsubstituted arylalkyl, substituted or unsubstituted heteroarylalkyl, substituted or unsubstituted aryl, or substituted or unsubstituted heteroaryl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is —CH₂—; R⁵ is hydrogen, substituted or unsubstituted arylalkyl, substituted or unsubstituted heteroarylalkyl, or substituted or unsubstituted aminoalkyl; at least one of R¹ and R² is substituted or unsubstituted arylalkyl, substituted or unsubstituted heteroarylalkyl, substituted or unsubstituted aryl, or substituted or unsubstituted heteroaryl; and at least one of R³ and R⁴ is alkoxyalkyl, substituted or unsubstituted aminoalkyl, substituted or unsubstituted aminoacyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is selected from the group consisting of —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, and —CH₂CH₂—N(R⁵)—S(O)₂—; L² is —CH₂—; R⁵ is hydrogen, substituted or unsubstituted arylalkyl, substituted or unsubstituted heteroarylalkyl, or substituted or unsubstituted aminoalkyl; at least one of R¹ and R² is substituted or unsubstituted arylalkyl, substituted or unsubstituted heteroarylalkyl, substituted or unsubstituted aryl, or substituted or unsubstituted heteroaryl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —C(O)—.

In another embodiment, the compounds of the present invention, or pharmaceutically acceptable salts, solvates, prodrugs, tautomers, or esters thereof, have the structure of formula (I), wherein L¹ is —C(O)—; L² is a covalent bond or alkylene.

In another embodiment, the compounds of the present invention, or pharmaceutically acceptable salts, solvates, prodrugs, tautomers, or esters thereof, have the structure of formula (I), wherein L¹ is —C(O)—; L² is a covalent bond or —CH₂— or —CH₂CH₂—.

In another embodiment, the compounds of the present invention, or pharmaceutically acceptable salts, solvates, prodrugs, tautomers, or esters thereof, have the structure of formula (I), wherein L¹ is —C(O)—; L² is a covalent bond; and one of R³ and R⁴ is substituted or unsubstituted arylalkyl, substituted or unsubstituted heteroarylalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —C(O)—; L² is a covalent bond; one of R³ and R⁴ is substituted or unsubstituted arylalkyl, substituted or unsubstituted heteroarylalkyl, or substituted or unsubstituted aminoalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is a covalent bond; and at least one of R¹ and R² is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is a covalent bond; one of R¹ and R² is substituted or unsubstituted arylalkyl, or substituted or unsubstituted heteroarylalkyl; and one of R¹ and R² is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is a covalent bond; and R¹ and R² are independently substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is a covalent bond; and one of R³ and R⁴ is substituted or unsubstituted arylalkyl, substituted or unsubstituted heteroarylalkyl, or substituted or unsubstituted aminoalkyl; and at least one of R¹ and R² is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is a covalent bond; and one of R³ and R⁴ is substituted or unsubstituted arylalkyl, substituted or unsubstituted heteroarylalkyl, or substituted or unsubstituted aminoalkyl; one of R³ and R⁴ is hydrogen; and at least one of R¹ and R² is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is a covalent bond; and one of R³ and R⁴ is substituted or unsubstituted arylalkyl, substituted or unsubstituted heteroarylalkyl, or substituted or unsubstituted aminoalkyl; one of R¹ and R² is substituted or unsubstituted arylalkyl, or substituted or unsubstituted heteroarylalkyl; and one of R¹ and R² is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is a covalent bond; and one of R³ and R⁴ is substituted or unsubstituted arylalkyl, substituted or unsubstituted heteroarylalkyl, or substituted or unsubstituted aminoalkyl; one of R³ and R⁴ is hydrogen; one of R¹ and R² is substituted or unsubstituted arylalkyl, or substituted or unsubstituted heteroarylalkyl; and one of R¹ and R² is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is a covalent bond; and one of R³ and R⁴ is substituted or unsubstituted arylalkyl, substituted or unsubstituted heteroarylalkyl, or substituted or unsubstituted aminoalkyl; R¹ and R² are independently substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is a covalent bond; and one of R³ and R⁴ is substituted or unsubstituted arylalkyl, substituted or unsubstituted heteroarylalkyl, or substituted or unsubstituted aminoalkyl; one of R³ and R⁴ is hydrogen; R¹ and R² are independently substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is alkylene.

In another embodiment, the compounds of the present invention, or pharmaceutically acceptable salts, solvates, prodrugs, tautomers, or esters thereof, have the structure of formula (I), wherein L¹ is —C(O)—; L² is alkylene; and at least one of R¹, R², R³, and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is alkylene; and one of R¹ and R² is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is alkylene; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is alkylene; and at least one of R¹ and R² is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is alkylene; one of R¹ and R² is substituted or unsubstituted arylalkyl, or substituted or unsubstituted heteroarylalkyl; and one of R¹ and R² is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is alkylene; and R¹ and R² are independently substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is alkylene; and at least one of R³ and R⁴ is substituted or unsubstituted aryl, substituted or unsubstituted heteroaryl, substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is alkylene; one of R³ and R⁴ is substituted or unsubstituted aryl, substituted or unsubstituted heteroaryl, substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is alkylene; and R³ and R⁴ are independently substituted or unsubstituted aryl, substituted or unsubstituted heteroaryl, substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is alkylene; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —C(O)—; L² is alkylene; and at least one of R³ and R⁴ is alkoxyalkyl or substituted or unsubstituted aminoalkyl, substituted or unsubstituted aminoacylalkyl, or substituted or unsubstituted aminoacylalkyl.

In another embodiment, the compounds of the present invention, or pharmaceutically acceptable salts, solvates, prodrugs, tautomers, or esters thereof, have the structure of formula (I), wherein L¹ is —C(O)—; L² is alkylene; one of R³ and R⁴ is alkoxyalkyl or substituted or unsubstituted aminoalkyl, substituted or unsubstituted aminoacylalkyl, or substituted or unsubstituted aminoacylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is alkylene; and R³ and R⁴ are independently alkoxyalkyl or substituted or unsubstituted aminoalkyl, substituted or unsubstituted aminoacylalkyl, or substituted or unsubstituted aminoacylalkyl.

In another embodiment, the compounds of the present invention, or pharmaceutically acceptable salts, solvates, prodrugs, tautomers, or esters thereof, have the structure of formula (I), wherein L¹ is —C(O)—; L² is alkylene; at least one of R¹ and R² is substituted or unsubstituted arylalkyl, or substituted or unsubstituted heteroarylalkyl; and at least one of R³ and R⁴ is substituted or unsubstituted aryl, substituted or unsubstituted heteroaryl, substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is alkylene; at least one of R¹ and R² is substituted or unsubstituted arylalkyl, or substituted or unsubstituted heteroarylalkyl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —C(O)—; L² is alkylene; at least one of R¹ and R² is substituted or unsubstituted arylalkyl, or substituted or unsubstituted heteroarylalkyl; and at least one of R³ and R⁴ is alkoxyalkyl or substituted or unsubstituted aminoalkyl, substituted or unsubstituted aminoacylalkyl, or substituted or unsubstituted aminoacylalkyl.

In another embodiment, the compounds of the present invention, or pharmaceutically acceptable salts, solvates, prodrugs, tautomers, or esters thereof, have the structure of formula (I), wherein L¹ is —C(O)—; L² is alkylene; one of R¹ and R² is substituted or unsubstituted arylalkyl, or substituted or unsubstituted heteroarylalkyl; one of R¹ and R² is hydrogen; and at least one of R³ and R⁴ is substituted or unsubstituted aryl, substituted or unsubstituted heteroaryl, substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is alkylene; R¹ and R² are independently substituted or unsubstituted arylalkyl, or substituted or unsubstituted heteroarylalkyl; and at least one of R³ and R⁴ is substituted or unsubstituted aryl, substituted or unsubstituted heteroaryl, substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is alkylene; one of R¹ and R² is substituted or unsubstituted arylalkyl, or substituted or unsubstituted heteroarylalkyl; one of R¹ and R² is hydrogen; one of R³ and R⁴ is substituted or unsubstituted aryl, substituted or unsubstituted heteroaryl, substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is alkylene; R¹ and R² are independently substituted or unsubstituted arylalkyl, or substituted or unsubstituted heteroarylalkyl; and R³ and R⁴ are independently substituted or unsubstituted aryl, substituted or unsubstituted heteroaryl, substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is alkylene; one of R¹ and R² is substituted or unsubstituted arylalkyl, or substituted or unsubstituted heteroarylalkyl; one of R¹ and R² is hydrogen; and R³ and R⁴ are independently substituted or unsubstituted aryl, substituted or unsubstituted heteroaryl, substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is alkylene; R¹ and R² are independently substituted or unsubstituted arylalkyl, or substituted or unsubstituted heteroarylalkyl; and one of R³ and R⁴ is substituted or unsubstituted aryl, substituted or unsubstituted heteroaryl, substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is alkylene; one of R¹ and R² is substituted or unsubstituted arylalkyl, or substituted or unsubstituted heteroarylalkyl; one of R¹ and R² is hydrogen; and at least one of R³ and R⁴ is alkoxyalkyl or substituted or unsubstituted aminoalkyl, substituted or unsubstituted aminoacylalkyl, or substituted or unsubstituted aminoacylalkyl.

In another embodiment, the compounds of the present invention, or pharmaceutically acceptable salts, solvates, prodrugs, tautomers, or esters thereof, have the structure of formula (I), wherein L¹ is —C(O)—; L² is alkylene; R¹ and R² are independently substituted or unsubstituted arylalkyl, or substituted or unsubstituted heteroarylalkyl; and at least one of R³ and R⁴ is alkoxyalkyl or substituted or unsubstituted aminoalkyl, substituted or unsubstituted aminoacylalkyl, or substituted or unsubstituted aminoacylalkyl.

In another embodiment, the compounds of the present invention, or pharmaceutically acceptable salts, solvates, prodrugs, tautomers, or esters thereof, have the structure of formula (I), wherein L¹ is —C(O)—; L² is alkylene; one of R¹ and R² is substituted or unsubstituted arylalkyl, or substituted or unsubstituted heteroarylalkyl; one of R¹ and R² is hydrogen; one of R³ and R⁴ is alkoxyalkyl or substituted or unsubstituted aminoalkyl, substituted or unsubstituted aminoacylalkyl, or substituted or unsubstituted aminoacylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is alkylene; R¹ and R² are independently substituted or unsubstituted arylalkyl, or substituted or unsubstituted heteroarylalkyl; and R³ and R⁴ are independently alkoxyalkyl or substituted or unsubstituted aminoalkyl, substituted or unsubstituted aminoacylalkyl, or substituted or unsubstituted aminoacylalkyl.

In another embodiment, the compounds of the present invention, or pharmaceutically acceptable salts, solvates, prodrugs, tautomers, or esters thereof, have the structure of formula (I), wherein L¹ is —C(O)—; L² is alkylene; at least one of R¹ and R² is substituted or unsubstituted arylalkyl, or substituted or unsubstituted heteroarylalkyl; one of R³ and R⁴ is alkoxyalkyl or substituted or unsubstituted aminoalkyl, substituted or unsubstituted aminoacylalkyl, or substituted or unsubstituted aminoacylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is alkylene; at least one of R¹ and R² is substituted or unsubstituted arylalkyl, or substituted or unsubstituted heteroarylalkyl; and R³ and R⁴ are independently alkoxyalkyl or substituted or unsubstituted aminoalkyl, substituted or unsubstituted aminoacylalkyl, or substituted or unsubstituted aminoacylalkyl.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; and at least one of R¹, R², R³, and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; and one of R¹ and R² is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; and at least one of R¹ and R² is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; one of R¹ and R² is substituted or unsubstituted arylalkyl, or substituted or unsubstituted heteroarylalkyl; and one of R¹ and R² is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; and R¹ and R² are independently substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; and at least one of R³ and R⁴ is substituted or unsubstituted aryl, substituted or unsubstituted heteroaryl, substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; one of R³ and R⁴ is substituted or unsubstituted aryl, substituted or unsubstituted heteroaryl, substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; and R² and R⁴ are independently substituted or unsubstituted aryl, substituted or unsubstituted heteroaryl, substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; and at least one of R³ and R⁴ is alkoxyalkyl or substituted or unsubstituted aminoalkyl, substituted or unsubstituted aminoacylalkyl, or substituted or unsubstituted aminoacylalkyl.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; one of R³ and R⁴ is alkoxyalkyl or substituted or unsubstituted aminoalkyl, substituted or unsubstituted aminoacylalkyl, or substituted or unsubstituted aminoacylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; and R³ and R⁴ are independently alkoxyalkyl or substituted or unsubstituted aminoalkyl, substituted or unsubstituted aminoacylalkyl, or substituted or unsubstituted aminoacylalkyl.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; at least one of R¹ and R² is substituted or unsubstituted arylalkyl, or substituted or unsubstituted heteroarylalkyl; and at least one of R³ and R⁴ is substituted or unsubstituted aryl, substituted or unsubstituted heteroaryl, substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; at least one of R¹ and R² is substituted or unsubstituted arylalkyl, or substituted or unsubstituted heteroarylalkyl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; at least one of R¹ and R² is substituted or unsubstituted arylalkyl, or substituted or unsubstituted heteroarylalkyl; and at least one of R³ and R⁴ is alkoxyalkyl or substituted or unsubstituted aminoalkyl, substituted or unsubstituted aminoacylalkyl, or substituted or unsubstituted aminoacylalkyl.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; one of R¹ and R² is substituted or unsubstituted arylalkyl, or substituted or unsubstituted heteroarylalkyl; one of R¹ and R² is hydrogen; and at least one of R³ and R⁴ is substituted or unsubstituted aryl, substituted or unsubstituted heteroaryl, substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; R¹ and R² are independently substituted or unsubstituted arylalkyl, or substituted or unsubstituted heteroarylalkyl; and at least one of R³ and R⁴ is substituted or unsubstituted aryl, substituted or unsubstituted heteroaryl, substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; one of R¹ and R² is substituted or unsubstituted arylalkyl, or substituted or unsubstituted heteroarylalkyl; one of R¹ and R² is hydrogen; one of R³ and R⁴ is substituted or unsubstituted aryl, substituted or unsubstituted heteroaryl, substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; R¹ and R² are independently substituted or unsubstituted arylalkyl, or substituted or unsubstituted heteroarylalkyl; and R³ and R⁴ are independently substituted or unsubstituted aryl, substituted or unsubstituted heteroaryl, substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; one of R¹ and R² is substituted or unsubstituted arylalkyl, or substituted or unsubstituted heteroarylalkyl; one of R¹ and R² is hydrogen; and R³ and R⁴ are independently substituted or unsubstituted aryl, substituted or unsubstituted heteroaryl, substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; R¹ and R² are independently substituted or unsubstituted arylalkyl, or substituted or unsubstituted heteroarylalkyl; and one of R³ and R⁴ is substituted or unsubstituted aryl, substituted or unsubstituted heteroaryl, substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; one of R¹ and R² is substituted or unsubstituted arylalkyl, or substituted or unsubstituted heteroarylalkyl; one of R¹ and R² is hydrogen; and at least one of R³ and R⁴ is alkoxyalkyl or substituted or unsubstituted aminoalkyl, substituted or unsubstituted aminoacylalkyl, or substituted or unsubstituted aminoacylalkyl.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; R¹ and R² are independently substituted or unsubstituted arylalkyl, or substituted or unsubstituted heteroarylalkyl; and at least one of R³ and R⁴ is alkoxyalkyl or substituted or unsubstituted aminoalkyl, substituted or unsubstituted aminoacylalkyl, or substituted or unsubstituted aminoacylalkyl.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; one of R¹ and R² is substituted or unsubstituted arylalkyl, or substituted or unsubstituted heteroarylalkyl; one of R¹ and R² is hydrogen; one of R³ and R⁴ is alkoxyalkyl or substituted or unsubstituted aminoalkyl, substituted or unsubstituted aminoacylalkyl, or substituted or unsubstituted aminoacylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; R¹ and R² are independently substituted or unsubstituted arylalkyl, or substituted or unsubstituted heteroarylalkyl; and R³ and R⁴ are independently alkoxyalkyl or substituted or unsubstituted aminoalkyl, substituted or unsubstituted aminoacylalkyl, or substituted or unsubstituted aminoacylalkyl.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; at least one of R¹ and R² is substituted or unsubstituted arylalkyl, or substituted or unsubstituted heteroarylalkyl; one of R³ and R⁴ is alkoxyalkyl or substituted or unsubstituted aminoalkyl, substituted or unsubstituted aminoacylalkyl, or substituted or unsubstituted aminoacylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is —CH₂— or —CH₂CH₂—; at least one of R¹ and R² is substituted or unsubstituted arylalkyl, or substituted or unsubstituted heteroarylalkyl; and R³ and R⁴ are independently alkoxyalkyl or substituted or unsubstituted aminoalkyl, substituted or unsubstituted aminoacylalkyl, or substituted or unsubstituted aminoacylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; and L² is —C(O)—, —S(O)—, —S(O)₂—, or alkylene.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; and L² is methylene, i.e., —CH₂—.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is —C(O)—, —S(O)—, or —S(O)₂—; and at least one of R¹ and R² is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is —C(O)—, —S(O)—, or —S(O)₂—; one of R¹ and R² is substituted or unsubstituted arylalkyl, or substituted or unsubstituted heteroarylalkyl; and one of R¹ and R² is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is —C(O)—, —S(O)—, or —S(O)₂—; and R¹ and R² are independently substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is —C(O)—, —S(O)—, or —S(O)₂—; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is —C(O)—, —S(O)—, or —S(O)₂—; one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is —C(O)—, —S(O)—, or —S(O)₂—; and R³ and R⁴ are independently substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is —C(O)—, —S(O)—, or —S(O)₂—; at least one of R¹ and R² is substituted or unsubstituted arylalkyl, or substituted or unsubstituted heteroarylalkyl; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is —C(O)—, —S(O)—, or —S(O)₂—; one of R¹ and R² is substituted or unsubstituted arylalkyl, or substituted or unsubstituted heteroarylalkyl; one of R¹ and R² is hydrogen; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is —C(O)—, —S(O)—, or —S(O)₂—; R¹ and R² are independently substituted or unsubstituted arylalkyl, or substituted or unsubstituted heteroarylalkyl; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is —C(O)—, —S(O)—, or —S(O)₂—; at least one of R¹ and R² is substituted or unsubstituted arylalkyl, or substituted or unsubstituted heteroarylalkyl; one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is —C(O)—, —S(O)—, or —S(O)₂—; at least one of R¹ and R² is substituted or unsubstituted arylalkyl, or substituted or unsubstituted heteroarylalkyl; and R³ and R⁴ are independently substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is —C(O)—, —S(O)—, or —S(O)₂—; one of R¹ and R² is substituted or unsubstituted arylalkyl, or substituted or unsubstituted heteroarylalkyl; and one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; R³ and R⁴ are both hydrogen, and R¹ and R² are independently substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; R³ and R⁴ are both hydrogen, and R¹ and R² are independently substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted arylalkyl, or substituted or unsubstituted heteroarylalkyl; and one of R¹ and R² is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted aminoalkyl; and one of R¹ and R² is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R², taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R³ and R⁴ is substituted or unsubstituted arylamino, or substituted or unsubstituted heteroarylamino.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R³ and R⁴ is substituted or unsubstituted arylamino, or substituted or unsubstituted heteroarylamino; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R³ and R⁴ is substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; one of R³ and R⁴ is substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R³ and R⁴ is substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; one of R³ and R⁴ is substituted or unsubstituted alkyl, alkoxyalkyl, or substituted or unsubstituted aminoalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R³ and R⁴ are independently substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R³ and R⁴ is substituted or unsubstituted aminoacylalkyl, substituted or unsubstituted arylacyl, or substituted or unsubstituted heteroarylacyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; one of R³ and R⁴ is substituted or unsubstituted aminoacylalkyl, substituted or unsubstituted arylacyl, or substituted or unsubstituted heteroarylacyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted arylalkyl, or substituted or unsubstituted heteroarylalkyl; and at least one of R³ and R⁴ is substituted or unsubstituted arylamino, or substituted or unsubstituted heteroarylamino.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted arylalkyl, or substituted or unsubstituted heteroarylalkyl; and one of R¹ and R² is hydrogen; and at least one of R³ and R⁴ is substituted or unsubstituted arylamino, or substituted or unsubstituted heteroarylamino.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and at least one of R³ and R⁴ is substituted or unsubstituted arylamino, or substituted or unsubstituted heteroarylamino.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted aminoalkyl; and at least one of R³ and R⁴ is substituted or unsubstituted arylamino, or substituted or unsubstituted heteroarylamino.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted aminoalkyl; and one of R¹ and R² is hydrogen; and at least one of R³ and R⁴ is substituted or unsubstituted arylamino, or substituted or unsubstituted heteroarylamino.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and at least one of R³ and R⁴ is substituted or unsubstituted arylamino, or substituted or unsubstituted heteroarylamino.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R², taken together with the nitrogen atom to which they are both attached, form a 5- to 18-membered saturated heterocyclic ring containing at least two nitrogen atoms; and at least one of R³ and R⁴ is substituted or unsubstituted arylamino, or substituted or unsubstituted heteroarylamino.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted arylalkyl, or substituted or unsubstituted heteroarylalkyl; and one of R³ and R⁴ is substituted or unsubstituted arylamino, or substituted or unsubstituted heteroarylamino; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted arylalkyl, or substituted or unsubstituted heteroarylalkyl; and one of R¹ and R² is hydrogen; and one of R³ and R⁴ is substituted or unsubstituted arylamino, or substituted or unsubstituted heteroarylamino; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and one of R³ and R⁴ is substituted or unsubstituted arylamino, or substituted or unsubstituted heteroarylamino; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted aminoalkyl; and one of R³ and R⁴ is substituted or unsubstituted arylamino, or substituted or unsubstituted heteroarylamino; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted aminoalkyl; and one of R¹ and R² is hydrogen; and one of R³ and R⁴ is substituted or unsubstituted arylamino, or substituted or unsubstituted heteroarylamino; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and one of R³ and R⁴ is substituted or unsubstituted arylamino, or substituted or unsubstituted heteroarylamino; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R², taken together with the nitrogen atom to which they are both attached, form a 5- to 18-membered saturated heterocyclic ring containing at least two nitrogen atoms; and one of R³ and R⁴ is substituted or unsubstituted arylamino, or substituted or unsubstituted heteroarylamino; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted arylalkyl, or substituted or unsubstituted heteroarylalkyl; and at least one of R³ and R⁴ is substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted arylalkyl, or substituted or unsubstituted heteroarylalkyl; and one of R¹ and R² is hydrogen; and at least one of R³ and R⁴ is substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and at least one of R³ and R⁴ is substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted aminoalkyl; and at least one of R³ and R⁴ is substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted aminoalkyl; and one of R¹ and R² is hydrogen; and at least one of R³ and R⁴ is substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and at least one of R³ and R⁴ is substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R², taken together with the nitrogen atom to which they are both attached, form a 5- to 18-membered saturated heterocyclic ring containing at least two nitrogen atoms; and at least one of R³ and R⁴ is substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted arylalkyl, or substituted or unsubstituted heteroarylalkyl; one of R³ and R⁴ is substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted arylalkyl, or substituted or unsubstituted heteroarylalkyl; and one of R¹ and R² is hydrogen; one of R³ and R⁴ is substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; one of R³ and R⁴ is substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted aminoalkyl; one of R³ and R⁴ is substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted aminoalkyl; and one of R¹ and R² is hydrogen; one of R³ and R⁴ is substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; one of R³ and R⁴ is substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R², taken together with the nitrogen atom to which they are both attached, form a 5- to 18-membered saturated heterocyclic ring containing at least two nitrogen atoms; one of R³ and R⁴ is substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted arylalkyl, or substituted or unsubstituted heteroarylalkyl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted arylalkyl, or substituted or unsubstituted heteroarylalkyl; and one of R¹ and R² is hydrogen; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted aminoalkyl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted aminoalkyl; and one of R¹ and R² is hydrogen; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R², taken together with the nitrogen atom to which they are both attached, form a 5- to 18-membered saturated heterocyclic ring containing at least two nitrogen atoms; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted arylalkyl, or substituted or unsubstituted heteroarylalkyl; and at least one of R³ and R⁴ is substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted arylalkyl, or substituted or unsubstituted heteroarylalkyl; and one of R¹ and R² is hydrogen; and at least one of R³ and R⁴ is substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and at least one of R³ and R⁴ is substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted aminoalkyl; and at least one of R³ and R⁴ is substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted aminoalkyl; and one of R¹ and R² is hydrogen; and at least one of R³ and R⁴ is substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and at least one of R³ and R⁴ is substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R², taken together with the nitrogen atom to which they are both attached, form a 5- to 18-membered saturated heterocyclic ring containing at least two nitrogen atoms; and at least one of R³ and R⁴ is substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted arylalkyl, or substituted or unsubstituted heteroarylalkyl; one of R³ and R⁴ is substituted or unsubstituted alkyl, alkoxyalkyl, or substituted or unsubstituted aminoalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted arylalkyl, or substituted or unsubstituted heteroarylalkyl; and one of R¹ and R² is hydrogen; one of R³ and R⁴ is substituted or unsubstituted alkyl, alkoxyalkyl, or substituted or unsubstituted aminoalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; one of R³ and R⁴ is substituted or unsubstituted alkyl, alkoxyalkyl, or substituted or unsubstituted aminoalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted aminoalkyl; one of R³ and R⁴ is substituted or unsubstituted alkyl, alkoxyalkyl, or substituted or unsubstituted aminoalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted aminoalkyl; and one of R¹ and R² is hydrogen; one of R³ and R⁴ is substituted or unsubstituted alkyl, alkoxyalkyl, or substituted or unsubstituted aminoalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; one of R³ and R⁴ is substituted or unsubstituted alkyl, alkoxyalkyl, or substituted or unsubstituted aminoalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R², taken together with the nitrogen atom to which they are both attached, form a 5- to 18-membered saturated heterocyclic ring containing at least two nitrogen atoms; one of R³ and R⁴ is substituted or unsubstituted alkyl, alkoxyalkyl, or substituted or unsubstituted aminoalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted arylalkyl, or substituted or unsubstituted heteroarylalkyl; and R³ and R⁴ are independently substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted arylalkyl, or substituted or unsubstituted heteroarylalkyl; and one of R¹ and R² is hydrogen; and R³ and R⁴ are independently substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and R³ and R⁴ are independently substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted aminoalkyl; and R³ and R⁴ are independently substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted aminoalkyl; and one of R¹ and R² is hydrogen; and R³ and R⁴ are independently substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and R³ and R⁴ are independently substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R², taken together with the nitrogen atom to which they are both attached, form a 5- to 18-membered saturated heterocyclic ring containing at least two nitrogen atoms; and R³ and R⁴ are independently substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted arylalkyl, or substituted or unsubstituted heteroarylalkyl; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted arylalkyl, or substituted or unsubstituted heteroarylalkyl; and one of R¹ and R² is hydrogen; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted aminoalkyl; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted aminoalkyl; and one of R¹ and R² is hydrogen; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R², taken together with the nitrogen atom to which they are both attached, form a 5- to 18-membered saturated heterocyclic ring containing at least two nitrogen atoms; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted arylalkyl, or substituted or unsubstituted heteroarylalkyl; and one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted arylalkyl, or substituted or unsubstituted heteroarylalkyl; and one of R¹ and R² is hydrogen; and one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted aminoalkyl; and one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted aminoalkyl; and one of R¹ and R² is hydrogen; and one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R², taken together with the nitrogen atom to which they are both attached, form a 5- to 18-membered saturated heterocyclic ring containing at least two nitrogen atoms; and one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted arylalkyl, or substituted or unsubstituted heteroarylalkyl; and at least one of R³ and R⁴ is substituted or unsubstituted aminoacylalkyl, substituted or unsubstituted arylacyl, or substituted or unsubstituted heteroarylacyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted arylalkyl, or substituted or unsubstituted heteroarylalkyl; and one of R¹ and R² is hydrogen; and at least one of R³ and R⁴ is substituted or unsubstituted aminoacylalkyl, substituted or unsubstituted arylacyl, or substituted or unsubstituted heteroarylacyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and at least one of R³ and R⁴ is substituted or unsubstituted aminoacylalkyl, substituted or unsubstituted arylacyl, or substituted or unsubstituted heteroarylacyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted aminoalkyl; and at least one of R³ and R⁴ is substituted or unsubstituted aminoacylalkyl, substituted or unsubstituted arylacyl, or substituted or unsubstituted heteroarylacyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted aminoalkyl; and one of R¹ and R² is hydrogen; and at least one of R³ and R⁴ is substituted or unsubstituted aminoacylalkyl, substituted or unsubstituted arylacyl, or substituted or unsubstituted heteroarylacyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; and at least one of R³ and R⁴ is substituted or unsubstituted aminoacylalkyl, substituted or unsubstituted arylacyl, or substituted or unsubstituted heteroarylacyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R², taken together with the nitrogen atom to which they are both attached, form a 5- to 18-membered saturated heterocyclic ring containing at least two nitrogen atoms; and at least one of R³ and R⁴ is substituted or unsubstituted aminoacylalkyl, substituted or unsubstituted arylacyl, or substituted or unsubstituted heteroarylacyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted arylalkyl, or substituted or unsubstituted heteroarylalkyl; one of R³ and R⁴ is substituted or unsubstituted aminoacylalkyl, substituted or unsubstituted arylacyl, or substituted or unsubstituted heteroarylacyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted arylalkyl, or substituted or unsubstituted heteroarylalkyl; and one of R¹ and R² is hydrogen; one of R³ and R⁴ is substituted or unsubstituted aminoacylalkyl, substituted or unsubstituted arylacyl, or substituted or unsubstituted heteroarylacyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; one of R³ and R⁴ is substituted or unsubstituted aminoacylalkyl, substituted or unsubstituted arylacyl, or substituted or unsubstituted heteroarylacyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted aminoalkyl; one of R³ and R⁴ is substituted or unsubstituted aminoacylalkyl, substituted or unsubstituted arylacyl, or substituted or unsubstituted heteroarylacyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and one of R¹ and R² is substituted or unsubstituted aminoalkyl; and one of R¹ and R² is hydrogen; one of R³ and R⁴ is substituted or unsubstituted aminoacylalkyl, substituted or unsubstituted arylacyl, or substituted or unsubstituted heteroarylacyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl; one of R³ and R⁴ is substituted or unsubstituted aminoacylalkyl, substituted or unsubstituted arylacyl, or substituted or unsubstituted heteroarylacyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R², taken together with the nitrogen atom to which they are both attached, form a 5- to 18-membered saturated heterocyclic ring containing at least two nitrogen atoms; one of R³ and R⁴ is substituted or unsubstituted aminoacylalkyl, substituted or unsubstituted arylacyl, or substituted or unsubstituted heteroarylacyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted arylalkyl, or substituted or unsubstituted heteroarylalkyl; and one of R¹ and R² is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted aminoalkyl; and one of R¹ and R² is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R², taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R³ and R⁴ is substituted or unsubstituted arylamino, or substituted or unsubstituted heteroarylamino.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R³ and R⁴ is substituted or unsubstituted arylamino, or substituted or unsubstituted heteroarylamino; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R³ and R⁴ is substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; one of R³ and R⁴ is substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R³ and R⁴ is substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; one of R³ and R⁴ is substituted or unsubstituted alkyl, alkoxyalkyl, or substituted or unsubstituted aminoalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R³ and R⁴ are independently substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R³ and R⁴ is substituted or unsubstituted aminoacylalkyl, substituted or unsubstituted arylacyl, or substituted or unsubstituted heteroarylacyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; one of R³ and R⁴ is substituted or unsubstituted aminoacylalkyl, substituted or unsubstituted arylacyl, or substituted or unsubstituted heteroarylacyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted arylalkyl, or substituted or unsubstituted heteroarylalkyl; and at least one of R³ and R⁴ is substituted or unsubstituted arylamino, or substituted or unsubstituted heteroarylamino.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted arylalkyl, or substituted or unsubstituted heteroarylalkyl; and one of R¹ and R² is hydrogen; and at least one of R³ and R⁴ is substituted or unsubstituted arylamino, or substituted or unsubstituted heteroarylamino.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and at least one of R³ and R⁴ is substituted or unsubstituted arylamino, or substituted or unsubstituted heteroarylamino.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted aminoalkyl; and at least one of R³ and R⁴ is substituted or unsubstituted arylamino, or substituted or unsubstituted heteroarylamino.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted aminoalkyl; and one of R¹ and R² is hydrogen; and at least one of R³ and R⁴ is substituted or unsubstituted arylamino, or substituted or unsubstituted heteroarylamino.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and at least one of R³ and R⁴ is substituted or unsubstituted arylamino, or substituted or unsubstituted heteroarylamino.

In another embodiment, the compounds of the present invention, or pharmaceutically acceptable salts, solvates, prodrugs, tautomers, or esters thereof, have the structure of formula (I), wherein L¹ is —S(O)— or S(O)₂—; L² is methylene; and R¹ and R², taken together with the nitrogen atom to which they are both attached, form a 5- to 18-membered saturated heterocyclic ring containing at least two nitrogen atoms; and at least one of R³ and R⁴ is substituted or unsubstituted arylamino, or substituted or unsubstituted heteroarylamino.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted arylalkyl, or substituted or unsubstituted heteroarylalkyl; and one of R³ and R⁴ is substituted or unsubstituted arylamino, or substituted or unsubstituted heteroarylamino; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted arylalkyl, or substituted or unsubstituted heteroarylalkyl; and one of R¹ and R² is hydrogen; and one of R³ and R⁴ is substituted or unsubstituted arylamino, or substituted or unsubstituted heteroarylamino; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and one of R³ and R⁴ is substituted or unsubstituted arylamino, or substituted or unsubstituted heteroarylamino; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted aminoalkyl; and one of R³ and R⁴ is substituted or unsubstituted arylamino, or substituted or unsubstituted heteroarylamino; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted aminoalkyl; and one of R¹ and R² is hydrogen; and one of R³ and R⁴ is substituted or unsubstituted arylamino, or substituted or unsubstituted heteroarylamino; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and one of R³ and R⁴ is substituted or unsubstituted arylamino, or substituted or unsubstituted heteroarylamino; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R², taken together with the nitrogen atom to which they are both attached, form a 5- to 18-membered saturated heterocyclic ring containing at least two nitrogen atoms; and one of R³ and R⁴ is substituted or unsubstituted arylamino, or substituted or unsubstituted heteroarylamino; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted arylalkyl, or substituted or unsubstituted heteroarylalkyl; and at least one of R³ and R⁴ is substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted arylalkyl, or substituted or unsubstituted heteroarylalkyl; and one of R¹ and R² is hydrogen; and at least one of R³ and R⁴ is substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and at least one of R³ and R⁴ is substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted aminoalkyl; and at least one of R³ and R⁴ is substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted aminoalkyl; and one of R¹ and R² is hydrogen; and at least one of R³ and R⁴ is substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and at least one of R³ and R⁴ is substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R², taken together with the nitrogen atom to which they are both attached, form a 5- to 18-membered saturated heterocyclic ring containing at least two nitrogen atoms; and at least one of R³ and R⁴ is substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted arylalkyl, or substituted or unsubstituted heteroarylalkyl; one of R³ and R⁴ is substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted arylalkyl, or substituted or unsubstituted heteroarylalkyl; and one of R¹ and R² is hydrogen; one of R³ and R⁴ is substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; one of R³ and R⁴ is substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted aminoalkyl; one of R³ and R⁴ is substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted aminoalkyl; and one of R¹ and R² is hydrogen; one of R³ and R⁴ is substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; one of R³ and R⁴ is substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R², taken together with the nitrogen atom to which they are both attached, form a 5- to 18-membered saturated heterocyclic ring containing at least two nitrogen atoms; one of R³ and R⁴ is substituted or unsubstituted aryl, or substituted or unsubstituted heteroaryl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted arylalkyl, or substituted or unsubstituted heteroarylalkyl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted arylalkyl, or substituted or unsubstituted heteroarylalkyl; and one of R¹ and R² is hydrogen; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted aminoalkyl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted aminoalkyl; and one of R¹ and R² is hydrogen; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R², taken together with the nitrogen atom to which they are both attached, form a 5- to 18-membered saturated heterocyclic ring containing at least two nitrogen atoms; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted arylalkyl, or substituted or unsubstituted heteroarylalkyl; and at least one of R³ and R⁴ is substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted arylalkyl, or substituted or unsubstituted heteroarylalkyl; and one of R¹ and R² is hydrogen; and at least one of R³ and R⁴ is substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and at least one of R³ and R⁴ is substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted aminoalkyl; and at least one of R³ and R⁴ is substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted aminoalkyl; and one of R¹ and R² is hydrogen; and at least one of R³ and R⁴ is substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and at least one of R³ and R⁴ is substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R², taken together with the nitrogen atom to which they are both attached, form a 5- to 18-membered saturated heterocyclic ring containing at least two nitrogen atoms; and at least one of R³ and R⁴ is substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted arylalkyl, or substituted or unsubstituted heteroarylalkyl; one of R³ and R⁴ is substituted or unsubstituted alkyl, alkoxyalkyl, or substituted or unsubstituted aminoalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted arylalkyl, or substituted or unsubstituted heteroarylalkyl; and one of R¹ and R² is hydrogen; one of R³ and R⁴ is substituted or unsubstituted alkyl, alkoxyalkyl, or substituted or unsubstituted aminoalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; one of R³ and R⁴ is substituted or unsubstituted alkyl, alkoxyalkyl, or substituted or unsubstituted aminoalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted aminoalkyl; one of R³ and R⁴ is substituted or unsubstituted alkyl, alkoxyalkyl, or substituted or unsubstituted aminoalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted aminoalkyl; and one of R¹ and R² is hydrogen; one of R³ and R⁴ is substituted or unsubstituted alkyl, alkoxyalkyl, or substituted or unsubstituted aminoalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; one of R³ and R⁴ is substituted or unsubstituted alkyl, alkoxyalkyl, or substituted or unsubstituted aminoalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R², taken together with the nitrogen atom to which they are both attached, form a 5- to 18-membered saturated heterocyclic ring containing at least two nitrogen atoms; one of R³ and R⁴ is substituted or unsubstituted alkyl, alkoxyalkyl, or substituted or unsubstituted aminoalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted arylalkyl, or substituted or unsubstituted heteroarylalkyl; and R³ and R⁴ are independently substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted arylalkyl, or substituted or unsubstituted heteroarylalkyl; and one of R¹ and R² is hydrogen; and R³ and R⁴ are independently substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and R³ and R⁴ are independently substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted aminoalkyl; and R³ and R⁴ are independently substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted aminoalkyl; and one of R¹ and R² is hydrogen; and R³ and R⁴ are independently substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and R³ and R⁴ are independently substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R², taken together with the nitrogen atom to which they are both attached, form a 5- to 18-membered saturated heterocyclic ring containing at least two nitrogen atoms; and R³ and R⁴ are independently substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted arylalkyl, or substituted or unsubstituted heteroarylalkyl; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted arylalkyl, or substituted or unsubstituted heteroarylalkyl; and one of R¹ and R² is hydrogen; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted aminoalkyl; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted aminoalkyl; and one of R¹ and R² is hydrogen; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R², taken together with the nitrogen atom to which they are both attached, form a 5- to 18-membered saturated heterocyclic ring containing at least two nitrogen atoms; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted arylalkyl, or substituted or unsubstituted heteroarylalkyl; and one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted arylalkyl, or substituted or unsubstituted heteroarylalkyl; and one of R¹ and R² is hydrogen; and one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted aminoalkyl; and one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted aminoalkyl; and one of R¹ and R² is hydrogen; and one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R², taken together with the nitrogen atom to which they are both attached, form a 5- to 18-membered saturated heterocyclic ring containing at least two nitrogen atoms; and one of R³ and R⁴ is substituted or unsubstituted arylalkyl, or substituted or unsubstituted heteroarylalkyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted arylalkyl, or substituted or unsubstituted heteroarylalkyl; and at least one of R³ and R⁴ is substituted or unsubstituted aminoacylalkyl, substituted or unsubstituted arylacyl, or substituted or unsubstituted heteroarylacyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted arylalkyl, or substituted or unsubstituted heteroarylalkyl; and one of R¹ and R² is hydrogen; and at least one of R³ and R⁴ is substituted or unsubstituted aminoacylalkyl, substituted or unsubstituted arylacyl, or substituted or unsubstituted heteroarylacyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and at least one of R³ and R⁴ is substituted or unsubstituted aminoacylalkyl, substituted or unsubstituted arylacyl, or substituted or unsubstituted heteroarylacyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted aminoalkyl; and at least one of R³ and R⁴ is substituted or unsubstituted aminoacylalkyl, substituted or unsubstituted arylacyl, or substituted or unsubstituted heteroarylacyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted aminoalkyl; and one of R¹ and R² is hydrogen; and at least one of R³ and R⁴ is substituted or unsubstituted aminoacylalkyl, substituted or unsubstituted arylacyl, or substituted or unsubstituted heteroarylacyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; and at least one of R³ and R⁴ is substituted or unsubstituted aminoacylalkyl, substituted or unsubstituted arylacyl, or substituted or unsubstituted heteroarylacyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R², taken together with the nitrogen atom to which they are both attached, form a 5- to 18-membered saturated heterocyclic ring containing at least two nitrogen atoms; and at least one of R³ and R⁴ is substituted or unsubstituted aminoacylalkyl, substituted or unsubstituted arylacyl, or substituted or unsubstituted heteroarylacyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted arylalkyl, or substituted or unsubstituted heteroarylalkyl; one of R³ and R⁴ is substituted or unsubstituted aminoacylalkyl, substituted or unsubstituted arylacyl, or substituted or unsubstituted heteroarylacyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted arylalkyl, or substituted or unsubstituted heteroarylalkyl; and one of R¹ and R² is hydrogen; one of R³ and R⁴ is substituted or unsubstituted aminoacylalkyl, substituted or unsubstituted arylacyl, or substituted or unsubstituted heteroarylacyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; one of R³ and R⁴ is substituted or unsubstituted aminoacylalkyl, substituted or unsubstituted arylacyl, or substituted or unsubstituted heteroarylacyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and at least one of R¹ and R² is substituted or unsubstituted aminoalkyl; one of R³ and R⁴ is substituted or unsubstituted aminoacylalkyl, substituted or unsubstituted arylacyl, or substituted or unsubstituted heteroarylacyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and one of R¹ and R² is substituted or unsubstituted aminoalkyl; and one of R¹ and R² is hydrogen; one of R³ and R⁴ is substituted or unsubstituted aminoacylalkyl, substituted or unsubstituted arylacyl, or substituted or unsubstituted heteroarylacyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R² are independently substituted or unsubstituted arylalkyl, or substituted or unsubstituted heteroarylalkyl; one of R³ and R⁴ is substituted or unsubstituted aminoacylalkyl, substituted or unsubstituted arylacyl, or substituted or unsubstituted heteroarylacyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is methylene; and R¹ and R², taken together with the nitrogen atom to which they are both attached, form a 5- to 18-membered saturated heterocyclic ring containing at least two nitrogen atoms; one of R³ and R⁴ is substituted or unsubstituted aminoacylalkyl, substituted or unsubstituted arylacyl, or substituted or unsubstituted heteroarylacyl; and one of R³ and R⁴ is hydrogen.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen or methyl; the other of R¹ or R² is substituted or unsubstituted arylalkyl, substituted or unsubstituted alkyl, or substituted or unsubstituted heteroarylalkyl; R⁵ is substituted or unsubstituted aminoalkyl, substituted or unsubstituted heterocyclylalkyl, substituted or unsubstituted heteroarylalkyl, or substituted or unsubstituted hydroxyalkyl; one of R³ and R⁴ is hydrogen or alkyl; the other of R³ and R⁴ is substituted or unsubstituted arylalkyl, substituted or unsubstituted heteroarylalkyl, or R³ and R⁴, taken together with the nitrogen atom to which they are both attached, form a 5- to 18-membered saturated heterocyclic ring containing at least one nitrogen atom.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted arylalkyl, R⁵ is substituted or unsubstituted aminoalkyl; one of R³ and R⁴ is hydrogen; the other of R³ and R⁴ is substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is methylene-N(R⁵)—C(O)—; L² is methylene; one of R¹ or R² is hydrogen; the other of R¹ or R² is substituted or unsubstituted 1-naphthylalkyl, R⁵ is substituted or unsubstituted morpholinoalkyl; one of R³ and R⁴ is hydrogen; the other of R³ and R⁴ is substituted or unsubstituted pyridylalkyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted arylalkyl, R⁵ is substituted or unsubstituted aminoalkyl; one of R³ and R⁴ is hydrogen; the other of R³ and R⁴ is substituted or unsubstituted arylalkyl.

In another embodiment, the compounds of the present invention, or pharmaceutically acceptable salts, solvates, prodrugs, tautomers, or esters thereof, have the structure of formula (I), wherein L¹ is methylene-N(R⁵)—C(O)—; L¹ is methylene; one of R¹ or R² is hydrogen; the other of R¹ or R² is substituted or unsubstituted 1-naphthylethyl, R⁵ is substituted or unsubstituted aminopropyl; one of R³ and R⁴ is hydrogen; the other of R³ and R⁴ is substituted or unsubstituted benzyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted arylalkyl, R⁵ is substituted or unsubstituted heterocyclylalkyl; one of R³ and R⁴ is hydrogen; the other of R³ and R⁴ is substituted or unsubstituted arylalkyl.

In another embodiment, the compounds of the present invention, or pharmaceutically acceptable salts, solvates, prodrugs, tautomers, or esters thereof, have the structure of formula (I), wherein L¹ is methylene-N(R⁵)—C(O)—; L² is methylene; one of R¹ or R² is hydrogen; the other of R¹ or R² is substituted or unsubstituted 1-naphthylethyl, R⁵ is substituted or unsubstituted N-(2-oxo)-pyrrolidinylalkyl; one of R³ and R⁴ is hydrogen; the other of R³ and R⁴ is substituted or unsubstituted benzyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted arylalkyl, R⁵ is substituted or unsubstituted heteroarylalkyl; one of R³ and R⁴ is hydrogen; the other of R³ and R⁴ is substituted or unsubstituted arylalkyl.

In another embodiment, the compounds of the present invention, or pharmaceutically acceptable salts, solvates, prodrugs, tautomers, or esters thereof, have the structure of formula (I), wherein L¹ is methylene-N(R⁵)—C(O)—; L² is methylene; one of R¹ or R² is hydrogen; the other of R¹ or R² is substituted or unsubstituted 1-naphthylethyl, R⁵ is substituted or unsubstituted imidazolylalkyl; one of R³ and R⁴ is hydrogen; the other of R³ and R⁴ is substituted or unsubstituted benzyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted arylalkyl, R⁵ is substituted or unsubstituted heterocyclylalkyl; and R³ and R⁴, taken together with the nitrogen atom to which they are both attached, form a 5- to 18-membered saturated heterocyclic ring containing at least one nitrogen atom.

In another embodiment, the compounds of the present invention, or pharmaceutically acceptable salts, solvates, prodrugs, tautomers, or esters thereof, have the structure of formula (I), wherein L¹ is methylene-N(R⁵)—C(O)—; L² is methylene; one of R¹ or R² is hydrogen; the other of R¹ or R² is substituted or unsubstituted 1-naphthylethyl, R⁵ is substituted or unsubstituted morpholinoalkyl; and R³ and R⁴, taken together with the nitrogen atom to which they are both attached, form a 6-membered saturated heterocyclic ring containing two nitrogen atoms.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted arylalkyl, R⁵ is substituted or unsubstituted alkyl; one of R³ and R⁴ is hydrogen; the other of R³ and R⁴ is substituted or unsubstituted arylalkyl.

In another embodiment, the compounds of the present invention, or pharmaceutically acceptable salts, solvates, prodrugs, tautomers, or esters thereof, have the structure of formula (I), wherein L¹ is methylene-N(R⁵)—C(O)—; L² is methylene; one of R¹ or R² is hydrogen; the other of R¹ or R² is substituted or unsubstituted 1-naphthylethyl, R⁵ is tert-butyl; one of R³ and R⁴ is hydrogen; the other of R³ and R⁴ is substituted or unsubstituted benzyl.

In another embodiment, the compounds of the present invention, or pharmaceutically acceptable salts, solvates, prodrugs, tautomers, or esters thereof, have the structure of formula (I), wherein L¹ is methylene-N(R⁵)—C(O)—; L² is methylene; one of R¹ or R² is hydrogen; the other of R¹ or R² is substituted or unsubstituted 1-naphthylethyl, R⁵ is substituted or morpholinoalkyl; one of R³ and R⁴ is hydrogen; the other of R³ and R⁴ is substituted or unsubstituted benzyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted arylalkyl, R⁵ is substituted or unsubstituted heterocyclylalkyl; one of R³ and R⁴ is hydrogen; the other of R³ and R⁴ is substituted or unsubstituted arylalkyl.

In another embodiment, the compounds of the present invention, or pharmaceutically acceptable salts, solvates, prodrugs, tautomers, or esters thereof, have the structure of formula (I), wherein L¹ is methylene-N(R⁵)—C(O)—; L² is methylene; one of R¹ or R² is hydrogen; the other of R¹ or R² is substituted or unsubstituted benzyl, R⁵ is morpholinoalkyl; one of R³ and R⁴ is hydrogen; the other of R³ and R⁴ is substituted or unsubstituted benzyl.

In another embodiment, the compounds of the present invention, or pharmaceutically acceptable salts, solvates, prodrugs, tautomers, or esters thereof, have the structure of formula (I), wherein L¹ is methylene-N(R⁵)—C(O)—; L¹ is methylene; one of R¹ or R² is hydrogen; the other of R¹ or R² is substituted or unsubstituted naphthylmethyl, R⁵ is morpholinoalkyl; one of R³ and R⁴ is hydrogen; the other of R³ and R⁴ is substituted or unsubstituted imidazolylalkyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted alkyl, R⁵ is substituted or unsubstituted heterocyclylalkyl; one of R³ and R⁴ is hydrogen; the other of R³ and R⁴ is substituted or unsubstituted arylalkyl.

In another embodiment, the compounds of the present invention, or pharmaceutically acceptable salts, solvates, prodrugs, tautomers, or esters thereof, have the structure of formula (I), wherein L¹ is methylene-N(R⁵)—C(O)—; L² is methylene; one of R¹ or R² is hydrogen; the other of R¹ or R² is substituted or unsubstituted 1,2-dimethylpropyl; R⁵ is morpholinoalkyl; one of R³ and R⁴ is hydrogen; the other of R³ and R⁴ is substituted or unsubstituted benzyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted arylalkyl, R⁵ is substituted or unsubstituted heterocyclylalkyl; one of R³ and R⁴ is hydrogen; the other of R³ and R⁴ is substituted or unsubstituted alkyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted arylalkyl, R⁵ is substituted or unsubstituted heterocyclylalkyl; one of R³ and R⁴ is aminoalkyl; the other of R³ and R⁴ is substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted arylalkyl; R⁵ is substituted or unsubstituted heterocyclylalkyl; and R³ and R⁴, taken together with the nitrogen atom to which they are both attached, form a 5-membered saturated heterocyclic ring containing at least one nitrogen atom.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted 1-naphthylethyl; R⁵ is substituted or unsubstituted pyrrolidinoalkyl; one of R³ and R⁴ is H; and the other of R³ and R⁴ is substituted or unsubstituted benzyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted 1-naphthylethyl; R⁵ is substituted or unsubstituted morpholinoalkyl; and R³ and R⁴, taken together with the nitrogen atom to which they are both attached, form a 5-membered saturated heterocyclic ring containing at least one nitrogen atom.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted 1-naphthylethyl; R⁵ is substituted or unsubstituted morpholinoalkyl; one of R³ and R⁴ is H; and the other of R³ and R⁴ is morpholinoalkyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted 1-naphthylethyl; R⁵ is substituted or unsubstituted morpholinoalkyl; one of R³ and R⁴ is H; and the other of R³ and R⁴ is imidazolylalkyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted 1-naphthylethyl; R⁵ is substituted or unsubstituted aminoalkyl; one of R³ and R⁴ is H; and the other of R³ and R⁴ is substituted or unsubstituted benzyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted 1-naphthylethyl; R⁵ is substituted or unsubstituted hydroxyalkyl; one of R³ and R⁴ is H; and the other of R³ and R⁴ is substituted or unsubstituted benzyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is alkyl; the other of R¹ or R² is substituted or unsubstituted cycloalkyl; R⁵ is substituted or unsubstituted aminoalkyl; one of R³ and R⁴ is H; and the other of R³ and R⁴ is substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is methyl; the other of R¹ or R² is substituted or unsubstituted tetrahydroisoquinolyl; R⁵ is substituted or unsubstituted aminoalkyl; one of R³ and R⁴ is H; and the other of R³ and R⁴ is substituted or unsubstituted pyridylalkyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted cycloalkyl; R⁵ is substituted or unsubstituted aminoalkyl; one of R³ and R⁴ is H; and the other of R³ and R⁴ is substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is hydrogen; the other of R¹ or R² is substituted or unsubstituted tetrahydroisoquinolyl; R⁵ is substituted or unsubstituted aminoalkyl; one of R³ and R⁴ is H; and the other of R³ and R⁴ is substituted or unsubstituted pyridylalkyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is alkyl; the other of R¹ or R² is substituted or unsubstituted cycloalkyl; R⁵ is substituted or unsubstituted aminoalkyl; one of R³ and R⁴ is H; and the other of R³ and R⁴ is substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is methyl; the other of R¹ or R² is substituted or unsubstituted tetrahydroisoquinolyl; R⁵ is substituted or unsubstituted aminoalkyl; one of R³ and R⁴ is H; and the other of R³ and R⁴ is substituted or unsubstituted benzyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is alkyl; the other of R¹ or R² is substituted or unsubstituted cycloalkyl; R⁵ is substituted or unsubstituted aminoalkyl; one of R³ and R⁴ is H; and the other of R³ and R⁴ is substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is alkylene; one of R¹ or R² is methyl; the other of R¹ or R² is substituted or unsubstituted tetrahydroisoquinolyl; R⁵ is substituted or unsubstituted aminoalkyl; one of R³ and R⁴ is H; and the other of R³ and R⁴ is substituted or unsubstituted imidazolylalkyl.

In another embodiment, the compounds of the present invention, or pharmaceutically acceptable salts, solvates, prodrugs, tautomers, or esters thereof, have the structure of formula (I), wherein L¹ is —S(O)₂—; L² is alkylene; one of R¹ and R² is H or alkyl; the other of R¹ or R² is substituted or unsubstituted aryl, substituted or unsubstituted arylalkyl, or R¹ and R², taken together with the nitrogen atom to which they are both attached, form a 5- to 18-membered saturated heterocyclic ring containing at least one nitrogen atom; one of R³ and R⁴ is H or alkyl; the other of R³ or R⁴ is substituted or unsubstituted aryl, substituted or unsubstituted arylalkyl, or R³ and R⁴, taken together with the nitrogen atom to which they are both attached, form a 5- to 18-membered saturated heterocyclic ring containing at least one nitrogen atom.

In another embodiment, the compounds of the present invention, or pharmaceutically acceptable salts, solvates, prodrugs, tautomers, or esters thereof, have the structure of formula (I), wherein L¹ is —S(O)₂—; L² is alkylene; one of R¹ and R² is H or alkyl; the other of R¹ or R² is substituted or unsubstituted aryl, substituted or unsubstituted arylalkyl; one of R³ and R⁴ is H or alkyl; the other of R³ or R⁴ is substituted or unsubstituted aryl, substituted or unsubstituted arylalkyl.

In another embodiment, the compounds of the present invention, or pharmaceutically acceptable salts, solvates, prodrugs, tautomers, or esters thereof, have the structure of formula (I), wherein L¹ is —S(O)₂—; L² is alkylene; R¹ and R², taken together with the nitrogen atom to which they are both attached, form a 5- to 18-membered saturated heterocyclic ring containing at least one nitrogen atom; one of R³ and R⁴ is H or alkyl; the other of R³ or R⁴ is substituted or unsubstituted aryl, substituted or unsubstituted arylalkyl.

In another embodiment, the compounds of the present invention, or pharmaceutically acceptable salts, solvates, prodrugs, tautomers, or esters thereof, have the structure of formula (I), wherein L¹ is —S(O)₂—; L² is alkylene; R¹ and R², taken together with the nitrogen atom to which they are both attached, form a 5- to 18-membered saturated heterocyclic ring containing at least one nitrogen atom; and R³ and R⁴, taken together with the nitrogen atom to which they are both attached, form a 5- to 18-membered saturated heterocyclic ring containing at least one nitrogen atom.

In another embodiment, the compounds of the present invention, or pharmaceutically acceptable salts, solvates, prodrugs, tautomers, or esters thereof, have the structure of formula (I), wherein L¹ is —S(O)₂—; L² is alkylene; one of R¹ and R² is H or alkyl; the other of R¹ or R² is substituted or unsubstituted aryl, substituted or unsubstituted arylalkyl, or R¹ and R², taken together with the nitrogen atom to which they are both attached, form a 5- to 18-membered saturated heterocyclic ring containing at least one nitrogen atom; one of R³ and R⁴ is H or alkyl; the other of R³ or R⁴ is substituted or unsubstituted aryl, substituted or unsubstituted arylalkyl, or R³ and R⁴, taken together with the nitrogen atom to which they are both attached, form a 5- to 18-membered saturated heterocyclic ring containing at least one nitrogen atom.

In another embodiment, the compounds of the present invention, or pharmaceutically acceptable salts, solvates, prodrugs, tautomers, or esters thereof, have the structure of formula (I), wherein L¹ is —S(O)₂—; L² is alkylene; one of R¹ and R² is H or alkyl; the other of R¹ or R² is substituted or unsubstituted aryl, substituted or unsubstituted arylalkyl; one of R³ and R⁴ is H or alkyl; the other of R³ or R⁴ is substituted or unsubstituted aryl, substituted or unsubstituted arylalkyl.

In another embodiment, the compounds of the present invention, or pharmaceutically acceptable salts, solvates, prodrugs, tautomers, or esters thereof, have the structure of formula (I), wherein L¹ is —S(O)₂—; L² is alkylene; R¹ and R², taken together with the nitrogen atom to which they are both attached, form a 5- to 18-membered saturated heterocyclic ring containing at least one nitrogen atom; one of R³ and R⁴ is H or alkyl; the other of R³ or R⁴ is substituted or unsubstituted aryl, substituted or unsubstituted arylalkyl.

In another embodiment, the compounds of the present invention, or pharmaceutically acceptable salts, solvates, prodrugs, tautomers, or esters thereof, have the structure of formula (I), wherein L¹ is —S(O)₂—; L² is alkylene; R¹ and R², taken together with the nitrogen atom to which they are both attached, form a 5- to 18-membered saturated heterocyclic ring containing at least one nitrogen atom; and R³ and R⁴, taken together with the nitrogen atom to which they are both attached, form a 5- to 18-membered saturated heterocyclic ring containing at least one nitrogen atom.

In another embodiment, the compounds of the present invention, or pharmaceutically acceptable salts, solvates, prodrugs, tautomers, or esters thereof, have the structure of formula (I), wherein L¹ is —S(O)₂—; L² is alkylene; R³ and R⁴ are each independently H, substituted or unsubstituted alkyl; substituted or unsubstituted heterocyclyl, substituted or unsubstituted cycloalkyl, or heteroarylalkyl, or R³ and R⁴, taken together with the nitrogen atom to which they are both attached, form a 5- to 18-membered saturated heterocyclic ring containing at least one nitrogen atom; R¹ and R², taken together with the nitrogen atom to which they are both attached, form a 5- to 18-membered saturated heterocyclic ring containing at least one nitrogen atom.

In another embodiment, the compounds of the present invention, or pharmaceutically acceptable salts, solvates, prodrugs, tautomers, or esters thereof, have the structure of formula (I), wherein L¹ is —S(O)₂—; L² is alkylene; R³ and R⁴ are each independently H, substituted or unsubstituted alkyl; substituted or unsubstituted heterocyclyl, substituted or unsubstituted cycloalkyl, or heteroarylalkyl, or R³ and R⁴, taken together with the nitrogen atom to which they are both attached, form a 5- to 18-membered saturated heterocyclic ring containing at least one nitrogen atom; R¹ and R¹², taken together with the nitrogen atom to which they are both attached, form a 5- to 18-membered saturated heterocyclic ring containing at least one nitrogen atom.

In another embodiment, the compounds of the present invention, or pharmaceutically acceptable salts, solvates, prodrugs, tautomers, or esters thereof, have the structure of formula (I), wherein L¹ is —S(O)₂—; L² is alkylene; R³ and R⁴ are each independently H, substituted or unsubstituted alkyl; substituted or unsubstituted heterocyclyl, substituted or unsubstituted cycloalkyl, or heteroarylalkyl, or R³ and R⁴, taken together with the nitrogen atom to which they are both attached, form a 5- to 18-membered saturated heterocyclic ring containing at least one nitrogen atom; R¹ and R¹², taken together with the nitrogen atom to which they are both attached, form tetrahydroisoquinolyl.

In another embodiment, the compounds of the present invention, or pharmaceutically acceptable salts, solvates, prodrugs, tautomers, or esters thereof, have the structure of formula (I), wherein L¹ is —S(O)₂—; L² is alkylene; R³ and R⁴ are each independently H, substituted or unsubstituted alkyl; substituted or unsubstituted heterocyclyl, substituted or unsubstituted cycloalkyl, or heteroarylalkyl, or R³ and R⁴, taken together with the nitrogen atom to which they are both attached, form a 5- to 18-membered saturated heterocyclic ring containing at least one nitrogen atom; R¹ and R², taken together with the nitrogen atom to which they are both attached, form isoindolyl.

In another embodiment, the compounds of the present invention, or pharmaceutically acceptable salts, solvates, prodrugs, tautomers, or esters thereof, have the structure of formula (I), wherein L¹ is —S(O)₂—; L² is alkylene; R³ and R⁴ are each independently H, substituted or unsubstituted alkyl; substituted or unsubstituted heterocyclyl, substituted or unsubstituted cycloalkyl, or heteroarylalkyl, or R³ and R⁴, taken together with the nitrogen atom to which they are both attached, form a 5- to 18-membered saturated heterocyclic ring containing at least one nitrogen atom; R¹ and R², taken together with the nitrogen atom to which they are both attached, form 5,6,7,8-tetrahydro-1,7-naphthyridyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is C(O); one of R¹ or R² is hydrogen; the other of R¹ or R² is substituted or unsubstituted arylalkyl; R⁵ is substituted or unsubstituted aminoalkyl; one of R³ and R⁴ is H; and the other of R³ and R⁴ is substituted or unsubstituted arylamino, alkoxy, or substituted or unsubstituted amino.

In another embodiment, the compounds of the present invention, or pharmaceutically acceptable salts, solvates, prodrugs, tautomers, or esters thereof, have the structure of formula (I), wherein L¹ is methylene-N(R⁵)—C(O)—; L² is C(O); one of R¹ or R² is hydrogen; the other of R¹ or R² is substituted or unsubstituted 1-naphthylethyl; R⁵ is substituted or unsubstituted morpholinoalkyl; one of R³ and R⁴ is H; and the other of R³ and R⁴ is substituted or unsubstituted phenylamino, methoxy, urethanyl, or amino.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is —CH₂CH₂—; one of R¹ or R² is hydrogen; the other of R¹ or R² is substituted or unsubstituted arylalkyl; R⁵ is substituted or unsubstituted aminoalkyl; one of R³ and R⁴ is H; and the other of R³ and R⁴ is H, alkoxycarbonyl, substituted or unsubstituted aryl-S(O)₂—, or substituted or unsubstituted arylalkyl.

In another embodiment, the compounds of the present invention, or pharmaceutically acceptable salts, solvates, prodrugs, tautomers, or esters thereof, have the structure of formula (I), wherein L¹ is alkylene-N(R⁵)—C(O)—; L² is —CH₂CH₂—; one of R¹ or R² is hydrogen; the other of R¹ or R² is substituted or unsubstituted 1-naphthylethyl; R⁵ is substituted or unsubstituted morpholinoalkyl; one of R³ and R⁴ is H; and the other of R³ and R⁴ is H, tert-butoxycarbonyl, substituted or unsubstituted phenyl-S(O)₂—, or substituted or unsubstituted benzyl.

In another embodiment, the compounds of the present invention, or pharmaceutically acceptable salts, solvates, prodrugs, tautomers, or esters thereof, have the structure of formula (I), wherein L¹ is —N(R⁵)—C(O)—, —N(R⁵)—S(O)—, —N(R⁵)—S(O)₂—, -alkylene-N(R⁵)—C(O)—, -alkylene-N(R⁵)—S(O)—, or -alkylene-N(R⁵)—S(O)₂—; and R³ and R⁴ are not both hydrogen.

In another embodiment, the compounds of the present invention, or pharmaceutically acceptable salts, solvates, prodrugs, tautomers, or esters thereof, have the structure of formula (I), wherein L¹ is —N(R⁵)—C(O)—, —N(R⁵)—S(O)—, or —N(R⁵)—S(O)₂—.

In another embodiment, the compounds of the present invention, or pharmaceutically acceptable salts, solvates, prodrugs, tautomers, or esters thereof, have the structure of formula (I), wherein L¹ is -alkylene-N(R⁵)—C(O)—, -alkylene-N(R⁵)—S(O)—, or -alkylene-N(R⁵)—S(O)₂—.

In another embodiment, the compounds of the present invention, or pharmaceutically acceptable salts, solvates, prodrugs, tautomers, or esters thereof, have the structure of formula (I), wherein L¹ is —CH₂—N(R⁵)—C(O)—, —CH₂—N(R⁵)—S(O)—, —CH₂—N(R⁵)—S(O)₂—, —CH₂CH₂—N(R⁵)—C(O)—, —CH₂CH₂—N(R⁵)—S(O)—, or —CH₂CH₂—N(R⁵)—S(O)₂—

In another embodiment, the compounds of the present invention, or pharmaceutically acceptable salts, solvates, prodrugs, tautomers, or esters thereof, have the structure of formula (I), wherein L¹ is —N(R⁵)—C(O)—, —N(R⁵)—S(O)—, —N(R⁵)—S(O)₂—, -alkylene-N(R⁵)—C(O)—, -alkylene-N(R⁵)—S(O)—, or -alkylene-N(R⁵)—S(O)₂—; and R³ and R⁴ are not both hydrogen; and R⁵ is hydrogen, substituted or unsubstituted alkyl, substituted or unsubstituted arylalkyl, substituted or unsubstituted heteroarylalkyl, substituted or unsubstituted heterocyclyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —N(R⁵)—C(O)—, —N(R⁵)—S(O)—, —N(R⁵)—S(O)₂—, -alkylene-N(R⁵)—C(O)—, -alkylene-N(R⁵)—S(O)—, or -alkylene-N(R⁵)—S(O)₂—; and R³ and R⁴ are not both hydrogen; and L² is alkylene.

In another embodiment, the compounds of the present invention, or pharmaceutically acceptable salts, solvates, prodrugs, tautomers, or esters thereof, have the structure of formula (I), wherein L¹ is —N(R⁵)—C(O)—, —N(R⁵)—S(O)—, —N(R⁵)—S(O)₂—, -alkylene-N(R⁵)—C(O)—, -alkylene-N(R⁵)—S(O)—, or -alkylene-N(R⁵)—S(O)₂—; and R³ and R⁴ are not both hydrogen; and L²-CH₂— or —CH₂CH₂—.

In another embodiment, the compounds of the present invention, or pharmaceutically acceptable salts, solvates, prodrugs, tautomers, or esters thereof, have the structure of formula (I), wherein L¹ is —N(R⁵)—C(O)—, —N(R⁵)—S(O)—, —N(R⁵)—S(O)₂—, -alkylene-N(R⁵)—C(O)—, -alkylene-N(R⁵)—S(O)—, or -alkylene-N(R⁵)—S(O)₂—; R³ and R⁴ are not both hydrogen; and at least one of R¹, R², R³, and R⁴ is hydrogen.

In another embodiment, the compounds of the present invention, or pharmaceutically acceptable salts, solvates, prodrugs, tautomers, or esters thereof, have the structure of formula (I), wherein L¹ is —N(R⁵)—C(O)—, —N(R⁵)—S(O)—, —N(R⁵)—S(O)₂—, -alkylene-N(R⁵)—C(O)—, -alkylene-N(R⁵)—S(O)—, or -alkylene-N(R⁵)—S(O)₂—; R³ and R⁴ are not both hydrogen; and at least one of R¹ and R² is substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —N(R⁵)—C(O)—, —N(R⁵)—S(O)—, —N(R⁵)—S(O)₂—, -alkylene-N(R⁵)—C(O)—, -alkylene-N(R⁵)—S(O)—, or -alkylene-N(R⁵)—S(O)₂—; and R³ and R⁴, taken together with the nitrogen atom to which they are both attached, form a 5- to 18-membered saturated heterocyclic ring containing at least onenitrogen atom.

In another embodiment, the compounds of the present invention, or pharmaceutically acceptable salts, solvates, prodrugs, tautomers, or esters thereof, have the structure of formula (I), wherein L¹ is —N(R⁵)—C(O)—, —N(R⁵)—S(O)—, —N(R⁵)—S(O)₂—, -alkylene-N(R⁵)—C(O)—, -alkylene-N(R⁵)—S(O)—, or -alkylene-N(R⁵)—S(O)₂—; and at least one of R³ and R⁴ is amino, alkoxy, alkoxyalkyl, substituted or unsubstituted aminoalkyl, substituted or unsubstituted aminoacyl, substituted or unsubstituted cycloalkyl, substituted or unsubstituted alkoxycarbonyl, substituted or unsubstituted heterocyclyl, substituted or unsubstituted arylalkyl, substituted or unsubstituted heteroarylalkyl, substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —C(O)—.

In another embodiment, the compounds of the present invention, or pharmaceutically acceptable salts, solvates, prodrugs, tautomers, or esters thereof, have the structure of formula (I), wherein L¹ is —C(O)—; and L² is a covalent bond.

In another embodiment, the compounds of the present invention, or pharmaceutically acceptable salts, solvates, prodrugs, tautomers, or esters thereof, have the structure of formula (I), wherein L¹ is —C(O)—; L² is a covalent bond; and at least one of R³ and R⁴ is substituted or unsubstituted arylalkyl, substituted or unsubstituted heteroarylalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —C(O)—; L² is a covalent bond; and at least one of R¹ and R² is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; and L² is alkylene.

In another embodiment, the compounds of the present invention, or pharmaceutically acceptable salts, solvates, prodrugs, tautomers, or esters thereof, have the structure of formula (I), wherein L¹ is —C(O)—; and L² is —CH₂—, or —CH₂CH₂—.

In another embodiment, the compounds of the present invention, or pharmaceutically acceptable salts, solvates, prodrugs, tautomers, or esters thereof, have the structure of formula (I), wherein L¹ is —C(O)—; L² is alkylene; and at least one of R¹, R², R³, and R⁴ is hydrogen.

In another embodiment, the compounds of the present invention, or pharmaceutically acceptable salts, solvates, prodrugs, tautomers, or esters thereof, have the structure of formula (I), wherein L¹ is —C(O)—; L² is alkylene; and at least one of R³ and R⁴ is substituted or unsubstituted aryl, substituted or unsubstituted heteroaryl, substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —C(O)—; L² is alkylene; and R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —C(O)—; L² is alkylene; and at least one of R³ and R⁴ is alkoxyalkyl or substituted or unsubstituted aminoalkyl, substituted or unsubstituted aminoacylalkyl, or substituted or unsubstituted aminoacylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—.

In another embodiment, the compounds of the present invention, or pharmaceutically acceptable salts, solvates, prodrugs, tautomers, or esters thereof, have the structure of formula (I), wherein L² is —C(O)—, —S(O)—, or —S(O)₂—.

In another embodiment, the compounds of the present invention, or pharmaceutically acceptable salts, solvates, prodrugs, tautomers, or esters thereof, have the structure of formula (I), wherein L² is —C(O)—, —S(O)—, or —S(O)₂—; and at least one of R¹ and R² is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L² is —C(O)—, —S(O)—, or —S(O)₂—; and at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; and L² is alkylene.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; and L² is —CH₂—.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R³ and R⁴ are both hydrogen, and R¹ and R² are independently substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R¹ and R² is substituted or unsubstituted aminoalkyl, or substituted or unsubstituted aminoacyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R² are independently substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and R¹ and R², taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R³ and R⁴ is substituted or unsubstituted arylamino, or substituted or unsubstituted heteroarylamino.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R³ and R⁴ is substituted or unsubstituted aryl, or substituted or unsubstituted heteroaryl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; R³ and R⁴, taken together with the nitrogen atom to which they are both attached, form a 5- to 18-membered saturated heterocyclic ring containing at least two nitrogen atoms.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; at least one of R³ and R⁴ is substituted or unsubstituted alkyl, alkoxyalkyl, or substituted or unsubstituted aminoalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; at least one of R³ and R⁴ is substituted or unsubstituted arylalkyl, or substituted or unsubstituted heteroarylalkyl.

In another embodiment, the compounds of the present invention, or pharmaceutically acceptable salts, solvates, prodrugs, tautomers, or esters thereof, have the structure of formula (I), wherein L¹ is —S(O)— or —S(O)₂—; L² is alkylene; and at least one of R³ and R⁴ is substituted or unsubstituted aminoacylalkyl, substituted or unsubstituted arylacyl, or substituted or unsubstituted heteroarylacyl.

### DEFINITIONS

The term “organism” refers to any living entity comprised of at least one cell. A living organism can be as simple as, for example, a single eukaryotic cell or as complex as a mammal, including a human being.

The term “chemokine receptor modulator” means a substance including but not limited to a molecule, polypeptide, polynucleotide, inhibitory polynucleotide, or siRNA, that interferes or inhibits the biological activity of the chemokine receptors including, but not limited to, the binding of a ligand to the receptor.

The term “chemokine peptide antagonist” means a polypeptide that specifically binds to a chemokine receptor, particularly polypeptides that are not an antibody.

Representative chemokine peptide antagonists include T140 and derivatives of T140. Exemplary derivatives of T140 include, but are not limited to, TN14003, TC14012, and TE14011 as well as those found in Tamamura, H. et al. Synthesis of potent CXCR4 inhibitors possessing low cytotoxicity and improved biostability based on T140 derivatives, Org. Biomol. Chem. 1:3656-3662, 2003, which is incorporated by reference herein in its entirety.

The term “therapeutically effective amount” or “effective amount”, as used herein, means an amount of a compound or composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, and the particular active ingredient(s) being employed, and like factors within the knowledge and expertise of the attending physician. For example, in reference to cancer or pathologies related to unregulated cell division, a therapeutically effective amount refers to that amount which has the effect of (1) reducing the size of a tumor, (2) inhibiting (that is, slowing to some extent, preferably stopping) aberrant cell division, for example cancer cell division, (3) preventing or reducing the metastasis of cancer cells, and/or, (4) relieving to some extent (or, preferably, eliminating) one or more symptoms associated with a pathology related to or caused in part by unregulated or aberrant cellular division, including for example, cancer, or angiogenesis.

A “pharmaceutical composition” refers to a mixture of one or more of the compounds described herein, or pharmaceutically acceptable salts thereof, with other chemical components, such as physiologically acceptable carriers and excipients. One purpose of a pharmaceutical composition is to facilitate administration of a compound to an organism.

As used herein, a “pharmaceutically acceptable carrier” or “pharmaceutically acceptable excipient” refers to a carrier or diluent that does not cause significant irritation to an organism and does not abrogate the biological activity and proper-ties of the administered compound.

An “excipient” refers to an inert substance added to a pharmaceutical composition to further facilitate administration of a compound, such as binders, anti-adherents, coatings, disintegrants, fillers, diluents, flavors, colors, glidants, lubricants, preservatives, sorbitans, and sweeteners. Examples, without limitation, of excipients include calcium carbonate, calcium phosphate, various sugars and types of starch, cellulose derivatives, gelatin, vegetable oils and polyethylene glycols.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to HIV or cancer, these terms simply mean that the life expectancy of an individual affected with HIV or cancer will be increased or that one or more of the symptoms of the disease will be reduced.

The term “prodrug” refers to an agent, including nucleic and polypeptides, which is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceufical Properties through Prodrugs and Analogs, APhA; Acad. Pharm. Sci.; E. B. Rocke, ed. (1977). Bioreversible Carriers in Drug in Drug Design, Theory and Application, APhA; H. Bundgaard, 15 ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of P-Lactam antibiotics, Pharm. Biotech. 11:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. 1. Lee and E. M. Topp, Eds., Transport Processes in Pharmaceutical Systems, Marcell Dekker, p. 185-21 8; Balant et al. (1990) Prodrugs for the improvement of drug absorption via different routes of administration, Eur. J. Drug Metab. Pharmacokinet., 15(2):143-53; Balimane and Sinko (1999). Involvement of multiple transporters in the oral absorption of nucleoside analogues, Adv. Drug Delivery Rev., 39(1-3): 1 83-209; Browne (1997). Fosphenyloin (Cerebyx), Clin. Neuropharmacol. 20(1): 1-1 2; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1 996). Improved oral drug delivery: solubility limitations overcome by the use of prodrugs, Adv. Drug Delivery Rev, 19(2): 115-130; Fleisher et al. (1985). Design of prodrugs for improved gastrointestinal absorption by intestinal enzyme targeting, Methods Enzymol. 112:360-81; Farquhar D, et al. (1983). Biologically Reversible Phosphate-Protective Groups, J. Pharm. Sci., 72(3): 24-325; Han, H. K. et al. (2000). Targeted prodrug design to optimize drug delivery, AAPS PharmSci., 2(1): E6; Sadzuka Y. (2000). Effective prodrug liposome and conversion to active metabolite, Curr. Drug Metab., 1(1):31-48; D. M. Lambert (2000) Rationale and applications of lipids as prodrug carriers, Eur. J. Pharm. Sci, 11 Suppl2:S15-27; Wang, W. et al. (1999) Prodrug approaches to the improved delivery of peptide drugs. Gurr. Pharm. Des., 5(4):265-87.

As used herein, the term “topically active agents” refers to compositions of the present disclosure that elicit pharmacological responses at the site of application (contact) to a host.

As used herein, the term “topically” refers to application of the compositions of the present disclosure to the surface of the skin and mucosal cells and tissues.

The term “nucleic acid” is a term of art that refers to a string of at least two base-sugar-phosphate combinations. For naked DNA delivery, a polynucleotide contains more than 120 monomeric units since it must be distinguished from an oligonucleotide. However, for purposes of delivering RNA, RNAi and siRNA, either single or double stranded, a polynucleotide contains 2 or more monomeric units. Nucleotides are the monomeric units of nucleic acid polymers. The term includes deoxyribonucleic acid (DNA) and ribonucleic acid (RNA) in the form of a messenger RNA, anti-sense, plasmid DNA, parts of a plasmid DNA or genetic material derived from a virus. Anti-sense is a polynucleotide that interferes with the function of DNA and/or RNA. Natural nucleic acids have a phosphate backbone, artificial nucleic acids may contain other types of backbones, but contain the same bases. RNA may be in the form of an tRNA (transfer RNA), snRNA (small nuclear RNA), rRNA (ribosomal RNA), mRNA (messenger RNA), anti-sense RNA, RNAi, siRNA, and ribozymes. The term also includes PNAs (peptide nucleic acids), phosphorothioates, and other variants of the phosphate backbone of native nucleic acids.

The term “siRNA” means a small inhibitory ribonucleic acid. The siRNA are typically less than 30 nucleotides in length and can be single or double stranded. The ribonucleotides can be natural or artificial and can be chemically modified. Longer siRNAs can comprise cleavage sites that can be enzymatically or chemically cleaved to produce siRNAs having lengths less than 30 nucleotides, typically 21 to 23 nucleotides. siRNAs share sequence homology with corresponding target mRNAs. The sequence homology can be 100 percent or less but sufficient to result is sequence specific association between the siRNA and the targeted mRNA.

The term “inhibitory nucleic acid” means an RNA, DNA, or combination thereof that interferes or interrupts the translation of mRNA. Inhibitory nucleic acids can be single or double stranded. The nucleotides of the inhibitory nucleic acid can be chemically modified, natural or artificial.

The term “prophylactically effective amount” refers to an amount effective, at dosages and for periods of time necessary, to achieve the desired prophylactic result, such as modulation of chemokine receptor activity. A prophylactically effective amount can be determined as described herein for an effective amount. Typically, since a prophylactic dose is used in subjects prior to or at an earlier stage of disease, the prophylactically effective amount will be less than a therapeutically effective amount.

Abbreviations used include: CXCR4, CXC Chemokine receptor-4; SDF-1 stromal-derived factor-1; FACS, fluorescence-activated cell sorter; VEGF, vascular endothelial growth factor; MTT, methylthiazoletetrazolium; RT-PCR, Reverse transcription Polymerase Chain Reaction; MAb, monoclonal antibody; PE, R-Phycoerithrin; SCID, Severe Combined Immunodeficient; CC₅₀, 50% cytotoxic concentration; EC₅₀, 50% effective concentration; SI, selective index (CC₅₀/EC₅₀); DCIS, Ductal carcinoma in situ, H&E, hematoxylin and eosin; siRNA, small interfering RNA; HPRT, hypoxanthine-guanine-phosphoribosyltransferase.

The term “alkyl”, as used herein, unless otherwise specified, includes but is not limited to a saturated straight or branched, primary, secondary, or tertiary hydrocarbon of C₁ to C₂₀ or C₁ to C₁₀ and specifically includes methyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. The term optionally includes substituted alkyl groups. Moieties with which the alkyl group can be substituted are selected from the group consisting of halo (e.g., trifluoromethyl), hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “alkenyl” refers to an alkyl, as defined herein, in which at least one C—C (single) bond is replaced with a C═C (double) bond. The alkenyl can be branched or straight chain, and can have one, two or more C═C double bonds, which can be conjugated or unconjugated.

The term “alkynyl” refers to an alkyl, as defined herein, in which at least one C—C (single) bond is replaced with a C≡C (triple) bond. The alkynyl can be branched or straight chain, and can have one, two or more C≡C triple bonds.

Whenever the terms “C₁-C₅ alkyl”, “C₂-C₅ alkenyl”, “C₁-C₅ alkoxy”, “C₂-C₅ alkenoxy”, “C₂-C₅ alkynyl”, and “C₂-C₅ alkynoxy”, “C₃-C₅” are used, these are considered to include, independently, each member of the group, such that, for example, C₁-C₅ alkyl includes straight, branched and C₁, C₂, C₃, C₄ and C₅ alkyl functionalities; C₂-C₅ alkenyl includes straight and branched C₂, C₃, C₄ and C₅ alkenyl functionalities; C₁-C₈ alkoxy includes straight and branched, C₁, C₂, C₃, C₄ and C₅ alkoxy functionalities; C₂-C₅ alkenoxy includes straight and branched C₂, C₃, C₄ and C₅ alkenoxy functionalities; C₂-C₅ alkynyl includes straight and branched C₁, C₂, C₃, C₄ and C₅ alkynyl functionalities; and C₂-C₅ alkynoxy includes straight and branched C₂, C₃, C₄ and C₅ alkynoxy functionalities, etc.

The term “lower alkyl”, as used herein, and unless otherwise specified, includes a C₁ to C₄ saturated straight or branched alkyl group, optionally including substituted forms. Unless otherwise specifically stated in this application, when alkyl is a suitable moiety, lower alkyl is preferred. Similarly, when alkyl or lower alkyl is a suitable moiety, unsubstituted alkyl or lower alkyl is preferred.

The term “alkylene”, as used herein, means an organic radical formed from an unsaturated aliphatic hydrocarbon. Typically, an alkylene can be represented by the following formula: —C(RR′)ₙ—, wherein n is an integer of one or more, and R and R′ is hydrogen, halo, hydroxyl, amino, cyano (i.e., —CN), nitro, alkoxy, alkylamino, arylamino, sulfate, sulfonic acid, phosphonic acid, phosphate, phosphonate, either unprotected, or protected as necessary, as known to those skilled in the art. Preferably, n is an integer from 1 to 20. More preferably, n is an integer from 1 to 6. The alkylene can be straight, branched, or cyclic. Non-limiting examples of alkylene include —CH₂— (methylene), —CH₂CH₂— (ethylene), —CH₂CH₂CH₂— (propylene), etc.

The term “amino” includes an amine group (i.e., —NH₂) as well as an amine group substituted with one or more alkyl groups (as defined herein), substituted alkyl groups (e.g., hydroxyalkyl, alkoxyalkyl, thioalkyl, alkylthioalkyl, etc.), one or two aryl groups (as defined herein), one or two heteroaryl groups (as defined herein), one or two arylalkyl groups (as defined herein), one or two heteroarylalkyl groups (as defined herein), combinations of H, alkyl, aryl, heteroaryl, arylalkyl, and heteroarylalkyl groups. When the amino group has one or more alkyl, aryl, heteroaryl, arylalkyl, or heteroarylalkyl groups, the alkyl, aryl, heteroaryl, arylalkyl, or heteroarylalkyl groups can be unsubstituted or substituted. The terms “alkylamino” or “arylamino” refer to an amino group that has one or two alkyl or aryl substituents, respectively. The terms “arylalkylamino” or “heteroarylalkylamino” refer to an amino group that has one or two arylalkyl or heteroaryl alkyl groups, respectively.

The term “amino” can also include amino groups substituted with acyl groups such as —C(O)-alkyl, —C(O)-aryl, —C(O)-heteroaryl, —C(O)—O-alkyl, —C(O)—O-aryl, —C(O)—O-heteroaryl, —C(O)—N(R)-alkyl, —C(O)—N(R)-aryl, —C(O)—N(R)-heteroaryl; sulfonyl groups such as —S(O)₂-alkyl, —S(O)₂-aryl, —S(O)₂-heteroaryl, —S(O)₂—O-alkyl, —S(O)₂—O-aryl, —S(O)₂—O-heteroaryl, —S(O)₂—N(R)-alkyl, —S(O)₂—N(R)-aryl, —S(O)₂—N(R)-heteroaryl, etc. (wherein R is H, alkyl, aryl, heteroaryl). When the substituent on the amino group is an acyl group, the moiety can also be referred to as an “amido” group (i.e., when the acyl group is —C(O)-alkyl, —C(O)-aryl, or —C(O)-heteroaryl), a “urea” moiety (i.e., when the acyl group is —C(O)—N(R)-alkyl, —C(O)—N(R)-aryl, or —C(O)—N(R)-heteroaryl), or a “urethane” moiety (i.e., when the acyl group is —C(O)—O-alkyl, —C(O)—O-aryl, or —C(O)—O-heteroaryl).

Unless stated to the contrary, a substitutent is bound to a structure through the last named moiety of the substituent. For example, an “arylalkyl” substituent is bound to a structure through the “alkyl” moiety of the substituent.

The term “aminoalkyl”, as used herein, means an amino groups bonded to the parent moiety through an alkyl moiety (i.e., amino-alkyl-), wherein the amino and alkyl portions of the aminoalkyl are each as defined herein. Non-limiting examples of aminoalkyl include H₂N—(CH₂)₂—CH₂—, H₂N—(CH₂)₃—CH₂—, (CH₃)₂N—CH₂CH₂—, CH₃—O—CH₂CH₂NH—CH₂—, aryl-NH—(CH₂)₃—CH₂—, heteroaryl-NH—(CH₂)₃—CH₂—, H₂N—C(O)—NH—(CH₂)₂—CH₂—, etc. The term aminoalkyl can also refer to nitrogen containing heterocycles attached to an alkylene through the nitrogen atom of the heterocycle, e.g., pyrrolidine-CH₂—, piperidine-CH₂CH₂—, morpholine-CH₂CH₂—, etc.

The term “amido”, “aminoacyl”, or “aminocarbonyl”, as used herein, means amino-C(O)—, wherein the amino moiety is any amino as defined herein. Non-limiting examples of aminoacyl include phenyl-NH—C(O)—, piperazine-C(O)—, pyrrolidine-C(O)—, (CH₃—O—CH₂CH₂)₂N—C(O)—, pyridine-CH₂—NH—C(O)—, phenyl-CH₂—NH—C(O)—, etc.

The term “aminoacylalkyl”, as used herein, means amino-C(O)-alkyl-, wherein the amino-C(O) moiety and alkyl moiety are as defined herein.

The term “arylamino”, as used herein, means aryl-amino-, wherein the amino moiety is any amino as defined herein. Non-limiting examples of arylamino include phenyl-NH—, halo substituted phenyl-NH—, etc.

The term “heteroarylamino”, as used herein, means heteroaryl-amino-, wherein the amino moiety is any amino as defined herein. Non-limiting examples of arylamino include pyrimidine-NH—, halo substituted pyrimidine-NH—, haloalkyl substituted pyrimidine-NH—, etc.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis.

The term “aryl”, as used herein, means an aromatic monocyclic or multicyclic ring system comprising about 6 to about 14 carbon atoms, preferably about 6 to about 10 carbon atoms. Non-limiting examples of suitable aryl groups include phenyl, biphenyl, or naphthyl. The term aryl refers to unsubstituted aryl groups or aryl groups substituted with one or more substituents which may be the same or different. The aryl group can be substituted with one or more substituents, including but not limited to substituents selected from the group consisting of hydroxyl, thiol, amino, alkylamino, arylamino, alkoxy, aryloxy, nitro, cyano, sulfonic acid, halo (F, Cl, I, Br), carboxy, ester, acyl, alkyl (i.e., any of the alkyl groups described herein, such as methyl, ethyl, propyl, butyl, etc.), alkenyl (i.e., any of the alkenyl groups described hererein, such as vinyl, allyl, 1-propenyl, 1-butenyl, 2-butenyl, etc.), alkynyl (i.e., any of the alkynyl groups described herein, such as 1-ethynyl, 1-propynyl, 2-propynyl, etc.), haloalkyl (i.e., any of the haloalkyl groups described herein), sulfate, sulfonate, sulfonic esters and amides, phosphoric acid, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term “alkaryl” or “alkylaryl” refers to an alkyl group with an aryl substituent. In one embodiment, the “alk” or “alkyl” portion of the alkaryl is a lower alkyl group. Non-limiting examples of suitable alkylaryl groups include o-tolyl, p-tolyl and xylyl. The bond to the parent moiety is through the aryl.

The term “aralkyl” or “arylalkyl” refers to an aryl group attached to an alkyl group. In one embodiment, the “alk” or “alkyl” portion of the aralkyl is a lower alkyl group. Non-limiting examples of suitable aralkyl groups include benzyl, 2-phenethyl and naphthalenylmethyl. The bond to the parent moiety is through the alkyl. The aryl portion of the arylalkyl group may be substituted or unsubstituted.

The term “alkoxy”, as used herein, means alkyl-O—, wherein the alkyl moiety of the alkoxy group is an alkyl group as defined herein.

The term “cycloalkyl” means a non-aromatic mono- or multicyclic fused ring system comprising 3 to 10 ring carbon atoms, preferably 3 to 7 ring carbon atoms, more preferably 3 to 6 ring carbon atoms. The cycloalkyl can be optionally substituted with one or more substituents which may be the same or different. Non-limiting examples of suitable monocyclic cycloalkyls include cyclopropyl, cyclobutyl, cyclopentyl, cyclohexyl and the like. Non-limiting examples of suitable multicyclic cycloalkyls include 1-decalinyl, norbornenyl, adamantyl and the like. Suitable substituents for cycloalkyls include substituents selected from the group consisting of hydroxyl, thiol, amino, alkylamino, arylamino, alkoxy, aryloxy, nitro, cyano, sulfonic acid, halo (F, Cl, I, Br), carboxy, ester, acyl, alkyl (i.e., any of the alkyl groups described herein, such as methyl, ethyl, propyl, butyl, etc.), alkenyl (i.e., any of the alkenyl groups described hererein, such as vinyl, allyl, 1-propenyl, 1-butenyl, 2-butenyl, etc.), alkynyl (i.e., any of the alkynyl groups described herein, such as 1-ethynyl, 1-propynyl, 2-propynyl, etc.), haloalkyl (i.e., any of the haloalkyl groups described herein), sulfate, sulfonate, sulfonic esters and amides, phosphoric acid, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, herein incorporated by reference in its entirety. Substituents can also include fused aromatic rings, e.g.:

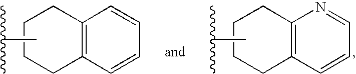

wherein the fused aromatic or heteroaromatic ring can itself be unsubstituted or substituted with one or more substituents as described herein.

The term “halo”, as used herein, includes chloro, bromo, iodo, and fluoro.

The term “haloalkyl”, as used herein, means an alkyl as defined above wherein one or more hydrogen atoms on the alkyl are replaced by a halo defined above. Non-limiting examples of haloalkyl groups include —CF₃, —CH₂CF₃, etc.

The term “hydroxyalkyl”, as used herein, means an alkyl group having at least one hydroxy substituent. Non-limiting examples of hydroxyalkyl groups include hydroxyethyl, 3-hydroxypropyl, 2-hydroxy propyl, etc.

The term “alkoxyalkyl”, as used herein, means alkyl-O-alkyl-, wherein each of the alkyl moieties is as defined herein. The skilled practitioner will recognize that a divalent alkyl group (i.e., an alkyl group bonded to two other moieties) can also be referred to as an “alkylene” group. An alkylene group is an alkyl group in which one of the C—H bonds is replaced with a covalent bond to another moiety. Non-limiting examples of alkoxyalkyl groups include CH₃—O—CH₂CH₂—, CH₃—O—CH₂CH₂CH₂—, CH₃CH₂—O—CH₂CH₂—, CH₃CH₂—O—CH₂CH₂CH₂—, t-Bu-O—CH₂CH₂—, etc.

The term “acyl” refers to a carbonyl group (—C(O)—). For example, arylacyl refers to groups such as phenyl-C(O)—, alkylacyl refers to acetyl, aminoacyl refers to H₂N—C(O)—(wherein the N atom can be substituted with aryl, alkyl, heterocyclyl, etc), etc. When the acyl group forms, for example, a ketone, a carboxy group, a carbonate group, a urea group, a thio ester, etc, the non-carbonyl moiety of the such a group is selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen, C₁ to C₄ alkyl or C₁ to C₄ alkoxy, sulfonate esters such as alkyl or aralkyl sulphonyl including methanesulfonyl, the mono, di or triphosphate ester, trityl or monomethoxytrityl, substituted benzyl, trialkylsilyl (e.g. dimethyl-t-butylsilyl) or diphenylmethylsilyl. In one embodiment, aryl groups in the esters comprise a phenyl group. The term “lower acyl” refers to an acyl group in which the non-carbonyl moiety is a lower alkyl.

The term “carboxy”, as used herein, means —C(O)OH or an ester thereof.

The term “alkoxycarbonyl”, as used herein, means —C(O)—O-alkyl, wherein the alkyl moiety is any alkyl as defined herein.

The term “alkylthioalkyl”, as used herein, means alkyl-5-alkyl-, wherein each of the alkyl moieties is as defined herein. Non-limiting examples of alkylthioalkyl groups include CH₃—S—CH₂CH₂—, CH₃—S—CH₂CH₂CH₂—, CH₃CH₂—S—CH₂CH₂—, CH₃CH₂—S—CH₂CH₂CH₂—, t-Bu-S—CH₂CH₂—, etc.

The term “alkylamino”, as used herein, means alkyl-amino-, wherein the amino moiety can be any amino as defined herein.

The term “a 5- to 18-membered saturated heterocyclic ring containing at least one nitrogen atom” means a saturated monocyclic or multicyclic ring system comprising 5 to 18 atoms as the members constituting the ring system wherein the 5 to 18 atoms are chosen from carbon, nitrogen, sulfur, or phosphorous and at least one of the 5 to 18 atoms are nitrogen. The term encompasses a 6-18 membered saturated heterocyclic ring containing one or more, e.g., 2 nitrogen atoms. The multicyclic ring system can be fused or bridged multicyclic rings. The 5- to 18-membered saturated heterocyclic ring can optionally be substituted at any substitutable position (including at a heteroatom) by groups including substituted or unsubstituted alkyl (e.g., hydroxyalkyl, haloalkyl, alkoxyalkyl, etc.), halo, hydroxyl, oxo, amino (as defined herein, e.g., —NH₂, amido, sulfonamido, urea moiety, urethane moiety), aminoacyl (as defined herein), aminoalkyl (as defined herein), amino-S(O)₂—, alkyl-S(O)₂—, arylamino (as defined herein), heteroarylamino (as defined herein), alkylamino (as defined herein), alkoxy, alkoxycarbonyl, aryloxy, nitro, cyano, aryl, heteroaryl, carboxy (as defined herein), sulfonic acid, sulfate, phosphonic acid, phosphate, or phosphonate, either unprotected, or protected as necessary, as known to those skilled in the art. Non-limiting examples of substituted or unsubstituted 5- to 18-membered saturated heterocyclic rings containing at least one nitrogen atom include the following:

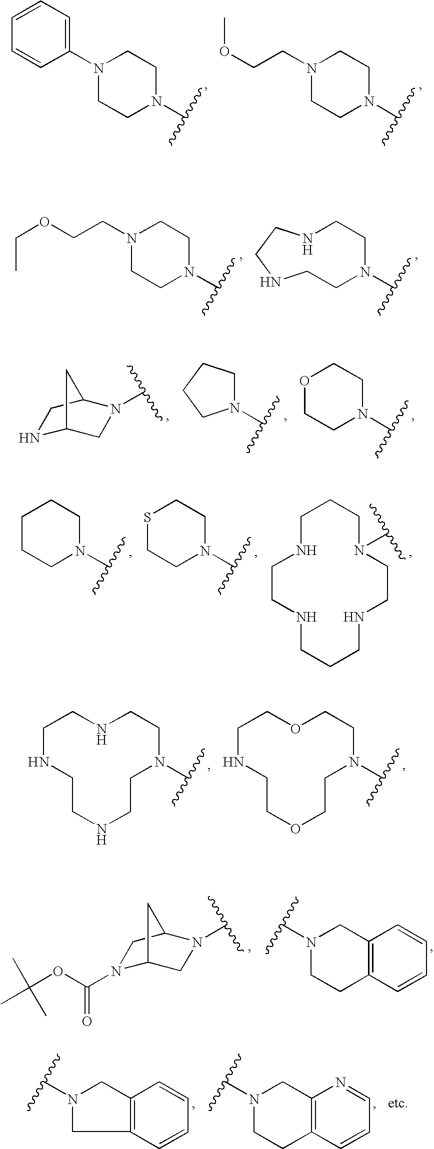

With reference to the number of moieties (e.g., substituents, groups or rings) in a compound, unless otherwise defined, the phrases “one or more” and “at least one” mean that there can be as many moieties as chemically permitted, and the determination of the maximum number of such moieties is well within the knowledge of those skilled in the art.

The term “pharmaceutically acceptable salt, solvate, ester or prodrug” is used throughout the specification to describe any pharmaceutically acceptable form (such as an ester, phosphate ester, salt of an ester or a related group, or hydrate) of a compound which, upon administration to a patient, provides the compound described in the specification. Pharmaceutically acceptable salts include those derived from pharmaceutically acceptable inorganic or organic bases and acids such as hydrochloric acid, hydrobromic acid, sulfuric acid, nitric acid, phosphoric acid, methanesulfonic acid, ethanesulfonic acid, p-toluensulfonic acid, salicylic acid, malic acid, maleic acid, succinic acid, tartaric acid, citric acid and the like. Suitable salts include those derived from alkali metals such as potassium and sodium, alkaline earth metals such as calcium and magnesium, among numerous other acids well known in the art, for example as described herein.

Prodrugs and solvates of the compounds of the invention are also contemplated herein. The term “prodrug”, as employed herein, denotes a compound that is a drug precursor (e.g., has one or more biologically labile protecting group(s) on a functional moiety of the active compound) which, upon administration to a subject, undergoes chemical conversion by metabolic or chemical processes (e.g., oxidation, reduction, amidation, deamination, hydroxylation, dehydroxylation, hydrolysis, dehydrolysis, alkylation, dealkylation, acylation, deacylation, phosphorylation, dephosphorylation, etc.) to yield an active compound or a salt and/or solvate thereof. A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

The term “solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

The term “heterocyclic” or “heterocyclyl” refers to a cyclic group that may be unsaturated, partially or fully saturated and wherein there is at least one heteroatom, such as oxygen, sulfur, nitrogen, or phosphorus in the ring. Heterocyclic or heterocyclyl groups include heteroaryl groups. Non-limiting examples of non-aromatic heterocyclyls include piperidinyl, piperazinyl, morpholinyl, pyrrolidinyl, morpholino, thiomorpholino, oxiranyl, pyrazolinyl, dioxolanyl, 1,4-dioxanyl, aziridinyl, tetrahydrofuranyl, pyrrolinyl dihydrofuranyl, dioxanyl, tetrahydropyranyl, dihydropyranyl, indolinyl, imidazolyl, tetraazacyclotetradecanyl, dioxadiazacyclododecanyl, diazepanyl, etc., wherein each of the aforementioned heterocyclyls can be unsubstituted or substituted at any substitutable position (including a heteroatom) with one or more substituents.

The term “heteroaryl” or “heteroaromatic”, as used herein, refers to an aromatic ring that includes at least one sulfur, oxygen, nitrogen or phosphorus in the aromatic ring. Nonlimiting examples of heteroaromatics are furanyl, pyridyl, pyrimidinyl, benzoxazolyl, 1,2,4-oxadiazolyl, 1,3,4-oxadiazolyl, 1,3,4-thiadiazole, indazolyl, 1,3,5-triazinyl, thienyl, tetrazolyl, benzofuranyl, quinolyl, isoquinolyl, benzothienyl, isobenzofuryl, indolyl, isoindolyl, benzimidazolyl, purine, carbazolyl, oxazolyl, thiazolyl, benzothiazolyl, isothiazolyl, 1,2,4-thiadiazolyl, isooxazolyl, pyrrolyl, quinazolinyl, cinnolinyl, phthalazinyl, xanthinyl, hypoxanthinyl, pyrazole, 1,2,3-triazole, 1,2,4-triazole, 1,2,3-oxadiazole, thiazine, pyridazine, benzothiophenyl, isopyrrole, thiophene, pyrazine, or pteridinyl wherein said heteroaryl or heterocyclic group can be optionally substituted with one or more substituent. In one embodiment, heterocyclyl and heteraromatic groups include purine and pyrimidines.

Substituted aromatic or heteroaromatic rings (including aromatic or heteroaromatic portions of functional groups such as arylalkyl or heteroarylalkyl groups) can be substituted with one or more substituents. Non-limiting examples of such substituents selected from the group consisting of hydroxyl, thiol, amino, alkylamino, arylamino, alkoxy, aryloxy, nitro, cyano, sulfonic acid, halo (F, Cl, I, Br), carboxy, ester, acyl, alkyl (i.e., any of the alkyl groups described herein, such as methyl, ethyl, propyl, butyl, etc.), alkenyl (i.e., any of the alkenyl groups described hererein, such as vinyl, allyl, 1-propenyl, 1-butenyl, 2-butenyl, etc.), alkynyl (i.e., any of the alkynyl groups described herein, such as 1-ethynyl, 1-propynyl, 2-propynyl, etc.), haloalkyl (i.e., any of the haloalkyl groups described herein), sulfate, sulfonate, sulfonic esters and amides, phosphoric acid, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

Functional oxygen and nitrogen groups (e.g., on a aryl or heteroaryl group) can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acycl groups such as acetyl and propionyl, methanesulfonyl, and p-toluenelsulfonyl.

The term “heteroarylalkyl”, as used herein, means heteroaryl-alkyl-, wherein the heteroaryl and alkyl moieties can be any heteroaryl or alkyl defined herein. Non-limiting examples of heteroarylalkyl include pyridine-methyl- and benzimidazole-methyl-.

The term “heterocyclylalkyl”, as used herein, means heterocyclyl-alkyl-, wherein the alkyl moiety may attach to the heterocyclyl ring at any available position, and the heterocyclyl and alkyl moieties can be any heterocyclyl or alkyl defined herein. Non-limiting examples of heteroarylalkyl include pyrrolidine-methyl- and piperidine-methyl.

The term “arylacyl”, as used herein, means —C(O)-aryl, wherein the aryl moiety is any aryl as defined herein.

The term “heteroarylacyl”, as used herein, means —C(O)-heteroaryl, wherein the heteroaryl moiety is any heteroaryl as defined herein.

The term purine or pyrimidine includes, but is not limited to, adenine, N₆-alkylpurines, N₆-acylpurines (wherein acyl is C(O)(alkyl, aryl, alkylaryl, or arylalkyl), N₆-benzylpurine, N₆-halopurine, N₆-vinylpurine, N₆-acetylenic purine, N₆-acyl purine, N₆-hydroxyalkyl purine, N₆-thioalkyl purine, N₂-alkylpurines, N₂-alkyl-6-thiopurines, thymine, cytosine, 5-fluorocytosine, 5-methylcytosine, 6-azapyrimidine, including 6-azacytosine, 2- and/or 4-mercaptopyrmidine, uracil, 5-halouracil, including 5-fluorouracil, C₅-alkylpyrimidines, C₅-benzylpyrimidines, C₅-halopyrimidines, C₅-vinylpyrimidine, C₅-acetylenic pyrimidine, C₅-acyl pyrimidine, C₅-hydroxyalkyl purine, C₅-amidopyrimidine, C₅-cyanopyrimidine, C₅-nitropyrimidine, C₅-aminopyrimidine, N₂-alkylpurines, N₂-alkyl-6-thiopurines, 5-azacytidinyl, 5-azauracilyl, triazolopyridinyl, imidazolopyridinyl, pyrrolopyrimidinyl, and pyrazolopyrimidinyl. Purine bases include, but are not limited to, guanine, adenine, hypoxanthine, 2,6-diaminopurine, and 6-chloropurine.

Compounds of the present invention, and salts, solvates and prodrugs thereof, may exist in their tautomeric form (for example, as an amide or imino ether). All such tautomeric forms are contemplated herein as part of the present invention.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, racemates or prodrugs of the inventive compounds.

Polymorphic forms of the compounds of the present invention, and of the salts, solvates and/or prodrugs of the compounds of the present invention, are intended to be included in the present invention.

The compounds of the present are those compounds of formula (I), or pharmaceutically acceptable salts, solvates, prodrugs, tautomers, stereioisomers, and esters thereof, having sufficient chemical stability for formulation in a pharmaceutical composition. It should also be noted that any carbon or heteroatom with unsatisfied valences in the text, schemes, examples and Tables herein is assumed to have the hydrogen atom(s) to satisfy the valences.

Formulations

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates) undecanoates, and the like. Additionally, acids which are generally considered suitable for the formation of pharmaceutically useful salts from basic pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine and the like. Basic nitrogen-containing groups may be quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

The active compound can also be provided as a prodrug, which is converted into a biologically active form in vivo. A prodrug may be converted into the parent drug by various mechanisms, including enzymatic processes and metabolic hydrolysis: T. Higuchi and V. Stella, Pro-drugs as Novel Delivery Systems, Vol. 14 of A.C.S. Symposium Series (1987) Harper, N.J. (1962) in Jucker, ed. Progress in Drug Research, 4:221-294; Morozowich et al. (1977) in E. B. Roche ed. Design of Biopharmaceutical Properties through Prodrugs and Analogs, APhA (Acad. Pharm. Sci.); E. B. Roche, ed. (1977) Bioreversible Carriers in Drug in Drug Design, Theory and Application, APhA; H. Bundgaard, ed. (1985) Design of Prodrugs, Elsevier; Wang et al. (1999) Curr. Pharm. Design. 5(4):265-287; Pauletti et al. (1997) Adv. Drug. Delivery Rev. 27:235-256; Mizen et al. (1998) Pharm. Biotech. 11:345-365; Gaignault et al. (1996) Pract. Med. Chem. 671-696; M. Asghamejad (2000) in G. L. Amidon, P. I. Lee and E. M. Topp, Eds., Transport Proc. Pharm. Sys., Marcell Dekker, p. 185-218; Balant et al. (1990) Eur. J. Drug Metab. Pharmacokinet., 15(2): 143-53; Balimane and Sinko (1999) Adv. Drug Deliv. Rev., 39(1-3):183-209; Browne (1997). Clin. Neuropharm. 20(1): 1-12; Bundgaard (1979) Arch. Pharm. Chemi. 86(1): 1-39; H. Bundgaard, ed. (1985) Design of Prodrugs, New York: Elsevier; Fleisher et al. (1996) Adv. Drug Delivery Rev, 19(2): 115-130; Fleisher et al. (1985) Methods Enzymol. 112: 360-81; Farquhar D, et al. (1983) J. Pharm. Sci., 72(3): 324-325; Han, H. K. et al. (2000) AAPS Pharm Sci., 2(1): E6; Sadzuka Y. (2000) Curr. Drug Metab., 1:31-48; D. M. Lambert (2000) Eur. J. Pharm. Sci., 11 Suppl 2:S1 5-27; Wang, W. et al. (1999) Curr. Pharm. Des., 5(4):265, each of which is incorporated herein by reference in its entirety.

The active compound can also be provided as a lipid prodrug. Nonlimiting examples of U.S. patents that disclose suitable lipophilic substituents that can be covalently incorporated into the compound or in lipophilic preparations, include U.S. Pat. Nos. 5,149,794 (Sep. 22, 1992, Yatvin et al.); 5,194,654 (Mar. 16, 1993, Hostetler et al., 5,223,263 (Jun. 29, 1993, Hostetler et al.); 5,256,641 (Oct. 26, 1993, Yatvin et al.); 5,411,947 (May 2, 1995, Hostetler et al.); 5,463,092 (Oct. 31, 1995, Hostetler et al.); 5,543,389 (Aug. 6, 1996, Yatvin et al.); 5,543,390 (Aug. 6, 1996, Yatvin et al.); 5,543,391 (Aug. 6, 1996, Yatvin et al.); and 5,554,728 (Sep. 10, 1996; Basava et al.).

Preferably, the pharmaceutical preparation is in a unit dosage form. In such form, the preparation is subdivided into suitably sized unit doses containing appropriate quantities of the active compound, e.g., an effective amount to achieve the desired purpose.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 0.1 to about 95 percent active compound. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Method of Treatment

The compounds described herein, are particularly useful for the treatment or prevention of a disorder associated with chemokine receptor binding or activation, and particularly for the treatment of HIV or AIDS in a host in need thereof.

In one embodiment, a method of treating or preventing HIV infection or reduction of symptoms associated with AIDS is provided including administering a compound of at least one of Formula (I)-(V) to a host. In certain embodiments, the compound can be provided to a host before treatment of infection with another compound. In a separate embodiment, the compound is provided to a patient that has been treated for HIV infection to reduce the likelihood of recurrence, or reduce mortality associated with AIDS related symptoms. In another embodiment, the compound is administered to a host at high risk of suffering from HIV infections.

Hosts, including humans suffering from, or at risk for, HIV infection can be treated by administering an effective amount of the active compound or a pharmaceutically acceptable prodrug or salt thereof in the presence of a pharmaceutically acceptable carrier or diluent.

The administration can be prophylactically for the prevention of HIV infection or reduction of symptoms associated with AIDS. The active materials can be administered by any appropriate route, for example, orally, parenterally, intravenously, intradermally, subcutaneously, or topically, in liquid or solid form. However, the compounds are particularly suited to oral delivery.

An exemplary dose of the compound will be in the range from about 1 to 50 mg/kg, preferably 1 to 20 mg/kg, of body weight per day, more generally 0.1 to about 100 mg per kilogram body weight of the recipient per day. The effective dosage range of the pharmaceutically acceptable salts and prodrugs can be calculated based on the weight of the parent compound to be delivered. If the salt, ester or prodrug exhibits activity in itself, the effective dosage can be estimated as above using the weight of the salt, ester, solvate, or prodrug, or by other means known to those skilled in the art.

The amount and frequency of administration of the compounds of the invention and/or the pharmaceutically acceptable salts thereof will be regulated according to the judgment of the attending clinician considering such factors as age, condition and size of the patient as well as the condition and/or severity of the symptoms being treated. A typical recommended daily dosage regimen for oral administration can range from about 0.1 mg/day to about 2000 mg/day, in one to four divided doses.

In a separate embodiment, a method for the treatment or prevention of HIV infection or reduction of symptoms associated with AIDS by administering a compound of the present invention, or a pharmaceutically acceptable salt, solvate, prodrug, or ester thereof to a host in need of treatment is provided. The compounds of the invention, or a pharmaceutically acceptable salt, solvate, prodrug, or ester thereof can be administered to a host in need thereof to reduce the severity of AIDS related disorders. In one embodiment of the invention, the host is a human.

In another embodiment, the invention provides a method of treating symptoms associated with other infections associated with chemokine receptor activation, for example, liver diseases associated with flavivirus or pestivirus infection, and in particular, HCV or HBV, by contacting a cell with a compound of the present invention, or a pharmaceutically acceptable salt, solvate, prodrug, or ester thereof. The cell can be in a host animal, in particular in a human.

The compounds can treat or prevent HIV infection, or reduce the severity of AIDS related symptoms and diseases in any host. However, typically the host is a mammal and more typically is a human. In certain embodiments the host has been diagnosed with AIDS prior to administration of the compound, however in other embodiments, the host is merely infected with HIV and asymptomatic.

Generally, the disclosure provides compositions and methods for treating or preventing a chemokine receptor mediated pathology by administering a compound of the present invention, or a pharmaceutically acceptable salt, solvate, prodrug, or ester thereof to a host in a therapeutic amount, for example in an amount sufficient to inhibit chemokine signal transduction in a cell expressing a chemokine receptor or homologue thereof.

Another embodiment provides uses of a compound of the present invention, or a pharmaceutically acceptable salt, solvate, prodrug, or ester thereof for the treatment of, or for the manufacture of a medicament for the treatment of chemokine receptor mediated pathologies including, but not limited to cancer. Still another embodiment provides uses of a chemokine peptide antagonist for the manufacture of medicament for the prevention of tumor cell metastasis in a mammal.

The compounds, or pharmaceutically acceptable salts, solvates, prodrugs, or esters thereof of the present invention described herein can be used to treat or prevent cancer, in particular the spread of cancer within an organism. Cancer is a general term for diseases in which abnormal cells divide without control. Cancer cells can invade nearby tissues and can spread through the bloodstream and lymphatic system to other parks of the body. It has been discovered that the administration of a chemokine receptor antagonist to a host, for example a mammal, inhibits or reduces the metastasis of tumor cells, in particular breast cancer and prostate cancer.

There are several main types of cancer, and the disclosed compounds or compositions can be used to treat any type of cancer. For example, carcinoma is cancer that begins in the skin or in tissues that line or cover internal organs. Sarcoma is cancer that begins in bone, cartilage, fat, muscle, blood vessels, or other connective or supportive tissue.

Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. A solid tumor is an abnormal mass of tissue that usually does not contain cysts or liquid areas. A single tumor may even have different populations of cells within it with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors. The compositions described herein can be used to reduce, inhibit, or diminish the proliferation of tumor cells, and thereby assist in reducing the size of a tumor.

Representative cancers that may treated with the disclosed compositions and methods include, but are not limited to, bladder cancer, breast cancer, colorectal cancer, endometrial cancer, head & neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, ependymoma, Ewing's sarcoma family of tumors, germ cell tumor, extracranial cancer, Hodgkin's disease, leukemia, acute lymphoblastic leukemia, acute myeloid leukemia, liver cancer, medulloblastoma, neuroblastoma, brain tumors generally, non-Hodgkin's lymphoma, ostessarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, small-cell lung cancer, among others.

A tumor can be classified as malignant or benign. In both cases, there is an abnormal aggregation and proliferation of cells. In the case of a malignant tumor, these cells behave more aggressively, acquiring properties of increased invasiveness.

Ultimately, the tumor cells may even gain the ability to break away from the microscopic environment in which they originated, spread to another area of the body (with a very different environment, not normally conducive to their growth) and continue their rapid growth and division in this new location. This is called metastasis. Once malignant cells have metastasized, achieving cure is more difficult.

Benign tumors have less of a tendency to invade and are less likely to metastasize. They do divide in an uncontrolled manner, though. Depending on their location, they can be just as life threatening as malignant lesions. An example of this would be a benign tumor in the brain, which can grow and occupy space within the skull, leading to increased pressure on the brain. The compositions provided herein can be used to treat benign or malignant tumors.

Pharmaceutical Compositions

In one embodiment, pharmaceutical compositions including at least one compound of the present invention, or a pharmaceutically acceptable salt, solvate, prodrug, or ester thereof is provided. In certain embodiments, at least a second active compound is administered in combination or alternation with the first compound.

The second active compound can be an antiviral, particularly an agent active against a HIV and in a particular embodiment, active against HIV-1. Hosts, including humans suffering from or at risk of contracting HIV can be treated by administering an effective amount of a pharmaceutical composition of the active compound.

In another embodiment, the second active compound can be a chemotherapeutic agent, for example an agent active against a primary tumor. Hosts, including humans suffering from or at risk for a proliferative disorder can be treated by administering an effective amount of a pharmaceutical composition of the active compound.

The compound of the present invention, or a pharmaceutically acceptable salt, solvate, prodrug, or ester thereof is conveniently administered in unit any suitable dosage form, including but not limited to one containing 7 to 3000 mg, preferably 70 to 1400 mg of active ingredient per unit dosage form. A oral dosage of 50-1000 mg is usually convenient. Ideally the active ingredient should be administered to achieve peak plasma concentrations of the active compound of from about 1 μM to 100 mM or from 0.2 to 700 μM, or about 1.0 to 10 μM.

The concentration of active compound in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

A preferred mode of administration of the active compound is oral. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compound can be administered as a component of an elixir, suspension, syrup, wafer, chewing gum or the like. A syrup may contain, in addition to the active compounds, sucrose as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories, or antiviral compounds, or with additional chemotherapeutic agents. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

In a preferred embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art. The materials can also be obtained commercially from Alza Corporation. If administered intravenously, preferred carriers are physiological saline or phosphate buffered saline (PBS).

Liposomal suspensions (including liposomes targeted to infected cells with monoclonal antibodies to viral antigens) are also preferred as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives is then introduced into the container. The container is then swirled by hand to free lipid material from the sides of the container and to disperse lipid aggregates, thereby forming the liposomal suspension.

Combination and Alternation Therapy

In one embodiment, the compounds described herein are administered in combination or alternation with another active compound.

In another embodiment, the second active compound is a compound that is used as an anti-HIV agent, including but not limited to a nucleoside or nonnucleoside reverse transcriptase inhibitor, a protease inhibitor, a fusion inhibitor, cytokine and interferon. The compound provided in combination or alternation can, as a nonlimiting example, be selected from the following lists:

| Brand Name | Generic Name |
|---|---|
| Agenerase | amprenavir |
| Combivir | lamivudine and zidovudine |
| Crixivan | indinavir, IDV, MK-639 |
| Emtriva | FTC, emtricitabine |
| Epivir | lamivudine, 3TC |
| Epzicom | abacavir/lamivudine |
| Fortovase | saquinavir |
| Fuzeon | enfuvirtide, T-20 |
| Hivid | zalcitabine, ddC, dideoxycytidine |
| Invirase | saquinavir mesylate, SQV |
| Kaletra | lopinavir and ritonavir |
| Lexiva | Fosamprenavir Calcium |
| Norvir | ritonavir, ABT-538 |
| Rescriptor | delavirdine, DLV |
| Retrovir | zidovudine, AZT, azidothymidine, ZDV |
| Reyataz | atazanavir sulfate |
| Sustiva | efavirenz |
| Trizivir | abacavir, zidovudine, and lamivudine |
| Truvada | tenofovir disoproxil/emtricitabine |
| Videx EC | enteric coated didanosine |
| Videx | didanosine, ddI, dideoxyinosine |
| Viracept | nelfinavir mesylate, NFV |
| Viramune | nevirapine, BI-RG-587 |
| Viread | tenofovir disoproxil fumarate |
| Zerit | stavudine, d4T |
| Ziagen | abacavir |

Further active agents include: GW5634 (GSK), (+)Calanolide A (Sarawak Med.), Capravirine (Agouron), MIV-150 (Medivir/Chiron), TMC125 (Tibotec), RO033-4649 (Roche), TMC114 (Tibotec), Tipranavir (B-1), GW640385 (GSK/Vertex), Elvucitabine (Achillion Ph.), Alovudine (FLT) (B-I), MIV-210 (GSK/Medivir), Racivir (Pharmasset), SPD754 (Shire Pharm.), Reverset (Incyte Corp.), FP21399 (Fuji Pharm.), AMD070 (AnorMed), GW873140 (GSK), BMS-488043 (BMS), Schering C/D (417690), PRO542 (Progenics Pharm), TAK-220 (Takeda), TNX-355 (Tanox), UK-427,857 (Pfizer).

Further active agents include: Attachment and Fusion Inhibitors (i.e. AMD070, BMS-488043, FP21399, GW873140, PRO542, Schering C, SCH 417690, TAK-220, TNX-355 and UK-427,857); Integrase Inhibitors; Maturation Inhibitors (i.e. PA457); Zinc Finger Inhibitors (i.e. azodicarbonamide (ADA)); Antisense Drugs (i.e. HGTV43 by Enzo Therapeutics, GEM92 by Hybridon); Immune Stimulators (i.e. Ampligen by Hemispherx Biopharma, IL-2 (Proleukin) by Chiron Corporation, Bay 50-4798 by Bayer Corporation, Multikine by Cel-Sci Corporation, IR103 combo); Vaccine-Like Treatment (i.e. HRG214 by Virionyx, DermaVir, VIR201 (Phase I/IIa)).

In one embodiment, the compounds of the invention are administered in combination with another active agent. The compounds can also be administered concurrently with the other active agent. In this case, the compounds can be administered in the same formulation or in a separate formulation. There is no requirement that the compounds be administered in the same manner. For example, the second active agent can be administered via intravenous injection while the compounds of the invention may be administered orally. In another embodiment, the compounds of the invention are administered in alternation with at least one other active compound. In a separate embodiment, the compounds of the invention are administered during treatment with an active agent, such as, for example, an agent listed above, and administration of the compounds of the invention is continued after cessation of administration of the other active compound.

The compounds of the invention can be administered prior to or after cessation of administration of another active compound. In certain cases, the compounds may be administered before beginning a course of treatment for viral infection or for secondary disease associated with HIV infections, for example. In a separate embodiment, the compounds can be administered after a course of treatment to reduce recurrence of viral infections.

In another embodiment, the active compound is a compound that is used as a chemotherapeutic. A compound provided in combination or alternation can, for example, be selected from the following list:

| | | | |
|---|---|---|---|
| 13-cis-Retinoic Acid | 2-Amino-6-Mercaptopurine | 2-CdA | 2-Chlorodeoxyadenosine |
| 5-fluorouracil | 5-FU | 6-TG | 6-Thioguanine |
| 6-Mercaptopurine | 6-MP | Accutane | Actinomycin-D |
| Adriamycin | Adrucil | Agrylin | Ala-Cort |
| Aldesleukin | Alemtuzumab | Alitretinoin | Alkaban-AQ |
| Alkeran | All-transretinoic acid | Alpha interferon | Altretamine |
| Amethopterin | Amifostine | Aminoglutethimide | Anagrelide |
| Anandron | Anastrozole | Arabinosylcytosine | Ara-C |
| Aranesp | Aredia | Arimidex | Aromasin |
| Arsenic trioxide | Asparaginase | ATRA | Avastin |
| BCG | BCNU | Bevacizumab | Bexarotene |
| Bicalutamide | BiCNU | Blenoxane | Bleomycin |
| Bortezomib | Busulfan | Busulfex | C225 |
| Calcium Leucovorin | Campath | Camptosar | Camptothecin-11 |
| Capecitabine | Carac | Carboplatin | Carmustine |
| Carmustine wafer | Casodex | CCNU | CDDP |
| CeeNU | Cerubidine | cetuximab | Chlorambucil |
| Cisplatin | Citrovorum Factor | Cladribine | Cortisone |
| Cosmegen | CPT-11 | Cyclophosphamide | Cytadren |
| Cytarabine | Cytarabine liposomal | Cytosar-U | Cytoxan |
| Dacarbazine | Dactinomycin | Darbepoetin alfa | Daunomycin |
| Daunorubicin | Daunorubicin hydrochloride | Daunorubicin liposomal | DaunoXome |
| Decadron | Delta-Cortef | Deltasone | Denileukin diftitox |
| DepoCyt | Dexamethasone | Dexamethason Acetate | dexamethasone sodium phosphate |
| Dexasone | Dexrazoxane | DHAD | DIC |
| Diodex | Docetaxel | Doxil | Doxorubicin |
| Doxorubicin liposomal | Droxia | DTIC | DTIC-Dome |
| Duralone | Efudex | Eligard | Ellence |
| Eloxatin | Elspar | Emcyt | Epirubicin |
| Epoetin alfa | Erbitux | Erwinia-L-asparaginase | Estramustine |
| Ethyol | Etopophos | Etoposide | Etoposide phosphate |
| Eulexin | Evista | Exemestane | Fareston |
| Faslodex | Femara | Filgrastim | Floxuridine |
| Fludara | Fludarabine | Fluoroplex | Fluorouracil |
| Fluorouracil (cream) | Fluoxyrnesterone | Flutamide | Folinic Acid |
| FUDR | Fulvestrant | G-CSF | Gefitinib |
| Gemcitabine | Gemtuzumab ozogamicin | Gemzar | Gleevec |
| Gliadel wafer | Glivec | GM-CSF | Goserelin |
| granulocyte colony | Granulocyte macrophage colony | Halotestin | Herceptin |
| stimulating factor | stimulating factor | | |
| Hexadrol | Hexalen | Hexamethylmelamine | HMM |
| Hycamtin | Hydrea | Hydrocort Acetate | Hydrocortisone |
| Hydrocortisone sodium phosphate | Hydrocortisone sodium succinate | Hydrocortone phosphate | Hydroxyurea |
| Ibritumomab | Ibritumomab Tiuxetan | Idamycin | Idarubicin |
| Ifex | IFN-alpha | Ifosfamide | IL - 2 |
| IL-11 | Imatinib mesylate | Imidazole Carboxamide | Interferon alfa |
| Interferon Alfa-2b | Interleukin - 2 | Interleukin- 11 | Intron A (interferon |
| (PEG conjugate) | | | alfaL2b) |
| Iressa | Irinotecan | Isotretinoin | Kidrolase |
| Lanacort | L-asparaginase | LCR | Letrozole |
| Leucovorin | Leukeran | Leukine | Leuprolide |
| Leurocristine | Leustatin | Liposomal Ara-C | Liquid Pred |
| Lomustine | L-PAM | L-Sarcolysin | Lupron |
| Lupron Depot | Matulane | Maxidex | Mechlorethamine |
| Mechlorethamine hydrochloride | Medralone | Medrol | Megace |
| Megestrol | Megestrol Acetate | Melphalan | Mercaptopurine |
| Mesna | Mesnex | Methotrexate | Methotrexate Sodium |
| Methylprednisolone | Meticorten | Mitomycin | Mitomycin-C |
| Mitoxantrone | M-Prednisol | MTC | MTX |
| Mylocel | Mylotarg | Navelbine | Neosar |
| Neulasta | Neumega | Neupogen | Nilandron |
| Nilutamide | Nitrogen Mustard | Novaldex | Novantrone |
| Octreotide | Octreotide acetate | Oncospar | Oncovin |
| Ontak | Onxal | Oprevelkin | Orapred |
| Orasone | Oxaliplatin | Paclitaxel | Pamidronate |
| Panretin | Paraplatin | Pediapred | PEG Interferon |
| Pegaspargase | Pegfilgrastim | PEG-INTRON | PEG-L-asparaginase |
| Phenylalanine Mustard | Platinol | Platinol-AQ | Prednisolone |
| Prednisone | Prelone | Procarbazine | PROCRIT |
| Proleukin | Prolifeprospan 20 | Purinethol | Raloxifene |
| | with Carmustine implant | | |
| Rheumatrex | Rituxan | Rituximab | Roveron-A (interferon α-2a) |
| Rubex | Rubidomycin hydrochloride | Sandostatin | Sandostatin LAR |
| Sargramostim | Solu-Cortef | Solu-Medrol | STI-571 |
| Streptozocin | Tamoxifen | Targretin | Taxol |
| Taxotere | Temodar | Temozolomide | Teniposide |
| TESPA | Thalidomide | Thalomid | TheraCys |
| Thioguanine | Thioguanine Tabloid | Thiophosphoamide | Thioplex |
| Thiotepa | TICE | Toposar | Topotecan |
| Toremifene | Trastuzumab | Tretinoin | Trexall |
| Trisenox | TSPA | VCR | Velban |
| Velcade | VePesid | Vesanoid | Viadur |
| Vinorelbine | Vinorelbine tartrate | VLB | VM-26 |
| VP- 16 | Vumon | Xeloda | Zanosar |
| Zevalin | Zinecard | Zoladex | Zoledronic acid |
| Zometa | | | |

Diseases

The compounds described herein, are particularly useful for the treatment or prevention of a disorder associated with chemokine receptor binding or activation, and particularly HIV viral infections. However, numerous other diseases have been associated with chemokine receptor signaling.

Human and simian immunodeficiency viruses (HIV and SIV, respectively) enter cells through a fusion reaction triggered by the viral envelope glycoprotein (Env) and two cellular molecules: CD4 and a chemokine receptor, generally either CCR5 or CXCR5. (Alkhatib G, Combadiere C, Croder C, Feng Y, Kennedy P E, Murphy P M, Berger E A. CC CKR5. a RANTES, MIP-1alpha, MIP-1Beta receptor as a fusion cofactor for macrophage-tropic HIV-1. Science. 1996; 272: 1955-1988).

In approximately 50% of infected individuals, CXCR4-tropic (X4-tropic) viruses emerge later in HIV infection, and their appearance correlates with a more rapid CD4 decline and a faster progression to AIDS (Connor, et al. (1997) J Exp. Med. 185: 621-628). Dualtropic isolates that are able to use both CCR5 and CXCR4 are also seen and may represent intermediates in the switch from CCR5 to CXCR4 tropism (Doranz, et al. (1996) Cell. 85: 1149-1158).

In a separate embodiment, a method for the treatment of, prevention of, or reduced severity of liver disease associated with viral infections including administering at least one compound described herein is provided.

Chronic hepatitis C virus (HCV) and hepatitis B virus (HBC) infection is accompanied by inflammation and fibrosis eventually leading to cirrhosis. A study testing the expression and function of CXCR4 on liver-infiltrating lymphocytes (LIL) revealed an important role for the CXCL12/CXCR4 pathway in recruitment and retention of immune cells in the liver during chronic HCV and HBV infection (Wald, et al. (2004) European Journal of Immunology. 34(4): 1164-1174).

High levels of CXCR4 and TGF-. have been detected in liver samples obtained from patients infected with HCV. (Mitra, et al. (1999) Int. J. Oncol. 14: 917-925). In vitro, TGF-. has been shown to up-regulate the expression of CXCR4 on naïve T cells and to increase their migration. The CD69/TGF-./CXCR4 pathway may be involved in the retention of recently activated lymphocytes in the liver (Wald, et al. European Journal of Immunology. 2004; 34(4): 1164-1174).

### EXAMPLES

### First aspect

Compounds of formula (I), wherein L² is CH₂, L¹ is M¹-N(R⁵)-M², and X and Y are both hydrogen, can have the following general structure (IA):

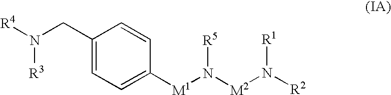

wherein M¹, M², R⁶, R¹, R², and —NR³R⁴ are defined herein below in Table 1. As shown below in Table 1, when M¹ is “-”, it denotes a covalent bond.

TABLE 1

| Cmpd. | —NR³R⁴ | M¹ | R⁵ | M² | R¹ | R² |
|---|---|---|---|---|---|---|
| A | 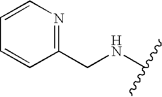 | CH₂ | H | C(O) | H | 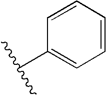 |
| B | 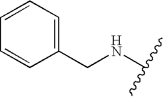 | CH₂ | H | C(O) | H | 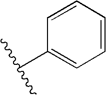 |
| C | 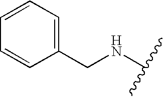 | — | H | C(O) | H | benzyl |
| D | 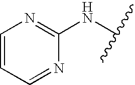 | — | H | C(O) | H | benzyl |
| E | 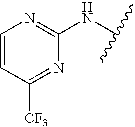 | — | H | C(O) | H | benzyl |
| F | 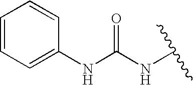 | CH₂ | 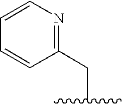 | C(O) | H | 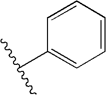 |
| G | 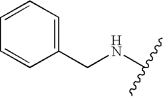 | — | H | C(O) | H | 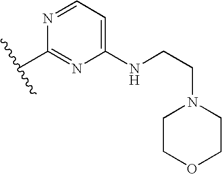 |
| H | 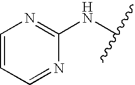 | — | H | C(O) | H | 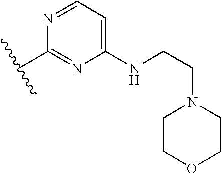 |
| I | 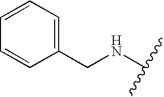 | — | H | C(O) | H | 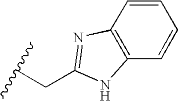 |
| J | 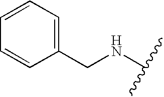 | CH₂CH₂ | 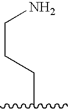 | C(O) | H | 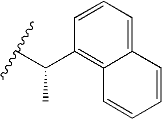 |
| K | 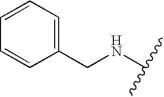 | CH₂CH₂ | 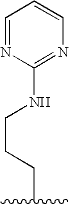 | C(O) | H | 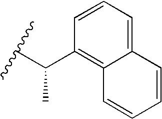 |
| L | 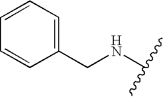 | — | H | C(O) | H | 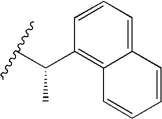 |
| M | 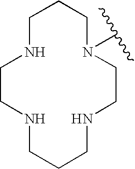 | — | H | C(O) | H | 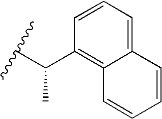 |
| N | 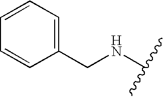 | — | benzyl | C(O) | H | 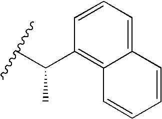 |
| O | 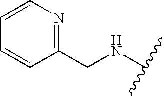 | CH₂ | benzyl | C(O) | H | 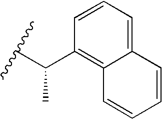 |
| P | 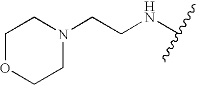 | — | H | C(O) | H | 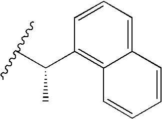 |
| Q | 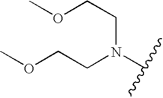 | CH₂ | H | C(O) | H | 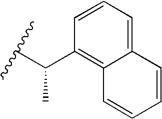 |
| R | 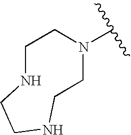 | CH₂ | H | C(O) | H | 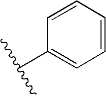 |
| S | 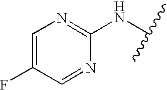 | — | H | C(O) | H | 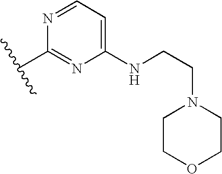 |
| T | 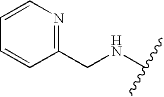 | — | H | C(O) | benzyl | 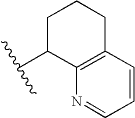 |
| U | 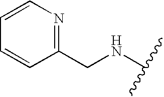 | — | H | C(O) | H | 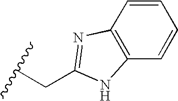 |
| V | 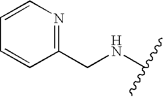 | CH₂CH₂ | 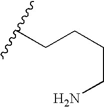 | C(O) | H | 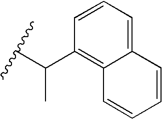 |
| W | 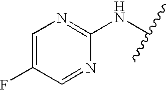 | — | H | C(O) | H | 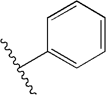 |
| X | 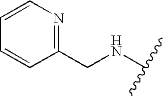 | — | H | C(O) | H | 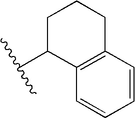 |
| Y | 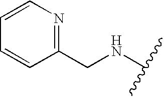 | — | benzyl | C(O) | H | 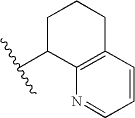 |
| Z | 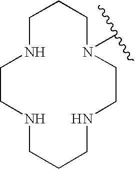 | — | H | C(O— | H | 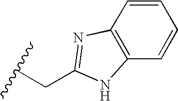 |
| AA | 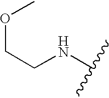 | CH₂CH₂ | 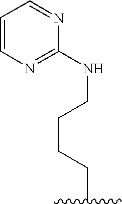 | C(O) | H | 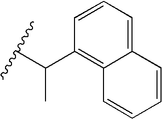 |
| AB | 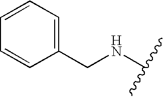 | CH₂ | H | S(O₂) | H | 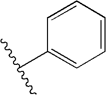 |
| AC | 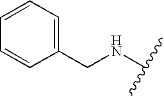 | CH₂ | 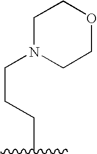 | C(O) | H | 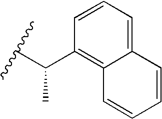 |
| AD | 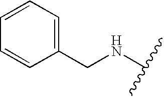 | CH₂ | 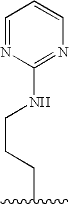 | C(O) | H | 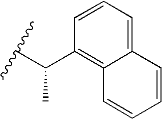 |
| AE | 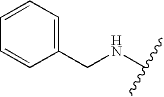 | CH₂ | 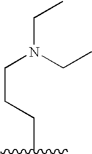 | C(O) | H | 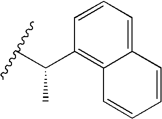 |
| AF | 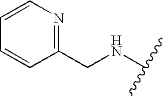 | CH₂ | 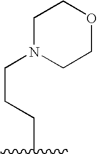 | C(O) | H | 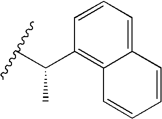 |
| AG | 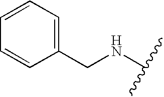 | CH₂ | 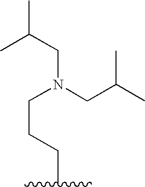 | C(O) | H | 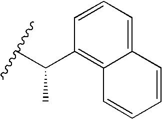 |
| AH | 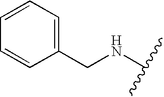 | CH₂ | 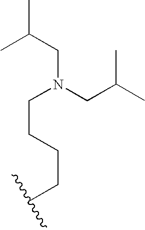 | C(O) | H | 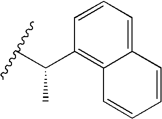 |
| AI | 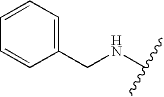 | CH₂ | 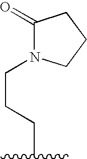 | C(O) | H | 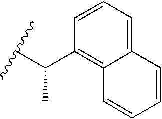 |
| AJ | 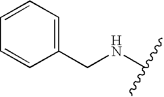 | CH₂ | 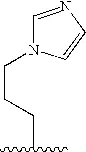 | C(O) | H | 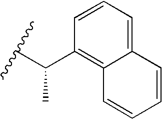 |
| AK | 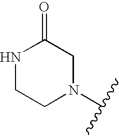 | CH₂ | 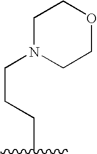 | C(O) | H | 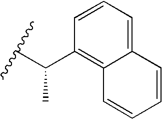 |
| AL | 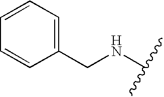 | CH₂ | 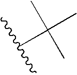 | C(O) | H | 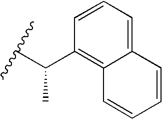 |
| AM | 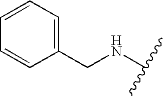 | CH₂ | 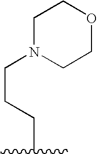 | C(O) | H | 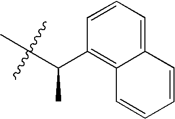 |
| AN | 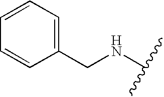 | CH₂ | 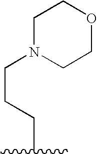 | C(O) | H | 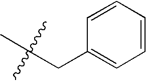 |
| AO | 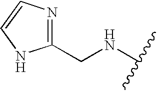 | CH₂ | 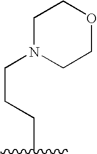 | C(O) | H | 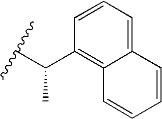 |
| AP | 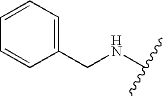 | CH₂ | 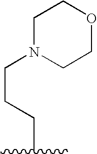 | C(O) | H | 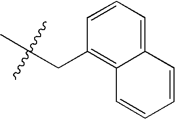 |
| AQ | 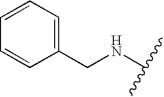 | CH₂ | 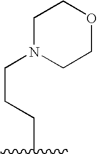 | C(O) | H | 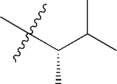 |
| AR | 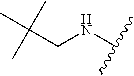 | CH₂ | 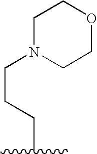 | C(O) | H | 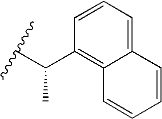 |
| AS | 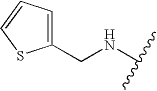 | CH₂ | 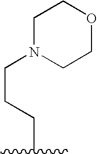 | C(O) | H | 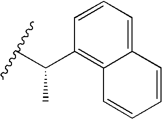 |
| AT | 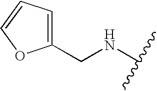 | CH₂ | 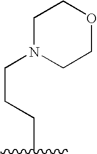 | C(O) | H | 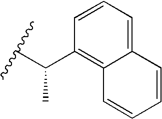 |
| AU | 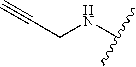 | CH₂ | 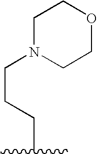 | C(O) | H | 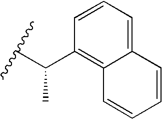 |
| AV | 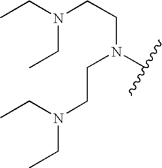 | CH₂ | 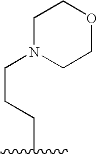 | C(O) | H | 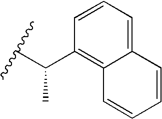 |
| AW | 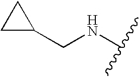 | CH₂ | 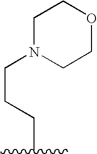 | C(O) | H | 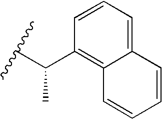 |
| AX | 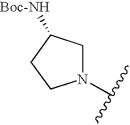 | CH₂ | 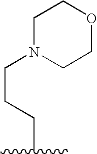 | C(O) | H | 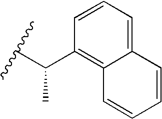 |
| AY | 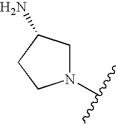 | CH₂ | 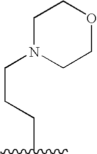 | C(O) | H | 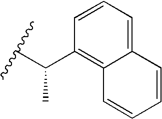 |
| AZ | 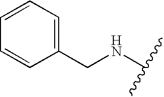 | CH₂ | 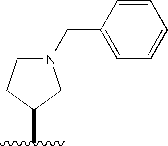 | C(O) | H | 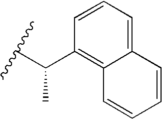 |
| BA | 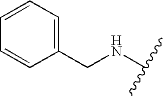 | CH₂ | 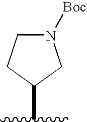 | C(O) | H | 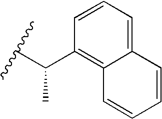 |
| BB | 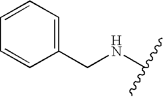 | CH₂ | 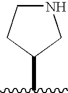 | C(O) | H | 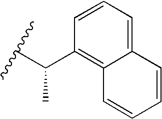 |
| BC | 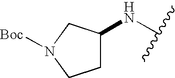 | CH₂ | 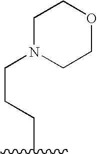 | C(O) | H | 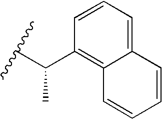 |
| BD | 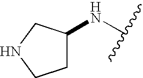 | CH₂ | 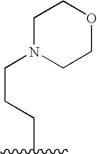 | C(O) | H | 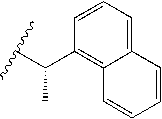 |
| BE | 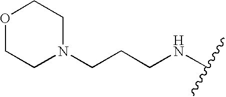 | CH₂ | 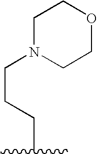 | C(O) | H | 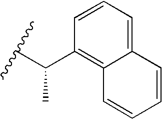 |
| BF | 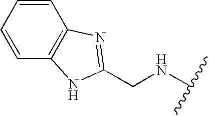 | CH₂ | 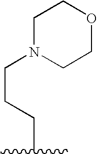 | C(O) | H | 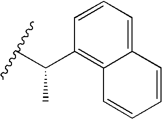 |
| BG | 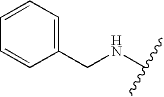 | CH₂ | 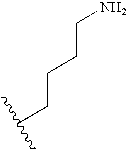 | C(O) | H | 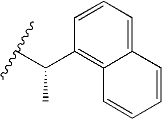 |
| BH | 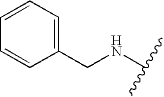 | CH₂ | 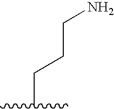 | C(O) | H | 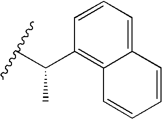 |
| BI | 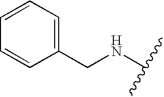 | CH₂ | 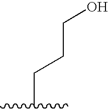 | C(O) | H | 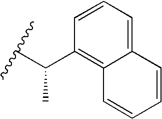 |
| BJ | 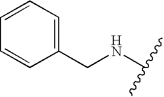 | CH₂ | 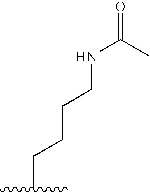 | C(O) | H | 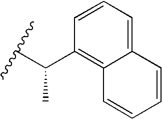 |
| BK | 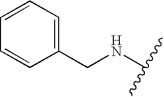 | CH₂ | 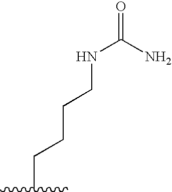 | C(O) | H | 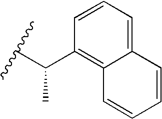 |
| BL | 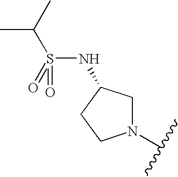 | CH₂ | 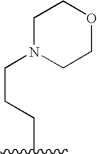 | C(O) | H | 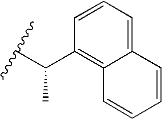 |
| BM | 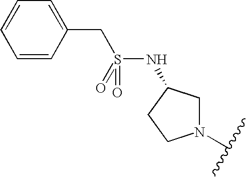 | CH₂ | 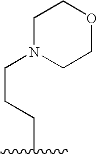 | C(O) | H | 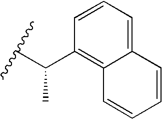 |
| BN | 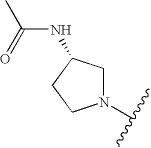 | CH₂ | 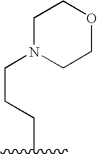 | C(O) | H | 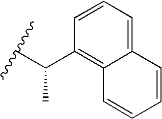 |
| BO | 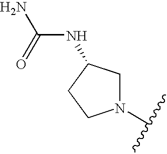 | CH₂ | 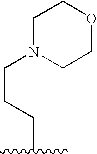 | C(O) | H | 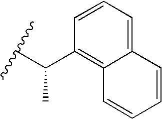 |
| BP | 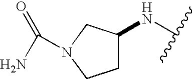 | CH₂ | 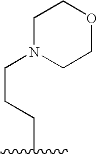 | C(O) | H | 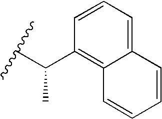 |
| BQ(a) | 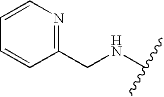 | CH₂ | 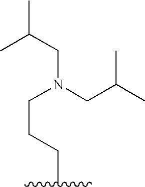 | C(O) | CH₃ | 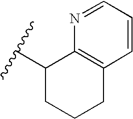 |
| BQ(b) | 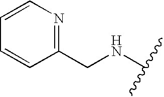 | CH₂ | 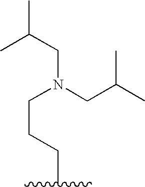 | C(O) | H | 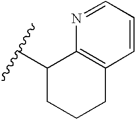 |
| BR | 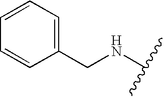 | CH₂ | 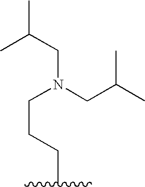 | C(O) | CH₃ | 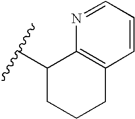 |
| BS | 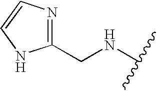 | CH₂ | 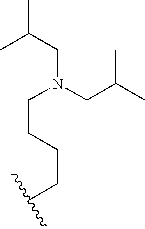 | C(O) | CH₃ | 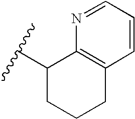 |

The compounds which comprise the first aspect of the present invention can be prepared by the procedure outlined herein below in Scheme I.

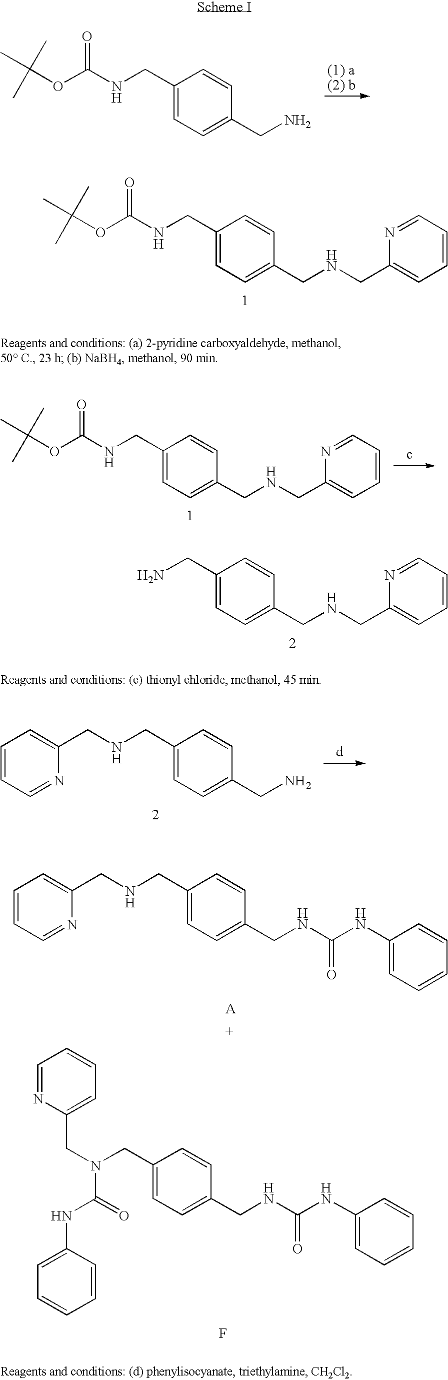

### Example 1

### 1-(4-((pyridin-2-ylmethylamino)methyl)benzyl)-3-phenylurea (A) and bisurea (F)

Preparation of tert-butyl 4-((pyridin-2-ylmethylamino)methyl)benzylcarbamate (1): To a solution of tert-butyl 4-(aminomethyl)benzylcarbamate (2.0 g, 8.5 mmol) in methanol (25 mL) was added 2-pyridine carboxaldehyde (0.8 mL, 8.5 mmol). The reaction mixture was heated to 50° C. and stirred for 23 hours. After cooling to room temperature, sodium borohydride (0.5 g, 1 2.7 mmol) was added to the reaction mixture portionwise and then stirred for 90 minutes at room temperature. The reaction was quenched by pouring into aqueous saturated NaHCO₃ solution and extracted with CHCl₃. The organic layer was dried (MgSO₄), filtered and concentrated in vacuo. The residue was purified over silica (2% methanol/CHCl₃ to 5% methanol/CHCl₃) to afford 2.0 g of the desired product as an orange oil: ¹H NMR (400 MHz, d₆-DMSO) δ 8.48 (d, J=4.8 Hz, 1H), 7.74 (ddd, J=7.6, 7.6, 1.6 Hz, 1H), 7.45 (d, J=7.6 Hz, 1H), 7.38 (t, J=6.4 Hz, 1H), 7.28 (d, J=7.6 Hz, 2H), 7.23 (dd, J=7.6, 6.0 Hz, 1H), 7.17 (d, J=8.0 Hz, 2H), 4.09 (d, J=6.4 Hz, 2H), 3.75 (s, 2H), 3.68 (s, 2H), 1.35 (s, 9H).

Preparation of N-(4-(aminomethyl)benzyl)(pyridin-2-yl)methanamine (2): To a solution of tert-butyl 4-((pyridin-2-ylmethylamino)methyl)benzylcarbamate, 1, (1.8 g, 5.6 mmol), in methanol (40 mL) was added thionyl chloride (5 mL) dropwise. After stirring the reaction for 45 minutes at room temperature, the mixture was concentrated in vacuo to afford 1.7 grams of the desired product as the hydrochloride salt which was used without further purification.

Preparation of 1-(4-((pyridin-2-ylmethylamino)methyl)benzyl)-3-phenylurea (A) and bis-urea (F): To a cold (0° C.) solution of N-(4-(aminomethyl)benzyl)(pyridin-2-yl)methanamine, 2, (0.40 g, 1.34 mmol) and triethylamine (0.93 mL, 6.69 mmol) in CH₂Cl₂ (15 mL) was added phenyl isocyanate (0.15 mL, 1.34 mmol). After stirring the reaction for 30 minutes at 0° C., the mixture was warmed to room temperature and stirred for an additional 20 minutes. The reaction was quenched by pouring into an aqueous saturated NaHCO₃ solution and extracted with CHCl₃. The organic layer was dried (MgSO₄), filtered and concentrated in vacuo. The residue was purified over silica (1% methanol/CHCl₃ to 10% methanol/CHCl₃) to afford 40 mg of the urea product A as an white solid along with 290 mg of the bis-urea product F as a sticky yellow solid. Analytical data for urea BB: ¹H NMR (400 MHz, d₆-DMSO) δ 8.51 (s, 1H), 8.47 (d, J=4.4 Hz, 1H), 7.74 (ddd, J=8.0, 8.0, 2.0 Hz, 1H), 7.44 (d, J=8.0 Hz, 1H), 7.39 (d, J=8.0 Hz, 2H), 7.30 (d, J=8.0 Hz, 2H), 7.25-7.19 (m, 4H), 6.88 (t, J=7.2 Hz, 1H), 6.56 (t, J=6.0 Hz, 1H), 4.27 (d, J=5.6 Hz, 2H), 3.75 (s, 2H), 3.69 (s, 2H); ESI⁺ MS: m/z (rel intensity) 347 (100, M+H). Analytical data for bis-urea BG: ¹H NMR (400 MHz, d₆-DMSO) δ 9.01 (s, 1H), 8.54 (d, J=7.6 Hz, 1H), 8.50 (s, 1H), 7.74 (ddd, J=8.0, 8.0, 2.0 Hz, 1H), 7.43 (dd, J=8.0, 0.8 Hz, 2H), 7.35 (dd, J=8.0, 1.2 Hz, 2H), 7.28-7.15 (m, 10H), 6.91 (t, J=7.2 Hz, 1H), 6.85 (t, J=7.2 Hz, 1H), 6.55 (t, J=5.6 Hz, 1H), 4.55 (d, J=5.1 Hz, 4H), 4.24 (d, J=6.0 Hz, 2H).

The compounds which comprise the first aspect of the present invention can also be prepared by the procedure outlined herein below in Scheme II.

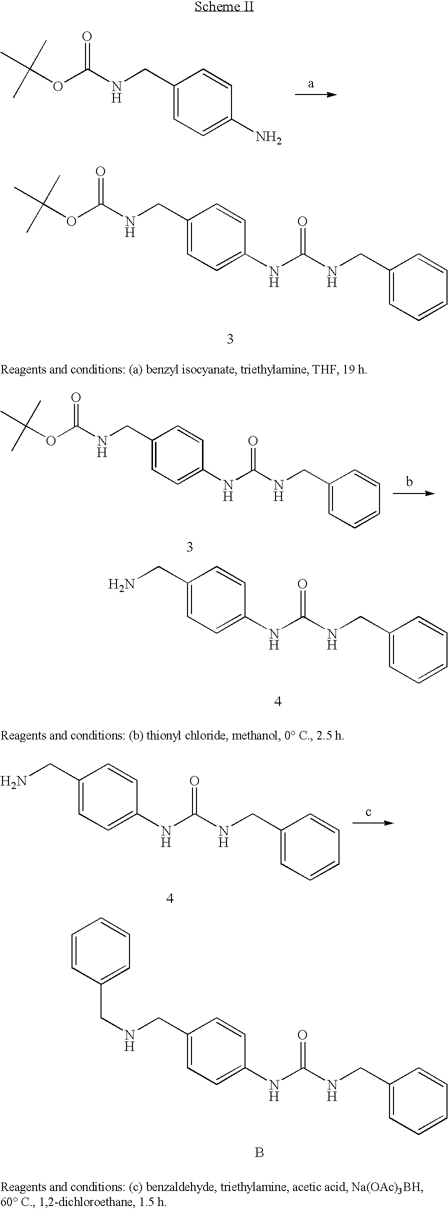

### Example 2

### 1-benzyl-3-(4-((benzylamino)methyl)phenyl)urea (B)

Preparation of tert-butyl 4-(3-benzylureido)benzylcarbamate (3): To a solution of tert-butyl 4-aminobenzylcarbamate (3.22 g, 14.10 mmol) in tetrahydrofuran (20 mL) was added benzyl isocyanate (1.82 mL, 14.81 mmol) and triethylamine (2.16 mL, 15.50 mmol). The mixture was stirred at ambient temperature for 18 hrs and diluted with hexanes (2 mL). The reaction mixture was concentrated under reduced pressure to effect precipitation. The solids were collected by filtration and washed with 50% EtOAc/hexanes to afford the crude product 3, which was used without further purification. Additional product was obtained by cooling the filtrate in an ice bath. After addition of equivolume amount of hexanes, the resulting solids were collected by filtration, washed with 50% EtOAc/hexanes, hexanes and the resulting solid was dried in vacuo to yield a total of 4.8 g of the desired product 3, which was used without further purification: ¹H NMR (300 MHz, CDCl₃) δ 8.58 (d, J=5.0 Hz, 1H), 7.93 (s, 1H), 7.82 (d, J=5.0 Hz, 1H), 4.00 (s, 3H); ESI⁺ MS: m/z (rel intensity) 172.1 (100, M+H).

Preparation of 1-(4-(aminomethyl)phenyl)-3-benzylurea (4): To a cold (0° C.) solution of tert-butyl 4-(3-benzylureido)benzylcarbamate, 3, (4.7 g, 13.3 mmol) in methanol (30 mL) was added thionyl chloride (7.7 mL, 106.2 mmole) dropwise. The reaction mixture was stirred at 0° C. for 2.5 hours and then concentrated under reduced pressure. The residue was washed with 10% EtOAc/hexanes, hexanes and dried in vacuo to afford 3.91 g of the desired amine 4 as the HCl salt: ¹H NMR (300 MHz, CDCl₃) δ 8.32 (d, J=5.3 Hz, 1H), 7.47 (d, J=5.3 Hz, 1H), 7.37 (s, 1H), 4.03 (s, 3H), 3.30 (q, J=7.2 Hz, 2H), 1.44 (t, J=7.2 Hz, 3H); ESI⁺ MS: m/z (rel intensity) 198.1 (100 μM+H).

Preparation of 1-benzyl-3-(4-((benzylamino)methyl)phenyl)urea (B): To a mixture of 1-(4-(aminomethyl)phenyl)-3-benzylurea, 4, (0.25 g, 0.87 mmol) in 1,2-dichloroethane (6 mL) at room temperature was added in succession: benzaldehyde (0.08 mL, 0.79 mmol), triethylamine (0.25 mL, 1.74 mmol) and 2 drops of glacial acetic acid. Methanol (3 mL) was then added to dissolve the remaining solids. After stirring the mixture for 15 minutes at room temperature, sodium triacetoxyborohydride (0.35 g, 1.66 mmol) was added and the mixture was heated to 60° C. with stirring. After 1.5 hr, the mixture was diluted with aqueous saturated NaHCO₃. The aqueous phase was extracted three times with EtOAc. The combined organic phases were washed with brine, dried (MgSO₄), filtered and concentrated in vacuo. The crude residue was purified by preparative HPLC (Polaris C18 column using acetonitrile/water with 0.1% TFA) and the product was free-based by partitioning with EtOAc/saturated NaHCO₃, washing with saturated NaHCO₃ solution, washing with brine and drying over MgSO₄. Concentration in vacuo from CH₂Cl₂/hexanes as final solvent afforded 129 mg of the desired product B: ¹H NMR (300 MHz, CDCl₃) δ 8.32 (d, J=5.3 Hz, 1H), 7.47 (d, J=5.3 Hz, 1H), 7.37 (s, 1H), 4.03 (s, 3H), 3.30 (q, J=7.2 Hz, 2H), 1.44 (t, J=7.2 Hz, 3H); ESI⁺ MS: m/z (rel intensity) 198.1 (100, M+H).

The compounds which comprise the first aspect of the present invention can also be prepared by the procedure outlined herein below in Scheme III.

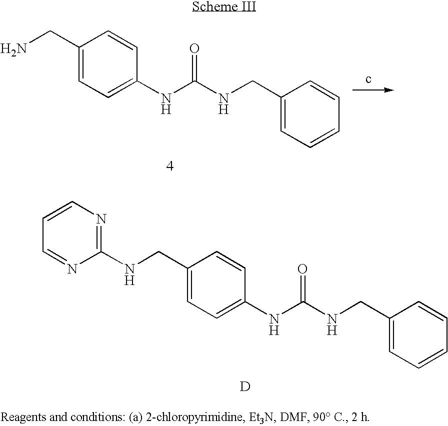

### Example 3

### 1-benzyl-3-(4-((pyrimidin-2-ylamino)methyl)phenyl)urea (D)

Preparation of 1-benzyl-3-(4-((pyrimidin-2-ylamino)methyl)phenyl)urea (D): To a solution of 1-(4-(aminomethyl)phenyl)-3-benzylurea, 4, (0.27 g, 0.93 mmol) in DMF (2 mL) was added 2-chloropyrimidine (0.12 g, 1.02 mmol) and triethylamine (0.45 mL, 3.22 mmol). The mixture was heated to 90° C. for 2 hours. The mixture was diluted with aqueous saturated NaHCO₃. The aqueous phase was extracted three times with EtOAc. The combined organic phases were washed with brine, dried (MgSO₄), filtered and concentrated in vacuo. The crude residue was purified by preparative HPLC (Polaris C18 column using acetonitrile/water with 0.1% TFA) and the product was free-based by partitioning with EtOAc/saturated NaHCO₃, washing with saturated NaHCO₃ solution, washing with brine and drying over MgSO₄. Concentration in vacuo from CH₂Cl₂/hexanes as final solvent afforded 74 mg of the desired product D: ¹H NMR (300 MHz, d₆-DMSO) δ 8.33 (d, J=5.1 Hz, 1H), 7.39 (d, J=5.1 Hz, 1H), 7.19 (s, 1H), 3.16 (q, J=7.3 Hz, 2H), 1.30 (t, J=7.3 Hz, 3H); ESI⁺ MS: m/z (rel intensity) 184.0 (100, M+H), ESI⁻ MS: m/z (rel intensity) 182 (100, M−H).

The compounds which comprise the first aspect of the present invention can also be prepared by the procedure outlined herein below in Scheme IV.

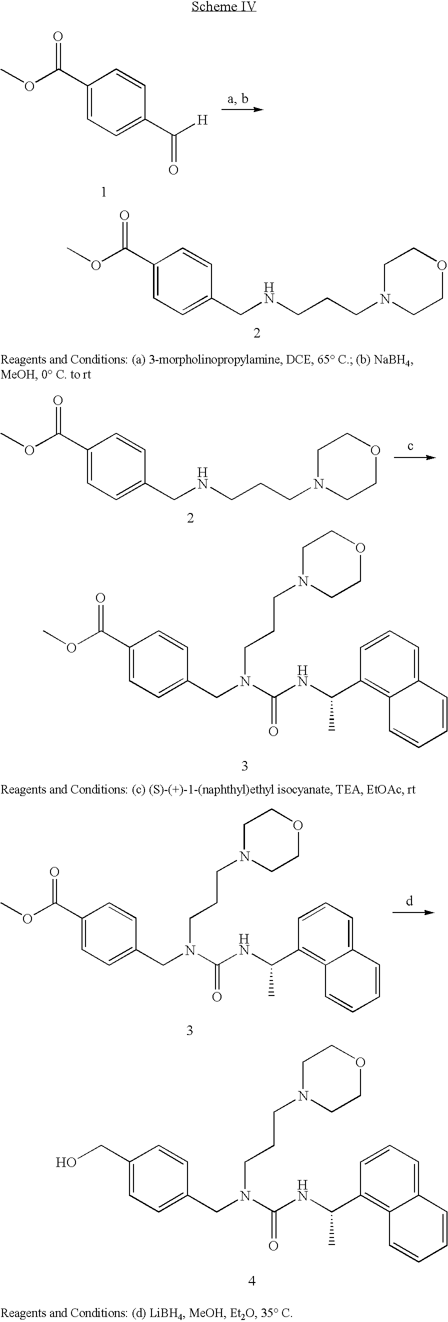

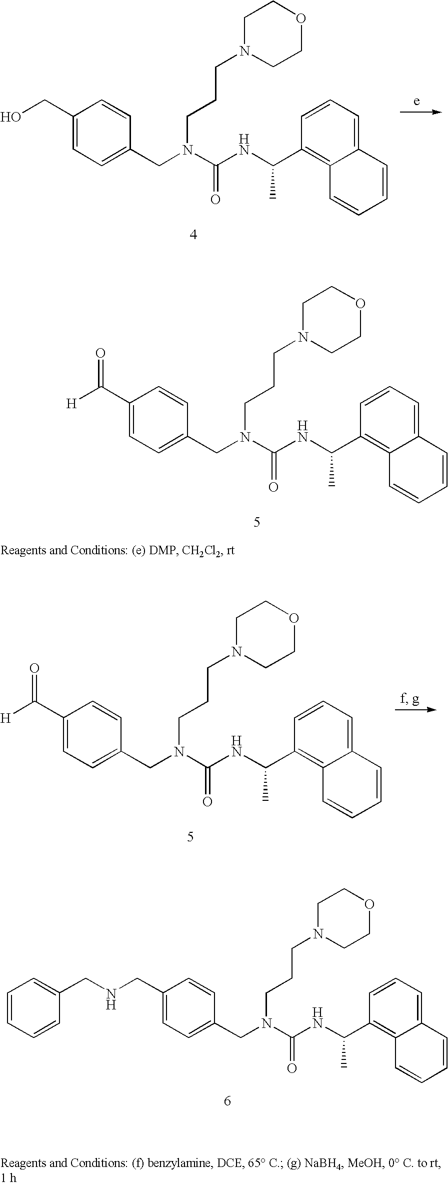

### Example 4

### (S)-1-(4-((benzylamino)methyl)benzyl)-1-(3-morpholinopropyl)-3-(1-(naphthalen-1-yl)ethyl)urea (6) (AC)

Preparation of methyl 4-((3-morpholinopropylamino)methyl)benzoate (2): To a solution of methyl-4-formylbenzoate (2.0 g, 12.2 mmol) in DCE (25 mL) was added 3-morpholino-propan-1-amine (2.12 g, 14.6 mmol). The reaction was stirred at 65° C. for 18 h. The mixture was cooled to 0° C. and sodium borohydride (0.91 g, 24.1 mmol) was added slowly. The reaction mixture was warmed to room temperature and stirred for 1 h. The solution was quenched with a saturated aqueous solution of sodium bicarbonate (15 mL), diluted with ethyl acetate (75 mL), and dried over magnesium sulfate. The organic solution was filtered, concentrated, and purified by silica gel chromatography (5% MeOH/CH₂Cl₂) to afford (2). ¹H NMR (400 MHz, d⁶-DMSO) δ 7.86 (d, J=8.8 Hz, 2H), 7.42 (d, J=8.4 Hz, 2H), 3.79 (s, 3H), 7.70 (s, 2H), 3.49 (t, J=4 Hz, 4H), 2.44 (t, J=6.8 Hz, 3H), 2.26-2.22 (m, 5H), 1.52 (t, J=7.2 Hz, 2H); ESI⁺ MS: m/z (rel intensity) 293.2 (100, [M+H]⁺).

Preparation of (S)-methyl 4-((1-(3-morpholinopropyl)-3-(1-(naphthalen-1-yl)ethyl)ureido)methyl)benzoate (3): To a solution of methyl 4-((3-morpholinopropylamino)methyl)benzoate, 2, (1.0 g, 3.42 mmol) in ethyl acetate (7 mL) was added triethylamine (0.95 mL, 6.84 mmol), (S)-(+)-1-(naphthyl)ethyl isocyanate (890 μL, 5.13 mmol). The reaction was stirred at room temperature for 18 h. The mixture was quenched with H₂O (3 mL), extracted with ethyl acetate (50 mL). The combined organic layers were washed with brine, dried over magnesium sulfate, filtered and concentrated in vacuo. The crude material was purified by silica gel chromatography (0-5% MeOH/CH₂Cl₂) to provide compound (3). ¹H NMR (400 MHz, d⁶-DMSO) δ 8.11 (d, J=8.0 Hz, 1H), 7.86 (d, J=8.4 Hz, 3H), 7.77 (d, J=7.6 Hz, 1H), 7.53-7.41 (m, 4H), 7.30 (d, J=8.4 Hz, 2H), 6.82 (d, J=8.0 Hz, 1H), 4.52 (s, 2H), 3.80 (s, 3H), 3.39 (bs, 4H), 3.31 (s, 1H), 3.20-3.08 (m, 2H), 2.11 (bs, 6H), 1.49 (bs, 5H); ESI⁺ MS: m/z (rel intensity) 490.2 (100, [M+H]⁺).

Preparation of (S)-1-(4-(hydroxymethyl)benzyl)-1-(3-morpholinopropyl)-3-(1-(naphthalen-1-yl)ethyl)urea (4): To a solution of (S)-methyl 4-((1-(3-morpholinopropyl)-3-(1-(naphthalen-1-yl)ethyl)ureido)methyl)benzoate, 3, (1.46 g, 3.0 mmol) in diethyl ether (10 mL) were added slowly lithium borohydride (0.39 g, 18.0 mmol) and methanol (0.73 mL, 18.0 mmol). The reaction was heated at 35° C. for 2 h. The mixture was cooled to room temperature and slowly quenched with a saturated aqueous solution of ammonium chloride (3 mL). The solution was diluted with ethyl acetate (50 mL). The combined organic layers were washed with brine, dried over magnesium sulfate, filtered and concentrated in vacuo. The crude material was purified by silica gel chromatography to afforded (4). ¹H NMR (400 MHz, d⁶-DMSO) δ 8.12 (d, J=8.0 Hz, 1H), 7.90 (d, J=7.2 Hz, 1H), 7.78 (d, J=7.6 Hz, 1H), 7.53-7.41 (m, 4H), 7.20 (d, J=8.0 Hz, 2H), 7.11 (d, J=8.0 Hz, 2H), 6.77 (bs, 1H), 4.43 (bs, 4H), 3.39 (bs, 4H), 3.30 (s, 2H), 3.12-3.05 (m, 2H), 2.11 (bs, 6H), 1.48 (bs, 5H); ESI⁺ MS: m/z (rel intensity) 462.2 (100, [M+H]⁺).

Preparation of (S)-1-(4-formylbenzyl)-1-(3-morpholinopropyl)-3-(1-(naphthalen-1-yl)ethyl)urea (5): To a solution of (S)-1-(4-(hydroxymethyl)benzyl)-1-(3-morpholinopropyl)-3-(1-(naphthalen-1-yl)ethyl)urea, 4, (0.59 g, 1.28 mmol) in dichloromethane (3 mL) was added Dess-Martin periodinane (0.70 g, 1.66 mmol). The reaction stirred at room temperature for 18 h. The mixture was quenched with a saturated aqueous solution of sodium bicarbonate, extracted with dichloromethane (10 mL). The combined organic layers were washed with brine, dried over magnesium sulfate, filtered and concentrated in vacuo. The crude material was purified by silica gel chromatography (5% MeOH/CHCl₃) to yield (5). ¹H NMR (400 MHz, d⁶-DMSO) δ 9.94 (s, 1H), 8.11 (d, J=7.6 Hz, 1H), 7.90-7.76 (m, 4H), 7.54-7.42 (m, 4H), 7.37 (d, J=6.8 Hz, 2H), 6.84 (d, J=7.6 Hz, 1H), 4.55 (s, 2H), 3.38 (bs, 3H), 3.32 (bs, 2H), 3.19-3.09 (m, 2H), 2.12 (bs, 6H), 1.53-1.47 (m, 5H); ESI⁺ MS: m/z (rel intensity) 460.2 (100, [M+H]⁺).

Preparation of (S)-1-(4-((benzylamino)methyl)benzyl)-1-(3-morpholinopropyl)-3-(1-(naphthalen-1yl)ethyl)urea (AC): To a solution of(S)-1-(4-formylbenzyl)-1-(3-morpholinopropyl)-3-(1-(naphthalen-1-yl)ethyl)urea, 5, (0.12 g, 0.26 mmol) in dichloroethane (1 mL) was added N-benzylamine (34 μL, 0.31 mmol). The reaction was stirred at 65° C. for 18 h. The mixture was cooled to 0° C. and sodium borohydride (0.19 g, 0.51 mmol) was added slowly. The reaction mixture was warmed to room temperature and stirred for 1 h. The solution was quenched with a saturated aqueous solution of sodium bicarbonate (0.5 mL), diluted with ethyl acetate (5 mL). The combined organic layers were dried over magnesium sulfate, filtered and concentrated in vacuo. The crude material was purified silica gel chromatography (5% MeOH/CH₂Cl₂) to afford AC. ¹H NMR (400 MHz, d⁶-DMSO) δ 8.12 (d, J=8.4 Hz, 1H), 7.89 (d, J=7.6 Hz, 1H), 7.76 (d, J=7.6 Hz, 1H), 7.53-7.41 (m, 4H), 7.30-7.18 (m, 6H), 7.11 (dd, J=2.8, 2.4 Hz, 2H), 6.75 (d, J=8.0 Hz, 1H), 5.67 (t, J=7.2 Hz, 1H), 4.42 (s, 3H), 3.61 (s, 2H), 3.39 (bs, 4H), 3.31 (s, 2H), 3.16-3.04 (m, 2H), 2.12-2.09 (m, 6H), 1.51-1.47 (m, 5H); ESI⁺ MS: m/z (rel intensity) 551.3 (100, [M+H]⁺).

Alternatively, the final reductive amination can be carried out in MeOH using NaBH₄ as the reducing agent.

The following compounds AF through AN were prepared using methods similar to those used to prepare compound AC using the appropriately substituted amines in steps b and f of Scheme IV, and are also non-limiting examples of compounds prepared using methods similar to those described in Scheme XX.

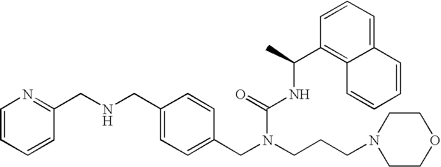

(S)-1-(3-morpholinopropyl)-3-(1-(naphthalen-1-yl)ethyl)-1-(4-((pyridin-2-ylmethylamino)methyl)benzyl)urea (AF): ¹H NMR (400 MHz, DMSO-d₆) δ 8.44 (d, J=4.4 Hz, 1H), 8.12 (d, J=8.0 Hz, 1H), 7.88 (d, J=8.0 Hz, 1H), 7.77-7.69 (m, 2H), 7.51-7.40 (m, 5H), 7.25-7.18 (m, 3H), 7.11 (d, J=8.0 Hz, 2H), 5.66 (t, J=7.2 Hz, 1H), 4.42 (s, 1H), 3.72 (s, 1H), 3.65 (s, 1H), 3.37 (bs, 3H), 3.30 (s, 2H), 3.12-3.06 (m, 2H), 2.46 (s, 4H), 2.10 (bs, 4H), 1.48 (d, J=6.4 Hz, 4H); ESI⁺ MS: m/z (rel intensity) 552.3 (40, [M+H]⁺).

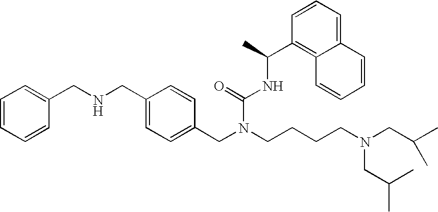

(S)-1-(4-((benzylamino)methyl)benzyl)-1-(4-(diisobutylamino)butyl)-3-(1-naphthalen-1-yl)ethyl)urea (AH): ¹H NMR (400 MHz, d⁶-DMSO) δ 8.11 (bs, 1H), 7.87 (bs, 1H), 7.73 (bs, 1H), 7.45-7.40 (m, 5H), 7.27 (bs, 3H), 7.17 (d, J=7.6 Hz, 2H), 7.07 (d, J=7.6 Hz, 2H), 6.70 (bs, 1H), 1.54-1.35 (m, 8H), 1.22 (bs, 2H), 0.76 (bs, 12H); ESI⁺ MS: m/z (rel intensity) 607.4 (60, [M+H]⁺).

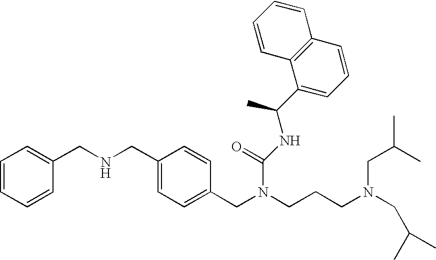

(S)-1-(4-((benzylamino)methyl)benzyl)-1-(3-(diisobutylamino)propyl)-3-(1-napthalen-1-yl)ethyl)urea (AG): ¹H NMR (400 MHz, d⁶-DMSO) δ 8.09 (d, J=8.0 Hz, 1H), 7.86 (d, J=6.8 Hz, 1H), 7.73 (d, J=8.0 Hz, 1H), 7.49-7.38 (m, 3H), 7.29-7.15 (m, 10H), 3.64 (d, J=6.8 Hz, 4H), 3.11 (bs, 2H), 2.93 (d, J=7.2 Hz, 2H), 2.24 (bs, 2H), 2.01 (d, J=7.2 Hz, 2H), 1.87 (s, 1H), 1.75-1.66 (m, 2H), 1.52 (bs, 2H), 1.42 (d, J=6.8 Hz, 3H), 0.76 (bs, 12H); ESI⁺ MS: m/z (rel intensity) 593.3 (100, [M+H]⁺).

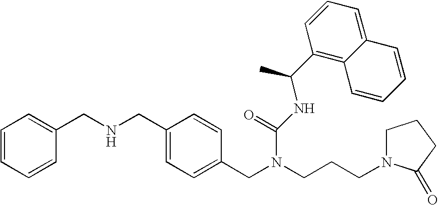

(S)-1-(4-((benzyl(amino)methyl)benzyl)-3-(1-(naphthalen-1-yl)ethyl)-1-(3-(2-oxopyrrolidin-1-yl)propyl)urea (AI): ¹H NMR (400 MHz, d⁶-DMSO) δ 8.13 (d, J=8.4 Hz, 1H), 7.88 (d, J=8.4 Hz, 1H), 7.74 (d, J=7.6 Hz, 1H), 7.53-7.41 (m, 4H), 7.30-7.20 (m, 6H), 7.10 (d, J=6.8 Hz, 2H), 6.84 (d, J=7.2 Hz, 1H), 5.66 (t, J=6.0 Hz, 1H), 4.43 (d, J=7.6 Hz, 2H), 3.64 (s, 4H), 3.12-3.01 (m, 6H), 2.09 (t, J=7.6 Hz, 2H), 1.86 (s, 1H), 1.77 (t, J=7.6 Hz, 2H), 1.53 (t, J=6.8 Hz, 2H), 1.46 (d, J=6.4 Hz, 3H); ESI⁺ MS: m/z (rel intensity) 549.2 (100, [M+H]⁺).

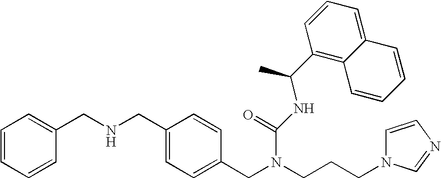

(S)-1-(3-(1H-imidazole-1-yl)propyl)-1-(4-((benzylamino)methyl)benzyl)-3-(1-(naphthalen-1-yl)ethyl)urea (AJ): ¹H NMR (400 MHz, d⁶-DMSO) δ 8.13 (d, J=8.0 Hz, 1H), 7.92 (d, J=8.4 Hz, 1H), 7.75 (d, J=8.0 Hz, 1H), 7.51-7.41 (m, 5H), 7.29 (bs, 3H), 7.20 (d, J=7.21 (d, J=7.6 Hz, 3H), 7.06-7.02 (m, 3H), 6.80 (s, 2H), 5.67 (t, J=7.2 Hz, 1H), 4.41 (s, 2H), 3.83 (t, J=6.4 Hz, 2H), 3.61 (d, J=4.8 Hz, 4H), 3.15-3.07 (m, 3H), 1.82 (t, J=6.8 Hz, 2H), 1.46 (d, J=6.4 Hz, 3H); ESI⁺ MS: m/z (rel intensity) 532.2 (100, [M+H]⁺).

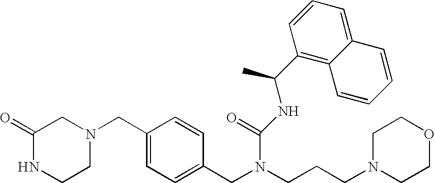

(S)-1-(3-morpholinopropyl)-3-(1-(naphthalen-1-yl)ethyl)-1-(4-((3-oxopiperazin-1-yl)methyl)benzyl)urea (AK): ¹H NMR (400 MHz, d⁶-DMSO) δ 8.13 (bs, NH), 7.89 (bs, 1H), 7.77-7.71 (m, 2H), 7.49 (bs, 4H), 7.19-7.13 (m, 3H), 6.76 (bs, 1H), 5.61 (bs, 1H), 4.43 (s, 2H), 3.45-3.30 (m, 8H), 3.08 (bs, 4H), 2.82 (s, 2H), 2.10 (bs, 6H), 1.48 (bs, 6H); ESI⁺ MS: m/z (rel intensity) 544.3 (40, [M+H]⁺).

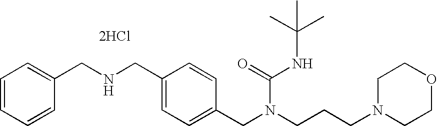

1-(4-((benzylamino)methyl)benzyl)-3-tert-butyl-1-(3-morpholinopropyl)urea (AL): ¹H NMR (400 MHz, CDCl₃) [symbol] 7.32-7.15 (m, 9H), 4.42-4.10 (m, 2H), 3.80-3.77 (m, 4H), 3.68 (t, J=4.4 Hz, 4H), 3.23 (t, J=6.8 Hz, 2H), 2.45-2.35 (m, 4H), 2.31 (t, J=6.4 Hz, 2H), 1.77 (bs, 1H), 1.72-1.65 (m, 2H), 1.28 (s, 9H); ESI⁺ MS: m/z (rel intensity) 453.3 (100, [M+H]⁺).

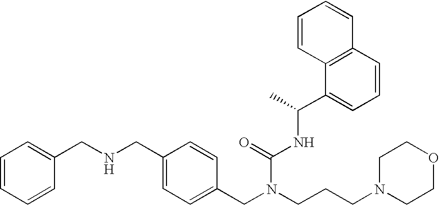

(R)-1-(4-(benzylamino)methyl)benzyl)-1-(3-morpholinopropyl)-3-(1-(naphthalen-1-yl)ethyl)urea (AM): ¹H NMR (400 MHz, CDCl₃) [symbol] 8.17 (d, J=8.4 Hz, 1H), 7.82 (d, J=8.0 Hz, 1H), 7.75 (d, J=8.0 Hz, 1H), 7.55-7.36 (m, 5H), 7.36-7.20 (m, 8H), 5.98 (bs, 1H), 5.92-5.80 (m, 1H), 4.51 (q, J=18.8 Hz, 2H), 3.79 (s, 2H), 3.78 (s, 2H), 3.60-3.25 (m, 2H), 3.25-3.05 (m, 4H), 2.23-2.10 (m, 4H), 2.00-1.88 (m, 2H), 1.82 (bs, 1H), 1.65 (d, J=6.8 Hz, 3H), 1.60-1.47 (m, 2H); ESI⁺ MS: m/z (rel intensity) 551.3 (100, [M+H]⁺).

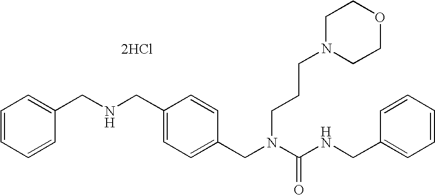

3-benzyl-1-(4-((benzylamino)methyl)benzyl)-1-(3-morpholinopropyl)urea (AN): ¹H NMR (400 MHz, CDCl₃) [symbol] 7.33-7.22 (m, 14H), 4.53 (s, 2H), 4.46 (d, J=5.6 Hz, 2H), 3.79 (s, 2H), 3.78 (m, 2H), 3.45-3.35 (m, 4H), 3.25 (t, J=5.6 Hz, 2H), 2.29 (d, J=6.4 Hz, 2H), 2.27-2.17 (m, 4H), 1.63-1.57 (m, 2H); ESI⁺ MS: m/z (rel intensity) 487.2 (100, [M+H]⁺).

Unless otherwise indicated, compounds of formula (IA), wherein M¹ is—CH₂CH₂— and R⁵ is substituted or unsubstituted alkylamino or substituted or unsubstituted heterocyclyl (e.g., compounds J, K, V and AA) can be prepared using the methods similar to those shown in Scheme IV, using the appropriately substituted reagents, for example, the methyl-4-formyl benzoate used in step a can be replaced with 4-(2-oxoethyl)benzaldehyde.

Those skilled in the art will recognize that, unless otherwise indicated, other compounds listed in Table 1 can be prepared using methods similar to those described in Schemes I-IV, using the appropriately substituted reagents.

Similarly, unless otherwise indicated, sulfonylureas such as compound AB can be prepared using methods analogous to those of Scheme II, except that tert-butyl-4-aminobenzylcarbamate is reacted with phenylsulfamoyl chloride to provide a protected sulfonylurea analog of compound 3, which can then be deprotected, reacted with an aldehyde, and reduced as in Scheme II.

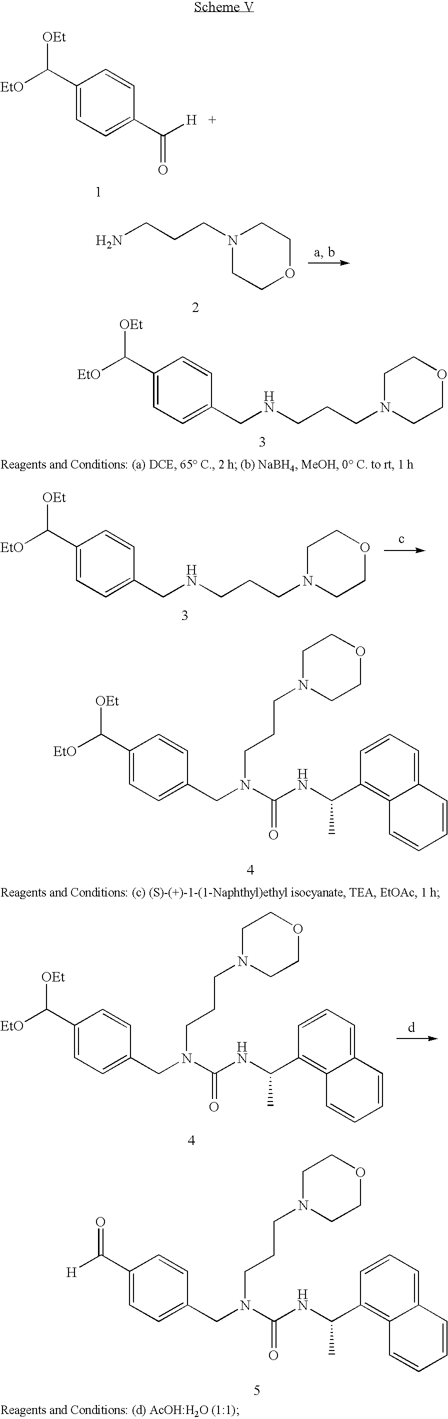

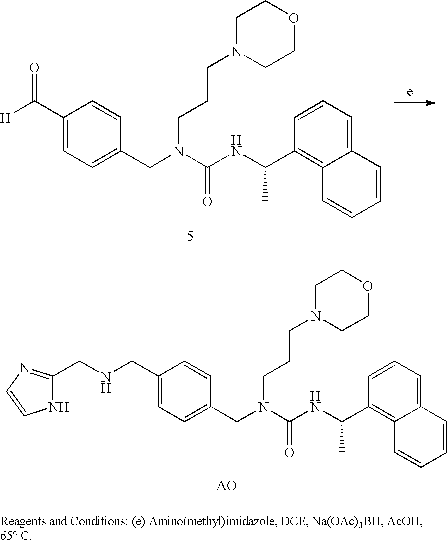

### Example 5

### (S)-1-(4-(((1H-imidazol-2-yl)methylamino)methyl)benzyl)-1-(3-morpholinopropyl)-3-(1-(naphthalen-1-yl)ethyl)urea (AO)

Preparation of N-(4-(diethoxymethyl)benzyl)-3-morpholinopropan-1-amine (3): To a solution of terephthaldehyde mono(diethyl acetal), 1, (1.44 mL, 7.20 mmol) in dichloroethane (15 mL) was added 3-morpholinopropylamine (1.27 mL, 8.64 mmol). The mixture was warmed to 65° C. and stirred for 1 h. The solvent was then removed by evaporation. The imine was dissolved at 0° C. in methanol (15 mL) and sodium borohydride (600 mg, 15.84 mmol) was added portion-wise. After the addition was complete, the reaction was warmed to room temperature and stirred for 20 min. The reaction was then quenched with a saturated aqueous solution of sodium bicarbonate (5 mL). The product was extracted with methylene chloride (3×15 mL). The combined organic layers were dried over potassium carbonate, filtered and concentrated. The crude product 3 (quantitative yield) was dried under vacuum and used as is in the next step: ¹H NMR (400 MHz, CDCl₃) δ 7.40 (d, J=8.0 Hz, 2H), 7.28 (d, J=8.0 Hz, 2H), 5.47 (s, 1H), 3.76 (s, 2H), 3.67 (t, J=4.8 Hz, 4H), 3.62-3.48 (m, 4H), 2.65 (t, J=6.8 Hz, 2H), 2.41-2.35 (m, 6H), 1.74-1.64 (m, 2H), 1.21 (t, J=6.8 Hz, 6H); ESI⁺ MS: m/z (rel intensity) 337.2 (100, [M+H]⁺).

Preparation of (S)-1-(4-(diethoxymethyl)benzyl)-1-(3-morpholinopropyl)-3-(1-(naphthalen-1-yl)ethyl)urea 4: To a solution of N-(4-(diethoxymethyl)benzyl)-3-morpholinopropan-1-amine, 3, (0.81 g, 2.40 mmol, 1) in ethyl acetate (10 mL) at room temperature was added triethylamine (0.67 mL, 4.80 mmol) and (S)-(+)-1-(1-naphthyl)ethyl isocyanate (440 μL, 2.52 mmol). The mixture was stirred at room temperature for 1 h. The solvent was then removed by evaporation. The crude product was purified by silica gel chromatography (0-20% MeOH/CH₂Cl₂) to afford 1.28 g (quantitative yield) of pure product 4: ¹H NMR (400 MHz, CDCl₃) δ 8.15 (d, J=8.4 Hz, 1H), 7.81 (d, J=8.0 Hz, 1H), 7.74 (d, J=8.0 Hz, 1H), 7.54-7.36 (m, 6H), 7.25 (d, J=7.6 Hz, 2H), 5.96 (bs, 1H), 5.90-5.80 (m, 1H), 5.45 (s, 1H), 4.51 (q, J=16.0 Hz, 2H), 3.64-3.40 (m, 4H), 3.36-3.26 (m, 2H), 3.24-3.04 (m, 4H), 2.20-2.08 (m, 4H), 1.95-1.86 (m, 2H), 1.64 (d, J=7.6 Hz, 3H), 1.58-1.46 (m, 2H), 1.26-1.18 (m, 6H); ESI⁺ MS: m/z (rel intensity) 534.2 (100, [M+H]⁺).

Preparation of (S)-1-(4-formylbenzyl)-1-(3-morpholinopropyl)-3-(1-(naphthalen-1-yl)ethyl)urea (5): A solution of (S)-1-(4-(diethoxymethyl)benzyl)-1-(3-morpholinopropyl)-3-(1-(naphthalen-1-yl)ethyl)urea, 4, (1.28 g, 2.40 mmol) in 1:1 H₂O:AcOH (5 mL: 5 mL) was stirred at room temperature for 10 min. A saturated aqueous solution of sodium bicarbonate was added. The product was extracted with ethyl acetate (3×10 mL). The combined organic layers were washed with brine, dried over magnesium sulfate, filtered and concentrated. The crude product 5 (1.10 g, quantitative yield) was dried under vacuum and used as is in the next step: ¹H NMR (400 MHz, CDCl₃) δ 9.93 (s, 1H), 8.11 (d, J=8.4 Hz, 1H), 7.82-7.70 (m, 4H), 7.51-7.24 (m, 7H), 5.90-5.65 (m, 2H), 4.63-4.52 (m, 2H), 3.90-3.60 (m, 4H), 3.27-3.22 (m, 1H), 2.75-2.60 (m, 4H), 2.60-2.40 (m, 2H), 1.90-1.80 (m, 3H), 1.64 (d, J=6.4 Hz, 3H); ESI⁺ MS: m/z (rel intensity) 460.2 (100, [M+H]⁺).

Preparation of (S)-1-(4-(((1H-imidazol-2-yl)methylamino)methyl)benzyl)-1-(3-morpholinopropyl)-3-(1-(naphthalen-1-yl)ethyl)urea (AO): A solution of (S)-1-(4-formylbenzyl)-1-(3-morpholinopropyl)-3-(1-(naphthalen-1-yl)ethyl)urea, 5 (0.63 g, 1.37 mmol) in 1,2-dichloroethane was treated with amino(methyl)imidazole (0.28 g, 1.64 mmol, 1.2), sodium triacetoxyborohydride (0.44 g, 2.05 mmol) and a couple drops of acetic acid. The resulting mixture was warmed to 65° C. and stirred for 18 h. A saturated aqueous solution of sodium bicarbonate (5 mL) was added. The product was extracted three times with 5 mL of ethyl acetate. The combined organic layers were washed with brine, dried over magnesium sulfate, filtered and concentrated. The crude material was purified by silica gel chromatography (0-5% MeOH/CHCl₃) to afford 0.28 g (38% yield) of pure product. The product was diluted in diethyl ether and 1 equiv. of 1N HCl were added to form the monohydrochloride salt AO: ¹H NMR (400 MHz, CDCl₃) δ 8.13 (d, J=8.0 Hz, 1H), 7.82 (d, J=7.6 Hz, 1H), 7.75 (d, J=8.0 Hz, 1H), 7.54-7.38 (m, 4H), 7.20-7.14 (m, 4H), 6.11 (bs, 1H), 5.90-5.80 (m, 1H), 4.48 (q, J=12.8 Hz, 2H), 3.79 (s, 2H), 3.70 (s, 2H), 3.36-3.25 (m, 2H), 3.22-3.05 (m, 4H), 2.21-2.05 (m, 4H), 1.98-1.86 (m, 2H), 1.65 (d, J=6.8 Hz, 3H), 1.58-1.46 (m, 2H); ESI⁺ MS: m/z (rel intensity) 541.3 (100, [M+H]⁺).

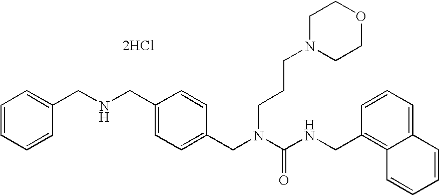

1-(4-((benzylamino)methyl)benzyl)-1-(3-morpholinopropyl)-3-(naphthalen-1-ylmethyl)urea (AP): ¹H NMR (400 MHz, d⁶-DMSO) δ 8.36 (bs, 1H), 8.13 (d, J=8.0 Hz, 1H), 7.89 (d, J=8.0 Hz, 1H), 7.77 (d, J=8.0 Hz, 1H), 7.60-7.15 (m, 12H), 4.72 (d, J=4.0 Hz, 2H), 4.20 (d, J=6.0 Hz, 2H), 4.10-4.05 (m, 4H), 3.90-3.80 (m, 2H), 3.78 (t, J=11.2 Hz, 2H), 3.30-3.18 (m, 4H), 3.00-2.85 (m, 4H), 1.95-1.80 (m, 2H); ESI⁺ MS: m/z (rel intensity) 537.2 (100, [M+H]⁺).

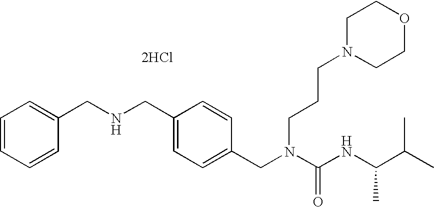

(S)-1-(4-((benzylamino)methyl)benzyl)-3-(3-methylbutan-2-yl)-1-(3-morpholinopropyl)urea (AQ): ¹H NMR (400 MHz, d⁶-DMSO) δ 8.29 (bs, 1H), 7.55-7.45 (m, 3H), 7.43-7.86 (m, 3H), 7.22 (d, J=7.6 Hz, 2H), 4.55-4.37 (m, 2H), 4.10-4.02 (m, 2H), 3.80-3.60 (m, 5H), 3.40-3.20 (m, 6H), 3.00-2.90 (m, 4H), 1.90-1.80 (m, 2H), 1.05 (t, J=7.2 Hz, 6H), 0.76 (d, J=6.4 Hz, 3H); ESI⁺ MS: m/z (rel intensity) 467.2 (100, [M+H]⁺).

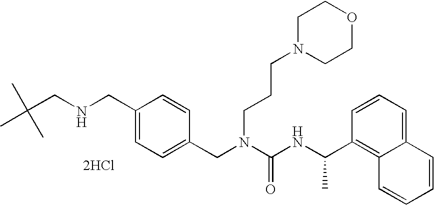

(S)-1-(4-((neopentylamino)methyl)benzyl)-1-(3-morpholinopropyl)-3-(1-(naphthalen-1-yl)ethyl)urea (AR): ¹H NMR (400 MHz, CDCl₃) δ 8.16 (d, J=8.4 Hz, 1H), 7.82 (d, J=8.0 Hz, 1H), 7.75 (d, J=7.6 Hz, 1H), 7.53-7.39 (m, 4H), 7.25 (d, J=8.4 Hz, 2H), 7.21 (d, J=8.4 Hz, 1H), 5.90-5.80 (m, 2H), 4.50 (q, J=16.0 Hz, 2H), 3.77 (s, 2H), 3.36-3.32 (m, 2H), 3.20-3.12 (m, 4H), 2.18-2.08 (m, 4H), 1.99-1.90 (m, 2H), 1.65 (d, J=7.2 Hz, 3H), 1.57-1.42 (m, 2H), 0.89 (s, 9H); ESI⁺ MS: m/z (rel intensity) 531 (50, [M+H]⁺).

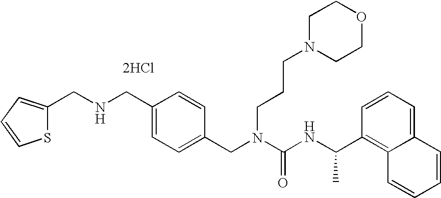

(S)-1-(3-morpholinopropyl)-3-(1-(naphthalen-1-yl)ethyl)-1-(4-((thiophen-2-ylmethylamino)methyl)benzyl urea (AS): ¹H NMR (400 MHz, d⁶-DMSO) δ 8.13 (d, J=8.8 Hz, 1H), 7.89 (d, J=7.6 Hz, 1H), 7.76 (d, J=8.4 Hz, 1H), 7.60 (d, J=5.6 Hz, 1H), 7.55-7.40 (m, 5H), 7.35-7.27 (m, 1H), 7.21 (d, J=7.2 Hz, 1H), 7.15-7.03 (m, 2H), 5.72-5.60 (m, 1H), 4.55-4.45 (m, 2H), 4.40-4.28 (m, 2H), 4.14-4.02 (m, 2H), 3.90-3.05 (m, 10H), 1.90-1.75 (m, 2H), 1.48 (d, J=6.4 Hz, 3H); ESI⁺ MS: m/z (rel intensity) 557.2 (100, [M+H]⁺).

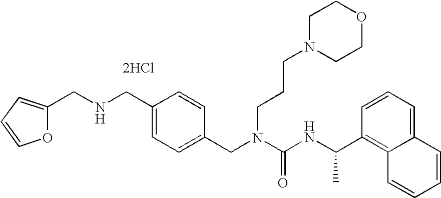

(S)-1-(4-((furan-2-ylmethylamino)methyl)benzyl)-1-(3-morpholinopropyl)-3-(1-(naphthalen-1-yl)ethyl urea (AT): ¹H NMR (400 MHz, d⁶-DMSO) δ 8.12 (d, J=7.6 Hz, 1H), 7.88 (d, J=8.8 Hz, 1H), 7.80-7.70 (m, 2H), 7.58-7.40 (m, 5H), 7.21 (d, J=6.8 Hz, 2H), 7.09 (d, J=6.0 Hz, 1H), 6.63 (s, 1H), 6.50 (s, 1H), 5.75-5.60 (m, 1H), 4.51 (s, 2H), 4.13 (s, 2H), 4.05 (s, 2H), 3.86-3.05 (m, 8H), 2.95-2.80 (m, 4H), 1.90-1.78 (m, 2H), 1.48 (d, J=6.0 Hz, 3H); ESI⁺ MS: m/z (rel intensity) 541.2 (100, [M+H]⁺).

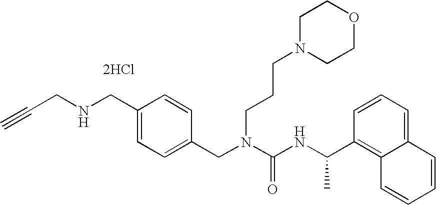

(S)-1-(3-morpholinopropyl)-3-(1-(naphthalen-1-yl)ethyl)-1-(4-(1-prop-2-ylnylamino)methyl)benzyl)urea (AU): ¹H NMR (400 MHz, d⁶-DMSO) δ 8.13 (d, J=8.0 Hz, 1H), 7.89 (d, J=7.2 Hz, 1H), 7.76 (d, J=8.0 Hz, 1H), 7.78-7.40 (m, 5H), 7.21 (d, J=8.0 Hz, 2H), 7.15-7.05 (m, 1H), 5.72 (s, 1H), 5.72-5.61 (m, 1H), 4.51 (s, 2H), 4.10 (bs, 2H), 3.90-3.60 (m, 6H), 3.40-3.00 (m, 4H), 2.97-2.80 (m, 4H), 1.90-1.75 (m, 2H), 1.48 (d, J=6.4 Hz, 3H); ESI⁺ MS: m/z (rel intensity) 499.2 (100, [M+H]⁺).

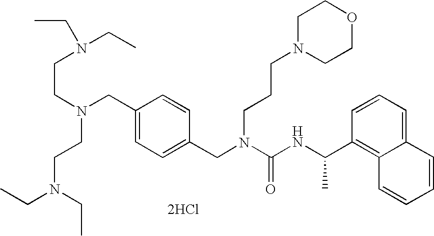

(S)-1-(4-((bis(2-(diethylamino)ethyl)amino)methyl)benzyl)-1-(3-morpholinopropyl)-3-(1-(naphthalen-1-yl)methyl)urea (AV): ¹H NMR (400 MHz, d⁶-DMSO) δ 8.13 (d, J=8.8 Hz, 1H), 7.89 (d, J=7.2 Hz, 1H), 7.76 (d, J=8.0 Hz, 1H), 7.56-7.40 (m, 4H), 7.33 (d, J=8.0 Hz, 2H), 7.16 (d, J=8.0 Hz, 2H), 7.06 (d, J=6.8 Hz, 1H), 5.72-5.61 (m, 1H), 4.49 (s, 2H), 3.90-3.65 (m, 4H), 3.61 (bs, 2H), 3.40-2.70 (m, 26H), 1.90-1.76 (m, 2H), 1.48 (d, J=6.8 Hz, 3H), 1.51 (t, J=7.6 Hz, 12H); ESI⁺ MS: m/z (rel intensity) 659.4 (100, [M+H]⁺).

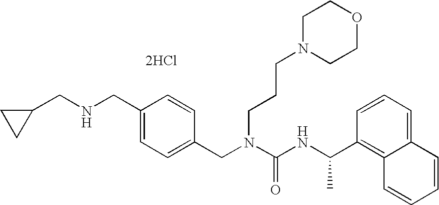

(S)-1-(4-((cyclopropylmethylamino)methyl)benzyl)-1-(3-morpholinopropyl)-3-(1-(naphthalen-1-yl)ethyl)urea (AW): ¹H NMR (400 MHz, d⁶-DMSO) δ 8.13 (d, J=8.4 Hz, 1H), 7.90 (d, J=7.6 Hz, 1H), 7.76 (d, J=8.0 Hz, 1H), 7.58-7.41 (m, 5H), 7.22 (d, J=8.0 Hz, 2H), 7.08 (d, J=8.0 Hz, 1H), 5.70-5.60 (m, 1H), 4.52 (s, 2H), 4.10-4.02 (m, 2H), 3.90-3.60 (m, 4H), 3.30-3.00 (m, 4H), 3.00-2.80 (m, 4H), 2.80-2.70 (m, 2H), 1.90-1.76 (m, 2H), 1.48 (d, J=6.4 Hz, 3H), 1.12-1.02 (m, 1H), 0.60-0.50 (m, 2H), 0.35-0.30 (m, 2H); ESI⁺ MS: m/z (rel intensity) 515.3 (100, [M+H]⁺).

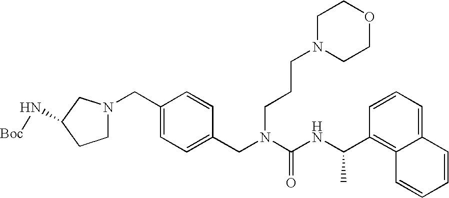

tert-butyl-(S)-1-(4-((1-(3-morpholinopropyl)-3-((S)-1-(naphthalen-1-yl)ethyl)ureido)methyl)benzyl)pyrrolidin-3-yl carbamate (AX): ¹H NMR (400 MHz, CDCl₃) δ 8.15 (d, J=8.0 Hz, 1H), 7.81 (d, J=7.6 Hz, 1H), 7.74 (d, J=8.0 Hz, 1H), 7.53-7.37 (m, 4H), 7.24-7.16 (m, 3H), 6.00-5.90 (m, 1H), 5.90-5.80 (m, 1H), 4.92-4.85 (m, 1H), 4.50 (q, J=12.8 Hz, 2H), 4.20-4.08 (m, 1H), 3.54 (s, 2H), 3.35-3.25 (m, 2H), 3.25-3.05 (m, 2H), 2.78-2.65 (m, 1H), 2.60-2.52 (m, 1H), 2.52-2.42 (m, 1H), 2.30-2.05 (m, 6H), 2.02-2.00 (m, 1H), 1.98-1.85 (m, 2H), 1.64 (d, J=6.4 Hz, 3H), 1.63-1.45 (m, 2H), 1.40 (s, 9H); ESI⁺ MS: m/z (rel intensity) 630.3 (100, [M+H]⁺).

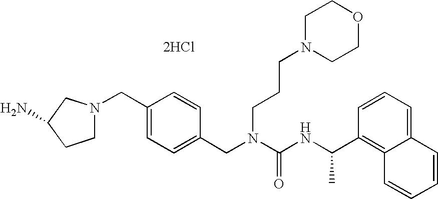

1-(4-(((S)-3-aminopyrrolidin-1-yl)methyl)benzyl)-1-(3-morpholinopropyl)-3-((S)-1-(naphthalen-1-yl)ethyl)urea (AY) (from Boc-removal of AX): ¹H NMR (400 MHz, d⁶-DMSO) δ 8.72 (bs, 1H), 8.57 (bs, 1H), 8.30 (bs, 1H), 8.13 (bs, 1H), 7.90 (bs, 1H), 7.76 (bs, 1H), 7.60-7.40 (m, 4H), 7.28-7.04 (m, 2H), 5.68 (bs, 1H), 4.58-4.30 (m, 4H), 3.90-3.60 (m, 4H), 3.35-3.05 (m, 8H), 3.00-2.80 (m, 4H), 2.30-1.94 (m, 2H), 1.92-1.70 (m, 3H), 1.53-1.40 (m, 3H); ESI⁺ MS: m/z (rel intensity) 530.3 (100, [M+H]⁺).

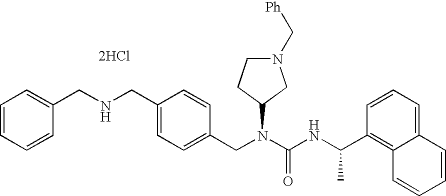

1-(4-((benzylamino)methyl)benzyl)-1-((R)-1-benzylpyrrolidin-3-yl)-3-((S)-1-(naphthalen-1-yl)ethyl)urea (AZ): ¹H NMR (400 MHz, d⁶-DMSO) δ 8.40-8.30 (m, 1H), 8.15-8.02 (m, 1H), 7.92-7.85 (m, 1H), 7.78-7.70 (m, 1H), 7.55-7.05 (m, 17H), 5.70-5.60 (m, 1H), 4.82-4.50 (m, 3H), 4.45-4.15 (m, 3H), 4.12-4.00 (m, 4H), 3.35-2.90 (m, 3H), 2.10-1.85 (m, 2H), 1.50-1.40 (m, 3H); ESI⁺ MS: m/z (rel intensity) 583.3 (100, [M+H]⁺).

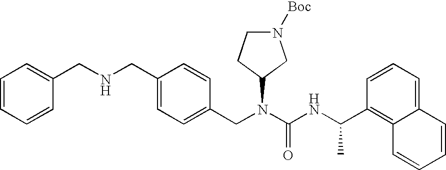

(S)-tert-butyl 3-(1-(4-((benzylamino)methyl)benzyl)-3-((S)-1-(naphthalen-1-yl)ethyl)ureido)pyrrolidine-1-carboxylate (BA): ¹H NMR (400 MHz, CDCl₃) δ 8.03 (d, J=8.4 Hz, 1H), 7.75 (d, J=8.0 Hz, 1H), 7.63 (d, J=8.0 Hz, 1H), 7.50-7.40 (m, 2H), 7.36-7.30 (m, 4H), 7.30-7.22 (m, 4H), 7.16 (d, J=8.0 Hz, 2H), 7.07 (d, J=8.4 Hz, 2H), 5.80-5.70 (m, 1H), 5.20-5.08 (m, 1H), 4.57 (d, J=6.8 Hz, 1H), 4.30 (s, 2H), 3.75 (s, 2H), 3.71 (s, 2H), 3.62 (t, J=10.4 Hz, 1H), 3.50-3.35 (m, 1H), 3.28-3.17 (m, 1H), 3.16-3.02 (m, 1H), 2.10-2.00 (m, 1H), 1.44 (d, J=6.0 Hz, 3H), 1.41 (s, 9H); ESI⁺ MS: m/z (rel intensity) 593.3 (100, [M+H]⁺).

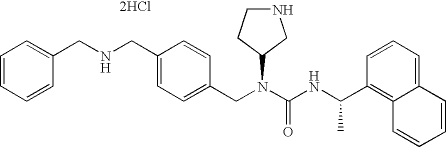

1-(4-((benzylamino)methyl)benzyl)-3-((S)-1-(naphthalen-1-yl)ethyl)-1-((S)-pyrrolidin-3-yl)urea (BB) (Boc removal of BA): ¹H NMR (400 MHz, d⁶-DMSO) δ 8.10 (d, J=8.0 Hz, 1H), 7.87 (d, J=7.6 Hz, 1H), 7.74 (t, J=4.4 Hz, 1H), 7.55-7.37 (m, 10H), 7.19 (d, J=8.0 Hz, 2H), 7.13 (d, J=7.2 Hz, 1H), 5.70-5.62 (m, 1H), 4.62 (q, J=16.0 Hz, 2H), 4.55-4.46 (m, 1H), 4.10-4.02 (m, 2H), 3.42 (bs, 2H), 3.30-3.20 (m, 1H), 3.20-3.08 (m, 1H), 3.00-2.85 (m, 2H), 2.00-1.80 (m, 2H), 1.44 (d, J=7.2 Hz, 3H); ESI⁺ MS: m/z (rel intensity) 493.2 (100, [M+H]⁺).

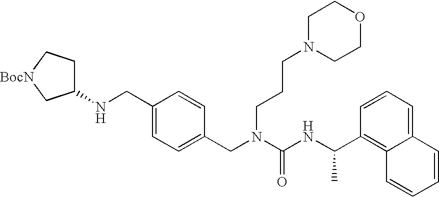

(S)-tert-butyl 3-(4-((1-(3-morpholinopropyl)-3-((S)-1-(naphthalen-1-yl)ethyl)ureido)methyl)benzylamino)pyrrolidine-1-carboxylate (BC): ¹H NMR (400 MHz, CDCl₃) δ 8.15 (d, J=8.4 Hz, 1H), 7.82 (d, J=8.0 Hz, 1H), 7.75 (d, J=8.0 Hz, 1H), 7.52-7.40 (m, 4H), 7.25-7.20 (m, 4H), 5.96 (bs, 1H), 5.90-5.80 (m, 1H), 4.50 (q, J=14.4 Hz, 2H), 3.60-3.25 (m, 7H), 3.25-3.04 (m, 5H), 2.20-2.10 (m, 4H), 2.08-1.90 (m, 2H), 1.74-1.48 (m, 3H), 1.64 (d, J=6.4 Hz, 3H), 1.43 (s, 9H); ESI⁺ MS: m/z (rel intensity) 630.3 (100, [M+H]⁺).

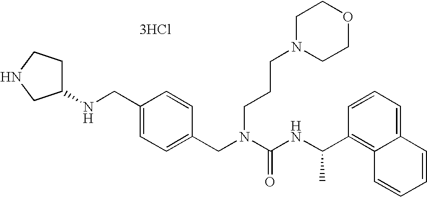

1-(3-morpholinopropyl)-3-((S)-1-(naphthalen-1-yl)ethyl)-1-(4-(((S)-pyrrolidin-3-ylamino)methyl)benzyl)urea (BD) (Boc deprotection of BC): ¹H NMR (400 MHz, d⁶-DMSO) δ 8.13 (d, J=8.4 Hz, 1H), 7.90 (d, J=8.4 Hz, 1H), 7.76 (d, J=8.4 Hz, 1H), 7.58-7.42 (m, 6H), 7.23 (d, J=8.0 Hz, 2H), 7.12-7.06 (m, 1H), 5.72-5.64 (m, 1H), 4.55-4.50 (m, 2H), 4.20-4.12 (m, 4H), 3.90-3.54 (m, 4H), 3.52-3.35 (m, 2H), 3.30-3.00 (m, 5H), 2.95-2.80 (m, 4H), 2.35-2.18 (m, 2H), 1.85-1.70 (m, 2H), 1.48 (d, J=7.2 Hz, 3H); ESI⁺ MS: m/z (rel intensity) 530.2 (100, [M+H]⁺).

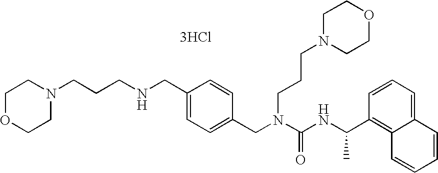

(S)-1-(3-morpholinopropyl)-1-(4-((3-morpholinopropylamino)methyl)benzyl)-3-(1-(naphthalen-1-yl)ethyl)urea (BE): ¹H NMR (400 MHz, d⁶-DMSO) δ 8.13 (d, J=8.0 Hz, 1H), 7.89 (d, J=8.0 Hz, 1H), 7.76 (d, J=8.0 Hz, 1H), 7.56-7.44 (m, 5H), 7.22 (d, J=7.6 Hz, 2H), 7.10 (d, J=7.2 Hz, 1H), 5.70-5.61 (m, 1H), 4.51 (bs, 2H), 4.15-4.03 (m, 2H), 3.95-3.60 (m, 6H), 3.40-2.80 (m, 14H), 2.46 (bs, 4H), 2.20-2.10 (m, 2H), 1.90-1.76 (m, 2H), 1.49 (d, J=6.8 Hz, 3H); ESI⁺ MS: m/z (rel intensity) 588.4 (100, [M+H]⁺).

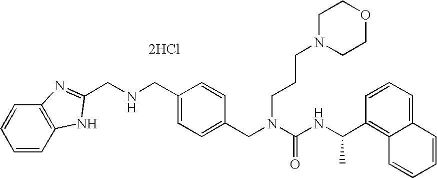

(S)-1-(4-(((1H-benzo[d]imidazol-2-yl)methylamino)methyl)benzyl)-1-(3-morpholinopropyl)-3-(1-(naphthalen-1-yl)ethyl)urea (BF): ¹H NMR (400 MHz, CDCl₃): δ 8.12 (d, J=8.4 Hz, 1H), 7.81 (d, J=8.0 Hz, 1H), 7.74 (d, J=8.0 Hz, 1H), 7.56-7.38 (m, 6H), 7.24-7.14 (m, 5H), 7.08-6.74 (m, 2H), 6.06 (bs, 1H), 5.88-5.80 (m, 1H), 4.45 (q, J=12.0 Hz, 2H), 4.06 (s, 2H), 3.79 (s, 2H), 3.38-3.30 (m, 2H), 3.26-3.02 (m, 4H), 2.22-2.10 (m, 4H), 2.00-1.92 (m, 2H), 1.65 (d, J=6.8 Hz, 3H), 1.58-1.50 (m, 2H); ESI⁺ MS: m/z (rel intensity) 591.2 (100, [M+H]⁺).

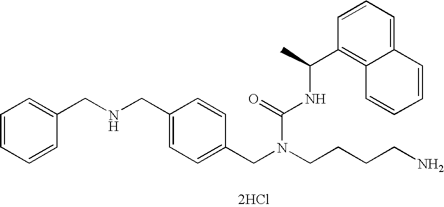

(S)-1-(4-aminobutyl)-1-(4-((benzylamino)methyl)benzyl)-3-(1-napthalen-1-yl)ethyl)urea (BG): ¹H NMR (400 MHz, d⁶-DMSO) δ 9.82 (bs, 2H), 8.12 (d, J=8.4 Hz, 1H), 8.03 (bs, 2H), 7.88 (d, J=8.4 Hz, 1H), 7.75 (d, J=8.0 Hz, 1H), 7.54-7.37 (m, 8H), 7.18 (d, J=8.0 Hz, 2H), 6.93 (d, J=7.6 Hz, 1H), 5.66 (t, J=6.4 Hz, 1H), 4.48 (s, 2H), 4.06 (bs, 4H), 3.48 (bs, 2H), 3.13 (bs, 2H), 2.67 (bs, 2H), 1.46 (bs, 6H); ESI⁺ MS: m/z (rel intensity) 495.2 (100, [M+H]⁺).

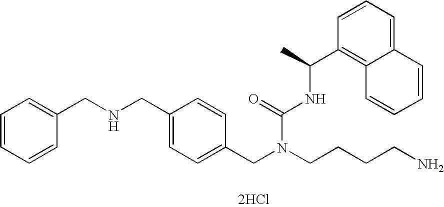

(S)-1-(3-aminopropyl)-1-(4-((benzylamino)methyl)benzyl)-3-(1-(napthalen-1-yl)ethyl)urea (BH): ¹H NMR (400 MHz, d⁶-DMSO) δ 9.80 (bs, 2H), 8.12 (d, J=8.4 Hz, 1H), 8.03 (bs, 2H), 7.88 (d, J=8.4 Hz, 1H), 7.75 (d, J=8.0 Hz, 1H), 7.54-7.37 (m, 8H), 7.19 (d, J=8.4 Hz, 2H), 7.06 (d, J=8.0 Hz, 1H), 5.67 (bs, 1H), 4.48 (bs, 2H), 4.06 (bs, 3H), 3.54 (s, 2H), 3.26 (bs, 2H), 2.69 (bs, 2H), 1.73 (bs, 2H), 1.48 (d, J=6.8 Hz, 3H); ESI⁺ MS: m/z (rel intensity) 481.2 (100, [M+H]⁺).

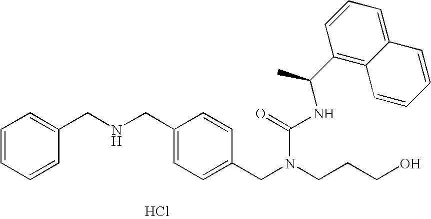

(S)-1-(4-((benzylamino)methyl)benzyl)-1-(3-hydroxypropyl)-3-(1-(naphthalen-1-yl)ethyl)urea (BI): ¹H NMR (400 MHz, d⁶-DMSO) δ 8.11 (bs, 1H), 7.87 (bs, 1H), 7.74 (bs, 1H), 7.47-7.38 (m, 8H), 7.27-7.20 (m, 4H), 6.87 (bs, 1H), 4.46 (bs, 2H), 4.19-3.86 (m, 8H), 3.53-3.12 (m, 4H), 2.04 (s, 1H), 1.45 (bs, 2H); ESI⁺ MS: m/z (rel intensity) 482.2 (100, [M+H]⁺).

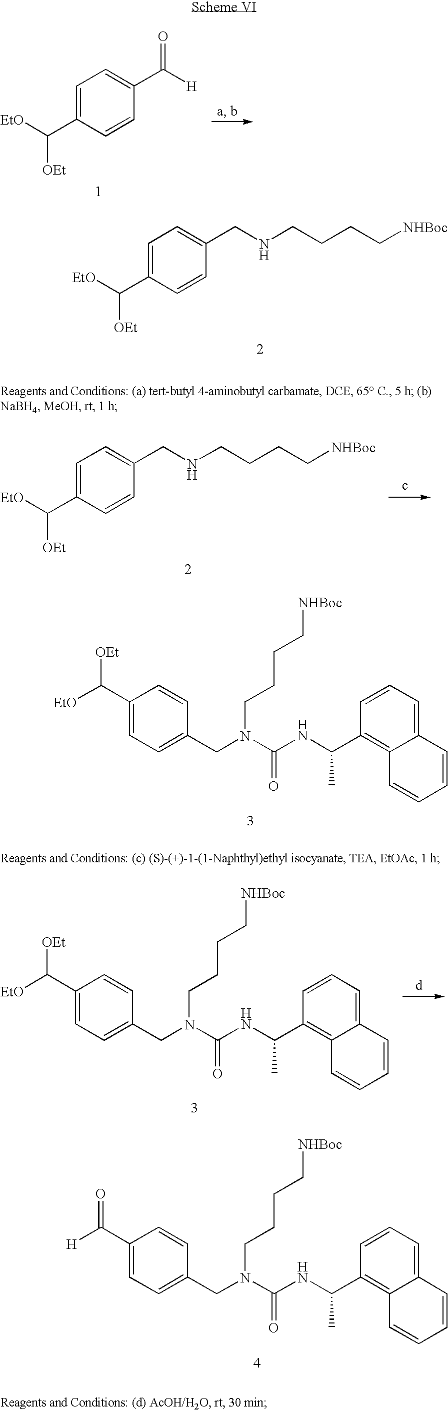

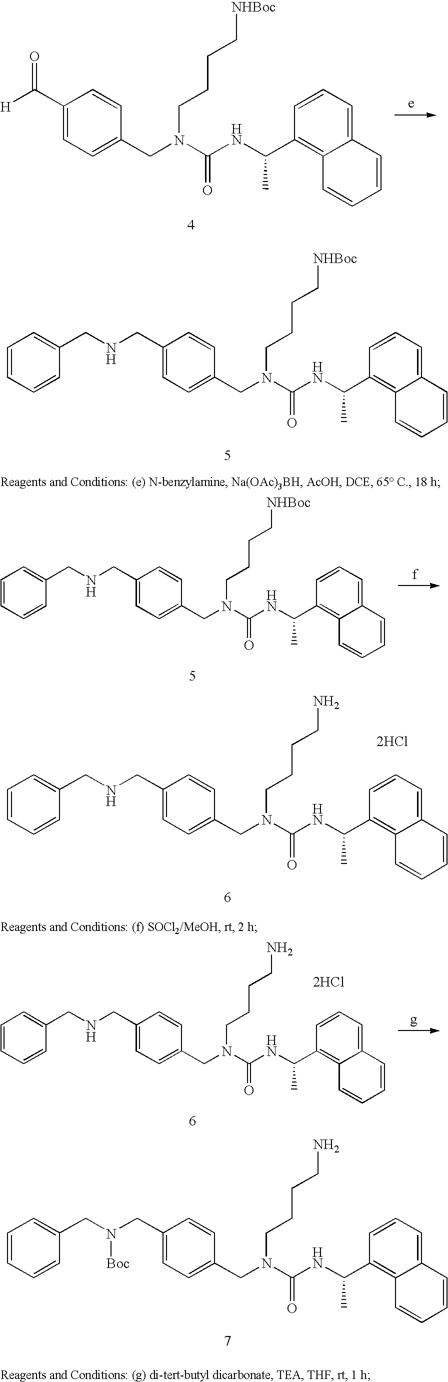

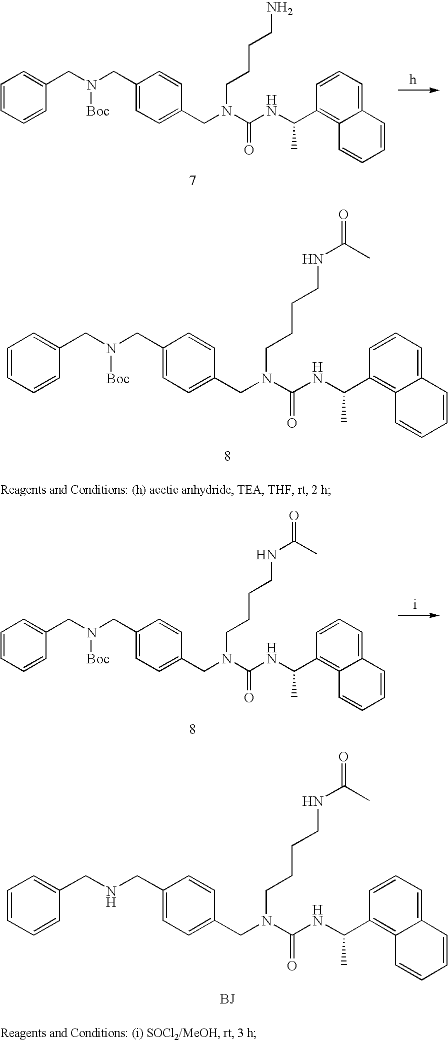

### Example 6

### (S)—N-(4-(1-(4-((benzylamino)methyl)benzyl)-3-(1-(naphthalen-1-yl)ethyl)ureido)butyl)acetamide (BJ)

Preparation of tert-butyl 4-(4-(diethoxymethyl)benzylamino)butylcarbamate (2): To a solution of terephthaldehyde mono(diethyl acetal), 1, (4.0 g, 19.2 mmol) in dichloroethane (40 mL) was added tert-butyl 4-aminobutyl carbamate (3.68 mL, 19.2 mmol). The reaction stirred at 65° C. for 18 h. The mixture was cooled to 0° C. and NaBH₄ (1.45 g, 38.4 mmol) was added slowly. The reaction mixture was warmed to room temperature and stirred for 1 h. The solution was quenched with a saturated aqueous solution of sodium bicarbonate (15 mL), diluted with ethyl acetate (100 mL). The layers were separated. The organic layer was dried over magnesium sulfate, filtered and concentrated in vacuo to yield, 2, (quantitative) as a crude product that was used without further purification in the next step. ESI⁺ MS: m/z (rel intensity) 381.2 (100, [M+H]⁺).

Preparation of (S)-tert-butyl 4-(1-(4-(diethoxymethyl)benzyl)-3-(1-(naphthalen-1-yl)ethyl)ureido)butylcarbamate (3): To a solution of tert-butyl 4-(4-(diethoxymethyl)benzylamino)butylcarbamate, 2, (7.8 g, 20.6 mmol) in ethyl acetate (40 ml) was added triethylamine (5.7 mL, 41.2 mmol) and (S)-(+)-1-(Naphthyl)ethyl isocyanate (5.4 mL, 30.9 mmol). The reaction stirred at room temperature for 18 h. The mixture was quenched with a saturated aqueous solution of sodium bicarbonate, extracted with ethyl acetate (150 mL). The combined organic layers were washed with brine, dried over magnesium sulfate, filtered and concentrated in vacuo to provide, 3, as a crude compound that was used without further purification in the next step: ESI⁺ MS: m/z (rel intensity) 600.2 (100, [M+Na]⁺).

Preparation of (S)-tert-butyl 4-(1-(4-formylbenzyl)-3-(1-(naphthalen-1-yl)ethyl)ureido)butylcarbamate (4): To a 1:1 ratio of AcOH/H₂O (30 mL) was added (S)-tert-butyl-4-(1-(4-(diethoxymethyl)benzyl)-3-(1-(naphthalen-1-yl)ethyl)ureido)-butylcarbamate, 3, (1.73 g, 3.00 mmol) and the reaction stirred at room temperature for 30 min. The mixture was concentrated in vacuo to afford (4) (quantitative yield) as a crude solid. ESI⁺ MS: m/z (rel intensity) 526.2 (100, [M+Na]⁺).

Preparation of (S)-tert-butyl 4-(1-(4-((benzylamino)methyl)benzyl)-3-(1-(naphthalen-1-yl)ethyl)ureido)butylcarbamate (5): To a solution of (S)-tert-butyl 4-(1-(4-formylbenzyl)-3-(1-(naphthalen-1-yl)ethyl)ureido)butylcarbamate, 4, (4.9 g, 9.8 mmol) in dichloroethane (20 mL) was added N-benzylamine (1.3 mL, 11.7 mmol), acetic acid (10 drops) and Na(OAc)₃BH (3.7 g, 17.6 mmol). The reaction mixture stirred at 65° C. for 18 h. The mixture was cooled to room temperature and quenched with a saturated aqueous solution of sodium bicarbonate. The product was extracted with ethyl acetate (100 mL). The combined organic layers were washed with brine (20 mL), dried over magnesium sulfate, filtered and concentrated in vacuo.

The crude material was purified by silica gel chromatography (5% MeOH/CHCl₃) to give 5 (1.51 g, 26% yield) as a pale yellow solid. ¹H NMR (400 MHz, d⁶-DMSO) δ 8.13 (d, J=8.4 Hz, 1H), 7.88 (d, J=8.0 Hz, 1H), 7.74 (d, J=8.0 Hz, 1H), 7.51-7.42 (m, 3H), 7.31-7.25 (m, 3H), 7.23-7.19 (m, 3H), 7.09 (d, J=7.6 Hz, 2H), 6.77-6.74 (m, 2H), 4.41 (s, 2H), 3.62 (d, J=5.6 Hz, 4H), 3.30 (bs, 1H), 3.13-3.07 (m, 3H), 2.82 (d, J=6.4 Hz, 2H), 1.46 (d, J=7.6 Hz, 3H), 1.37-1.25 (m, 12H); ESI⁺ MS: m/z (rel intensity) 595.3 (100, [M+H]⁺).

Preparation of (S)-1-(4-aminobutyl)-1-(4-((benzylamino)methyl)benzyl)-3-(1-(naphthalen-1-yl)ethyl)urea (6): To a solution of (S)-tert-butyl 4-(1-(4-((benzylamino)methyl)benzyl)-3-(1-(naphthalen-1-yl)ethyl)ureido)butylcarbamate, 5, (1.3 g, 2.2 mmol) in methanol (5 mL) was added thionyl chloride (1 mL). The reaction was stirred at room temperature for 18 h. The reaction was then concentrated and dried in vacuo, to yield 6 (95% yield) as the dihydrochloride salt of BG. ¹H NMR (400 MHz, d⁶-DMSO) δ 9.82 (bs, 2H), 8.12 (d, J=8.4 Hz, 1H), 8.03 (bs, 2H), 7.88 (d, J=8.4 Hz, 1H), 7.75 (d, J=8.0 Hz, 1H), 7.54-7.37 (m, 8H), 7.18 (d, J=8.0 Hz, 2H), 6.93 (d, J=7.6 Hz, 1H), 5.66 (t, J=6.4 Hz, 1H), 4.48 (s, 2H), 4.06 (bs, 4H), 3.48 (bs, 2H), 3.13 (bs, 2H), 2.67 (bs, 2H), 1.46 (bs, 6H); ESI⁺ MS: m/z (rel intensity) 495.2 (100, [M+H]⁺).

Preparation of (S)-tert-butyl 4-((1-(4-aminobutyl)-3-(1-(naphthalen-1-yl)ethyl)ureido)methyl)benzyl(benzyl)carbamate (7): To a solution of (S)-1-(4-aminobutyl)-1-(4-((benzylamino)methyl)benzyl)-3-(1-(naphthalen-1-yl)ethyl)urea, 6, (1.2 g, 2.0 mmol) in tetrahydrofuran (5 mL) was added triethylamine (1.0 mL, 7.2 mmol) and di-tert-butyl-dicarbonate (0.4 g, 2.0 mmol). The reaction was stirred at room temperature for 1 h. The mixture was quenched with a saturated aqueous solution of sodium bicarbonate. The product was extracted with ethyl acetate (25 mL). The combined organic layers were dried over magnesium sulfate, filtered and concentrated in vacuo to afford 7 (1.03 g, 85% yield) as crude product. Crude ¹H NMR (400 MHz, d⁶-DMSO) δ 8.14 (d, J=8.4 Hz, 1H), 7.88 (d, J=8.4 Hz, 1H), 7.74 (d, J=8.0 Hz, 1H), 7.52-7.41 (m, 4H), 7.30-7.18 (m, 6H), 7.08 (d, J=8.4 Hz, 2H), 6.78-6.74 (m, 1H), 4.41 (bs, 2H), 4.30 (bs, 1H), 4.23 (bs, 1H), 3.60 (d, J=5.6 Hz, 2H), 3.15-3.07 (m, 2H), 2.82 (d, J=6 Hz, 1H), 1.46 (d, J=6.4 Hz, 2H), 1.39-1.27 (m, 16H); ESI⁺ MS: m/z (rel intensity) 595.3 (100, [M+H]⁺).

Preparation of (S)-tert-butyl 4-((1-(4-acetamidobutyl)-3-(1-(naphthalen-1-yl)ethyl)ureido)methyl)benzyl(benzyl)carbamate (8): To a solution of (S)-tert-butyl 4-((1-(4-aminobutyl)-3-(1-(naphthalen-1-yl)ethyl)ureido)methyl)benzyl(benzyl)carbamate, 7, (0.50 g, 0.84 mmol) in tetrahydrofuran (2 mL) was added triethylamine (0.23 mL, 1.68 mmol) and acetic anhydride (0.09 mL, 1.00 mmol). The reaction was stirred at room temperature for 2 h. The solution was quenched with a saturated aqueous solution of sodium bicarbonate. The product was extracted with ethyl acteate (15 mL). The organic layer was dried over magnesium sulfate, filtered and concentrated in vacuo. The crude material was purified by silica gel chromatography (5% MeOH/CHCl₃) to give 8 (0.15 g, 28% yield): ¹H NMR (400 MHz, d⁶-DMSO) δ 8.12 (d, J=8.0 Hz, 1H), 7.88 (d, J=7.6 Hz, 1H), 7.75 (d, J=7.6 Hz, 1H), 7.51-7.42 (m, 3H), 7.33-7.23 (m, 3H), 7.17-7.05 (m, 5H), 6.75 (d, J=7.6 Hz, 2H), 4.41 (bs, 4H), 3.11-3.09 (m, 2H), 2.82 (d, J=6.4 Hz, 2H), 2.05 (s, 2H), 1.46 (d, J=6.8 Hz, 3H), 1.36-1.27 (m, 9H); ESI⁺ MS: m/z (rel intensity) 659.3 (100, [M+Na]⁺).

Preparation of (S)—N-(4-(1-(4-((benzylamino)methyl)benzyl)-3-(1-(naphthalen-1-yl)ethyl)ureido)butyl)acetamide (BJ): To a solution of (S)-tert-butyl-4-((1-(4-acetamidobutyl)-3-(1-(naphthalen-1-yl)ethyl)ureido)methyl)benzyl-(benzyl)carbamate, 8, (0.15 g, 0.23 mmol) in methanol (1 mL) was added thionyl chloride (0.50 mL). The reaction was stirred at room temperature for 3 h. The reaction was concentrated and dried in vacuo, to yield BJ (0.09 g, 67% yield) as the hydrochloride salt. ¹H NMR (400 MHz, d⁶-DMSO) δ 8.12 (bs, 1H), 7.89 (bs, 3H), 7.75 (bs, 1H), 7.45-7.08 (m, 10H), 6.85 (bs, 1H), 4.43 (bs, 6H), 3.53 (s, 2H), 3.12 (bs, 3H), 2.68 (bs, 2H), 2.05 (s, 2H), 1.45 (bs, 6H); ESI⁺ MS: m/z (rel intensity) 537.2 (100, [M+H]⁺).

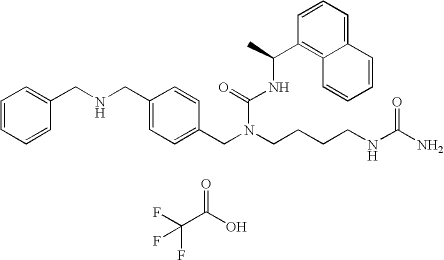

(S)-1-(4(-aminobutyl)urea)-1-(4-((benzylamino)methyl)benzyl)-3-(1-naphthalen-1-yl)ethyl)urea (BK): ¹H NMR (400 MHz, d⁶-DMSO) δ 8.13 (d, J=8.4 Hz, 1H), 7.88 (d, J=4.0 Hz, 1H), 7.75 (d, J=8.0 Hz, 1H), 7.53-7.42 (m, 3H), 7.30-7.06 (m, 8H), 6.76 (bs, 2H), 6.08 (s, NH₂), 5.65 (t, J=7.6 Hz, 1H), 4.40 (s, 2H), 4.28 (bs, 4H), 3.12-3.07 (m, 2H), 2.82 (bs, 2H), 1.95 (s, 1H), 1.45 (d, J=6.8 Hz, 4H), 1.28 (bs, 3H); ESI⁺ MS: m/z (rel intensity) 638.3 (60, [M+H]⁺).

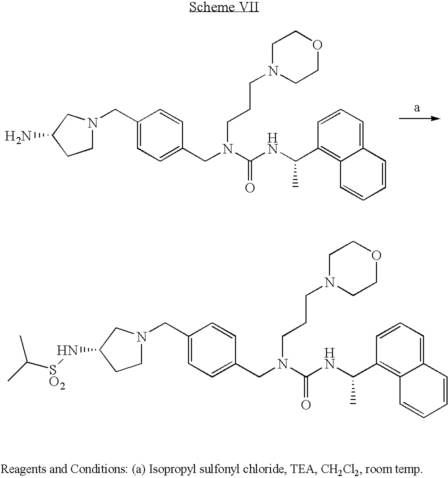

### Example 7

Preparation of N—((S)-1-(4-((1-(3-morpholinopropyl)-3-((S)-1-(naphthalen-1-yl)ethyl)ureido)methyl)benzyl)pyrrolidin-3-yl)propan-2-sulfonamide (BL): A suspension of BK (0.30 g, 0.52 mmol) in dichloromethane (5 mL) was treated with triethylamine (0.22 mL, 1.58 mmol) then with isopropyl sulfonyl chloride (0.08 mL, 0.63 mmol) dropwise. The resulting mixture was stirred at room temperature for 20 h. A saturated aqueous solution of sodium bicarbonate (5 mL) was added. The product was extracted three times with 5 mL of CH₂Cl₂. The combined organic layers were dried over potassium carbonate, filtered and concentrated. The crude material was purified by silica gel chromatography (0-20% MeOH/CH₂Cl₂) to afford pure product. The product was diluted in diethyl ether and 2 equiv. of 1N HCl were added to form the dihydrochloride salt BL: ¹H NMR (400 MHz, CDCl₃): δ 8.15 (d, J=8.4 Hz, 1H), 7.82 (d, J=6.4 Hz, 1H), 7.75 (d, J=8.0 Hz, 1H), 7.54-7.40 (m, 4H), 7.25-7.17 (m, 3H), 5.98 (bs, 1H), 5.90-5.80 (m, 1H), 4.52-4.47 (m, 2H), 4.00-3.90 (m, 1H), 3.60-3.55 (m, 2H), 3.58-3.30 (m, 2H), 3.26-3.05 (m, 4H), 2.90-1.50 (m, 21H), 1.33-1.29 (m, 3H); ESI⁺ MS: m/z (rel intensity) 636.3 (100, [M+H]⁺).

Compounds BM through BP listed below are non-limiting examples of the first aspect of the present invention which were prepared using methods similar to those described in Scheme VII (i.e., isopropyl sulfonyl chloride of was replaced by the appropriate electrophile).

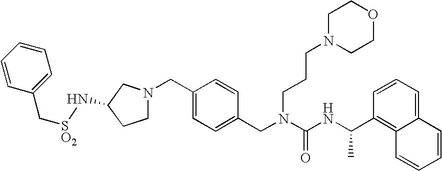

N—((S)-1-(4-((1-(3-morpholinopropyl)-3-((S)-1-(naphthalen-1-yl)ethyl)ureido)-methyl)benzyl)pyrrolidin-3-yl)-1-phenylmethane sulfonamide (BM): ¹H NMR (400 MHz, CDCl₃): δ 8.16 (d, J=8.4 Hz, 1H), 7.82 (d, J=8.4 Hz, 1H), 7.75 (d, J=7.6 Hz, 1H), 7.54-7.15 (m, 13H), 5.99 (bs, 1H), 5.90-5.82 (m, 1H), 4.55-4.44 (m, 2H), 4.18 (s, 2H), 3.62 (s, 4H), 3.49 (s, 2H), 3.36-3.25 (m, 2H), 3.24-3.05 (m, 4H), 2.72-2.60 (m, 2H), 2.54-2.40 (m, 2H), 2.25-2.05 (m, 4H), 2.00-1.85 (m, 4H), 1.65 (d, J=6.8 Hz, 3H), 1.65-1.50 (m, 3H); ESI⁺ MS: m/z (rel intensity) 684.2 (100, [M+H]⁺).

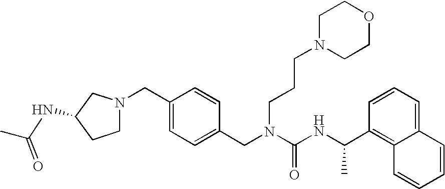

N—((S)-1-(4-((1-(3-morpholinopropyl)-3-((S)-1-(naphthalen-1-yl)ethyl)ureido)-methyl)benzyl)pyrrolidin-3-yl)acetamide (BN): ¹H NMR (400 MHz, CDCl₃): δ 8.16 (d, J=8.0 Hz, 1H), 7.82 (d, J=8.0 Hz, 1H), 7.75 (d, J=8.0 Hz, 1H), 7.53-7.38 (m, 4H), 7.24-7.22 (m, 4H), 5.90-5.82 (m, 1H), 4.51 (q, J=18.0 Hz, 2H), 4.48-4.40 (m, 1H), 3.65-3.53 (m, 2H), 3.36-3.30 (m, 2H), 3.25-3.10 (m, 4H), 2.95-2.86 (m, 1H), 2.65-2.55 (m, 1H), 2.55-2.48 (m, 1H), 2.30-1.90 (m, 9H), 1.92 (s, 3H), 1.65 (d, J=6.8 Hz, 3H), 1.62-1.50 (m, 2H); ESI⁺ MS: m/z (rel intensity) 572.3 (100, [M+H]⁺).

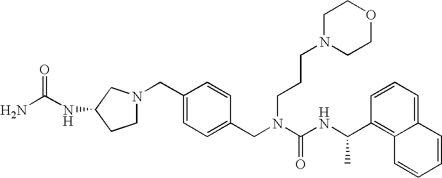

1-(4-(((S)-3-amino(pyrrolidin-1-yl)urea)methyl)benzyl)-1-(3-morpholinopropyl)-3-((S)-1-(naphthalen-1-yl)ethyl)urea (BO): ¹H NMR (400 MHz, CDCl₃) δ 8.16 (d, J=8.4 Hz, 1H), 7.82 (d, J=8.4 Hz, 1H), 7.75 (d, J=7.6 Hz, 1H), 7.52-7.36 (m, 4H), 7.24-7.20 (m, 3H), 5.90-5.82 (m, 1H), 4.56-4.43 (m, 2H), 3.64-3.45 (m, 2H), 3.38-3.30 (m, 2H), 3.25-3.10 (m, 4H), 2.75-2.60 (m, 1H), 2.52-2.40 (m, 1H), 2.30-2.10 (m, 4H), 2.00-2.1.90 (m, 2H), 1.70-1.50 (m, 10H); ESI⁺ MS: m/z (rel intensity) 573.3 (100, [M+H]⁺).

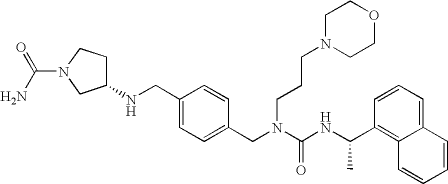

(S)-3-(4-((1-(3-morpholinopropyl)-3-((S)-1-(naphtalen-1-yl)ethyl)ureido)-methyl)benzylamino)pyrrolidine-1-carboxamide (BP): ¹H NMR (400 MHz, CDCl₃) δ 8.15 (d, J=8.4 Hz, 1H), 7.82 (d, J=7.6 Hz, 1H), 7.74 (d, J=7.2 Hz, 2H), 7.67 (d, J=7.2 Hz, 2H), 7.56-7.10 (m, 7H), 5.90-5.82 (m, 1H), 4.58-4.40 (m, 2H), 4.34 (bs, 2H), 3.85-3.60 (m, 6H), 3.60-3.42 (m, 2H), 3.42-3.25 (m, 4H), 3.25-3.05 (m, 4H), 2.22-2.05 (m, 2H), 2.05-1.85 (m, 2H), 1.65 (d, J=6.8 Hz, 3H), 1.62-1.50 (m, 2H); ESI⁺ MS: m/z (rel intensity) 573.3 (100, [M+H]⁺).

### Second Aspect

Compounds of formula (I), wherein L¹ is C(O), L² is alkylene, —C(O)—, or a covalent bond, and X and Y are both hydrogen, can have the following general structure (IB):

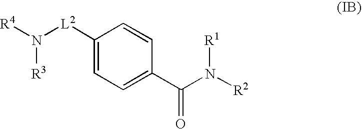

wherein R¹, R², L², R³ and R⁴ are defined herein below in Table 2. As shown below in Table 2, when L² is “-”, it denotes a covalent bond.

TABLE 2

| No. | 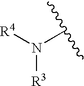 | L² | R¹ | R² |
|---|---|---|---|---|
| BT | 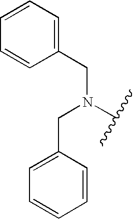 | CH₂ | H | benzyl |
| BU | 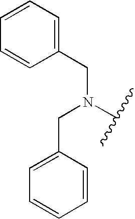 | CH₂ | H | 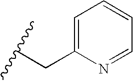 |
| BV | 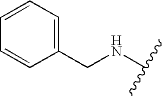 | CH₂ | H | 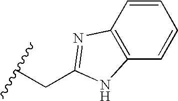 |
| BW | 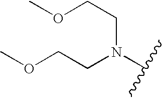 | CH₂ | H | 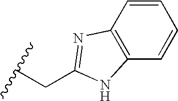 |
| BX | 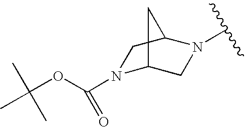 | CH₂ | H | 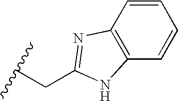 |
| BY | 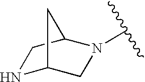 | CH₂ | H | 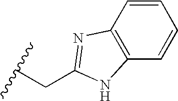 |
| BZ | 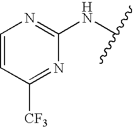 | CH₂ | benzyl | benzyl |
| CA | 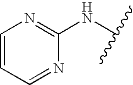 | CH₂ | benzyl | benzyl |
| CB | 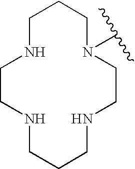 | CH₂ | H | benzyl |
| CC | 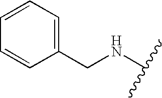 | CH₂ | CH₃ | 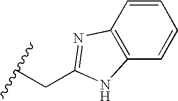 |
| CD | 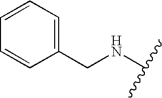 | CH₂CH₂ | 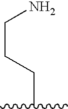 | 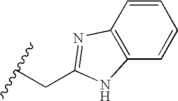 |
| CE | 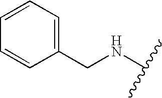 | CH₂CH₂ | 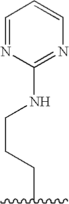 | 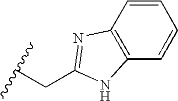 |
| CF | 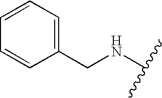 | — | benzyl | 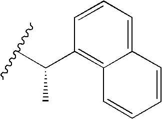 |
| CG | 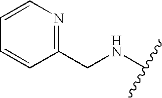 | CH₂ | benzyl | 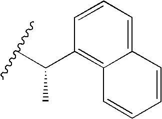 |
| CH | 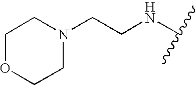 | — | H | 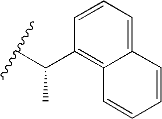 |
| CI | 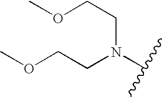 | CH₂ | H | 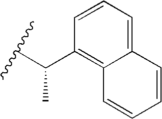 |
| CJ | 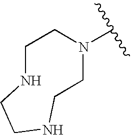 | CH₂ | H | 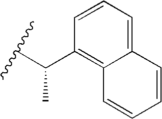 |
| CK | 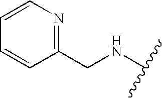 | CH₂ | H | 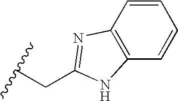 |
| CL | 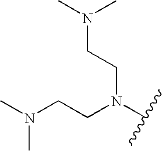 | CH₂ | H | 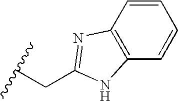 |
| CM | 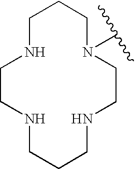 | CH₂ | H | 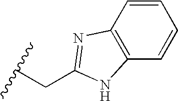 |
| CN | 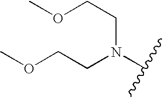 | CH₂ | H | 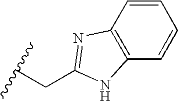 |
| CO | 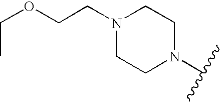 | CH₂ | H | 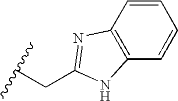 |
| CP | 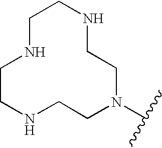 | CH₂ | H | 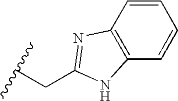 |
| CQ | 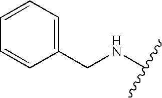 | CH₂ | CH₃ | 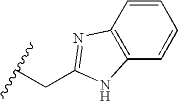 |
| CR | 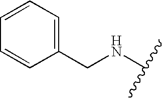 | CH₂ | 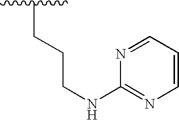 | 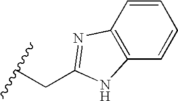 |
| CS | 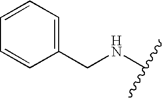 | CH₂ | 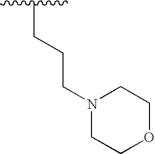 | 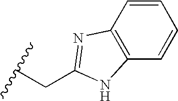 |
| CT | 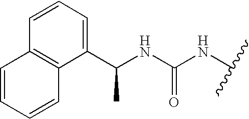 | CH₂ | H | 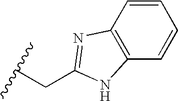 |

The compounds which comprise the third aspect of the present invention can be prepared by the procedure outlined herein below in Scheme VIII.

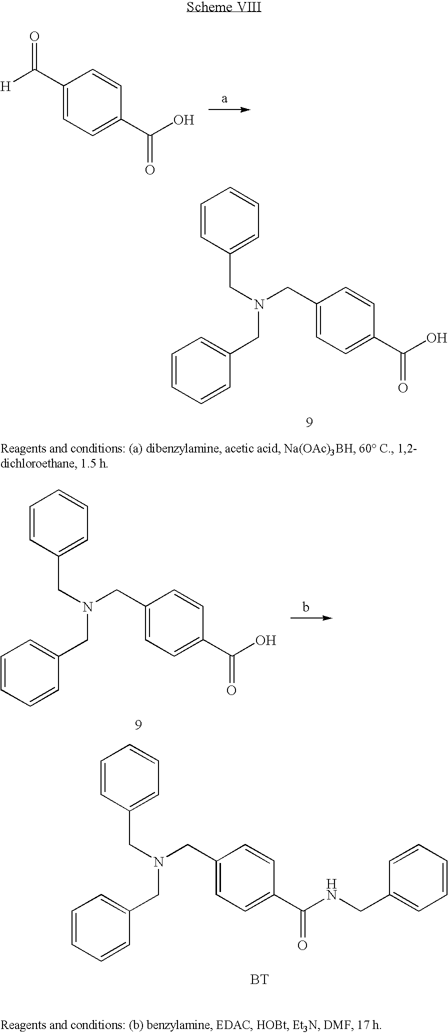

### Example 8

### 4-((benzylamino)methyl)-N′-phenylbenzohydrazide (BT)

Preparation of 4-((dibenzylamino)methyl)benzoic acid (9): To a solution of 4-formylbenzoic acid (2.3 g, 15.6 mmol) in 1,2-dichloroethane (40 mL) at room temperature was added dibenzylamine (3.0 mL, 15.6 mmol) and subsequently 4 drops of glacial acetic acid. After stirring the mixture for 15 minutes at room temperature, sodium triacetoxyborohydride (6.9 g, 32.7 mmol) was added and the mixture was heated to 60° C. with stirring. After 1.5 hours, the solvents were concentrated in vacuo and the residue was purified by preparative HPLC (Polaris C18 column using acetonitrile/water with 0.1% TFA). Concentration in vacuo from CH₂Cl₂/hexanes as final solvent afforded 2.72 g of the desired product 9: ¹H NMR (300 MHz, d₆-DMSO) δ 7.92 (s, J=5.0 Hz, 2H), 7.52 (s, 2H), 7.22-7.42 (m, 10H), 3.16 (s, 6H); ESI⁺ MS: m/z (rel intensity) 332 (100, M+H).

Preparation of 4-((benzylamino)methyl)-N′-phenylbenzohydrazide (BT): To a solution of 4-((dibenzylamino)methyl)benzoic acid, 9, (0.24 g, 0.72 mmol) in DMF (4 mL) was added N-(3-dimethylaminopropyl)-N′-ethylcarbodiimide hydrochloride (0.21 g, 1.08 mmol) and 1-hydroxybenzotriazole hydrate (0.15 g, 1.08 mmol). The mixture was allowed to stir for 15 minutes at room temperature. Subsequently, benzylamine (0.12 mL, 1.08 mmol) was added in one portion followed by triethylamine (0.30 mL, 2.16 mmol). After stirring the mixture for 17 hours at room temperature, the solution was diluted up to 10 mL with methanol, and then purified by preparative HPLC (Polaris C18 column using acetonitrile/water with 0.1% TFA) to give the product as a TFA salt after removal of the solvents in vacuo. The product was free-based by partitioning with EtOAc/saturated NaHCO₃, washing with saturated NaHCO₃ solution, washing with brine and drying over MgSO₄. The residue was taken up in MeOH (3 mL) to which excess of a 2N HCl in Et₂O solution was added. Concentration in vacuo from CH₂Cl₂/hexanes as final solvent afforded 101 mg of the desired product BT as the hydrochloride salt: ¹H NMR (300 MHz, CDCl₃) δ 9.15 (m, NH), 7.91 (m, 2H), 7.68 (m, 2H), 7.55 (m, 2H), 7.43 (m, 5H), 7.30 (m, 7H), 7.21 (m, 1H), 4.48 (d, J=5.0 Hz, 2H), 4.25 (m, 6H); ESI⁺ MS: m/z (rel intensity) 421 (100, M+H).

Compound BU listed below is a non-limiting example of the second aspect of the present invention, which was prepared using methods similar to those described in Scheme VIII (i.e., benzylamine in step b of Scheme VIII was replaced with 2-pyridylmethylamine).

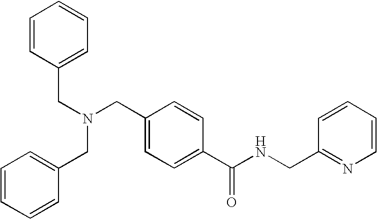

4-((dibenzylamino)methyl)-N-(pyridin-2-ylmethyl)benzamide (BU): ¹H NMR (300 MHz, CDCl₃) δ 9.55 (m, NH), 8.75 (m, 1H), 8.30 (m, 1H), 7.91 (m, 2H), 7.86-7.78 (m, 3H), 7.6 (m, 4H), 7.45 (m, 2H), 7.35 (m, 5H), 4.76 (d, J=5.0 Hz, 2H), 4.24 (m, 4H), 4.12 (m, 2H); ESI⁺ MS: m/z (rel intensity) 422 (100, M+H).

The compounds which comprise the second aspect of the present invention can also be prepared by the procedure outlined herein below in Scheme IX.

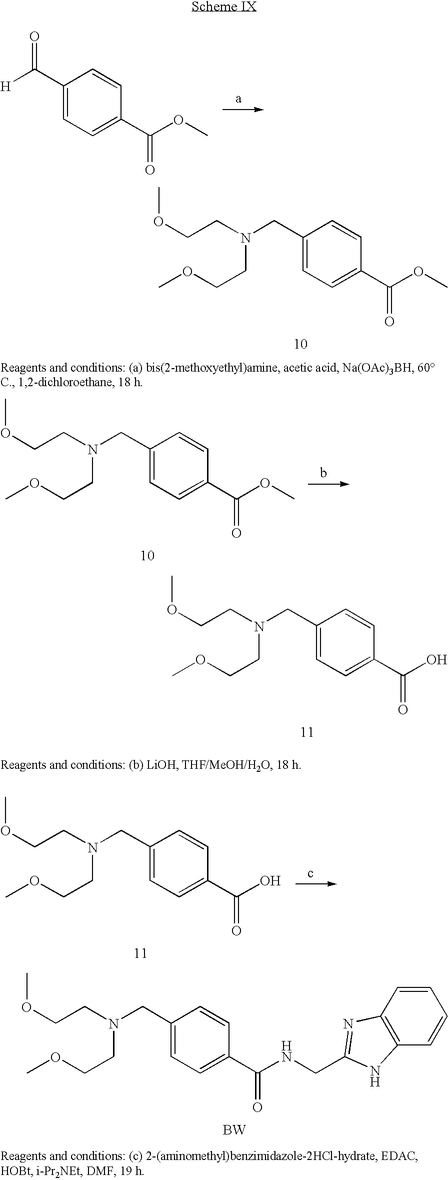

### Example 9

### N-((1H-benzo[d]imidazol-2-yl)methyl)-4-((bis(2-methoxyethyl)amino)methyl)benzamide (AI)

Preparation of methyl 4-((bis(2-methoxyethyl)amino)methyl)benzoate (10): To a solution of methyl-4-formyl benzoate (1.0 g, 6.1 mmol) and bis(2-methoxyethyl)amine (1.0 mL, 7.3 mmol) in 1,2-dichloroethane (30 mL) was added 5 drops of glacial acetic acid followed by sodium triacetoxy borohydride (2.8 g, 13.4 mmol). The reaction mixture was heated to 60° C. and stirred at this temperature for 18 hours. The solution was poured into aqueous saturated NaHCO₃. The aqueous phase was extracted twice with 20% isopropanol/CHCl₃. The combined organic phases were dried (MgSO₄), filtered and concentrated in vacuo. The resulting residue was purified over silica (0% methanol/CHCl₃ to 20% methanol/CHCl₃) to afford 1.27 g of the desired product as a colorless oil: ¹H NMR (400 MHz, d₆-DMSO) 67.89, (d, J=8.0 Hz, 2H), 7.46 (d, J=8.0 Hz, 2H), 3.83 (s, 3H), 3.72 (s, 2H), 3.39 (t, J=6.4 Hz, 4H), 3.19 (s, 6H), 2.63 (t, J=6.0 Hz, 4H); ESI⁺ MS: m/z (rel intensity) 282 (100, M+H).

Preparation of 4-((bis(2-methoxyethyl)amino)methyl)benzoic acid (11): To a solution of methyl 4-((bis(2-methoxyethyl)amino)methyl)benzoate, 10, (1.2 g, 4.3 mmol) in a 2:1:0.2 mixture of THF:H₂O:MeOH (32 mL) was added lithium hydroxide (2.0 g, 85.4 mmol). After stirring the reaction for 18 hours at room temperature, the mixture was acidified to pH 1 with 12N HCl. The resulting solution was concentrated in vacuo. The residue was triturated with hot CHCl₃, filtered, and washed with additional CHCl₃. The filtrate was concentrated in vacuo to afford the desired carboxylic acid 11 as the hydrochloride salt: ¹H NMR (400 MHz, CDCl₃) δ 11.66 (bs, 1H), 7.93 (d, J=6.4 Hz, 2H), 7.85 (m, 2H), 4.48 (m, 2H), 3.72-3.78 (m, 4H), 3.53 (bd m, 4H), 3.23 (s, 6H), ESI⁺ MS: m/z (rel intensity) 268 (100, M+H).

Preparation of N-((1H-benzo[d]imidazol-2-yl)methyl)-4-((bis(2-methoxyethyl)-amino)methyl)benzamide (BW): To a solution of 4-((bis(2-methoxyethyl)amino)methyl)benzoic acid hydrochloride, 11, (0.5 g, 1.9 mmol), N,N-diisopropylethyl amine (1.7 mL, 9.8 mmol) and 1-hydroxybenzotriazole hydrate (0.3 g, 2.3 mmol) in DMF (12 mL) was added N-(3-dimethylaminopropyl)-N′-ethylcarbodiimide hydrochloride (0.4 g, 2.3 mmol). The mixture was stirred at room temperature for 45 minutes. Subsequently, 2-(aminomethyl)benzimidazole dihydrochloride hydrate (0.8 g, 3.8 mmol) was added in one portion. After stirring the mixture for 17 hours at room temperature, the solution was poured into aqueous saturated NaHCO₃. The aqueous phase was extracted with EtOAc. The combined organic phases were dried (MgSO₄), filtered and concentrated in vacuo. The resulting residue was purified over silica (0% methanol/CHCl₃ to 10% methanol/CHCl₃) to afford 155 mg of the desired product as a white solid: ¹H NMR (400 MHz, d₆-DMSO) δ 12.23 (s, 1H), 9.14 (t, J=6.0 Hz, 1H), 7.89 (d, J=8.0 Hz, 2H), 7.54 (d, J=7.6 Hz, 1H), 7.42 (d, J=8.4 Hz, 3H), 7.14-7.11 (m, 2H), 4.68 (d, J=6.0 Hz, 2H), 3.70 (s, 2H), 3.40 (t, J=5.6 Hz, 4H), 3.20 (s, 6H), 2.63 (t, J=5.6 Hz, 4H); ESI⁺ MS: m/z (rel intensity) 397 (100, M+H).

Compounds BX and BY listed below are non-limiting examples of the second aspect of the present invention which were prepared using methods similar to those described in Scheme IX (i.e., bis(2-methoxyethyl)amine of step a, Scheme IX was replaced with tert-butyl 2,5-diazabicyclo[2.2.1]heptane-2-carboxylate). Compound BY can be synthesized by removal of the tert-butoxycarbonyl protecting group on compound BX.

tert-butyl 5-(4-(((1H-benzo[d]imidazol-2-yl)methyl)carbamoyl)benzyl)-2,5-diaza-bicyclo[2.2.1]heptane-2-carboxylate (BX): ¹H NMR (400 MHz, d₆-DMSO) δ 12.23 (s, 1H), 9.14 (t, J=6.0 Hz, 1H), 7.90 (d, J=8.4 Hz, 2H), 7.53 (d, J=7.2 Hz, 1H), 7.43 (d, J=8.4 Hz, 3H), 7.14-7.11 (m, 2H), 4.77 (d, J=10.8 Hz, 1H), 4.68 (d, J=5.6 Hz, 2H), 4.47 (d, J=6.0 Hz, 1H), 4.17 (d, J=14.8 Hz, 1H), 3.74 (s, 2H), 3.39 (m, 1H), 3.10 (dd, J=16.0, 9.2 Hz, 1H), 2.76 (t, J=7.6 Hz, 1H), 2.45 (t, J=10.0 Hz, 1H), 1.17 (d, J=13.2 Hz, 1H), 1.66-1.60 (m, 1H), 1.39 (s, 9H).

N-((1H-benzo[d]imidazol-2-yl)methyl)-4-(2,5-diaza-bicyclo[2.2.1]heptan-2-ylmethyl)benzamide (BY): ¹H NMR (400 MHz, d₆-DMSO) δ 8.01 (d, J=8.4 Hz, 2H), 7.42 (d, J=8.0 Hz, 4H), 7.10-7.05 (m, 2H), 4.75 (dd, J=15.2, 5.6 Hz, 1H), 4.60 (dd, J=16.0, 5.6 Hz, 1H), 4.44 (d, J=6.0 Hz, 1H), 4.12 (s, 1H), 3.81 (d, J=14.0 Hz, 1H), 3.68 (d, J=14.4 Hz, 1H), 3.52 (d, J=10.4 Hz, 1H), 3.03 (d, J=8.8 Hz, 1H), 2.79 (d, J=7.2 Hz, 1H), 2.50-2.44 (m, 1H), 1.72 (d, J=8.4 Hz, 1H), 1.56-1.51 (m, 1H); ESI⁺ MS: m/z (rel intensity) 362 (100, M+H).

Compounds of formula (IB), wherein L² is —CH₂CH₂— (e.g., compounds CD and CE) can be prepared using methods similar to those shown in General Scheme A. Those skilled in the art will recognize that the amine used in step c of General Scheme A can be synthesized by alkylation of 2-aminomethylbenzimidazole with N-(3-bromopropyl)-phthalimide similar to step a in Scheme IV.

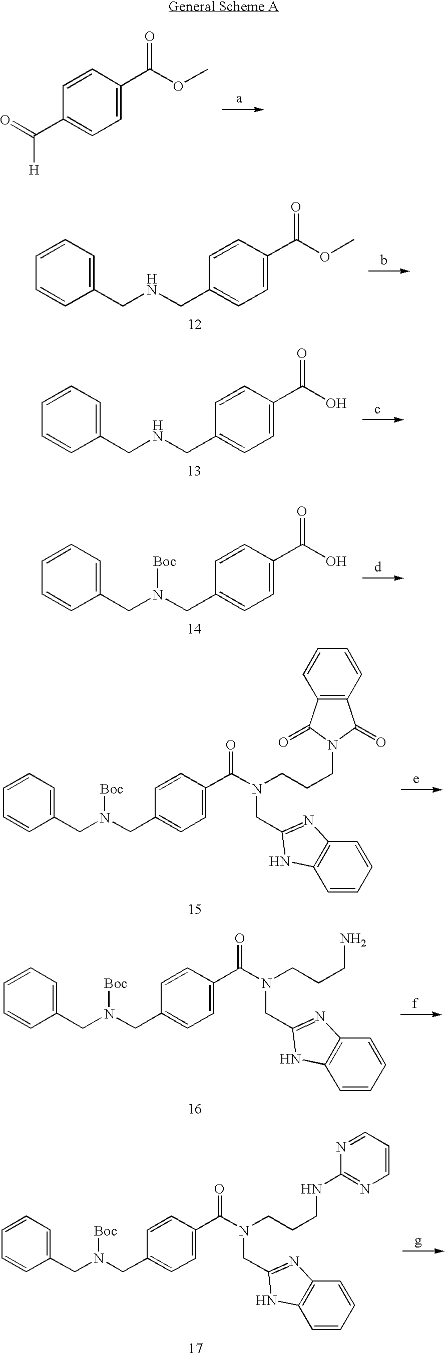

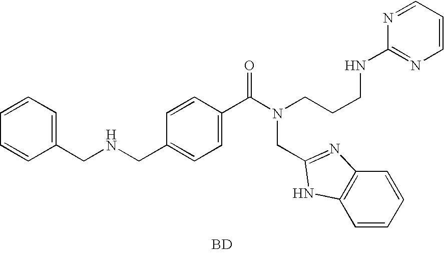

Reagents and conditions: (a) benzylamine, catalytic AcOH, 1,2-dichloroethane, 60° C., 2 h, then Na(OAc)₃BH; (b) LiOH, THF/MeOH/H₂O, 16 h; (c) di-tert-butyl-dicarboxylate, NaHCO₃, THF:H₂O (1:1 mixture); (d) 2-(3-((1H-benzo[d]imidazol-2-yl)methylamino)propyl)isoindoline-1,3-dione, EDAC, HOBt, i-Pr₂NEt, DMF, 19 h; (e) hydrazine, ethanol, 80° C.; (f) 2-chloropyrimidine, i-Pr₂NEt, DMF, 90° C.; (g) trifluoroacetic acid, CH₂Cl₂.

Those skilled in the art will recognize that the remaining compounds listed in Table 2 can also be prepared using methods similar to those described in Schemes VIII and IX, and General Scheme A, using appropriately substituted reagents.

### Third Aspect

Compounds of formula (I), wherein L² is —CH₂—, L¹ is C(O), R⁴ is —C(HR^{b})—C(O)—R^{a}, and X and Y are both hydrogen, can have the following general structure (IC):

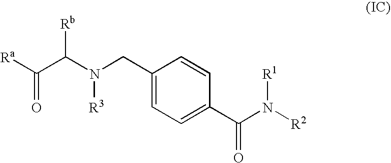

wherein R¹, R², R³, R^{a}, and R^{b} are defined herein below in Table 3.

TABLE 3

| Cmpd. | R^{a} | R^{b} | R³ | 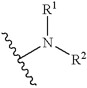 |
|---|---|---|---|---|
| CU | 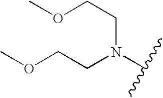 | H | CH₃ | 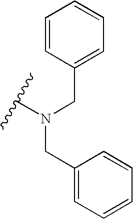 |
| CV | 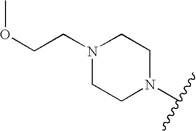 | H | CH₃ | 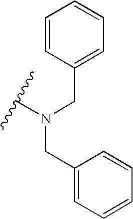 |
| CW | 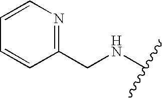 | H | CH₃ | 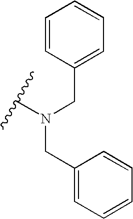 |
| CX | 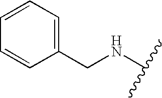 | H | CH₃ | 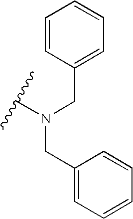 |
| CY | 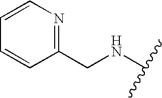 | Benzyl | H | 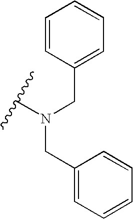 |
| CZ | 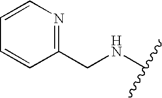 | 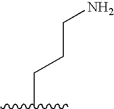 | H | 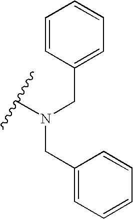 |
| DA | 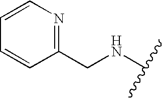 | 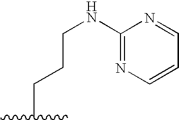 | H | 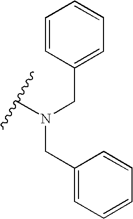 |
| DB | 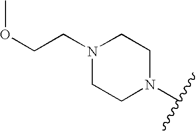 | Benzyl | H | 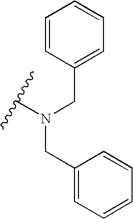 |

The compounds which comprise the third aspect of the present invention can be prepared by the procedure outlined herein below in Scheme X.

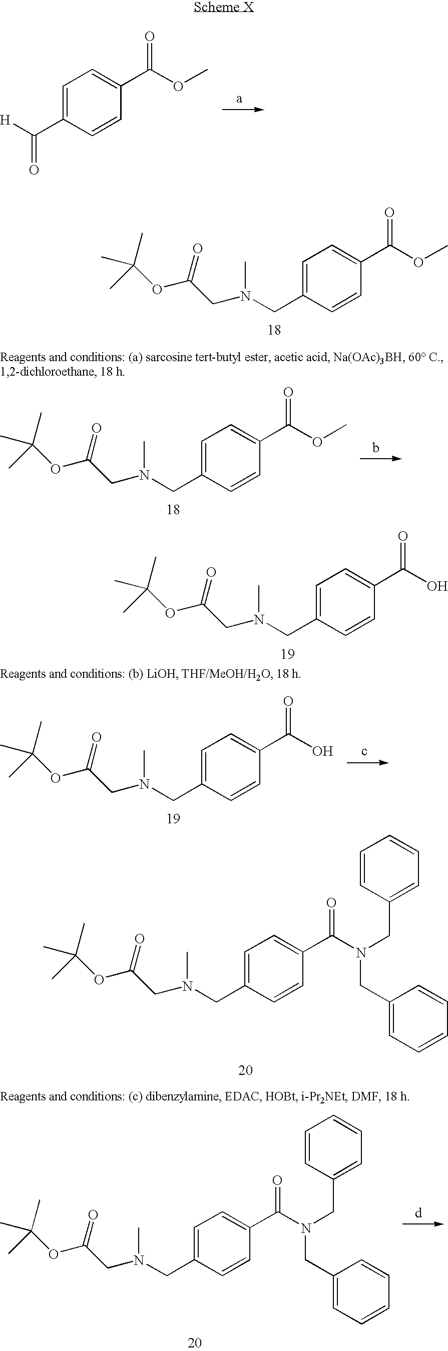

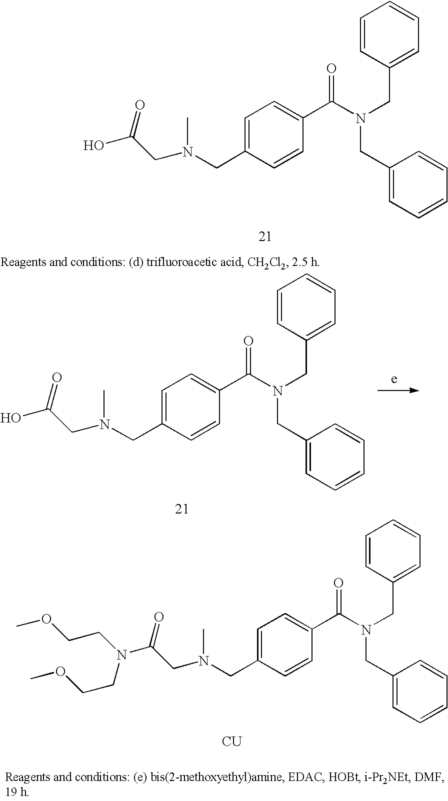

### Example 10

### N,N-dibenzyl-4-(((2-(bis(2-methoxyethyl)amino)-2-oxoethyl)(methyl)amino)-methyl)benzamide (CU)

Preparation of methyl 4-(((2-tert-butoxy-2-oxoethyl)(methyl)amino)-methyl)benzoate (18): To a solution of methyl-4-formyl benzoate (2.0 g, 12.2 mmol) and sarcosine tert-butyl ester (2.7 g, 14.6 mmol) in 1,2-dichloroethane (60 mL) was added 10 drops of glacial acetic acid followed by sodium triacetoxy borohydride (5.6 g, 26.8 mmol). The reaction mixture was heated to 60° C. and stirred at this temperature for 20 hours. The solution was poured into aqueous saturated NaHCO₃ and extracted twice with EtOAc. The combined organic phases were dried (MgSO₄), filtered and concentrated in vacuo to afford 1.32 g of the desired product that was used without further purification: ¹H NMR (400 MHz, d₆-DMSO) δ 7.91 (d, J=8.0 Hz, 2H), 7.44 (d, J=8.0 Hz, 2H), 3.83 (s, 3H), 3.69 (s, 2H), 3.18 (s, 2H), 2.23 (s, 3H), 1.42 (s, 9H); ESI⁺ MS: m/z (rel intensity) 294 (40, M+H).

Preparation of 4-(((2-tert-butoxy-2-oxoethyl)(methyl)amino)methyl)benzoic acid (19): To a solution of methyl 4-(((2-tert-butoxy-2-oxoethyl)(methyl)amino)-methyl)benzoate, 18, (1.0 g, 3.4 mmol) in a 2:1:0.2 mixture of THF:H₂O:MeOH (20 mL) was added lithium hydroxide (0.2 g, 6.8 mmol). After stirring the reaction for 18 hours at room temperature, the mixture was acidified to pH 4 with 1N HCl. The resulting solution was diluted with H₂O and extracted twice with EtOAc. The combined organic phases were dried (MgSO₄), filtered and concentrated in vacuo to afford the desired product as a white solid: ¹H NMR (400 MHz, d₆-DMSO) δ 7.85-7.87 (d, J=8.4 Hz, 2H), 7.37-7.39 (d, J=8.0 Hz, 2H), 2.70 (s, 2H), 3.20 (s, 2H), 2.20 (s, 3H), 1.40 (s, 9H); ESI⁺ MS: m/z (rel intensity) 280 (100, M+H).

Preparation of tert-butyl 2-((4-(dibenzylcarbamoyl)benzyl)(methyl)amino) acetate (20): To a solution of 4-(((2-tert-butoxy-2-oxoethyl)(methyl)amino)methyl)benzoic acid, 19, (0.4 g, 1.6 mmol), N,N-diisopropylethyl amine (1.4 mL, 7.8 mmol) and 1-hydroxybenzotriazole hydrate (0.3 g, 1.9 mmol) in DMF (10 mL) was added N-(3-dimethylaminopropyl)-N′-ethylcarbodiimide hydrochloride (0.4 g, 1.9 mmol). The mixture was stirred at 0° C. for 30 minutes, followed by the addition of dibenzylamine (0.5 mL, 2.4 mmol). After stirring the mixture for 18 hours at room temperature, the solution was diluted with saturated NaHCO₃ solution and extracted with EtOAc. The organic phase was dried (MgSO₄), filtered and concentrated in vacuo. The crude viscous oil was purified over silica (0% methanol/CHCl₃ to 5% methanol/CHCl₃) to yield 740 mg of the desired product: ¹H NMR (400 MHz, d₆-DMSO) δ 7.40-7.19 (m, 14H), 4.54 (s, 2H), 4.36 (s, 2H), 3.63-3.56 (d, J=15.2 Hz, 2H), 3.11 (s, 2H), 2.19 (s, 3H), 1.36 (s, 9H); ESI⁺ MS: m/z (rel intensity) 459 (100, M+H).

Preparation of 2-((4-(dibenzylcarbamoyl)benzyl)(methyl)amino)acetic acid (21): To a 1:1 mixture of TFA:CH₂Cl₂ (10 mL) was added, tert-butyl 2-((4-(dibenzylcarbamoyl)benzyl)-(methyl)amino)acetate, 20 (0.74 g, 1.61 mmol) and the reaction was stirred at room temperature for 2.5 hours. The solution was concentrated in vacuo to afford a brown viscous oil TFA salt. The crude product was used without further purification.

Preparation of N,N-dibenzyl-4-(((2-(bis(2-methoxyethyl)amino)-2-oxoethyl)(methyl)amino)methyl)benzamide (CU): To a solution of 2-((4-(dibenzylcarbamoyl)benzyl)(methyl)amino)acetic acid, 21, (0.4 g, 0.9 mmol), N,N-diisopropylethyl amine (1.5 mL, 8.9 mmol) and 1-hydroxybenzotriazole hydrate (0.2 g, 1.36 mmol) in DMF (10 mL) was added N-(3-dimethylaminopropyl)-N′-ethylcarbodiimide hydrochloride (0.2 g, 1.08 mmol). The mixture was stirred at 0° C. for 30 minutes, followed by the addition of bis(2-methoxyethyl)amine (0.2 mL, 1.36 mmol). After stirring the mixture for 18 hours at room temperature, the solution was diluted with saturated NaHCO₃ solution. The aqueous phase was extracted with EtOAc. The organic phase was dried (MgSO₄), filtered and concentrated in vacuo. The crude viscous oil was purified over silica (0% methanol/CHCl₃ to 10% methanol/CHCl₃) to yield the desired product: ¹H NMR (400 MHz, d₆-DMSO) δ 7.40-7.38 (d, J=8.4 Hz, 2H), 7.30-7.32 (d, J=8 Hz, 10H), 7.25 (m, 1H), 7.12 (m, 1H) 4.53 (s, 2H), 4.37 (s, 2H), 3.49-3.53 (m, 4H), 3.28-3.36 (m, 4H), 3.30 (s, 3H), 3.16 (m, 2H), 3.14 (s, 3H), 3.08 (s, 2H), 2.10 (s, 3H); ESI⁺ MS: m/z (rel intensity) 518 (100, M+H).

Compounds CV and CW listed below are non-limiting examples of the third aspect of the present invention which can be prepared using methods similar to those described in Scheme X.

N,N-dibenzyl-4-(((2-(4-(2-methoxyethyl)piperazin-1-yl)-2-oxoethyl)(methyl)amino)-methyl)benzamide (CV): ¹H NMR (400 MHz, d₆-DMSO) δ 7.92 (s, 1H), 7.40-7.12 (m, 13H), 4.54 (s, 2H), 4.36 (s, 2H), 3.48 (bs, 3H), 3.39-3.34 (m, 4H), 3.31 (s, 3H), 3.19-3.18 (bd, J=3.6 Hz, 3H), 3.13 (s, 2H), 2.31-2.27 (m, 4H), 2.07 (s, 3H); ESI⁺ MS: m/z (rel intensity) 529 (100, M+H).

N,N-dibenzyl-4-((methyl(2-oxo-2-(pyridin-2-ylmethylamino)ethyl)amino)-methyl)benzamide (CW): ¹H NMR (400 MHz, d₆-DMSO) δ 8.44-8.41 (m, 1H), 7.67-7.66 (t, J=1.6 Hz, 1H), 7.44-7.42 (d, J=8 Hz, 1H), 7.41-7.18 (m, 14H), 7.20-7.18 (d, J=7.6 Hz, 1H), 4.55 (s, 2H), 4.36-4.30 (d, J=5.6 Hz, 4H), 3.56 (s, 2H), 3.01 (s, 2H), 2.16 (s, 3H); ESI⁺ MS: m/z (rel intensity) 493 (100, M+H).

Those skilled in the art will recognize that the compounds listed in Table 3 can be prepared using methods similar to those described in Scheme X, using appropriately substituted reagents. For example, compound CX can be prepared by reacting compound 21 with benzylamine instead of bis(2-methoxyethyl)amine. Compounds CY-DB, in which R^{b} is benzyl or aminoalkyl and R³ is H can be prepared by using an appropriately substituted, protected amino acid in step a of Scheme X, instead of sarcosine tert-butyl ester.

### Fourth Aspect

Compounds of formula (I), wherein L² is —CH₂—, L¹ is S(O₂), and X and Y are both hydrogen, can have the following general structure (ID):

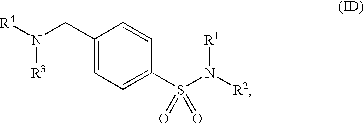

wherein R¹, R², and —NR³R⁴ are defined herein below in Table 4.

TABLE 4

| Compd. | —NR³R⁴ | R¹ | R² |
|---|---|---|---|
| DC | 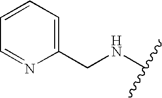 | benzyl | H |
| DD | 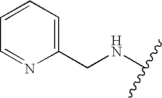 | benzyl | benzyl |
| DE | 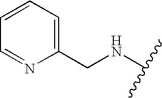 | 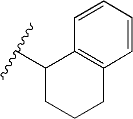 | H |
| DF | 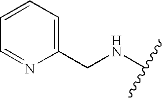 | 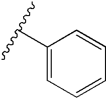 | H |
| DG | 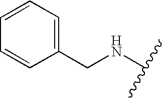 | benzyl | benzyl |
| DH | 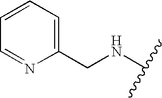 | 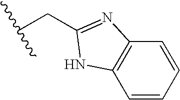 | benzyl |
| DI | 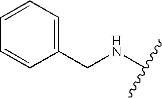 | 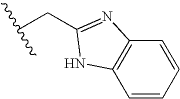 | benzyl |
| DJ | 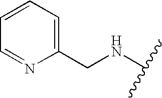 | 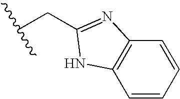 | 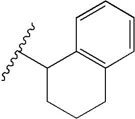 |
| DK | 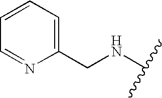 | 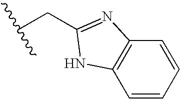 | 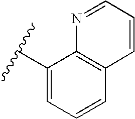 |
| DL | 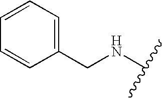 | 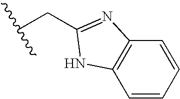 | 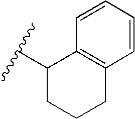 |
| DM | 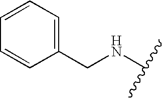 | 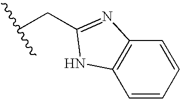 | 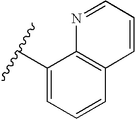 |
| DN | 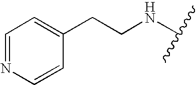 | benzyl | benzyl |
| DO | 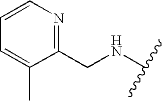 | benzyl | benzyl |
| DP | 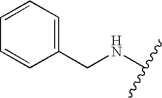 | 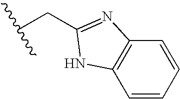 | 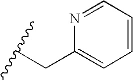 |
| DQ | 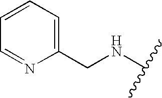 | 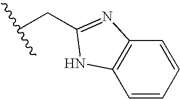 | H |
| DR | 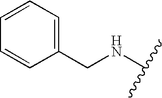 | 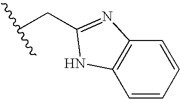 | H |
| DS | 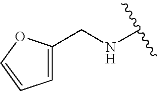 | benzyl | benzyl |
| DT | 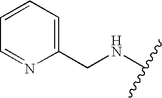 | 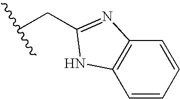 | 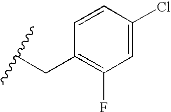 |
| DU | 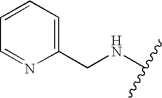 | 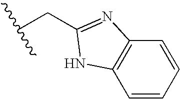 | 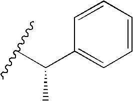 |
| DV | 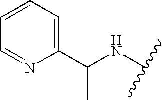 | benzyl | benzyl |
| DW | 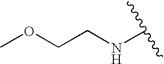 | benzyl | benzyl |
| DX | 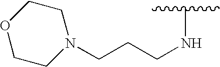 | benzyl | benzyl |
| DY | 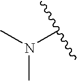 | benzyl | benzyl |
| DZ | 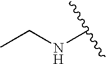 | benzyl | benzyl |
| EA | 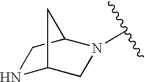 | benzyl | benzyl |
| EB | 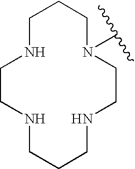 | benzyl | benzyl |
| EC | 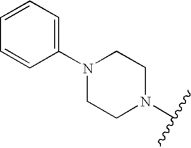 | benzyl | H |
| ED | 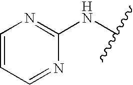 | benzyl | benzyl |
| EE | 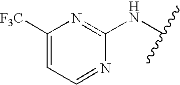 | benzyl | H |
| EF | 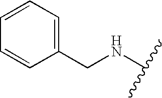 | 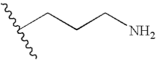 | benzyl |
| EG | 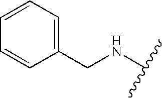 | 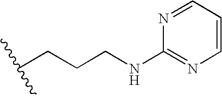 | benzyl |
| EH | 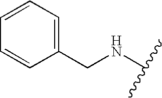 | 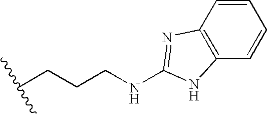 | benzyl |
| EI | 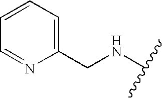 | 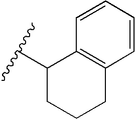 | benzyl |
| EJ | 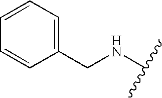 | 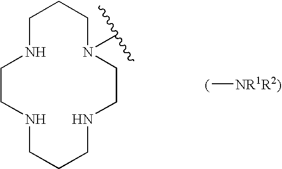 | |
| EK | 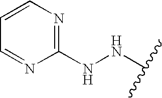 | benzyl | benzyl |
| EL | 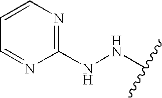 | 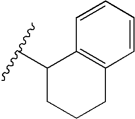 | benzyl |
| EM | 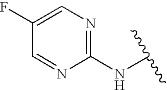 | H | benzyl |
| EN | 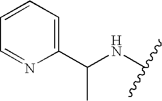 | 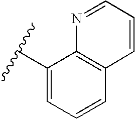 | benzyl |
| EO | 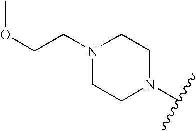 | benzyl | benzyl |
| EP | 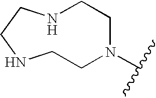 | benzyl | benzyl |
| EQ | 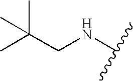 | benzyl | benzyl |
| ER | 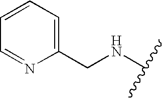 | 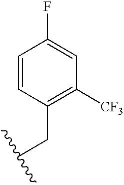 | 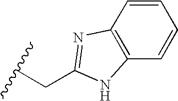 |
| ES | 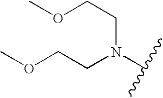 | benzyl | benzyl |
| ET | 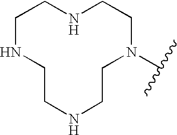 | benzyl | benzyl |
| EU | 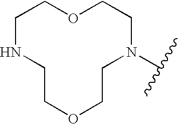 | benzyl | benzyl |
| EV | 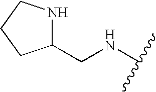 | benzyl | benzyl |
| EW | 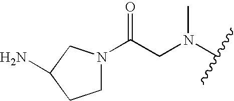 | benzyl | benzyl |
| EX | 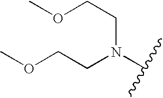 | 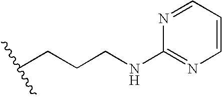 | 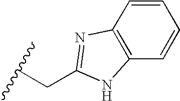 |
| EY | 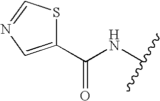 | benzyl | 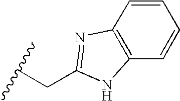 |
| EZ | 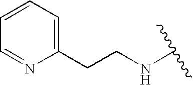 | benzyl | benzyl |
| FA | 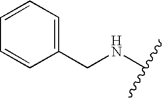 | 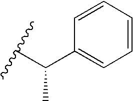 | 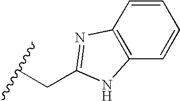 |
| FB | NH₂ | benzyl | 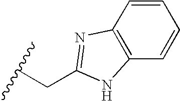 |
| FC | 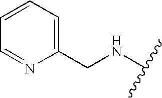 | H | 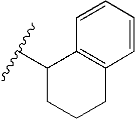 |
| FD | 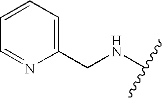 | 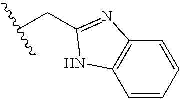 | 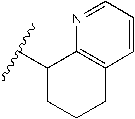 |
| FE | 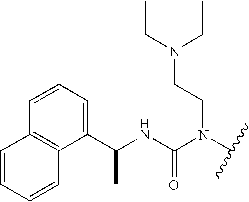 | benzyl | benzyl |
| FF | 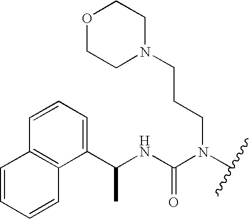 | benzyl | benzyl |
| FG | 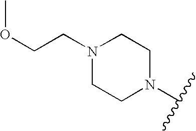 | H | 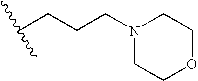 |
| FH | 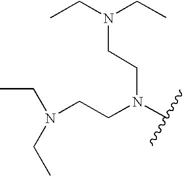 | benzyl | benzyl |
| FI | 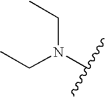 | methyl | benzyl |
| FJ | 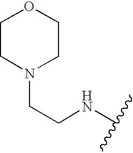 | benzyl | benzyl |
| FK | 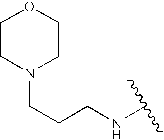 | H | 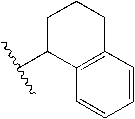 |
| FL | 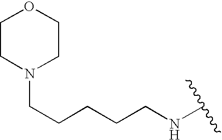 | benzyl | benzyl |
| FM | 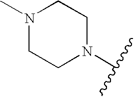 | H | 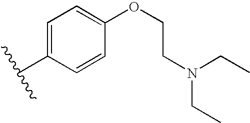 |
| FN | 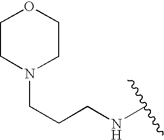 | methyl | benzyl |
| FO | 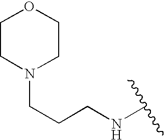 | 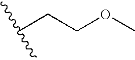 | 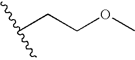 |
| FP | 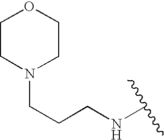 | ethyl | ethyl |
| FQ | 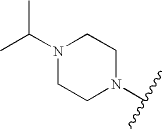 | H | 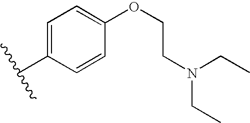 |
| FR | 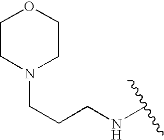 | 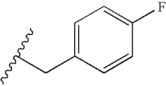 | 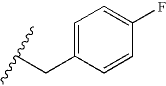 |
| FS | 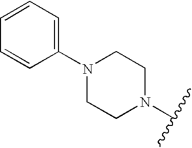 | methyl | benzyl |
| FT | 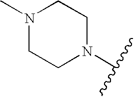 | methyl | benzyl |
| FU | 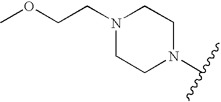 | methyl | benzyl |
| FV | 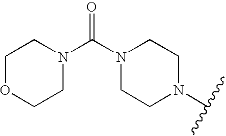 | methyl | benzyl |
| FW | 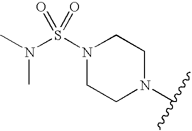 | methyl | benzyl |
| FX | 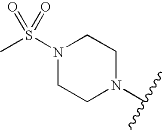 | methyl | benzyl |
| FY | 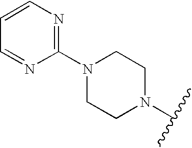 | methyl | benzyl |
| FZ | 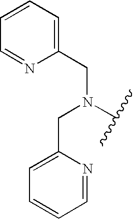 | methyl | benzyl |
| GA | 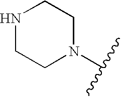 | methyl | benzyl |
| GB | 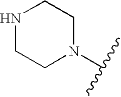 | H | benzyl |
| GC | 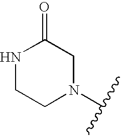 | methyl | benzyl |
| GD | 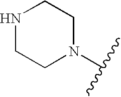 | methyl | 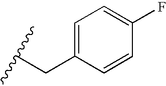 |
| GE | 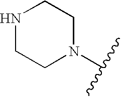 | 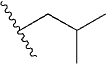 | benzyl |
| GF | 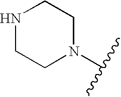 | methyl | 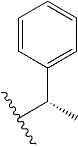 |
| GG | 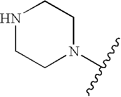 | methyl | 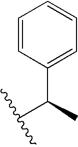 |
| GH | 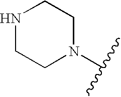 | 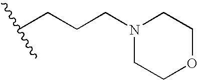 | benzyl |
| GI | 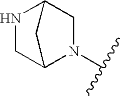 | methyl | benzyl |
| GJ | 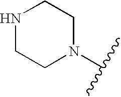 | H | 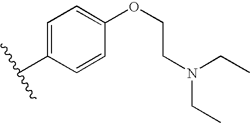 |
| GK | 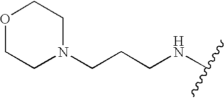 | H | 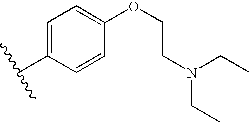 |
| GL | 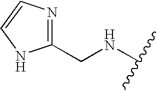 | methyl | benzyl |
| GM | 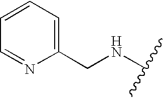 | methyl | benzyl |
| GN | 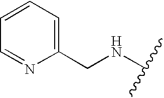 | 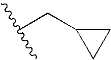 | benzyl |
| GO | 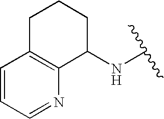 | 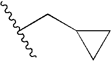 | benzyl |
| GP | 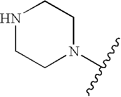 | 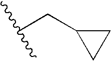 | benzyl |
| GQ | 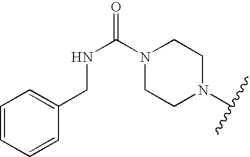 | methyl | benzyl |
| GR | 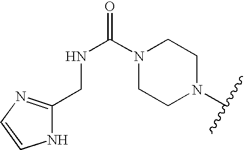 | methyl | benzyl |
| GS | 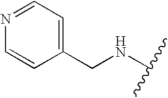 | benzyl | benzyl |
| GT | 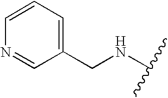 | benzyl | benzyl |
| GU | 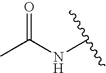 | H | 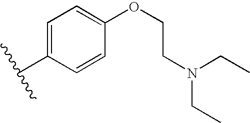 |
| GV | 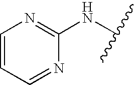 | H | 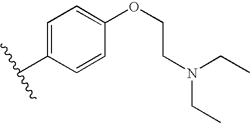 |
| GW(a) | 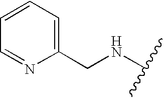 | 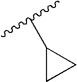 | benzyl |
| GW(b) | 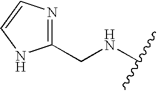 | methyl | benzyl |
| GX | 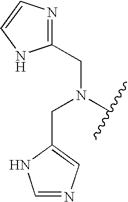 | methyl | benzyl |
| GY | 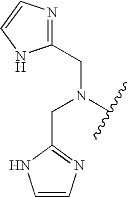 | methyl | benzyl |
| GZ | 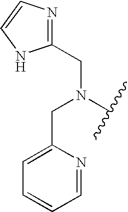 | methyl | benzyl |
| HA | 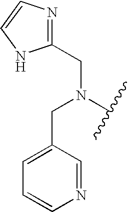 | methyl | benzyl |
| HB | 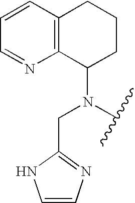 | methyl | benzyl |
| HC | 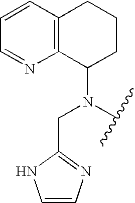 | H | benzyl |
| HD | 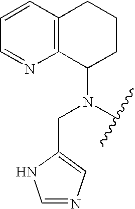 | H | benzyl |
| HE | 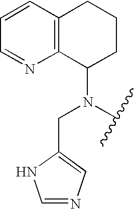 | methyl | benzyl |
| HF | 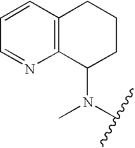 | methyl | benzyl |
| HG | 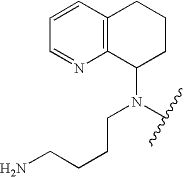 | methyl | benzyl |
| HH | 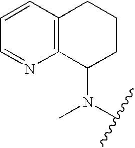 | H | benzyl |
| HI | 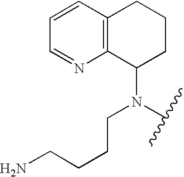 | H | benzyl |
| HJ | 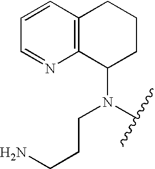 | methyl | benzyl |
| HK | 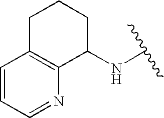 | H | benzyl |
| HL | 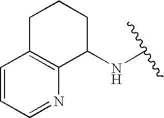 | methyl | benzyl |
| HM | 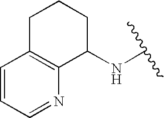 | 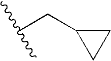 | benzyl |
| HN | 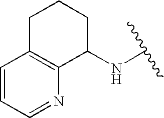 | methyl | 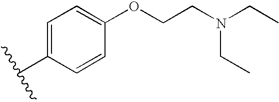 |
| HO | 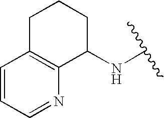 | methyl | 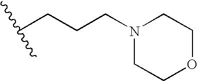 |
| HP | 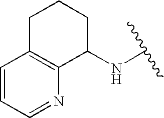 | methyl | 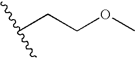 |
| HQ | 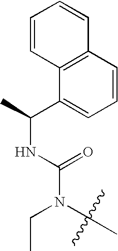 | benzyl | benzyl |
| HR | 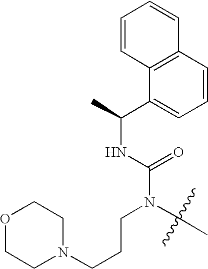 | H | 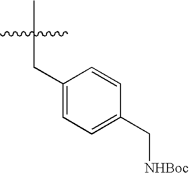 |
| HS | 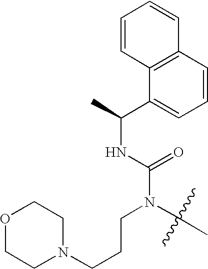 | H | 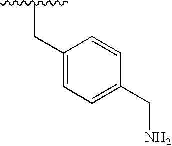 |
| HT | 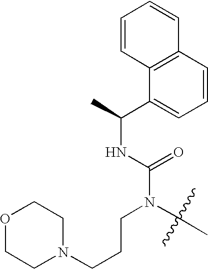 | 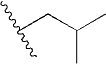 | 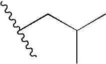 |
| HU | 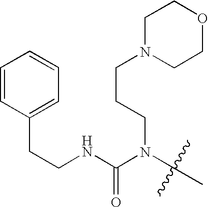 | benzyl | benzyl |
| HV | 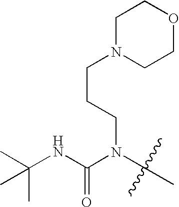 | benzyl | benzyl |
| HW | 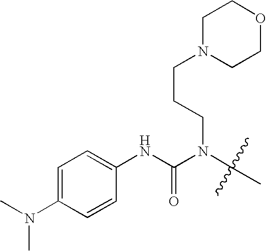 | benzyl | benzyl |
| HX | 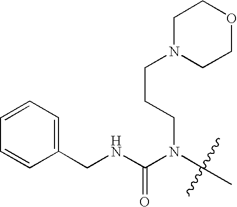 | benzyl | benzyl |
| HY | 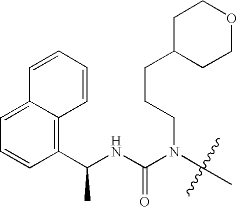 | methyl | benzyl |
| HZ | 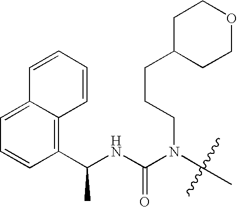 | 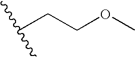 | 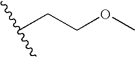 |
| IA | 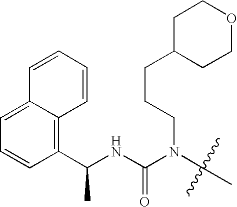 | ethyl | ethyl |
| IB | 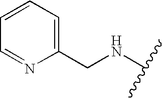 | benzyl | benzyl |
| IC | 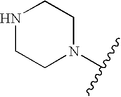 | methyl | benzyl |

The compounds which comprise the fourth aspect of the present invention can be prepared by the procedure outlined herein below in Scheme XI.

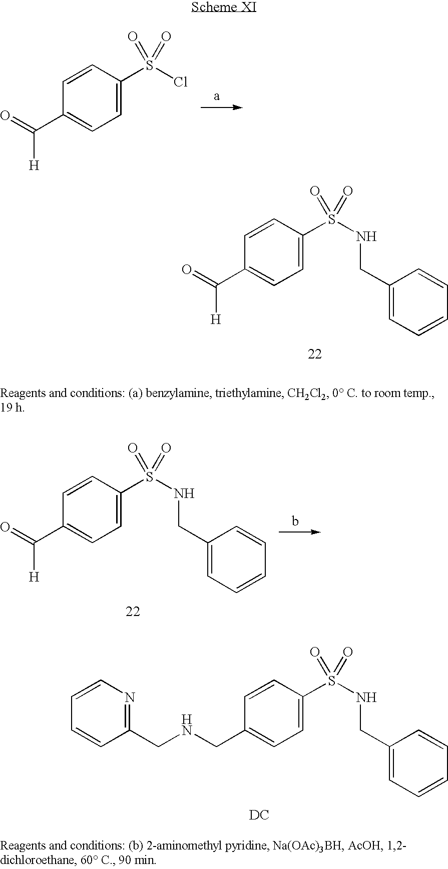

### Example 11

### N-benzyl-4-((pyridin-2-ylmethylamino)methyl)benzenesulfonamide (DC)

Preparation of N-benzyl-4-formylbenzenesulfonamide (22): To a cold (0° C.) solution of 4-formylbenzene-1-sulfonyl chloride (1.04 g, 5.08 mmol) in CH₂Cl₂ (15 mL) at was added dropwise benzylamine (0.58 mL, 5.34 mmol). Triethylamine (0.78 mL, 5.59 mmol) was then added and the mixture was allowed to stir at 0° C. for 30 minutes after which time the cooling bath was removed. After stirring the mixture for 3 hours at room temperature, the mixture was diluted with aqueous saturated NaHCO₃ solution. The aqueous layer was extracted three times with EtOAc. The combined organic phases were washed with brine, dried (MgSO₄), filtered and concentrated in vacuo. The crude residue (1.23 g) was used without further purification: ¹H NMR (300 MHz, CDCl₃) δ 10.04 (s, 1H), 8.39 (m, 2H), 7.21 (m, 2H), 7.13 (m, 5H), 5.13 (m, 1H, NH), 4.14 (d, J=5.0 Hz, 2H); ESI⁻ MS: m/z (rel intensity) 274 (100, M−H).

Preparation of N-benzyl-4-((pyridin-2-ylmethylamino)methyl)benzenesulfonamide (DC): To a solution of N-benzyl-4-formylbenzenesulfonamide, 22, (0.31 g, 1.13 mmol) in 1,2-dichloroethane (6 mL) at room temperature was added 2-aminomethylpyridine (0.13 mL, 1.25 mmol) in one portion followed by 2 drops of glacial acetic acid. After stirring the mixture for 15 minutes at room temperature, sodium triacetoxyborohydride (0.51 g, 2.39 mmol) was added and the mixture was heated to 60° C. with stirring. After heating for 1.5 hours, the solvents are concentrated in vacuo. The crude residue was purified by preparative HPLC (Polaris C18 column using acetonitrile/water with 0.1% TFA) and the solvents were removed in vacuo to afford the desired product: ¹H NMR (300 MHz, d₆-DMSO) δ 10.01 (s, NH, 2H), 8.69 (m, 1H), 8.68 (m, 1H), 7.98 (m, 1H), 7.86 (d, J=5.0 Hz, 2H), 7.79 (d, J=5.0 Hz, 2H), 7.63 (m, 1H), 7.48 (m, 1H), 7.31-7.21 (m, 4H), 4.34 (m, 4H), 4.01 (d, J=5.0 Hz, 2H); ESI⁺ MS: m/z (rel intensity) 368 (100, M+H).

Compounds DD, DE, DF, DG, DO, DR, DS, DV, DW, DX, DY, DZ, ER, and EY listed below are non-limiting examples of the fourth aspect of the present invention, each of which were prepared using methods similar to those described in Scheme XI, using appropriately substituted amino reagents.

N,N-dibenzyl-4-((pyridin-2-ylmethylamino)methyl)benzenesulfonamide (DD): ¹H NMR (300 MHz, d₆-DMSO) δ 10.03 (s, 1H), 8.70 (d, J=5.0 Hz, 1H), 7.99 (m, 2H), 7.84 (m, 2H), 7.62 (m, 1H), 7.52 (m, 1H), 7.22 (m, 6H), 7.08 (m, 5H), 4.39 (m, 4H), 4.31 (s, 4H); ESI⁺ MS: m/z (rel intensity) 458 (100, M+H).

4-((pyridin-2-ylmethylamino)methyl)-N-(1,2,3,4-tetrahydronaphthalen-1-yl)benzenesulfonamide (DE): ¹H NMR (300 MHz, d₆-DMSO) δ 9.74 (s, 2H), 8.68 (d, J=5.0 Hz, 1H), 8.21 (d, J=5.0 Hz, 1H), 7.96 (d, J=5.0 Hz, 1H), 7.90 (m, 2H), 7.79 (d, J=5.0 Hz, 2H), 7.56 (d, J=5.0 Hz, 2H), 7.47 (m, 2H), 7.08 (m, 1H), 4.38 (m, 4H), 4.01 (d, J=5.0 Hz, 2H), 2.67 (m, 1H), 1.58 (m, 2H), 1.26 (m, 2H), 0.87 (m, 2H); ESI⁺ MS: m/z (rel intensity) 408 (100, M+H).

N-phenyl-4-((pyridin-2-ylmethylamino)methyl)benzenesulfonamide (D° F.): ¹H NMR (300 MHz, d₆-DMSO) δ 10.43 (s, 2H), 8.63 (m, 1H), 7.87 (d, J=5.0 Hz, 2H), 7.70 (d, J=5.0 Hz, 2H), 7.48 (m, 4H), 7.25 (m, 1H), 7.12 (m, 2H), 7.05 (m, 1H), 4.33 (s, 2H), 4.30 (s, 2H); ESI⁺ MS: m/z (rel intensity) 354 (100, M+H).

N,N-dibenzyl-4-((benzylamino)methyl)benzenesulfonamide (DG): ¹H NMR (300 MHz, d₆-DMSO) δ 7.80 (d, J=5.0 Hz, 2H), 7.49 (d, J=5.0 Hz, 2H), 7.32 (m, 6H), 7.19 (m, 6H), 7.06 (m, 3H), 4.31 (s, 4H), 3.91 (s, 2H), 3.82 (s, 2H); ESI⁺ MS: m/z (rel intensity) 457 (100, M+H).

N,N-dibenzyl-4-(((3-methylpyridin-2-yl)methylamino)methyl)benzenesulfonamide (DO): ¹H NMR (300 MHz, d₆-DMSO) δ 9.60 (m, 1H, NH), 7.94 (m, 2H), 7.80 (m, 2H), 7.60 (m, 1H), 7.22 (m, 5H), 7.05 (m, 5H), 4.39 (m, 4H), 4.30 (s, 4H); ESI⁺ MS: m/z (rel intensity) 472 (100, M+H).

N-((1H-benzo[d]imidazol-2-yl)methyl)-4-((benzylamino)methyl)benzene-sulfonamide (DR): ¹H NMR (300 MHz, d₆-DMSO) δ 7.78 (m, 2H), 7.54 (m, 2H), 7.4 (m, 2H), 7.33 (m, 5H), 7.12 (m, 2H), 4.14 (s, 2H), 3.78 (s, 2H), 3.66 (s, 2H); ESI⁺ MS: m/z (rel intensity) 407 (100, M+H).

N,N-dibenzyl-4-((furan-2-ylmethylamino)methyl)benzenesulfonamide (DS): ¹H NMR (300 MHz, d₆-DMSO) δ 9.96 (m, NH), 7.92 (d, J=5.0 Hz, 2H), 7.78 (d, J=5.0 Hz, 2H), 7.21 (m, 5H), 7.06 (m, 5H), 6.68 (d, J=5.0 Hz, 1H), 6.54 (d, J=5.0 Hz, 1H), 4.30 (s, 4H), 4.26 (s, 2H), 4.22 (s, 2H); ESI⁺ MS: m/z (rel intensity) 447 (100, M+H).

N,N-dibenzyl-4-((1-(pyridin-2-yl)ethylamino)methyl)benzenesulfonamide (DV): ¹H NMR (300 MHz, d₆-DMSO) δ 10.20 (m, 1H), 8.70 (d, J=5.0 Hz, 2H), 7.92 (m, 2H), 7.88 (d, J=5.0 Hz, 2H), 7.77 (m, 2H), 7.66 (m, 1H), 7.50 (m, 1H), 7.20 (m, 5H), 7.08 (m, 5H), 4.30 (m, 4H), 4.04 (m, 1H), 1.63 (d, J=5.0 Hz, 3H); ESI⁺ MS: m/z (rel intensity) 472 (100, M+H).

N,N-dibenzyl-4-((2-methoxyethylamino)methyl)benzenesulfonamide (CI): ¹H NMR (300 MHz, d₆-DMSO) δ 9.40 (m, NH), 7.92 (d, J=5.0 Hz, 2H), 7.78 (d, J=5.0 Hz, 2H), 7.19 (m, 5H), 7.06 (m, 5H), 4.30 (m, 4H), 4.26 (s, 3H), 4.24 (s, 2H), 3.64 (m, 2H), 3.02 (m, 2H); ESI⁺ MS: m/z (rel intensity) 425 (100, M+H).

N,N-dibenzyl-4-((3-morpholinopropylamino)methyl)benzenesulfonamide (DX): ¹H NMR (400 MHz, d₆-DMSO) δ 7.78 (d, J=8.4 Hz, 2H), 7.46 (d, J=8 Hz, 2H), 7.19 (dd, J=3.2, 3.6 Hz, 6H), 7.03 (m, 4H), 4.30 (s, 4H), 3.86 (s, 2H), 3.69 (t, J=4.4, 4.8 Hz, 4H), 2.67 (t, J=7.2, 6.4 Hz, 2H), 2.41 (t, J=7.2 Hz, 6H), 1.83 (bs, 1H), 1.72 (m, J=7.2, 2H).

N,N-dibenzyl-4-((dimethylamino)methyl)benzenesulfonamide (DY): ¹H NMR (300 MHz, d₆-DMSO) δ 7.96 (d, J=5.0 Hz, 2H), 7.81 (d, J=5.0 Hz, 2H), 7.21 (m, 5H), 7.06 (m, 5H), 4.38 (s, 2H), 4.32 (s, 4H), 2.69 (s, 6H); ESI⁺ MS: m/z (rel intensity) 395 (100, M+H).

N,N-dibenzyl-4-((ethylamino)methyl)benzenesulfonamide (DZ): ¹H NMR (300 MHz, d₆-DMSO) δ 9.20 (m, 1H), 7.96 (d, J=5.0 Hz, 2H), 7.76 (d, J=5.0 Hz, 2H), 7.20 (m, 5H), 7.08 (m, 5H), 4.30 (m, 4H), 4.24 (s, 2H), 2.99 (m, 2H), 0.86 (m, 3H); ESI⁺ MS: m/z (rel intensity) 395 (100, M+H).

N,N-dibenzyl-4-((bis(2-methoxyethyl)amino)methyl)benzenesulfonamide (ER): ¹H NMR (400 MHz, CDCl₃) δ 7.77 (d, J=8.4 Hz, 2H), 7.49 (d, J=8.4 Hz, 2H), 7.19 (t, J=3.2 Hz, 6H), 7.02 (m, 4H), 4.34 (s, 4H), 3.82 (s, 2H), 3.51 (t, J=5.6, 6.0 Hz, 4H), 3.34 (s, 6H), 2.78 (dd, J=5.6, 6.0 Hz, 4H).

N,N-dibenzyl-4-((2-(pyridin-4-yl)ethylamino)methyl)benzenesulfonamide (EY): ¹H NMR (300 MHz, d₆-DMSO) δ 9.82 (m, 1H), 8.82 (d, J=5.0 Hz, 2H), 7.96 (d, J=5.0 Hz, 2H), 7.90 (d, J=5.0 Hz, 2H), 7.84 (d, J=5.0 Hz, 2H), 7.19 (m, 5H), 7.08 (m, 5H), 4.30 (m, 4H); ESI⁺ MS: m/z (rel intensity) 472 (100, M+H).

Various compounds which comprise the fourth aspect of the present invention can also be prepared by the procedure outlined herein below in Scheme XII.

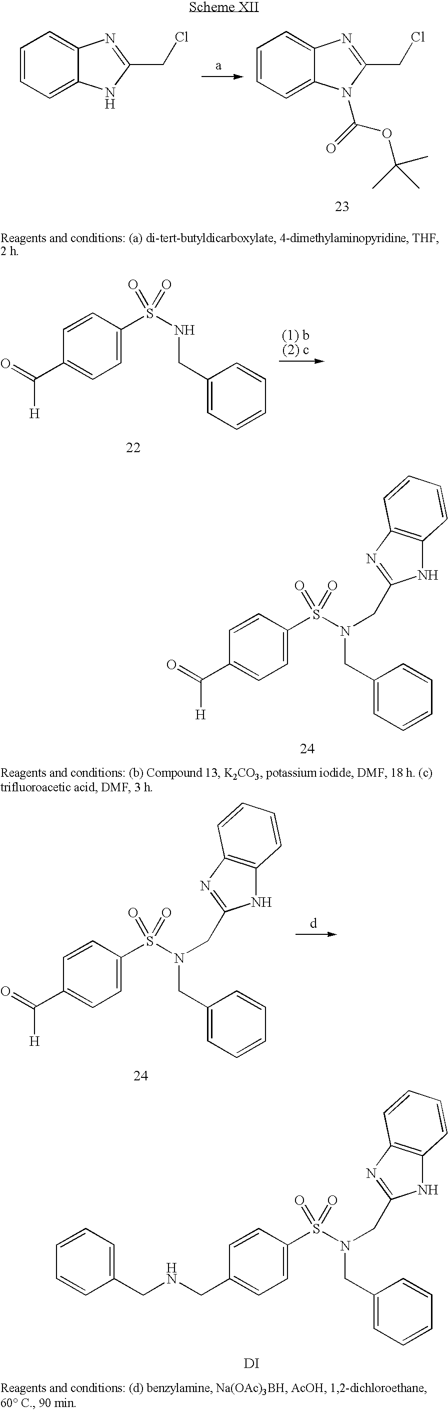

### Example 12

### N-((1H-benzo[d]imidazol-2-yl)methyl)-N-benzyl-4-((benzylamino)methyl)benzenesulfonamide (DI)

Preparation of tert-butyl 2-(chloromethyl)-1H-benzo[d]imidazole-1-carboxylate (23): To a solution of 2-chloromethylbenzimidazole (4.60 g, 27.61 mmol) in tetrahydrofuran (150 mL) was added di-tert-butyldicarboxylate (9.04 g, 41.41 mmol) and 4-N-dimethylaminopyridine (0.34 g, 2.76 mmol). After stirring the mixture for 2 h at 60° C., the mixture was diluted with aqueous saturated NaHCO₃ and extracted three times with CH₂Cl₂. The combined organic phases were washed with brine, dried (MgSO₄), filtered and concentrated in vacuo to afford 5.96 g of the desired product 23, which was used without further purification: ¹H NMR (300 MHz, CDCl₃) δ 7.78 (d, J=5.6, 2H), 7.55 (d, J=5.6, 2H), 7.22-7.12 (m, 4H), 4.86 (s, 2H), 1.53 (s, 9H); ESI⁺ MS: m/z (rel intensity) 267 (100, M+H).

Preparation of N-((1H-benzo[d]imidazol-2-yl)methyl)-N-benzyl-4-formylbenzenesulfonamide (24): To a solution of N-benzyl-4-formylbenzenesulfonamide, 22, (0.39 g, 1.42 mmol) in DMF (4 mL) was added tert-butyl 2-(chloromethyl)-1H-benzo[d]imidazole-1-carboxylate, 23 (397 mg, 1.49 mmole), potassium carbonate (0.59 g, 4.26 mmol) and potassium iodide (0.02 g, 0.14 mmol). After stirring for 18 hours at 60° C., the solution was decanted by pipet from the salts. To the mixture in DMF was slowly added trifluoroacetic acid to give a 2 mL:4 mL v/v mixture of TFA:DMF, and the mixture was stirred at room temperature for 3 hours. After most of the solvents were removed in vacuo, the residue was diluted to 10 mL in MeOH and purified by preparative HPLC (Polaris C18 column using acetonitrile/water with 0.1% TFA) to afford 396 mg of the desired product 24 as a TFA salt: ¹H NMR (300 MHz, CDCl₃) δ 10.04 (s, 1H), 8.02 (m, 4H), 7.50 (m, 2H), 7.38 (m, 2H), 7.13 (m, 2H), 6.92 (m, 2H), 4.92 (bs, NH), 4.45 (s, 2H), 3.43 (s, 2H); ESI⁺ MS: m/z (rel intensity) 406 (100, M+H).

Preparation of N-((1H-benzo[d]imidazol-2-yl)methyl)-N-benzyl-4-((benzylamino)-methyl)benzenesulfonamide (DI): To a solution of N-((1H-benzo[d]imidazol-2-yl)methyl)-N-benzyl-4-formylbenzenesulfonamide, 24, (0.22 g, 0.53 mmol) in 1,2-dichloroethane (8 mL) at room temperature was added benzylamine (0.64 mL, 0.58 mmol) in one portion followed by 1 drop of glacial acetic acid. After stirring the mixture for 15 minutes at room temperature, sodium triacetoxyborohydride (0.24 g, 1.12 mmol) was added and the mixture was heated to 60° C. with stirring. After 1.5 hr, the solvents were concentrated in vacuo. The crude residue was purified by preparative HPLC (Polaris C18 column using acetonitrile/water with 0.1% TFA) and the solvents were removed in vacuo to afford 130 mg of the desired product DI as the trifluoroacetic acid salt: ¹H NMR (300 MHz, d₆-DMSO) δ 9.48 (m, 1H), 7.96 (d, J=5.0 Hz, 2H), 7.68 (d, J=5.0 Hz, 2H), 7.57 (m, 2H), 7.47 (m, 6H), 7.32 (m, 2H), 7.22 (m, 2H), 7.08 (m, 2H), 4.71 (s, 2H), 4.51 (s, 2H), 4.27 (m, 2H), 4.18 (m, 2H); ESI⁺ MS: m/z (rel intensity) 497 (100, M+H).

Compounds DH, DJ, DK, DL, DM, DP, DT, DU, ER, and FD listed below are non-limiting examples of the first aspect of the present invention which were prepared using methods similar to those described in Scheme II, using appropriately substituted reagents.

N-((1H-benzo[d]imidazol-2-yl)methyl)-N-benzyl-4-((pyridin-2-ylmethylamino)methyl)benzenesulfonamide (DH): ¹H NMR (300 MHz, d₆-DMSO) δ 9.70 (m, NH), 8.66 (m, 1H), 7.94 (d, J=5.0 Hz, 2H), 7.70 (d, J=5.0 Hz, 2H), 7.52 (m, 4H), 7.47 (m, 2H), 7.25 (m, 5H), 7.16 (m, 1H), 4.66 (s, 2H), 4.53 (s, 2H), 4.30 (m, 4H); ESI⁺ MS: m/z (rel intensity) 498 (100, M+H).

N-((1H-benzo[d]imidazol-2-yl)methyl)-4-((pyridin-2-ylmethylamino)methyl)-N-(1,2,3,4-tetrahydronaphthalen-1-yl)benzenesulfonamide (DJ): ¹H NMR (300 MHz, d₆-DMSO) δ 9.60 (m, NH), 8.40 (m, 1H), 7.94 (d, J=5.0 Hz, 2H), 7.78 (m, 2H), 7.66 (m, 2H), 7.56 (m, 2H), 7.42 (m, 2H), 7.26 (m, 1H), 7.08 (m, 1H), 4.66 (s, 2H), 4.53 (s, 2H), 410-4.30 (m, 2H), 2.6 (m, 1H), 1.77 (m, 2H), 1.21 (m, 2H); ESI⁺ MS: m/z (rel intensity) 538 (100, M+H).

N-((1H-benzo[d]imidazol-2-yl)methyl)-4-((pyridin-2-ylmethylamino)methyl)-N-(quinolin-8-yl)benzenesulfonamide (DK): ¹H NMR (300 MHz, d₆-DMSO) δ 9.70 (m, NH), 8.66 (m, 1H), 8.54 (m, 1H), 8.42 (m, 1H), 8.06 (m, 2H), 7.94 (m, 2H), 7.60 (m, 4H), 7.45 (m, 2H), 7.47 (m, 2H), 4.32 (s, 2H), 4.28 (s, 2H), 4.30-4.10 (m, 2H); ESI⁺ MS: m/z (rel intensity) 535 (100, M+H).

N-((1H-benzo[d]imidazol-2-yl)methyl)-4-((benzylamino)methyl)-N-(1,2,3,4-tetrahydronaphthalen-1-yl)benzenesulfonamide (DL): ¹H NMR (300 MHz, CDCl₃) δ 8.28 (d, J=5.1 Hz, 1H), 7.15 (d, J=5.1 Hz, 1H), 7.02-7.07 (m, 4H), 6.87 (s, 1H), 4.00 (s, 2H), 3.68 (m, 4H), 3.24 (q, J=7.3 Hz, 2H), 2.10 (m, 2H), 1.42 (t, J=7.2 Hz, 3H); ESI⁺ MS: m/z (rel intensity) 374 (100, M+H).

N-((1H-benzo[d]imidazol-2-yl)methyl)-4-((benzylamino)methyl)-N-(quinolin-8-yl)benzenesulfonamide (DM): ¹H NMR (300 MHz, CDCl₃) δ 8.28 (d, J=5.1 Hz, 1H), 7.15 (d, J=5.1 Hz, 1H), 7.02-7.07 (m, 4H), 6.87 (s, 1H), 4.00 (s, 2H), 3.68 (m, 4H), 3.24 (q, J=7.3 Hz, 2H), 2.10 (m, 2H), 1.42 (t, J=7.2 Hz, 3H); ESI⁺ MS: m/z (rel intensity) 374 (100, M+H).

N-((1H-benzo[d]imidazol-2-yl)methyl)-4-((benzylamino)methyl)-N-(pyridin-2-ylmethyl)benzenesulfonamide (DP): ¹H NMR (300 MHz, d₆-DMSO) δ 9.60 (m, NH), 8.43 (m, 1H), 7.92 (d, J=5.0 Hz, 2H), 7.88-7.70 (m, 4H), 7.52-7.40 (m, 4H), 7.47 (m, 2H), 7.28-7.20 (m, 3H), 4.98 (s, 2H), 4.75 (s, 2H), 4.22 (m, 2H), 4.17 (m, 2H); ESI⁺ MS: m/z (rel intensity) 498 (100, M+H).

N-((1H-benzo[d]imidazol-2-yl)methyl)-N-(4-chloro-2-fluorobenzyl)-4-((pyridin-2-ylmethylamino)methyl)benzenesulfonamide (DT): ¹H NMR (300 MHz, d₆-DMSO) δ 9.90 (m, NH), 8.65 (m, NH), 7.96 (d, J=5.0 Hz, 2H), 7.76 (d, J=5.0 Hz, 2H), 7.68 (m, 4H), 7.46 (m, 4H), 7.20 (m, 2H), 4.88 (s, 2H), 4.60 (s, 2H), 4.33 (m, 2H), 4.10 (m, 2H); ESI⁺ MS: m/z (rel intensity) 550 (100, M+H).

(S)—N-((1H-benzo[d]imidazol-2-yl)methyl)-N-(1-phenylethyl)-4-((pyridin-2-ylmethylamino)methyl)benzenesulfonamide (DU): ¹H NMR (300 MHz, d₆-DMSO) δ 9.20 (m, NH), 8.64 (m, NH), 8.05 (d, J=5.0 Hz, 2H), 7.90 (m, 2H), 7.86 (d, J=5.0 Hz, 2H), 7.65 (m, 4H), 7.48 (m, 2H), 7.40 (m, 2H), 7.20 (m, 2H), 6.98 (m, 2H), 4.38 (s, 2H), 4.36 (s, 2H), 3.40-3.00 (m, 1H), 1.36 (d, J=5.0 Hz, 3H); ESI⁺ MS: m/z (rel intensity) 512 (100, M+H).

N-((1H-benzo[d]imidazol-2-yl)methyl)-N-(4-fluoro-2-trifluoromethylbenzyl)-4-((pyridin-2-ylmethylamino)methyl)benzenesulfonamide (ER): ¹H NMR (300 MHz, CDCl₃) δ 8.28 (d, J=5.1 Hz, 1H), 7.15 (d, J=5.1 Hz, 1H), 7.02-7.07 (m, 4H), 6.87 (s, 1H), 4.00 (s, 2H), 3.68 (m, 4H), 3.24 (q, J=7.3 Hz, 2H), 2.10 (m, 2H), 1.42 (t, J=7.2 Hz, 3H); ESI⁺ MS: m/z (rel intensity) 374 (100, M+H).

N-((1H-benzo[d]imidazol-2-yl)methyl)-4-((pyridin-2-ylmethylamino)methyl)-N-(5,6,7,8-tetrahydroquinolin-8-yl)benzenesulfonamide (FD): ¹H NMR (300 MHz, CDCl₃) δ 8.28 (d, J=5.1 Hz, 1H), 7.15 (d, J=5.1 Hz, 1H), 7.07-7.02 (m, 4H), 6.87 (s, 1H), 4.00 (s, 2H), 3.68 (m, 4H), 3.24 (q, J=7.3 Hz, 2H), 2.10 (m, 2H), 1.42 (t, J=7.2 Hz, 3H); ESI⁺ MS: m/z (rel intensity) 374.1 (100, M+H).

Furthermore, compound EM can be prepared using methods similar to those described in Scheme XII. Those skilled in the art will recognize that in step a, 4-formylbenzene-1-sulfonyl chloride is reacted with a 1,4,8,11-tetraazacyclotetradecane derivative rather than benzylamine.

In addition, compounds like FE and FF can be prepared by reacting compounds such as DX with an isocyanate reagent, as shown below in Scheme XIII.

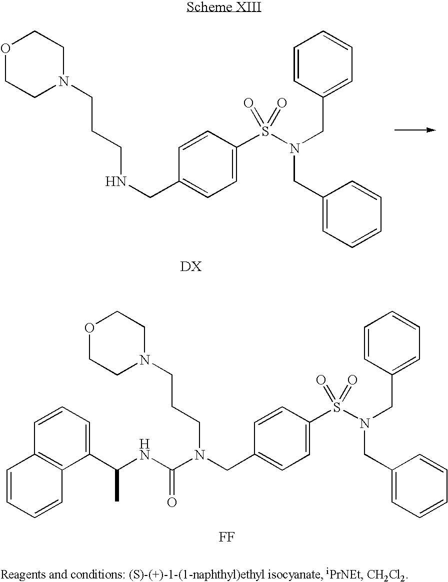

### Example 13

The following compounds FG, FH, FJ, FK, FL, FM, FN, FO, FP, FQ, FR, FS, FT, FU, FV, FW, FX, FY, FZ, GB, GA, GD and GI were prepared using methods similar to those used to prepare compound DC (e.g., for compound FI, diethylamine was used in step b of Scheme VII instead of pyridine-2-ylmethylamine; for compound DC and N-methyl-N-benzylamine was used instead of benzylamine in step a of Scheme XI), and are also non-limiting examples of the fourth aspect of the compounds of the present invention, each of which were prepared using methods similar to those described in Scheme XIII, using appropriately substituted amino reagents.

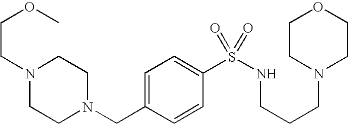

4-((4-(2-methoxyethyl)piperazin-1-yl)methyl)-N-(3-morpholinopropyl)benzenesulfonamide (FG): ¹H NMR (400 MHz, CDCl₃) [symbol] 7.78 (d, J=8.2 Hz, 2H), 7.46 (d, J=8.2 Hz, 2H), 3.72-3.67 (m, 4H), 3.56 (s, 2H), 3.50 (t, J=5.6 Hz, 2H), 3.33 (s, 3H), 3.08-3.04 (m, 2H), 2.58 (t, J=5.4 Hz, 4H), 2.50 (bs, 4H), 2.43-2.32 (m, 6H), 1.68-1.60 (m, 2H).

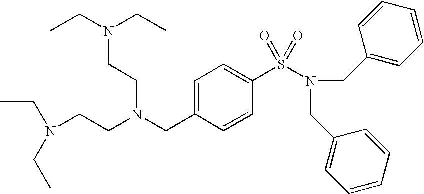

### N,N-dibenzyl-4-((bis(2-(diethylamino)ethyl)amino)-methyl)benzenesulfonamide (FH)

¹H NMR (400 MHz, CDCl₃) [symbol] 7.78 (d, J=8.4 Hz, 2H), 7.50 (d, J=8.4 Hz, 2H), 7.23-7.17 (m, 6H), 7.07-7.01 (m, 4H), 4.30 (s, 4H), 3.74 (s, 2H), 2.72-2.62 (m, 8H), 2.60 (q, J=7.2 Hz, 8H), 1.04 (t, J=7.2 Hz, 12H); ESI⁺ MS: m/z (rel intensity) 565.3 (80, [M+H]⁺).

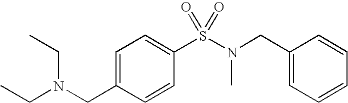

N-benzyl-4-((diethylamino)methyl)-N-methylbenzenesulfonamide (FI): ¹H NMR (400 MHz, CDCl₃) [symbol] 7.75 (d, J=8.8 Hz, 2H), 7.55 (d, J=8.4 Hz, 2H), 7.30-7.26 (m, 5H), 4.11 (s, 2H), 3.78 (s, 2H), 2.65 (q, J=6.8 Hz, 2H), 2.56 (s, 3H), 1.99 (s, 2H), 1.08 (t, J=6.8 Hz, 6H); ESI⁺ MS: m/z (rel intensity) 347.1 (100, [M+H]⁺).

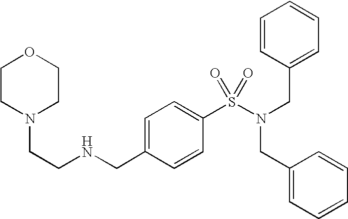

N,N-dibenzyl-4-((2-morpholinoethylamino)methyl)-benzenesulfonamide (FJ): ¹H NMR (400 MHz, CDCl₃) [symbol] 7.81 (d, J=8.4 Hz, 2H), 7.48 (d, J=8.4 Hz, 2H), 7.24-7.20 (m, 6H), 7.09-7.03 (m, 4H), 4.33 (s, 4H), 3.91 (s, 2H), 3.72 (t, J=4.8 Hz, 4H), 2.71 (t, J=5.6 Hz, 2H), 2.54 (t, J=6.0 Hz, 2H), 2.48-2.41 (m, 4H), 1.90 (s, 1H); ESI⁺ MS: m/z (rel intensity) 480.2 (100, [M+H]⁺).

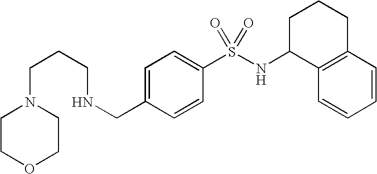

4-((3-morpholinopropylamino)methyl)-N-(1,2,3,4-tetrahydronaphthalen-1-yl)benzenesulfonamide (FK): ¹H NMR (400 MHz, CDCl₃) [symbol] 7.87 (d, J=8.6 Hz, 2H), 7.48 (d, J=8.6 Hz, 2H), 7.12-7.08 (m, 1H), 7.07 (t, J=7.2 Hz, 2H), 6.93 (d, J=7.6 Hz, 1H), 5.34 (bs, 1H), 4.43 (bs, 1H), 3.87 (s, 2H), 3.64 (t, J=4.8 Hz, 4H), 2.75-2.62 (m, 4H), 2.50-2.31 (m, 7H), 1.83-1.76 (m, 3H), 1.74-1.67 (m, 3H); ESI⁺ MS: m/z (rel intensity) 444.2 (100, [M+H]⁺).

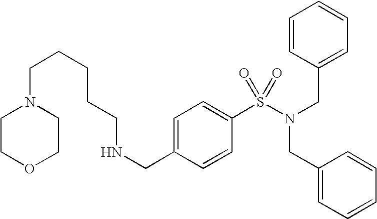

N,N-dibenzyl-4-((5-morpholinopentylamino)methyl)-benzenesulfonamide (FL): ¹H NMR (400 MHz, CDCl₃) [symbol] 7.81 (d, J=8.8 Hz, 2H), 7.47 (d, J=8.8 Hz, 2H), 7.23-7.18 (m, 6H), 7.07-7.01 (m, 4H), 4.32 (s, 4H), 3.88 (s, 2H), 3.72 (t, J=4.8 Hz, 4H), 2.64 (t, J=7.2 Hz, 2H), 2.44 (bs, 4H), 2.44-2.32 (m, 2H), 1.62-1.49 (m, 6H), 1.41-1.35 (m, 2H); ESI⁺ MS: m/z (rel intensity) 522.2 (100, [M+H]⁺).

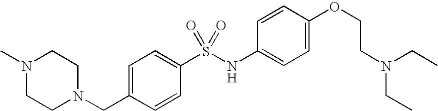

N-(4-(2-(diethylamino)ethoxy)phenyl)-4-((4-methylpiperazin-1-yl)methyl)benzenesulfonamide (FM): ¹H NMR (400 MHz, CDCl₃) [symbol] 7.56 (d, J=8.0 Hz, 2H), 7.28 (d, J=8.0 Hz, 2H), 6.89 (d, J=8.0 Hz, 2H), 6.65 (d, J=8.8 Hz, 2H), 5.48 (bs, 1H), 3.89 (t, J=6.0 Hz, 2H), 3.43 (s, 2H), 2.76 (t, J=6.0 Hz, 2H), 2.55 (q, J=7.2 Hz, 4H), 2.37 (bs, 6H), 2.20 (s, 3H), 0.97 (t, J=7.2 Hz, 6H); ESI⁺ MS: m/z (rel intensity) 461.2 (100, [M+H]⁺).

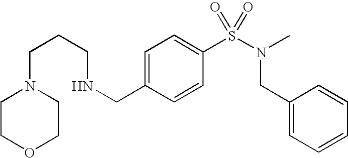

N-benzyl-N-methyl-4-((3-morpholinopropylamino)methyl)-benzenesulfonamide (FN): ¹H NMR (400 MHz, CDCl₃) [symbol] 7.73 (d, J=8.2 Hz, 2H), 7.48 (d, J=8.2 Hz, 2H), 7.27-7.22 (m, 5H), 4.06 (s, 2H), 3.83 (s, 2H), 3.62 (t, J=4.8 Hz, 4H), 2.69-2.65 (m, 4H), 2.52 (s, 3H), 2.39-2.36 (m, 6H), 1.72-1.65 (m, 2H).

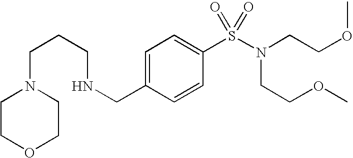

N,N-bis(2-methoxyethyl)-4-((3-morpholinopropylamino)-methyl)benzenesulfonamide (FO): ¹H NMR (400 MHz, CDCl₃) [symbol] 7.67 (d, J=8.2 Hz, 2H), 7.38 (d, J=8.2 Hz, 2H), 3.77 (s, 2H), 3.57 (t, J=4.8 Hz, 4H), 3.42 (t, J=6.0 Hz, 4H), 3.30-3.26 (m, 5H), 3.18 (s, 6H), 3.05 (bs, 2H), 2.60 (t, J=7.2 Hz, 2H), 2.36-2.30 (m, 6H), 2.66-1.59 (m, 2H); ESI⁺ MS: m/z (rel intensity) 430.1 (100, [M+H]⁺).

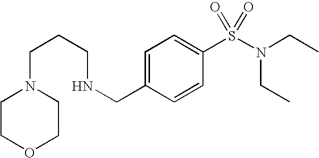

N,N-diethyl-4-((3-morpholinopropylamino)methyl)-benzenesulfonamide (FP): ¹H NMR (400 MHz, CDCl₃) [symbol] 7.65 (d, J=8.6 Hz, 2H), 7.37 (d, J=8.6 Hz, 2H), 3.76 (s, 2H), 3.59 (t, J=4.8 Hz, 4H), 3.13 (q, J=7.2 Hz, 4H), 2.60 (t, J=7.2 Hz, 2H), 2.34-2.30 (m, 6H), 2.18 (bs, 1H), 1.66-1.59 (m, 2H), 1.03 (t, J=7.2 Hz, 6H); ESI⁺ MS: m/z (rel intensity) 370.1 (100, [M+H]⁺).

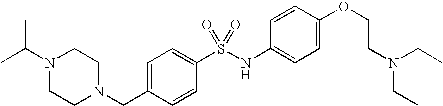

N-(4-(2-(diethylamino)ethoxy)phenyl)-4-((4-isopropylpiperazin-1-yl)methyl)benzenesulfonamide (FQ): ¹H NMR (400 MHz, CDCl₃) [symbol] 7.59 (d, J=8.0 Hz, 2H), 7.31 (d, J=8.0 Hz, 2H), 6.90 (d, J=8.4 Hz, 2H), 6.67 (d, J=8.4 Hz, 2H), 5.22 (bs, 1H), 3.91 (t, J=6.4 Hz, 2H), 3.45 (s, 2H), 2.78 (t, J=6.4 Hz, 2H), 2.61-2.55 (m, 6H), 2.54-2.45 (m, 3H), 2.45-2.34 (m, 3H), 1.00 (t, J=6.8 Hz, 12H); ESI⁺ MS: m/z (rel intensity) 489.2 (100, [M+H]⁺).

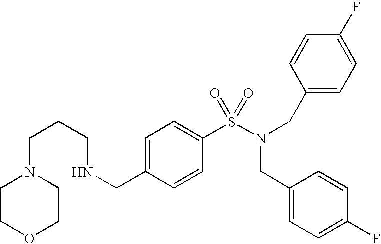

N,N-bis(4-fluorobenzyl)-4-((3-morpholinopropylamino)-methyl)benzenesulfonamide (FR): ¹H NMR (400 MHz, CDCl₃) [symbol] 7.77 (d, J=8.4 Hz, 2H), 7.48 (d, J=8.4 Hz, 2H), 6.99-6.97 (m, 4H), 6.87-6.83 (m, 4H), 4.23 (s, 4H), 3.88 (s, 2H), 3.66 (t, J=4.8 Hz, 2H), 3.59 (bs, 1H), 2.70 (t, J=7.2 Hz, 2H), 2.47-2.40 (m, 5H), 1.77-1.70 (m, 4H); ESI⁺ MS: m/z (rel intensity) 530.2 (100, [M+H]⁺).

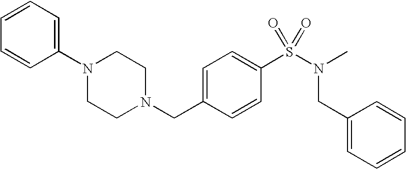

N-benzyl-N-methyl-4-((4-phenylpiperazin-1-yl)methyl)-benzenesulfonamide (FS): ¹H NMR (400 MHz, CDCl₃) [symbol] 7.80 (d, J=8.4 Hz, 2H), 7.58 (d, J=8.4 Hz, 2H), 7.33-7.24 (m, 8H), 6.92 (d, J=7.6 Hz, 2H), 6.86 (t, J=7.2 Hz, 1H), 4.15 (s, 2H), 3.67 (s, 2H), 3.25-3.23 (m, 4H), 2.69-2.64 (m, 4H), 2.60 (s, 3H); ESI⁺ MS: m/z (rel intensity) 436.2 (100, [M+H]⁺).

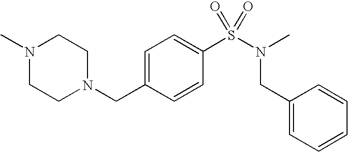

N-benzyl-N-methyl-4-((4-methylpiperazin-1-yl)methyl)benzenesulfonamide (FT): ¹H NMR (400 MHz, CDCl₃) [symbol] 7.74 (d, J=8.2 Hz, 2H), 7.49 (d, J=8.2 Hz, 2H), 7.34-7.24 (m, 5H), 4.11 (s, 2H), 3.56 (s, 2H), 2.56 (s, 3H), 2.51-2.44 (m, 8H), 2.28 (s, 2H); ESI⁺ MS: m/z (rel intensity) 374.1 (100, [M+H]⁺).

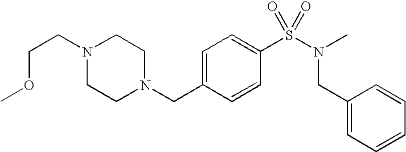

N-benzyl-4-((4-(2-methoxyethyl)piperazin-1-yl)methyl)-N-methylbenzenesulfonamide (FU): ¹H NMR (400 MHz, CDCl₃) [symbol] 7.75 (d, J=8.2 Hz, 2H), 7.49 (d, J=8.2 Hz, 2H), 7.32-7.24 (m, 5H), 4.11 (s, 2H), 3.56 (s, 2H), 3.49 (t, J=5.2 Hz, 2H), 3.32 (s, 2H), 2.57 (t, J=5.2 Hz, 2H), 2.57 (s, 3H), 2.55-2.45 (m, 8H); ESI⁺ MS: m/z (rel intensity) 418.2 (100, [M+H]⁺).

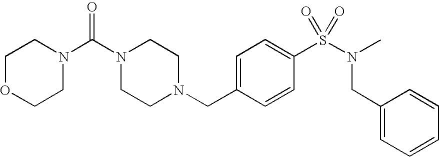

N-benzyl-N-methyl-4-((4-(morpholine-4-carbonyl)piperazin-1-yl)methyl)benzenesulfonamide (FV): ¹H NMR (400 MHz, CDCl₃) [symbol] 7.73 (d, J=8.2 Hz, 2H), 7.47 (d, J=8.2 Hz, 2H), 7.27-7.22 (m, 5H), 4.01 (s, 2H), 3.62-3.60 (m, 4H), 3.54 (s, 2H), 3.27-3.25 (m, 4H), 3.20-3.19 (m, 4H), 2.54 (s, 3H), 2.42-2.39 (m, 4H); ESI⁺ MS: m/z (rel intensity) 473.2 (100, [M+H]⁺).

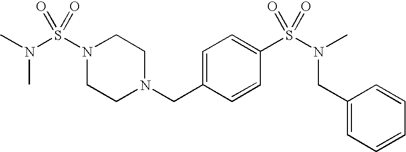

4-(4-(N-benzyl-N-methylsulfamoyl)benzyl)-N,N-dimethylpiperazine-1-sulfonamide (FW): ¹H NMR (400 MHz, CDCl₃) [symbol] 7.76 (d, J=8.2 Hz, 2H), 7.49 (d, J=8.2 Hz, 2H), 7.30-7.25 (m, 5H), 4.12 (s, 2H), 3.58 (s, 2H), 3.27-3.25 (m, 4H), 2.80 (s, 6H), 2.58 (s, 3H), 2.50-2.46 (m, 4H); ESI⁺ MS: m/z (rel intensity) 467.1 (100, [M+H]⁺).

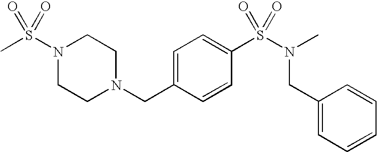

N-benzyl-N-methyl-4-((4-(methylsulfonyl)piperazin-1-yl)methyl)benzenesulfonamide (FX): ¹H NMR (400 MHz, CDCl₃) [symbol] 7.74 (d, J=8.0 Hz, 2H), 7.47 (d, J=8.0 Hz, 2H), 7.27-7.22 (m, 5H), 4.09 (s, 2H), 3.58 (s, 2H), 3.21 (bs, 4H), 2.74 (s, 3H), 2.55 (s, 3H), 2.53-2.51 (m, 4H); ESI⁺ MS: m/z (rel intensity) 438.1 (100, [M+H]⁺).

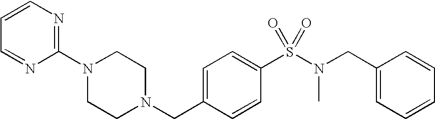

N-benzyl-N-methyl-4-((4-(pyrimidin-2-yl)piperazin-1-yl)methyl)benzenesulfonamide (FX): ¹H NMR (400 MHz, CDCl₃) [symbol] 8.26 (d, J=4.8 Hz, 1H), 7.77 (d, J=8.0 Hz, 2H), 7.52 (d, J=8.4 Hz, 2H), 7.29-7.24 (m, 5H), 6.44 (t, J=4.8 Hz, 1H), 4.12 (s, 2H), 3.83-3.80 (m, 4H), 3.58 (s, 2H), 2.58 (s, 3H), 2.50-2.47 (m, 4H); ESI⁺ MS: m/z (rel intensity) 438.1 (100, [M+H]⁺).

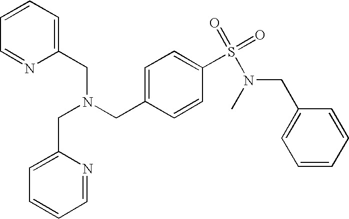

N-benzyl-4-((bis(pyridin-2-ylmethyl)amino)methyl)-N-methylbenzenesulfonamide (FZ): ¹H NMR (400 MHz, CDCl₃) δ 8.52-8.51 (m, 1H), 7.76 (d, J=8.8 Hz, 2H), 7.69-7.65 (m, 2H), 7.58 (d, J=6.8 Hz, 2H), 7.53 (d, J=7.6 Hz, 2H), 7.28-7.24 (m, 7H), 7.17-7.14 (m, 1H), 4.10 (s, 2H), 3.81 (s, 4H), 3.77 (s, 2H), 2.56 (s, 3H); ESI⁺ MS: m/z (rel intensity) 473.1 (100, [M+H]⁺).

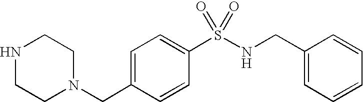

N-benzyl-4-(piperazin-1-ylmethyl)benzenesulfonamide (GB): ¹H NMR (400 MHz, CDCl₃) δ 7.78 (d, J=8.0 Hz, 2H), 7.45 (d, J=8.0 Hz, 2H), 7.24-7.18 (m, 5H), 4.13 (s, 2H), 3.51 (s, 2H), 2.87-2.85 (m, 4H), 2.38 (bs, 4H); ESI⁺ MS: m/z (rel intensity) 346.1 (100, [M+H]⁺).

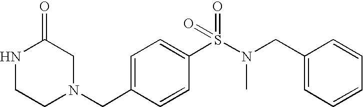

N-benzyl-N-methyl-4-((3-oxopiperazin-1-yl)methyl)benzenesulfonamide (GC): ¹H NMR (400 MHz, CDCl₃) δ 7.78 (d, J=8.4 Hz, 2H), 7.51 (d, J=8.4 Hz, 2H), 7.32-7.28 (m, 5H), 4.14 (s, 2H), 3.65 (s, 2H), 3.37 (bs, 2H), 3.14 (s, 2H), 2.67 (t, J=5.6 Hz, 2H), 2.58 (s, 3H); ESI⁺ MS: m/z (rel intensity) 374.1 (100, [M+H]⁺).

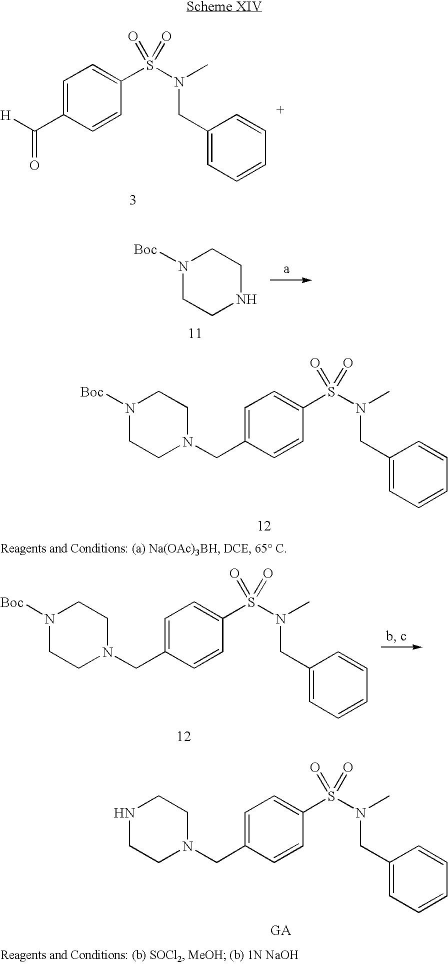

### Example 14

tert-butyl 4-(4-(N-benzyl-N-methylsulfamoyl)benzyl)piperazine-1-carboxylate (12): ¹H NMR (400 MHz, CDCl₃) [symbol] 7.75 (d, J=8.0 Hz, 2H), 7.50 (d, J=8.0 Hz, 2H), 7.28-7.24 (m, 5H), 4.11 (s, 2H), 3.56 (s, 2H), 3.45-3.39 (m, 4H), 2.57 (s, 3H), 2.40-2.33 (m, 4H), 1.43 (s, 9H).

Preparation of N-benzyl-N-methyl-4-(piperazin-1-ylmethyl)benzenesulfonamide (GA): A 50-mL round bottom flask was charged with Boc-protected amine 12 (0.94 g, 2.05 mmol) and methanol (8 mL). The solution was cooled to 0° C. and thionyl chloride (0.5 mL, 6.87 mmol) was added dropwise. The reaction was slowly warmed to room temperature and stirred for 18 h. The reaction was concentrated in vacuo. The solid was taken up in ethyl acetate (ca. 25 mL) and basified with a 1 N solution of sodium hydroxide (15 mL). The aqueous phase was extracted once with addition ethyl acetate (20 mL). The combined organic phases were washed with brine (15 mL), dried over anhydrous sodium sulfate and concentrated in vacuo. No further purification was necessary to yield GA (0.68 g, 1.89 mmol, 92% yield) as a white solid. ¹H NMR (400 MHz, CDCl₃) [symbol] 7.75 (d, J=8.0 Hz, 2H), 7.51 (d, J=8.0 Hz, 2H), 7.28-7.26 (m, 5H), 4.11 (s, 2H), 3.54 (s, 2H), 2.88-2.86 (m, 4H), 2.56 (s, 3H), 2.46 (bs, 1H), 2.41 (bs, 4H); ESI⁺ MS: m/z (rel intensity) 360.1 (100, [M+H]⁺).

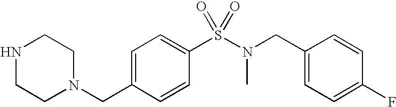

N-(4-fluorobenzyl)-N-methyl-4-(piperazin-1-ylmethyl)benzenesulfonamide (GD): ¹H NMR (400 MHz, CDCl₃) [symbol] 7.74 (d, J=8.2 Hz, 2H), 7.50 (d, J=8.2 Hz, 2H), 7.27-7.23 (m, 2H), 6.99 (t, J=8.4 Hz, 2H), 4.09 (s, 2H), 3.54 (s, 2H), 2.90 (t, J=5.2 Hz, 4H), 2.57 (s, 3H), 2.43 (bs, 4H), 2.23 (s, 3H); ESI⁺ MS: m/z (rel intensity) 378.1 (100, [M+H]⁺).

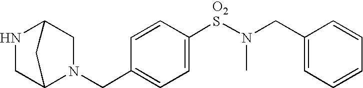

4-(2,5-diazabicyclo[2.2.1]heptan-2-ylmethyl)-N-benzyl-N-methylbenzenesulfonamide (GI): ¹H NMR (400 MHz, CDCl₃) [symbol] 7.64 (d, J=8.0 Hz, 2H), 7.51 (d, J=8.0 Hz, 2H), 7.34-7.22 (m, 5H), 4.11 (s, 2H), 3.78 (q, J=14.4 Hz, 2H), 3.63 (bs, 2H), 3.36 (s, 1H), 3.23 (d, J=10.4 Hz, 1H), 2.90-2.85 (m, 2H), 2.56 (s, 3H), 2.56-2.49 (m, 1H), 1.85 (d, J=9.6 Hz, 1H), 1.62 (d, J=9.6 Hz, 1H); ESI⁺ MS: m/z (rel intensity) 372.1 (100, [M+H]⁺).

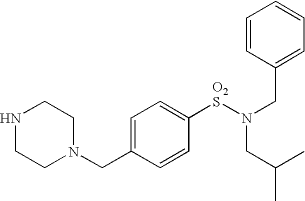

N-benzyl-N-isobutyl-4-(piperazin-1-ylmethyl)benzenesulfonamide (GE): ¹H NMR (400 MHz, CDCl₃): δ 7.74 (d, J=8.0 Hz, 2H), 7.45 (d, J=8.0 Hz, 2H), 7.23-7.18 (m, 5H), 4.29 (s, 2H), 3.53 (s, 2H), 2.90-2.87 (m, 6H), 2.39 (m, 4H), 1.61 (m, 1H), 0.71 (d, J=6.8 Hz, 6H); ESI⁺ MS: m/z (rel intensity) 402.2 (100, [M+H]⁺).

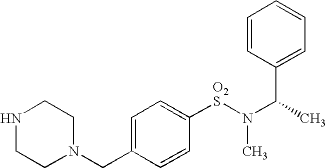

(S)—N-methyl-N-(1-phenylethyl)-4-(piperazin-1-ylmethyl)benzenesulfonamide (GF): ¹H NMR (400 MHz, CDCl₃): δ 7.77 (d, J=8.0 Hz, 2H), 7.46 (d, J=8.0 Hz, 2H), 7.28-7.22 (m, 5H), 5.27 (q, J=7.2 Hz, 1H), 3.54 (s, 2H), 2.93-2.90 (m, 4H), 2.57 (s, 3H), 2.46-2.36 (m, 4H), 1.29 (d, J=7.2 Hz, 3H); ESI⁺ MS: m/z (rel intensity) 374.1 (100, [M+H]⁺).

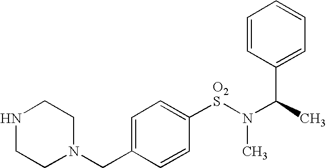

(R)—N-methyl-N-(1-phenylethyl)-4-(piperazin-1-ylmethyl)benzenesulfonamide (GG): ¹H NMR (400 MHz, CDCl₃): δ 7.77 (d, J=8.0 Hz, 2H), 7.46 (d, J=8.0 Hz, 2H), 7.28-7.22 (m, 5H), 5.27 (q, J=7.2 Hz, 1H), 3.54 (s, 2H), 2.93-2.90 (m, 4H), 2.57 (s, 3H), 2.46-2.36 (m, 4H), 1.29 (d, J=7.2 Hz, 3H); ESI⁺ MS: m/z (rel intensity) 374.1 (100, [M+H]⁺).

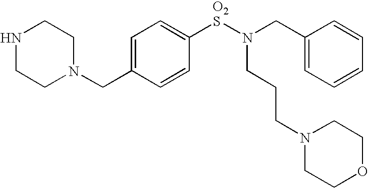

N-benzyl-N-(3-morpholinopropyl)-4-(piperazin-1-ylmethyl)benzenesulfonamide (GH): ¹H NMR (400 MHz, CDCl₃) [symbol] 7.76 (d, J=8.4 Hz, 2H), 7.47 (d, J=8.4 Hz, 2H), 7.30-7.24 (m, 5H), 4.30 (s, 2H), 3.56 (t, J=4.4 Hz, 4H), 3.53 (s, 2H), 3.14-3.10 (m, 2H), 2.89 (t, J=5.2 Hz, 4H), 2.50-2.30 (m, 4H), 2.20-2.10 (m, 6H), 1.58-1.45 (m, 2H); ESI⁺ MS: m/z (rel intensity) 473.2 (100, [M+H]⁺).

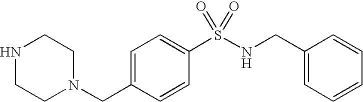

N-benzyl-4-(piperazin-1-ylmethyl)benzenesulfonamide (GB): ¹H NMR (400 MHz, CDCl₃) δ 7.78 (d, J=8.0 Hz, 2H), 7.45 (d, J=8.0 Hz, 2H), 7.24-7.18 (m, 5H), 4.13 (s, 2H), 3.51 (s, 2H), 2.87-2.85 (m, 4H), 2.38 (bs, 4H); ESI⁺ MS: m/z (rel intensity) 346.1 (100, [M+H]⁺).

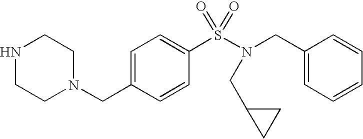

N-benzyl-N-(cyclopropylmethyl)-4-(piperazin-1-ylmethyl)-benzenesulfonamide (GP): ¹H NMR (400 MHz, CDCl₃) [symbol] 8.38 (d, J=8.0 Hz, 2H), 7.46 (d, J=8.4 Hz, 2H), 7.30-7.22 (m, 5H), 4.45 (s, 2H), 3.53 (m, 2H), 3.01 (d, J=6.8 Hz, 2H), 2.92-2.89 (m, 4H), 2.50-2.35 (m, 4H), 0.72-0.60 (m, 1H), 0.33-0.28 (m, 2H), (−) 0.04-(−) 0.08 (m, 2H); ESI⁺ MS: m/z (rel intensity) 400.2 (90, [M+H]⁺).

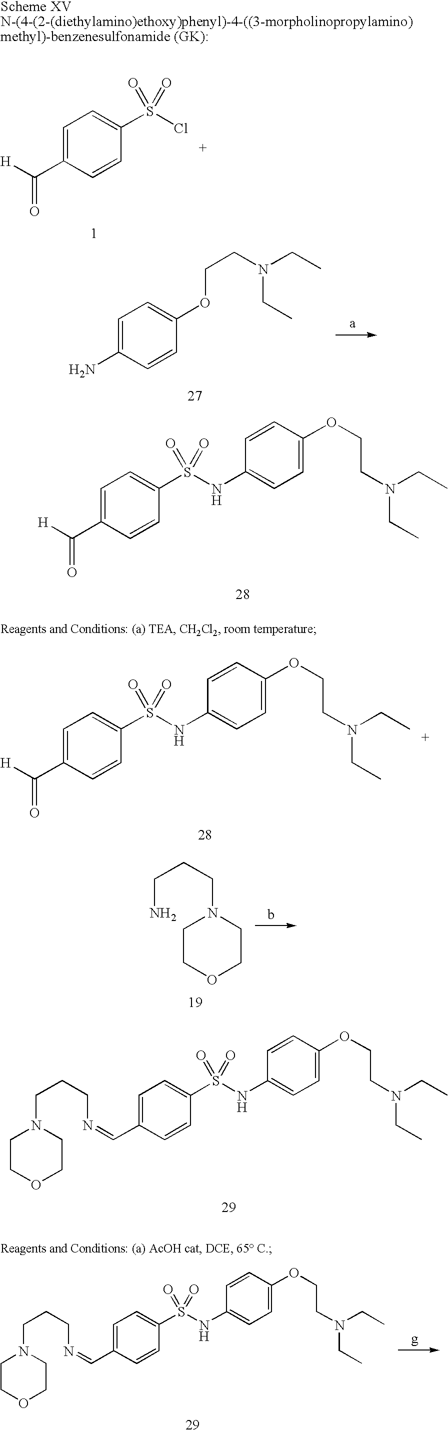

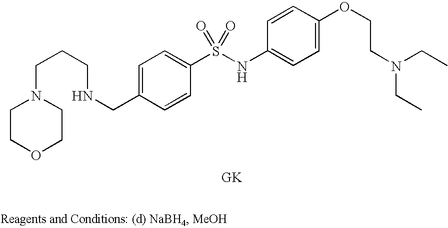

### Example 15

Preparation of N-(4-(2-(diethylamino)ethoxy)phenyl)-4-formylbenzenesulfonamide (28): ¹H NMR (400 MHz, CDCl₃) [symbol] 10.01 (s, 1H), 7.87 (d, J=8.4 Hz, 2H), 7.83 (d, J=8.4 Hz, 2H), 6.95 (d, J=8.8 Hz, 2H), 6.68 (d, J=8.8 Hz, 2H), 6.56 (bs, 1H), 3.95 (t, J=6.0 Hz, 2H), 2.85 (t, J=6.8 Hz, 2H), 2.65 (q, J=6.8 Hz, 4H), 1.04 (t, J=7.2 Hz, 6H).

Preparation of N-(4-(2-(diethylamino)ethoxy)phenyl)-4-((3-morpholinopropylimino)methyl)benzenesulfonamide (29): A 50-mL round bottom flask was charged with aldehyde 28 (0.95 g, 2.73 mmol), amine 19 (0.44 ml, 3.01 mmol), dichloroethane (11 mL) and a catalytic amount of acetic acid (1-2 drops). The reaction was then heated to 58° C. for 18 h. The reaction was then taken up in ethyl acetate (15 mL) and quenched with aqueous saturated solution of sodium bicarbonate (10 mL). The aqueous phase was extracted once with additional ethyl acetate (10 mL). The combined organic phases were washed with brine (15 mL) and dried over anhydrous sodium sulfate. The supernatant was decanted and concentrated in vacuo. The crude product was purified by silica gel chromatography (0-10% MeOH/CH₂Cl₂) to yield imine 29 (0.36 g, 28% yield) as a yellow oil. ¹H NMR (400 MHz, CDCl₃) [symbol] 8.26 (s, 1H), 7.87 (d, J=8.4 Hz, 2H), 7.83 (d, J=8.4 Hz, 2H), 6.95 (d, J=8.8 Hz, 2H), 6.68 (d, J=8.8 Hz, 2H), 3.95 (t, J=6.0 Hz, 2H), 3.66-3.64 (m, 6H), 2.82 (t, J=6.4 Hz, 2H), 2.65 (q, J=6.8 Hz, 4H), 2.41-2.36 (m, 6H), 1.70-1.68 (m, 2H), 1.03 (t, J=6.8 Hz, 6H).

Preparation of N-(4-(2-(diethylamino)ethoxy)phenyl)-4-((3-morpholinopropylamino)methyl)benzenesulfonamide (GK): A 50-mL round bottom flask was charged with imine 29 (0.36 g, 0.76 mmol) and methanol (8 mL). The solution was cooled to 0° C. and sodium borohydride (0.12 g, 3.17 mmol) was added slowly. The reaction was stirred at room temperature for 2 h, then quenched with a aqueous saturated solution of sodium bicarbonate (8 mL). The aqueous phase was extracted once with ethyl acetate (10 mL). The combined organic phases were washed with brine (10 mL) and dried over anhydrous sodium sulfate. The supernatant was decanted and concentrated in vacuo. The crude product was purified by silica gel chromatography (0-10% MeOH/CH₂Cl₂) to yield GK (0.33 g, 86% yield) as a yellow oil. ¹H NMR (400 MHz, CDCl₃) [symbol] 7.60 (d, J=8.8 Hz, 2H), 7.33 (d, J=8.0 Hz, 2H), 6.92 (d, J=8.8 Hz, 2H), 6.71 (d, J=8.8 Hz, 2H), 3.95 (t, J=6.4 Hz, 2H), 3.79 (s, 2H), 3.67-3.65 (m, 4H), 2.82 (t, J=6.4 Hz, 2H), 2.67-2.58 (m, 6H), 2.38 (m, 6H), 1.70-1.67 (m, 2H), 1.03 (t, J=7.2 Hz, 6H); ESI⁺ MS: m/z (rel intensity) 505.3 (80, [M+H]⁺).

Compounds GL, GM and GN listed below are non-limiting examples of the fourth aspect of the present invention, each of which were prepared using methods similar to those described in Scheme XV, using appropriately substituted amino reagents.

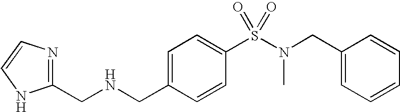

4-(((1H-imidazol-2-yl)methylamino)methyl)-N-benzyl-N-methylbenzenesulfonamide (GL): ¹H NMR (400 MHz, CDCl₃) δ 7.71 (d, J=8.0 Hz, 2H), 7.45 (d, J=7.6 Hz, 2H), 7.30-7.27 (m, 5H), 6.96 (s, 2H), 4.08 (s, 2H), 3.88 (s, 2H), 3.83 (s, 2H), 2.54 (s, 3H); ESI⁺ MS: m/z (rel intensity) 371.1 (100, [M+H]⁺).

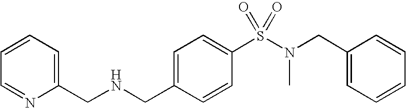

N-benzyl-N-methyl-4-((pyridin-2-ylmethylamino)methyl)benzenesulfonamide (GM): ¹H NMR (400 MHz, CDCl₃) δ 8.54 (bs, 1H), 7.77 (bs, 2H), 7.63 (bs, 1H), 7.54 (bs, 3H), 7.28 (bs, 6H), 7.16 (bs, 1H), 4.10 (s, 2H), 3.91 (s, 4H), 2.55 (s, 3H), 2.18 (bs, NH); ESI⁺ MS: m/z (rel intensity) 382.1 (100, [M+H]⁺).

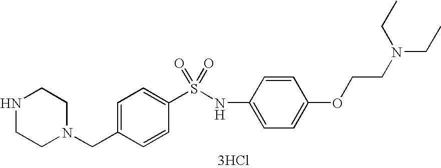

N-(4-(2-(diethylamino)ethoxy)phenyl)-4-(piperazin-1-ylmethyl)benzenesulfonamide (GJ): ¹H NMR (400 MHz, d⁶-DMSO) δ 7.70 (bs, 4H), 6.99 (d, J=8.8 Hz, 2H), 6.83 (d, J=8.8 Hz, 2H), 4.24 (bs, 2H), 3.55 (s, 2H), 3.38 (bs, 6H), 3.14-3.10 (m, 6H), 2.46 (s, 2H), 1.18 (t, J=7.6 Hz, 6H); ESI⁺ MS: m/z (rel intensity) 447.2 (80, [M+H]⁺).

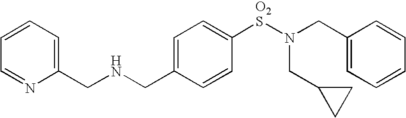

N-benzyl-N-(cyclopropylmethyl)-4-((pyridin-2-ylmethylamino)methyl)benzenesulfonamide (GN): ¹H NMR (400 MHz, CDCl₃) [symbol] 8.58-8.54 (m, 1H), 7.80 (d, J=8.4 Hz, 2H), 7.64 (dt, J=7.2, 1.6 Hz, 1H), 7.50 (d, J=8.4 Hz, 2H), 7.32-7.22 (m, 6H), 7.22-7.14 (m, 1H), 4.44 (s, 2H), 3.92 (s, 2H), 2.99 (s, 2H), 3.00 (d, J=6.8 Hz, 2H), 0.70-0.60 (m, 1H), 0.33-0.27 (m, 2H), (−) 0.04-(−) 0.07 (m, 2H); ESI⁺ MS: m/z (rel intensity) 422.1 (100, [M+H]⁺).

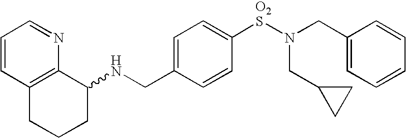

N-benzyl-N-(cyclopropylmethyl)-4-((5,6,7,8-tetrahydroquinolin-8-ylamino)-methyl)benzenesulfonamide (GO): ¹H NMR (400 MHz, CDCl₃) [symbol] 8.38 (d, J=4.4 Hz, 1H), 7.79 (d, J=8.4 Hz, 2H), 7.55 (d, J=8.4 Hz, 2H), 7.38 (d, J=7.6 Hz, 1H), 7.30-7.20 (m, 5H), 7.07 (dd, J=4.8, 7.6 Hz, 1H), 4.43 (s, 2H), 4.05-3.96 (m, 2H), 3.84 (t, J=4.8 Hz, 1H), 3.01-2.97 (m, 3H), 2.83-2.70 (m, 2H), 2.18-2.12 (m, 1H), 2.04-1.98 (m, 1H), 1.80-1.65 (m, 2H), 0.65-0.59 (m, 1H), 0.32-0.27 (m, 2H), (−) 0.05-(−) 0.09 (m, 2H); ESI⁺ MS: m/z (rel intensity) 462.2 (100, [M+H]⁺).

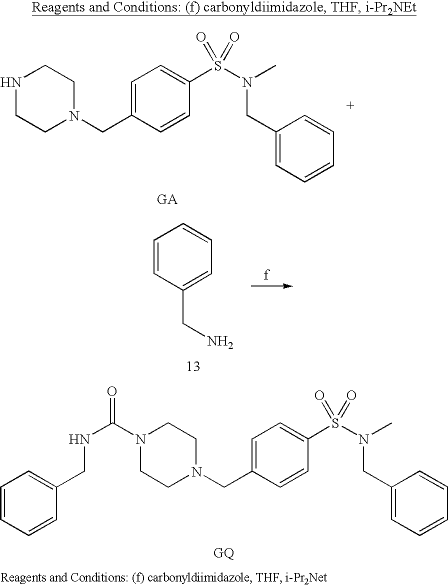

### Example 16

Preparation of N-benzyl-4-(4-(N-benzyl-N-methylsulfamoyl)benzyl)-piperazine-1-carboxamide (GQ): A 25-mL round bottom flask was charged with benzylamine (0.05 mL, 0.46 mmol), 1,1′-carbonyldiimidazole (0.07 g, 0.43 mmol), i-Pr₂NEt (0.08 mL, 0.46 mmol) and tetrahydrofuran (5 mL). The solution was stirred at room temperature for 15 min. GA (0.17 g, 0.47 mmol) was then added and the reaction was stirred for 18 h. The reaction was taken up in ethyl acetate (10 mL) and quenched with a saturated aqueous solution of sodium bicarbonate (15 mL). The aqueous phase was extracted once with ethyl acetate (10 mL). The combined organic phases were washed with brine (10 mL), dried over anhydrous sodium sulfate, filtered and concentrated in vacuo. The crude product was purified by silica gel chromatography (0-10% MeOH/CH₂Cl₂) to yield GQ 35 (0.16 g, 0.32 mmol, 69% yield) as a white solid. ¹H NMR (400 MHz, CDCl₃) [symbol] 7.77 (d, J=8.0 Hz, 2H), 7.50 (d, J=8.0 Hz, 2H), 7.31-7.24 (m, 10H), 4.85 (t, J=5.6 Hz, 1H), 4.75 (d, J=5.6 Hz, 2H), 4.12 (s, 2H), 3.56 (s, 2H), 3.39-3.36 (m, 4H), 2.57 (s, 3H), 2.42-2.39 (m, 4H); ESI⁺ MS: m/z (rel intensity) 493.1 (100, [M+H]⁺).

Compound GR listed below is a non-limiting example of the fourth aspect of the present invention, each of which was prepared using methods similar to those described in Scheme XVI, using appropriately substituted amino reagents.

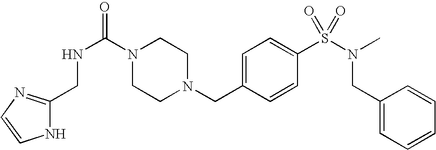

N-((1H-imidazol-2-yl)methyl)-4-(4-(N-benzyl-N-methylsulfamoyl)benzyl)-piperazine-1-carboxamide (GR): ¹H NMR (400 MHz, CDCl₃) [symbol] 7.76 (d, J=8.0 Hz, 2H), 7.50 (d, J=8.0 Hz, 2H), 7.35-7.31 (m, 5H), 6.90 (s, 2H), 4.31 (d, J=5.2 Hz, 2H), 4.12 (s, 2H), 3.54 (s, 2H), 3.47-3.44 (m, 4H), 2.58 (s, 3H), 2.44-2.39 (m, 4H); ESI⁺ MS: m/z (rel intensity) 483.1 (100, [M+H]⁺).

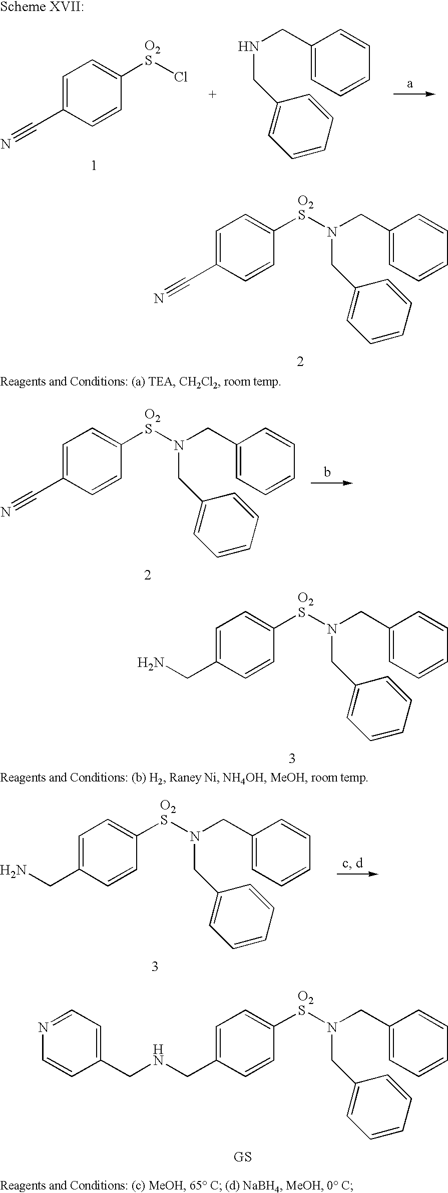

### Example 17

### N,N-dibenzyl-4-((pyridin-4-ylmethylamino)methyl)benzenesulfonamide (GS)

Preparation of N,N-dibenzyl-4-cyanobenzenesulfonamide (2): To a solution of 4-cyanobenzene sulfonyl chloride (2) (2.25 g, 11.15 mmol) in dichloromethane was added dibenzylamine (2.36 mL, 12.27 mmol) and triethylamine (1.87 mL, 13.39 mmol). The resulting mixture was stirred at room temperature for 18 h. A saturated aqueous solution of sodium bicarbonate (10 mL) was added. The product was extracted three times with 10 mL of dichloromethane. The combined organic layers were dried over potassium carbonate, filtered and concentrated. The crude material (4.04 g) was used in the next step without further purification: ¹H NMR (400 MHz, CDCl₃) [symbol] 7.83 (d, J=8.4 Hz, 2H), 7.73 (d, J=8.4 Hz, 2H), 7.30-7.20 (m, 6H), 7.10-7.03 (m, 4H), 4.36 (s, 4H); ESI⁺ MS: m/z (rel intensity) 385.1 (95, [M+H]⁺).

Preparation of 4-(aminomethyl)-N,N-dibenzylbenzenesulfonamide (3): To a solution of 2 (1.00 g, 2.75 mmol, 1.0 equiv.) in methanol was added ammonium hydroxide (1 mL). The resulting mixture was hydrogenated over Raney nickel (1 mL) for 24 h at room temperature. The reaction was then filtered through Celite. The solvents were removed by evaporation. The product was dissolved in ethyl acetate (15 mL) and H₂O (10 mL) was added. The layers were separated. The product was extracted twice more with ethyl acetate (5 mL). The combined organic layers were dried over magnesium sulfate, filtered and concentrated. The crude material (0.86 g, 85% yield) was used in the next step without further purification: ¹H NMR (400 MHz, CDCl₃) [symbol] 7.79 (d, J=8.4 Hz, 2H), 7.45 (d, J=8.0 Hz, 2H), 7.21-7.18 (m, 6H), 7.08-7.01 (m, 4H), 4.30 (s, 4H), 3.97 (s, 2H), 1.52 (bs, 2H); ESI⁺ MS: m/z (rel intensity) 367.1 (100, [M+H]⁺).

Preparation of N,N-dibenzyl-4-((pyridin-4-ylmethylamino)methyl)-benzenesulfonamide (GS): To a solution of 3 (0.40 g, 1.09 mmol) in methanol was added 4-pyridine carboxaldehyde (105 μL, 1.09 mmol). The resulting mixture was warmed to 65° C. and stirred for 2 h. The reaction was then cooled to 0° C. and sodium borohydride (0.17 g, 4.36 mmol) was added portion-wise. The reaction was slowly warmed to room temperature then a saturated aqueous solution of sodium bicarbonate (5 mL) was added. The product was extracted three times with 5 mL of dichloromethane. The combined organic layers were dried over potassium carbonate, filtered and concentrated. The crude material was purified by silica gel chromatography (0-5% MeOH/CHCl₃) to afford 0.34 g (68% yield) of pure product GS: ¹H NMR (400 MHz, CDCl₃) [symbol] 8.56 (d, J=6.0 Hz, 2H), 7.80 (d, J=8.0 Hz, 2H), 7.48 (d, J=8.0 Hz, 2H), 7.28 (d, J=6.0 Hz, 2H), 7.22-7.16 (m, 6H), 7.08-7.00 (m, 4H), 4.31 (s, 4H), 3.88 (s, 2H), 3.82 (s, 2H); ESI⁺ MS: m/z (rel intensity) 458.1 (100, [M+H]⁺).

Compound GT listed below is a non-limiting example of the fourth aspect of the present invention, which was prepared using methods similar to those described in Scheme XVII, using appropriately substituted amino reagents.

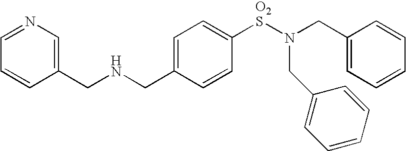

N,N-dibenzyl-4-((pyridin-3-ylmethylamino)methyl)benzenesulfonamide (GT): ¹H NMR (400 MHz, CDCl₃) [symbol] 8.56 (d, J=2.0 Hz, 1H), 8.49 (dd, J=1.6, 5.2 Hz, 1H), 7.77 (d, J=8.0 Hz, 2H), 7.71-7.66 (m, 1H), 7.46 (d, J=8.0 Hz, 2H), 7.27-7.22 (m, 1H), 7.20-7.14 (m, 6H), 7.06-7.00 (m, 4H), 4.30 (s, 4H), 3.86 (s, 2H), 3.79 (s, 2H); ESI MS: m/z (rel intensity) 458.1 (100, [M+H]⁺).

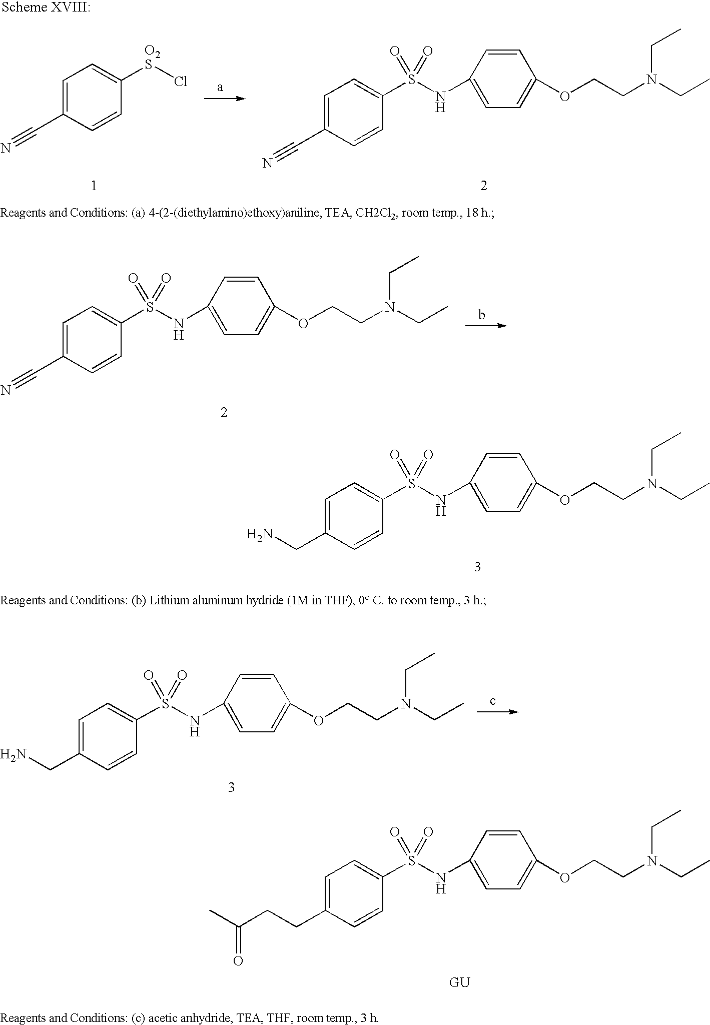

### Example 18

### N-(4-(N-(4-(2-(diethylamino)ethoxy)phenyl)sulfamoyl)benzyl)acetamide (GU)

Preparation of 4-cyano-N-(4-(2-(diethylamino)ethoxy)-phenyl)benzenesulfonamide (2): To a solution of (1) (1.0 g, 4.96 mmol) in dichloromethane (10 mL), was added triethylamine (760 μL, 5.45 mmol) and 4-(2-(diethylamino)ethoxy)aniline (1.14 g, 5.45 mmol). The mixture stirred at room temperature for 18 h. The reaction mixture was quenched with brine (1 mL), dried over magnesium sulfate, filtered and concentrated. The crude material was purified by silica gel chromatography (5% MeOH/CHCl₃) to afford (2) as a yellow solid (0.93 g, 50% yield): ¹HNMR (400 MHz, CDCl₃) δ 7.75 (d, J=6.8 Hz, 2H), 7.68 (d, J=8.4 Hz, 2H), 6.91 (d, J=8.4 Hz, 2H), 6.72 (d, J=8.8 Hz, 2H), 4.5 (bs, NH), 3.94 (t, J=6.4 Hz, 2H), 2.82 (t, J=6.4 Hz, 2H), 2.61 (q, J=7.2 Hz, 4H), 1.03 (t, J=6.8 Hz, 6H); ESI⁺ MS: m/z (rel intensity) 374.1 (100, [M+H]⁺).

Preparation of 4-(aminomethyl)-N-(4-(2-(diethylamino)ethoxy)-phenyl)benzenesulfonamide (3): To a solution of (2) (0.50 g, 1.34 mmol) in tetrahydrofuran (3 mL) at 0° C., was added a 1.0 M solution of lithium aluminum hydride in tetrahydrofuran (1.34 mL) dropwise. The ice bath was removed and the mixture stirred at room temperature for 3 h. The reaction was cooled to 0° C. and quenched with water (2 mL) for 10 minutes. A solution of 15% sodium hydroxide (2 mL) was added and the reaction stirred for 15 minutes at 0° C. The solution was diluted with water and extracted with ethyl acetate (2×50 mL). The combined organic layers were dried over magnesium sulfate, filtered and concentrated to yield compound (3) as a crude yellow solid (0.14 g, 27% yield): Crude ¹H NMR (400 MHz, CDCl₃) δ 7.60 (d, J=8.4 Hz, 2H), 7.32 (d, J=8.4 Hz, 2H), 6.91 (d, J=10.4 Hz, 2H), 6.70 (d, J=6.8 Hz, 2H), 3.96-3.92 (m, 4H), 2.83-2.79 (m, 2H), 2.63-2.56 (m, 4H), 1.04-1.00 (m, 6H).

Preparation of N-(4-(N-(4-(2-(diethylamino)ethoxy)phenyl)sulfamoyl)-benzyl)acetamide (GU): To a solution of (3) (0.30 g, 0.79 mmol) in dichloromethane (2 mL), was added triethylamine (0.12 mL, 0.87 mmol) and 4-fluorobenzenesulfonyl chloride (0.17 g, 0.87 mmol). The mixture stirred at room temperature for 18 h. The reaction mixture was quenched with brine (0.5 mL), dried over magnesium sulfate, filtered and concentrated. The crude material was purified by silica gel chromatography (10% MeOH/CHCl₃) to give GU (13 mg, 4% yield): ¹H NMR (400 MHz, CDCl₃) δ 7.53 (d, J=8.0 Hz, 2H), 7.23 (d, J=4.4 Hz, 2H), 6.91 (d, J=8.8 Hz, 2H), 6.69 (d, J=8.8 Hz, 2H), 6.25 (bs, 1H), 4.41 (d, J=6.4 Hz, 2H), 3.94 (t, J=6.0 Hz, 2H), 2.82 (t, J=6.0 Hz, 2H), 2.61 (q, J=6.8 Hz, 4H), 2.02 (s, 3H), 1.03 (t, J=6.8 Hz, 6H); ESI⁺ MS: m/z (rel intensity) 420.1 (100, [M+H]⁺).

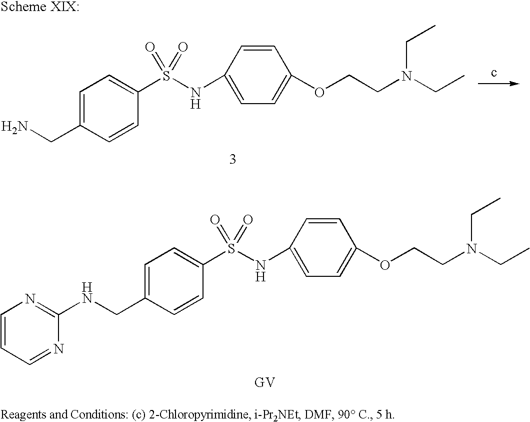

### Example 19

Preparation of N-(4-(2-(diethylamino)ethoxy)phenyl)-4-((pyrimidin-2-ylamino)methyl)benzenesulfonamide (GV): To a solution of (3) (0.20 g, 0.53 mmol) in dimethylformamide (1 mL), was added diisopropylethylamine (0.37 mL, 2.12 mmol) and 2-chloropyrimidine (0.06 g, 0.53 mmol). The mixture stirred at 85° C. for 5 h. The reaction mixture was quenched with a saturated aqueous solution of sodium bicarbonate (2 mL). The product was extracted with ethyl acetate. The combined organic layers were dried over magnesium sulfate, filtered, concentrated. The crude material was purified by silica gel chromatography (5% MeOH/CHCl₃) to yield GV (24 mg, 10% yield): ¹H NMR (400 MHz, CDCl₃) δ 8.24 (d, J=4.4 Hz, 2H), 7.57 (d, J=8.8 Hz, 2H), 7.32 (d, J=7.6 Hz, 2H), 6.91 (d, J=8.0 Hz, 2H), 6.70 (d, J=8.0 Hz, 2H), 6.00 (bs, 1H), 4.64 (d, J=5.6 Hz, 2H), 3.93 (t, J=6.4 Hz, 2H), 2.80 (t, J=6.4 Hz, 2H), 2.60 (q, J=7.2 Hz, 4H), 1.02 (t, J=7.2 Hz, 6H); ESI⁺ MS: m/z (rel intensity) 456.2 (60, [M+H]⁺).

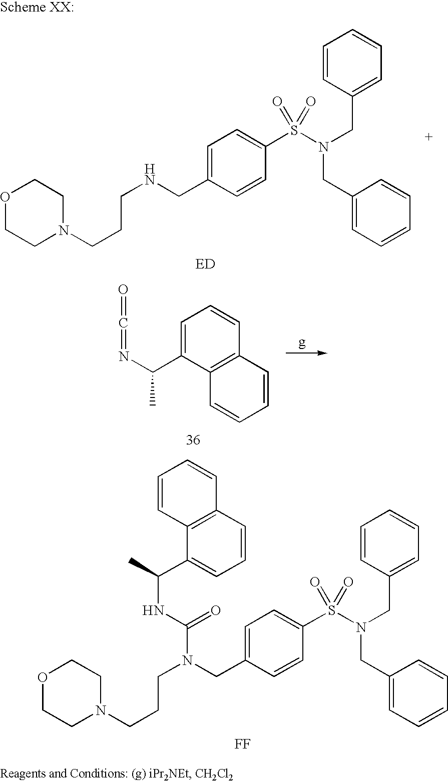

### Example 20

Preparation of (S)—N,N-dibenzyl-4-((1-(3-morpholinopropyl)-3-(1-(naphthalen-1-yl)ethyl)ureido)methyl)benzenesulfonamide (FF): A 25-mL round bottom flask was charged with amine ED (0.32 mL, 0.65 mmol), (S)-(+)-1-(1-naphthyl)ethyl isocyanate (0.11 g, 0.65 mmol), diisopropylethylamine (0.34 mL, 1.95 mmol) and dichloromethane (3 mL). The solution was stirred at room temperature for 18 h. The reaction was taken up in ethyl acetate (10 mL) and quenched with a saturated aqueous solution of sodium bicarbonate (10 mL). The aqueous phase was extracted once with ethyl acetate (10 mL) and the organic phases were combined, washed with brine (10 mL), dried over anhydrous sodium sulfate, filtered and concentrated in vacuo. The crude product was purified by silica gel chromatography (0-10% MeOH/CH₂Cl₂) to yield FF (0.25 g, 0.36 mmol, 56% yield). ¹H NMR (400 MHz, CDCl₃) [symbol] 8.17 (d, J=8.0 Hz, 1H), 7.84 (d, J=7.2 Hz, 1H), 7.78 (d, J=8.4 Hz, 3H), 7.55-7.40 (m, 6H), 7.22-7.15 (m, 6H), 7.08-7.01 (m, 4H), 6.33 (bd, J=7.2 Hz, 1H), 5.94-5.85 (m, 1H), 4.69 (d, J=16.0 Hz, 1H), 4.55 (d, J=16.0 Hz, 1H), 4.30 (s, 4H), 3.32-3.28 (m, 2H), 3.20-3.09 (m, 4H), 2.19-2.10 (m, 4H), 1.94-1.82 (m, 2H), 1.71 (d, J=6.4 Hz, 3H), 1.59-1.49 (m, 2H); ESI⁺ MS: m/z (rel intensity) 691.3 (100, [M+H]⁺).

Compounds HQ, HR, HS, HT, HV, HW, HX, HY, HZ and IA listed below are non-limiting examples of the fourth aspect of the present invention, each of which were prepared using methods similar to those described in Scheme XX, using appropriately substituted amino reagents.

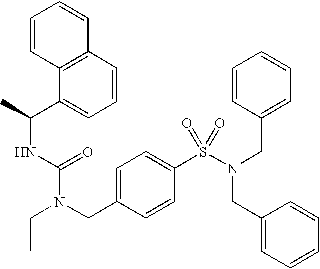

(S)—N,N-dibenzyl-4-((1-ethyl-3-(1-(naphthalen-1-yl)ethyl)ureido)methyl)-benzenesulfonamide (HQ): ¹H NMR (400 MHz, CDCl₃) [symbol] 8.19 (d, J=8.0 Hz, 1H), 7.88-7.86 (m, 1H), 7.80-7.78 (m, 1H), 7.74 (d, J=8.8 Hz, 2H), 7.56-7.44 (m, 5H), 7.34 (d, J=8.0 Hz, 2H), 7.23-7.21 (m, 6H), 7.08-7.05 (m, 4H), 5.88 (m, 1H), 4.85 (d, J=7.6 Hz, 1H), 4.56 (q, J=16.4 Hz, 2H), 4.32 (s, 4H), 3.20 (q, J=7.2 Hz, 2H), 1.67 (d, J=6.8 Hz, 3H), 1.07 (t, J=7.2 Hz, 3H).

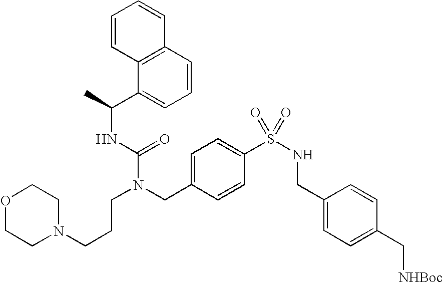

(S)-tert-butyl-4-((4-((1-(3-morpholinopropyl)-3-(1-(naphthalen-1-yl)ethyl)ureido) methyl)phenylsulfonamido)methyl)benzylcarbamate (HR): ¹H NMR (400 MHz, CDCl₃) [symbol] 8.14 (d, J=7.6 Hz, 1H), 7.84-7.67 (m, 1H), 7.79 (d, J=8.0 Hz, 1H), 7.76 (d, J=8.4 Hz, 3H), 7.56-7.44 (m, 5H), 7.33 (d, J=8.4 Hz, 2H), 7.13 (d, J=8.4 Hz, 2H), 7.09 (d, J=8.4 Hz, 2H), 6.43 (d, J=7.2 Hz, 1H), 5.90-5.82 (m, 1H), 5.62-5.54 (m, 1H), 5.18-5.10 (m, 1H), 4.66 (d, J=16.0 Hz, 1H), 4.50 (d, J=16.0 Hz, 1H), 4.19 (d, J=6.0 Hz, 2H), 4.08 (d, J=6.0 Hz, 2H), 3.33-3.28 (m, 2H), 3.15-3.08 (m, 4H), 2.20-2.12 (m, 4H), 1.90-1.86 (m, 2H), 1.70 (d, J=7.2 Hz, 3H), 1.58-1.53 (m, 2H), 1.43 (s, 9H); ESI⁺ MS: m/z (rel intensity) 730.4 (100, [M+H]⁺).

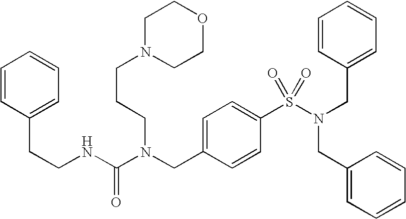

N,N-dibenzyl-4-((1-(3-morpholinopropyl)-3-phenethylureido)methyl)-benzenesulfonamide (HU): ¹H NMR (400 MHz, CDCl₃) [symbol] 7.77 (d, J=8.0 Hz, 2H), 7.35 (d, J=8.4 Hz, 2H), 7.28-7.24 (m, 5H), 7.20-7.18 (m, 4H), 7.05-7.03 (m, 4H), 4.62-4.35 (m, 2H), 4.55 (s, 2H), 4.29 (s, 4H), 3.55-3.48 (m, 4H), 3.46 (q, J=6.8 Hz, 2H), 3.354 (q, J=6.8 Hz, 4H), 3.11 (m, 2H), 2.85 (t, J=6.8 Hz, 2H), 2.74 (t, J=6.8 Hz, 4H), 2.29-2.21 (m, 6H), 1.60-1.52 (m, 2H); ESI⁺ MS: m/z (rel intensity) 641.3 (100, [M+H]⁺).

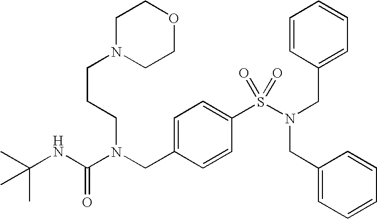

N,N-dibenzyl-4-((3-tert-butyl-1-(3-morpholinopropyl)ureido)methyl)-benzenesulfonamide (HV): ¹H NMR (400 MHz, CDCl₃) [symbol] 7.75 (d, J=8.4 Hz, 2H), 7.34 (d, J=8.2 Hz, 2H), 7.18-7.16 (m, 6H), 7.03-7.00 (m, 4H), 4.52 (s, 2H), 4.29 (s, 4H), 3.69 (t, J=5.2 Hz, 4H), 3.17 (t, J=6.4 Hz, 2H), 2.42-2.39 (m, 4H), 2.31 (t, J=6.4 Hz, 2H), 1.69-1.65 (m, 2H), 1.35 (s, 9H); ESI⁺ MS: m/z (rel intensity) 593.3 (100, [M+H]⁺).

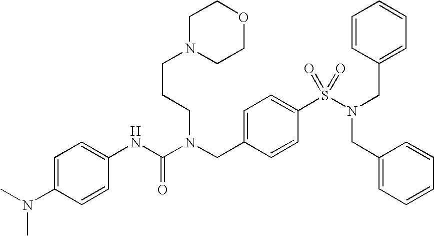

N,N-dibenzyl-4-((3-(4-(dimethylamino)phenyl)-1-(3-morpholinopropyl)-ureido)methyl)benzenesulfonamide (HW): ¹H NMR (400 MHz, CDCl₃) [symbol] 7.78 (d, J=8.4 Hz, 2H), 7.45 (d, J=8.0 Hz, 2H), 7.24 (d, J=8.2 Hz, 2H), 7.24-7.23 (m, 6H), 7.04-7.01 (m, 4H), 6.70 (d, J=8.8, 2H), 4.61 (s, 2H), 4.28 (s, 4H), 3.65-3.59 (m, 4H), 3.36-3.33 (m, 2H), 2.89 (s, 6H), 2.48-2.41 (m, 6H), 1.77-1.69 (m, 2H); ESI⁺ MS: m/z (rel intensity) 656.3 (60, [M+H]⁺).

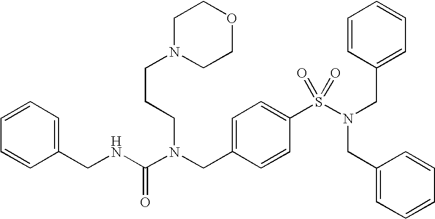

N,N-dibenzyl-4-((3-benzyl-1-(3-morpholinopropyl)ureido)methyl-)benzenesulfonamide (HX): ¹H NMR (400 MHz, CDCl₃) [symbol] 7.77 (d, J=8.4 Hz, 2H), 7.60-7.56 (m, 1H), 7.41 (d, J=8.0 Hz, 2H), 7.31 (d, J=4.0 Hz, 4H), 7.28-7.26 (m, 2H), 7.21-7.18 (m, 6H), 7.04-7.02 (m, 4H), 4.61 (s, 2H), 4.48 (d, J=5.2 Hz, 2H), 4.32 (d, J=5.6 Hz, 1H), 4.29 (s, 4H), 3.39 (bs, 4H), 3.26 (t, J=6.0 Hz, 2H), 2.31 (t, J=6.4 Hz, 4H), 2.23 (bs, 4H), 1.65-1.59 (m, 2H); ESI⁺ MS: m/z (rel intensity) 627.3 (60, [M+H]⁺).

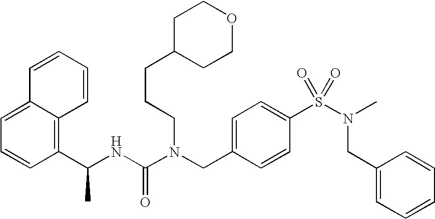

(S)—N-benzyl-N-methyl-4-((1-(3-morpholinopropyl)-3-(1-(naphthalen-1-yl)ethyl)ureido)methyl)benzenesulfonamide (HY): ¹H NMR (400 MHz, CDCl₃) [symbol] 8.16 (d, J=8.4 Hz, 1H), 7.83 (d, J=8.8 Hz, 1H), 7.78-7.75 (m, 3H), 7.55-7.46 (m, 2H), 7.44-7.42 (m, 4H), 7.34-7.29 (m, 5H), 6.38 (d, J=7.2 Hz, 1H), 5.90-5.86 (m, 1H), 4.68 (d, J=16.4 Hz, 1H), 4.53 (d, J=16.4 Hz, 1H), 4.11 (s, 2H), 3.32-3.28 (m, 2H), 3.16-3.11 (m, 4H), 2.56 (s, 3H), 2.19-2.13 (m, 4H), 1.90-1.87 (m, 2H), 1.70 (d, J=6.4 Hz, 3H), 1.58-1.52 (m, 2H); ESI⁺ MS: m/z (rel intensity) 615.2 (100, [M+H]⁺).

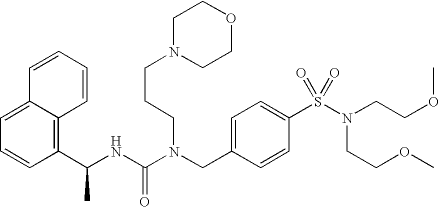

(S)—N,N-bis(2-methoxyethyl)-4-((1-(3-morpholinopropyl)-3-(1-(naphthalen-1-yl)ethyl)ureido)methyl)benzenesulfonamide (HZ): ¹H NMR (400 MHz, CDCl₃) [symbol] 8.14 (d, J=8.0 Hz, 1H), 7.84 (d, J=8.0 Hz, 1H), 7.78-7.74 (m, 3H), 7.53-7.43 (m, 4H), 7.42-7.37 (m, 2H), 7.26-7.24 (m, 1H), 6.35-6.26 (m, 1H), 5.91-5.83 (m, 1H), 4.64 (d, J=16.0 Hz, 1H), 4.52 (d, J=16.0 Hz, 1H), 3.52-3.49 (m, 4H), 3.38-3.35 (m, 4H), 3.27 (s, 6H), 3.13-3.08 (m, 4H), 2.18-2.08 (m, 4H), 1.91-1.83 (m, 2H), 1.68 (d, J=6.0 Hz, 3H), 1.53-1.49 (m, 3H); ESI⁺ MS: m/z (rel intensity) 627.3 (100, [M+H]⁺).

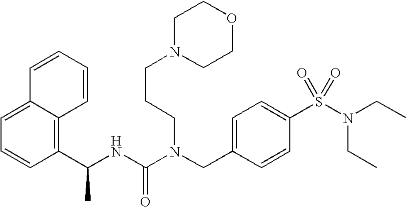

(S)—N,N-diethyl-4-((1-(3-morpholinopropyl)-3-(1-(naphthalen-1-yl)ethyl)ureido)methyl)benzenesulfonamide (IA): ¹H NMR (400 MHz, CDCl₃) [symbol] 8.14 (d, J=8.4 Hz, 1H), 7.84-7.81 (m, 1H), 7.76 (d, J=8.4 Hz, 1H), 7.76 (d, J=8.4 Hz, 1H), 7.52-7.42 (m, 5H), 7.36 (d, J=8.4 Hz, 1H), 6.32 (d, J=7.6 Hz, 1H), 5.90-5.83 (m, 1H), 4.65 (d, J=16.0 Hz, 1H), 4.50 (d, J=16.0 Hz, 1H), 3.29-3.25 (m, 2H), 3.19 (q, J=7.2 Hz, 4H), 3.13-3.08 (m, 4H), 2.15-2.08 (m, 4H), 1.87-1.83 (m, 2H), 1.68 (d, J=6.8 Hz, 3H), 1.52-1.48 (m, 2H), 11.10 (t, J=7.2 Hz, 6H); ESI⁺ MS: m/z (rel intensity) 567.2 (100, [M+H]⁺).

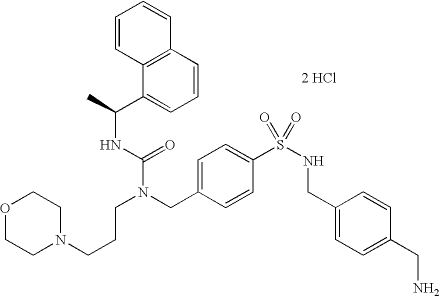

(S)—N-(4-(aminomethyl)benzyl)-4-((1-(3-morpholinopropyl)-3-(1-(naphthalen-1-yl)ethyl)ureido)methyl)benzenesulfonamide (HS): A 25-mL round bottom flask was charged with Boc-protected amine HR (0.09 g, 0.12 mmol) and methanol (1.5 mL). The solution was cooled to 0° C. and thionyl chloride (0.5 mL, 6.87 mmol) was added drop-wise. The reaction was slowly warmed to room temperature and stirred for 18 h. The reaction was concentrated in vacuo to yield HS (0.10 g, 99% yield). ¹H NMR (400 MHz, CDCl₃) [symbol] 8.29-8.22 (m, 2H), 8.18 (t, J=6.8 Hz, 1H), 8.15-8.11 (m, 1H), 7.89 (d, J=7.2 Hz, 1H), 7.77-7.72 (m, 3H), 7.54-7.42 (m, 4H), 7.36 (d, J=8.0 Hz, 3H), 7.25 (d, J=8.0 Hz, 2H), 7.13 (d, J=7.2 Hz, 1H), 5.71-5.64 (m, 1H), 4.6 (s, 2H), 5.14 (m, 1H), 4.60 (s, 2H), 3.95-3.91 (m, 4H), 3.86-3.78 (m, 2H), 3.68-3.59 (m, 2H), 3.55 (s, 8H), 3.31-3.25 (m, 3H), 3.13 (s, 2H), 3.06-2.82 (m, 4H), 2.47 (s, 9H), 1.91-1.81 (m, 2H), 1.49 (d, J=6.8 Hz, 2H), 1.41 (d, J=6.8 Hz, 1); ESI⁺ MS: m/z (rel intensity) 630.3 (100, [M+H]⁺).

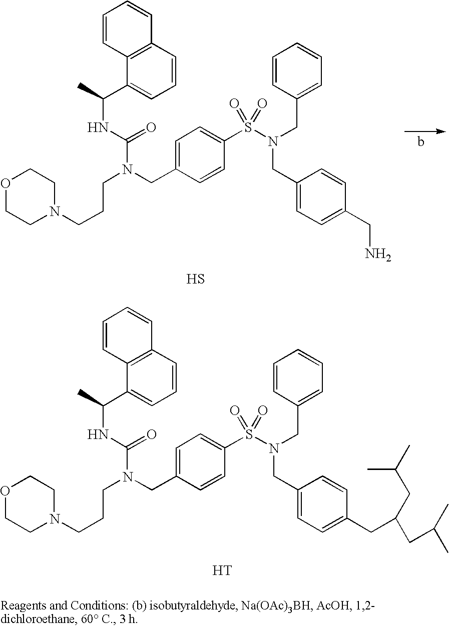

(S)—N-(4-((diisobutylamino)methyl)benzyl)-4-((1-(3-morpholinopropyl)-3-(1-(naphthalen-1-yl)ethyl)ureido)methyl)benzenesulfonamide (HT): A 5-mL round bottom flask was charged with isobutyraldehyde (0.01 ml, 0.11 mmol), amine HS (0.02 g, 0.03 mmol), dichloroethane (0.5 mL) and a catalytic amount of acetic acid (1-2 drops). The reaction was heated to 58° C. for 4 h. Sodium tri(acetoxy) borohydride (0.02 g, 0.10 mmol) was then added in one portion. The reaction was stirred at this temperature for 18 h. The reaction was then taken up in ethyl acetate (5 mL) and quenched with a saturated aqueous solution of sodium bicarbonate (2 mL). The aqueous phase was extracted once with ethyl acetate (2 mL). The combined organic layers were washed with brine (5 mL) and dried over anhydrous sodium sulfate. The supernatant was decanted and concentrated in vacuo. The crude product was purified by silica gel chromatography (0-10% MeOH/CH₂Cl₂) to yield HT: ¹H NMR (400 MHz, CDCl₃) [symbol] 8.19 (d, J=8.4 Hz, 1H), 7.89-7.85 (m, 1H), 7.79 (d, J=8.0 Hz, 1H), 7.76 (d, J=8.4 Hz, 3H), 7.57-7.44 (m, 5H), 7.33 (d, J=8.4 Hz, 2H), 7.13 (d, J=8.4 Hz, 2H), 7.09 (d, J=8.4 Hz, 2H), 6.41-6.34 (m, 1H), 5.94-5.87 (m, 1H), 4.72 (d, J=16.0 Hz, 1H), 4.64 (t, J=6.4 Hz, 1H), 4.57 (d, J=16.0 Hz, 1H), 4.11 (d, J=6.0 Hz, 2H), 3.43 (s, 2H), 3.34-3.30 (m, 2H), 3.21-3.09 (m, 4H), 2.22-2.13 (m, 4H), 2.05 (d, J=7.2 Hz, 4H), 1.94-1.87 (m, 2H), 1.79-1.72 (m, 2H), 1.73 (d, J=7.2 Hz, 3H), 1.71-1.62 (m, 2H), 1.62-1.51 (m, 2H), 0.85 (d, J=6.4 Hz, 12H).

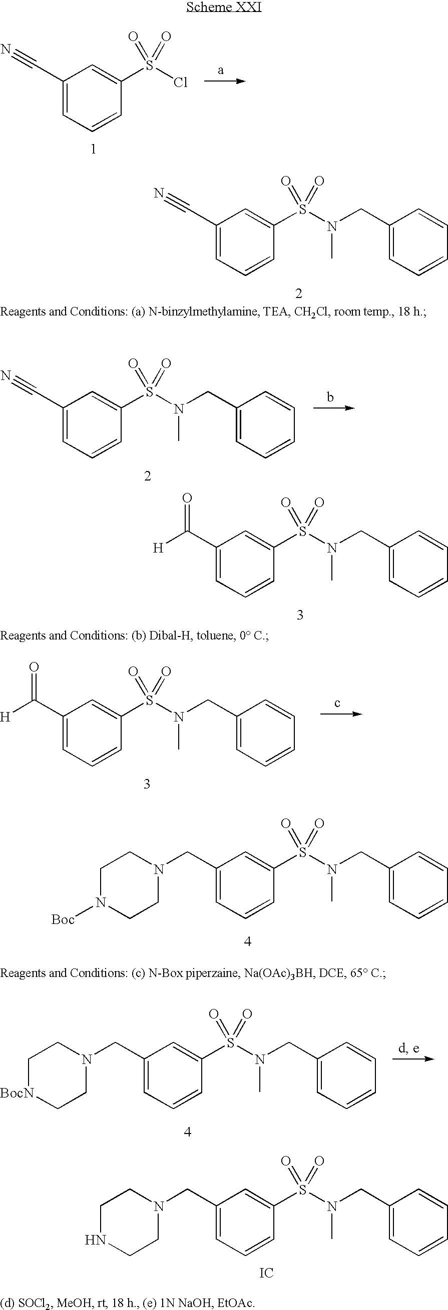

### Example 21

### N-benzyl-N-methyl-3-(piperazin-1-ylmethyl)benzenesulfonamide (IC)

Preparation of N-benzyl-3-cyano-N-methylbenzenesulfonamide (2): To a solution of 3-cyanobenzene sulfonyl chloride (1) (2.0 g, 10.0 mmol) in dichloromethane (20 mL) was added triethylamine (1.52 mL, 11.0 mmol) and N-benzylmethylamine (1.40 mL, 11.0 mmol). The reaction stirred at room temperature for 4 h. The mixture was quenched with brine (5 mL), dried over magnesium sulfate, filtered and concentrated to yield (2) as a crude yellow solid: ¹H NMR (400 MHz, CDCl₃) δ 8.08-8.02 (m, 2H), 7.88-7.85 (m, 1H), 7.71-7.66 (m, 1H), 7.31-7.24 (m, 5H), 4.18 (s, 2H), 2.65 (s, 3H); ESI⁺ MS: m/z (rel intensity) 309.0 (100, [M+Na]⁺).

Preparation of N-benzyl-3-formyl-N-methylbenzenesulfonamide (3): To a 1.0 M solution of diisobutylaluminum hydride in toluene at 0° C. was added (2) (0.55 g, 1.92 mmol). The resulting mixture was stirred at 0° C. for 1 h. A saturated aqueous solution of Rochelle's salt was then added and the mixture was stirred at room temperature for 16 h. The layers were separated. The aqueous layer was extracted once more with ethyl acetate. The combined organic layers were dried over magnesium sulfate, filtered and concentrated. The crude material was purified by silica gel chromatography (0-5% MeOH/CHCl₃) to afford (3) (0.34 g, 61% yield): ESI⁺ MS: m/z (rel intensity) 312.0 (100, [M+Na]⁺).

Preparation of tert-butyl 4-(3-(N-benzyl-N-methylsulfamoyl)benzyl)piperazine-1-carboxylate (4): To a solution of (3) (0.34 g, 1.17 mmol) in dichloroethane (10 mL) was N-Boc piperazine (0.24 g, 1.29 mmol) and two drops of acetic acid. The resulting mixture was warmed to 65° C. and stirred for 1 h. Sodium triacetoxyborohydride (0.37 g, 1.76 mmol) was then added. The reaction was stirred at 65° C. for 3 h. The reaction was cooled to room temperature and quenched with a saturated aqueous solution of sodium bicarbonate (10 mL). The product was extracted with dichloromethane (15 mL). The combined organic layers were dried over potassium carbonate, filtered and concentrated. The crude material was purified by silica gel chromatography (0-5% MeOH/CHCl₃) to afford 4 (0.30 g, 56% yield): ¹H NMR (400 MHz, CDCl₃) δ 7.80-7.78 (m, 1H), 7.72-7.68 (m, 1H), 7.58-7.54 (m, 1H), 7.48 (t, J=7.6 Hz, 1H), 7.30-7.24 (m, 5H), 4.11 (s, 2H), 3.55 (s, 2H), 3.39 (t, J=5.2 Hz, 4H), 2.57 (s, 3H), 2.36 (t, J=4.8 Hz, 4H), 1.42 (s, 9H); ESI⁺ MS: m/z (rel intensity) 460.2 (100, [M+H]⁺).

Preparation of N-benzyl-N-methyl-3-(piperazin-1-ylmethyl)benzenesulfonamide (IC): To a solution of (4) (0.30 g, 0.65 mmol) in methanol (5 mL) was added thionyl chloride (1 mL). The resulting mixture was stirred at room temperature for 18 h. The white precipitate formed was isolated by filtration and dried to afford the dihydrochloride salt of IC (0.21 g, 75% yield). The dihydrochloride salt (0.11 g) was diluted in 1 N NaOH and ethyl acteate. The layers were separated. The aqueous layer was extracted once more with more ethyl acetate. The combined organic layers were dried over magnesium sulfate, filtered and concentrated to afford IC (0.09 g, 98% yield): ¹H NMR (400 MHz, CDCl₃) δ 7.79 (bs, 1H), 7.72-7.68 (m, 1H), 7.59-7.54 (m, 1H), 7.48 (t, J=7.6 Hz, 1H), 7.32-7.24 (m, 5H), 4.11 (s, 2H), 3.55 (s, 2H), 3.12 (bs, 1H), 2.90 (t, J=4.8 Hz, 4H), 2.57 (s, 3H), 2.48-2.38 (m, 4H); ESI⁺ MS: m/z (rel intensity) 360.2 (100, [M+H]⁺).

Compound IB listed below is a non-limiting example of the fourth aspect of the present invention, which was prepared using methods similar to those described in Scheme XXI, using appropriately substituted amino reagents.

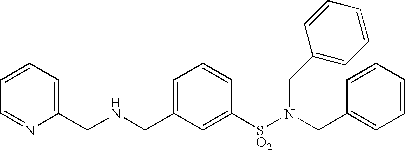

N,N-dibenzyl-3-((pyridin-2-ylmethylamino)methyl)benzenesulfonamide (IB): ¹H NMR (400 MHz, CDCl₃) [symbol] 8.55 (d, J=4.8 Hz, 1H), 7.78 (s, 1H), 7.72 (d, J=7.6 Hz, 1H), 7.66-7.58 (m, 2H), 7.45 (t, J=7.6 Hz, 1H), 7.27-7.22 (m, 2H), 7.22-7.13 (m, 6H), 7.08-7.01 (m, 4H), 4.31 (s, 4H), 3.86 (d, J=7.6 Hz, 2H); ESI⁺ MS: m/z (rel intensity) 458.1 (100, [M+H]⁺).

In another embodiment, the compounds of formula (ID) can have the following general structure:

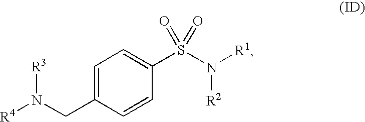

wherein —NR¹R², and —NR³R⁴ are defined herein below in Table 5. In these various embodiments, R¹ and R², taken together with nitrogen atom to which they are both shown attached, may form 5-membered to 18-membered, substituted or unsubstituted saturated heterocyclic ring system. In addition or in the alternative, in these various embodiments, R³ and R⁴, taken together with nitrogen atom to which they are both shown attached, form 5-membered to 18-membered, substituted or unsubstituted saturated heterocyclic ring system

TABLE 5

| Compd. | —NR³R⁴ | —NR¹R² |
|---|---|---|
| ID | 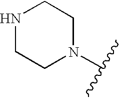 | 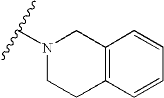 |
| IE | 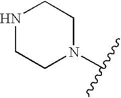 | 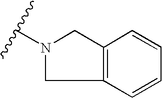 |
| IF | 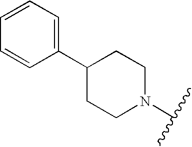 | 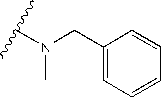 |
| IG | 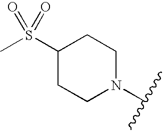 | 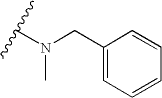 |
| IH | 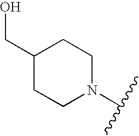 | 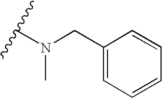 |
| II | 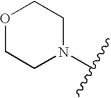 | 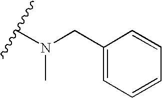 |
| IJ | 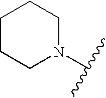 | 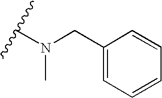 |
| IK | 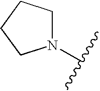 | 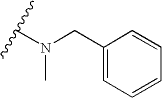 |
| IL | 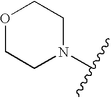 | 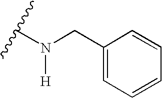 |
| IM | 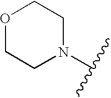 | 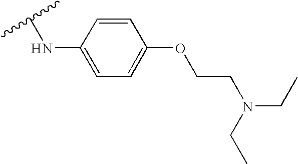 |
| IN | 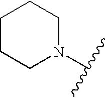 | 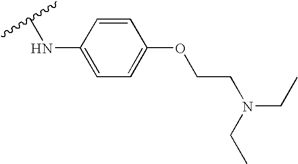 |
| IO | 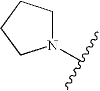 | 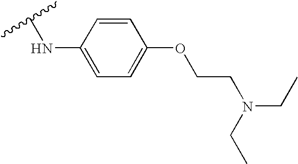 |
| IP | 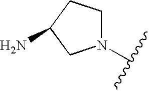 | 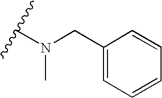 |
| IQ | 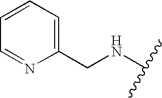 | 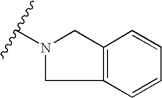 |
| IR | 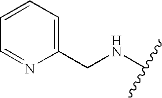 | 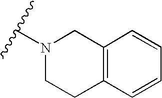 |
| IS | 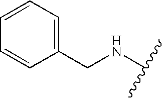 | 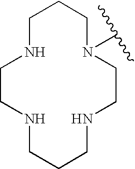 |
| IT | 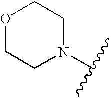 | 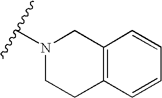 |
| IU | 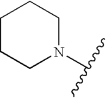 | 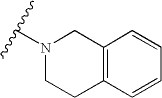 |
| IV | 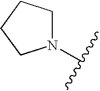 | 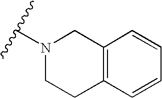 |
| IW | 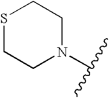 | 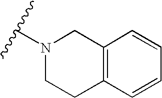 |
| IX | 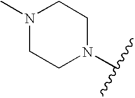 | 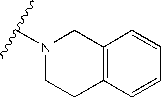 |
| IY | 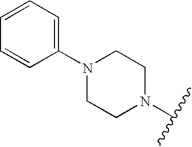 | 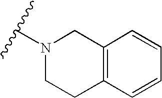 |
| IZ | 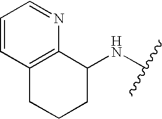 | 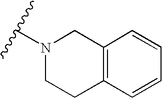 |
| JA | 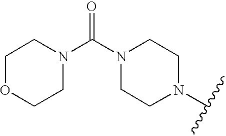 | 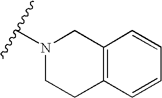 |
| JB | 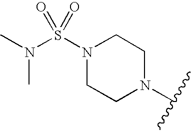 | 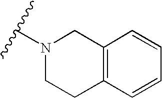 |
| JC | 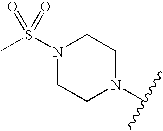 | 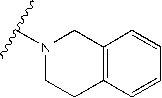 |
| JD | 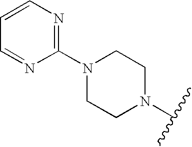 | 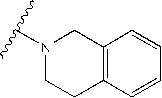 |
| JE | 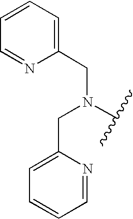 | 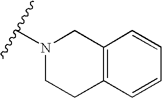 |
| JF | 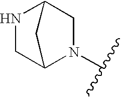 | 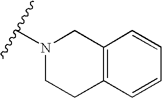 |
| JG | 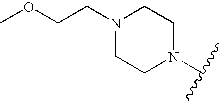 | 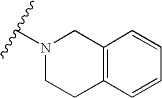 |
| JH | 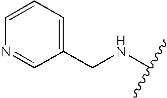 | 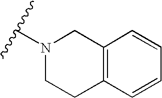 |
| JI | 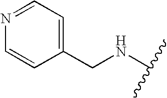 | 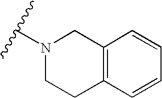 |
| JJ | 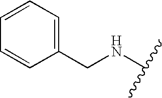 | 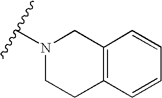 |
| JK | 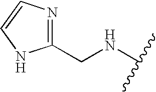 | 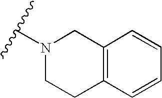 |
| JL | 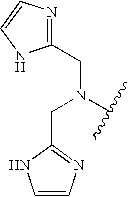 | 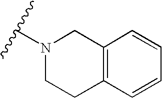 |
| JM | 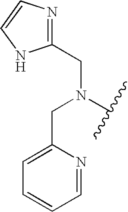 | 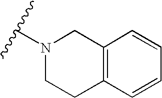 |
| JN | 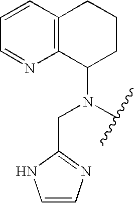 | 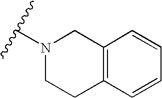 |
| JO | 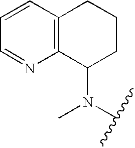 | 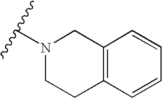 |
| JP | 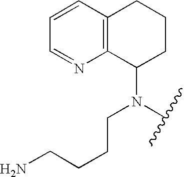 | 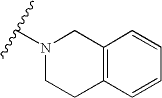 |
| JQ | 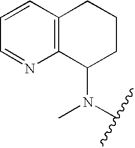 | 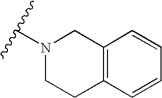 |
| JR | 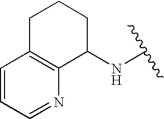 | 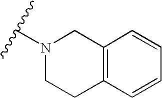 |
| JS | 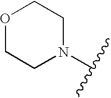 | 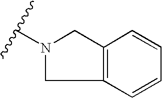 |
| JT | 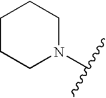 | 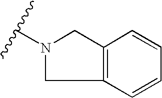 |
| JU | 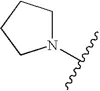 | 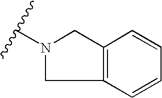 |
| JV | 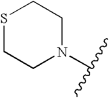 | 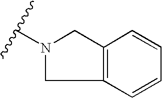 |
| JW | 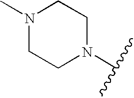 | 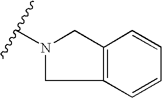 |
| JX | 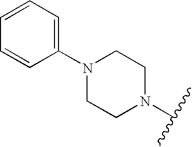 | 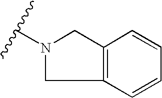 |
| JY | 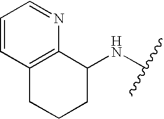 | 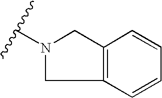 |
| JZ | 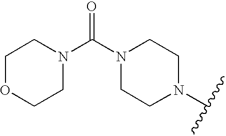 | 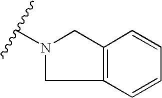 |
| KA | 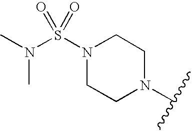 | 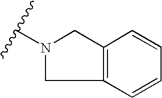 |
| KB | 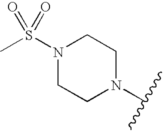 | 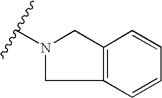 |
| KC | 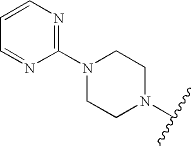 | 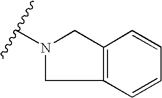 |
| KD | 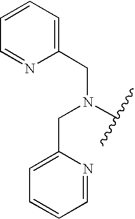 | 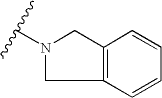 |
| KE | 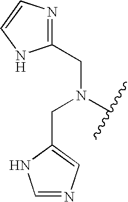 | 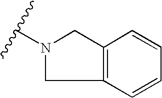 |
| KF | 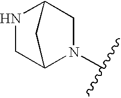 | 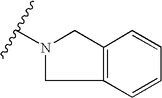 |
| KG | 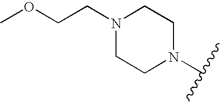 | 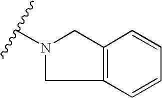 |
| KH | 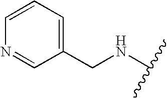 | 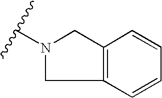 |
| KI | 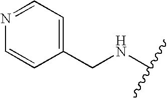 | 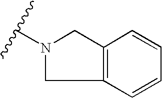 |
| KJ | 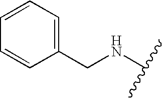 | 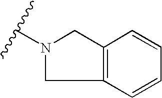 |
| KK | 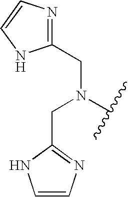 | 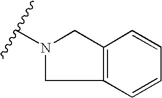 |
| KL | 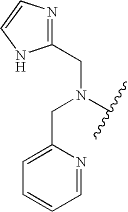 | 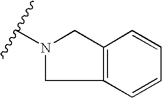 |
| KM | 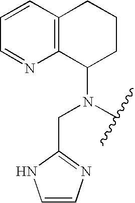 | 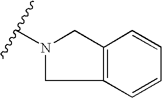 |
| KN | 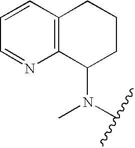 | 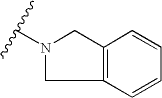 |
| KO | 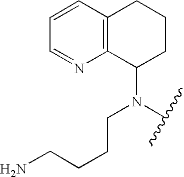 | 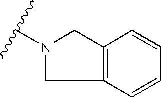 |
| KP | 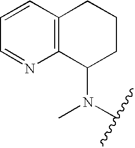 | 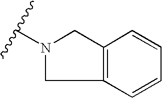 |
| KQ | 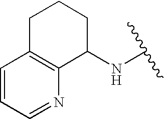 | 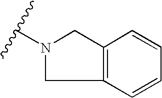 |
| KR | 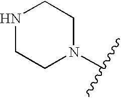 | 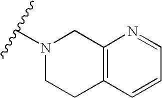 |
| KS | 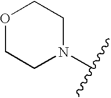 | 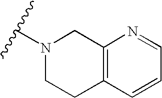 |
| KT | 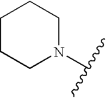 | 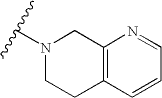 |
| KU | 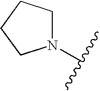 | 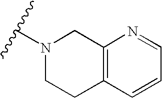 |
| KV | 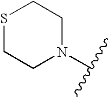 | 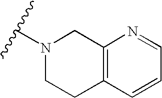 |
| KW | 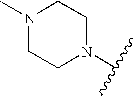 | 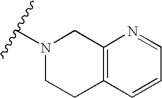 |
| KX | 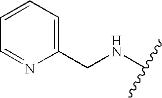 | 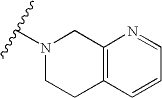 |
| KY | 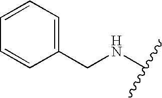 | 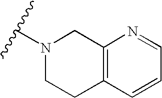 |
| KZ | 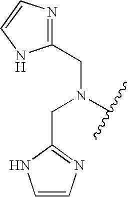 | 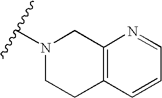 |
| LA | 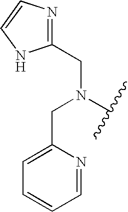 | 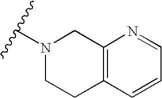 |
| LB | 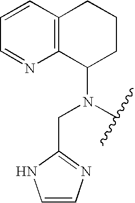 | 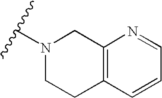 |
| LC | 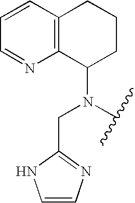 | 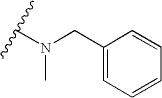 |
| LD | 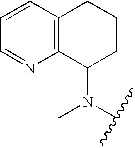 | 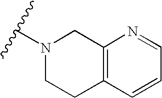 |
| LE | 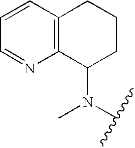 | 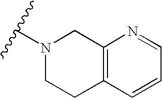 |
| LF | 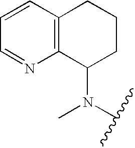 | 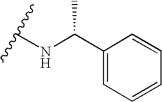 |
| LG | 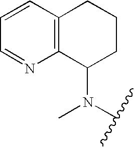 | 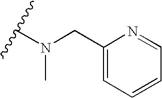 |
| LH | 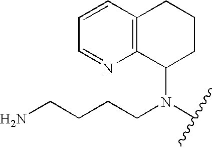 | 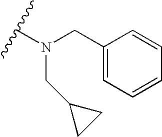 |
| LI | 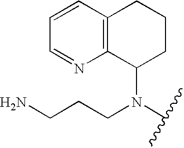 | 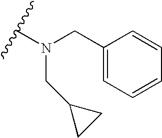 |
| LJ | 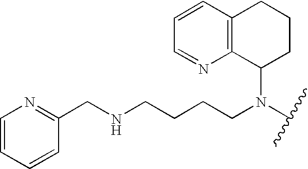 | 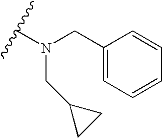 |
| LK | 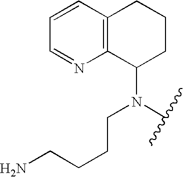 | 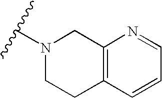 |
| LM | 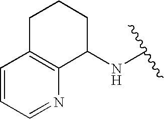 | 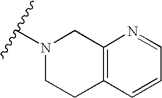 |
| LN | 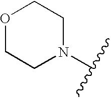 | 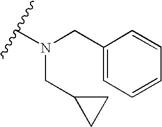 |
| LO | 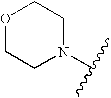 | 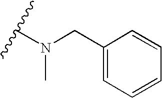 |
| LP | 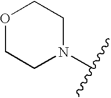 | 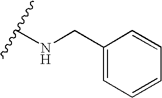 |
| LQ | 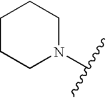 | 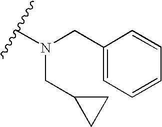 |
| LR | 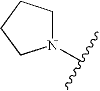 | 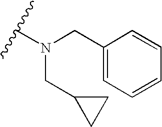 |
| LS | 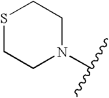 | 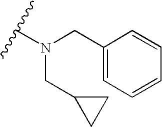 |
| LT | 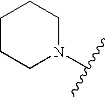 | 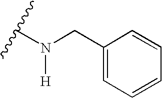 |
| LU | 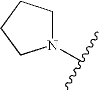 | 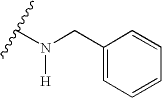 |
| LV | 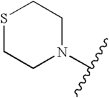 | 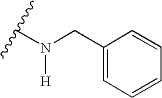 |

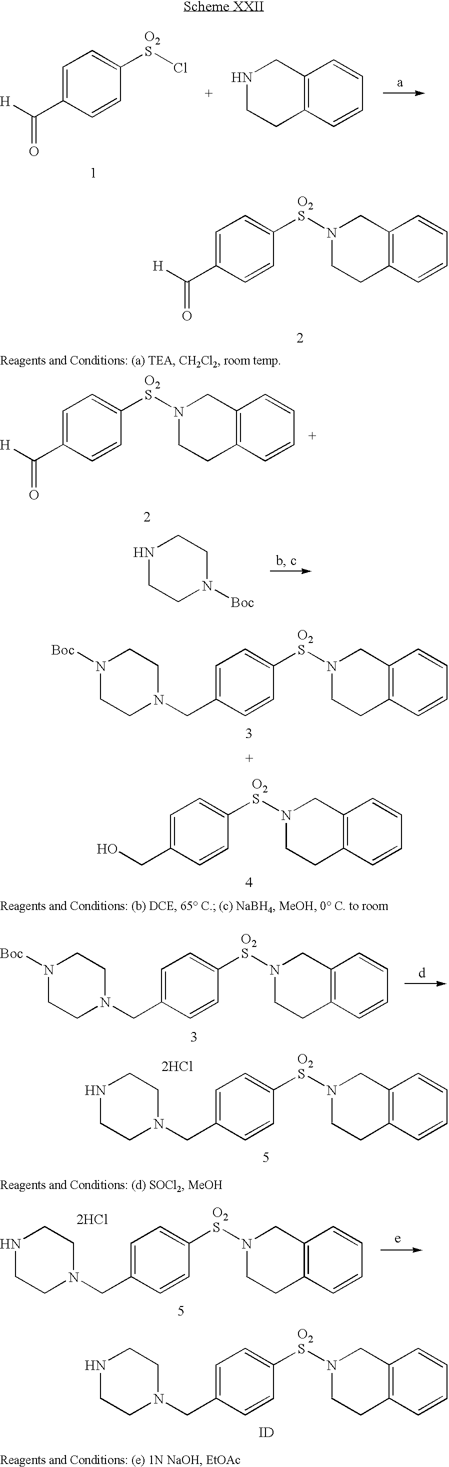

### Example 22

### 2-(4-(piperazin-1-ylmethyl)phenylsulfonyl)-1,2,3,4-tetrahydroisoquinoline (ID)

### Preparation of 4-(3,4-dihydroisoquinolin-2(1H)-ylsulfonyl)benzaldehyde (2)

A solution of 4-formylbenzene sulfonyl chloride (1) (0.40 g, 1.95 mmol) in dichloromethane (5 mL) was treated with 1,2,3,4-tetrahydroisoquinoline (0.28 mL, 2.15 mmol) and triethylamine (0.33 mL, 2.34 mmol). The resultant mixture was stirred at room temperature for 1 h. A saturated aqueous solution of sodium bicarbonate (10 mL) was added. The product was extracted three times with 10 mL of dichloromethane. The combined organic layers were dried over potassium carbonate, filtered and concentrated. The crude material (0.67 g) was used in the next reaction without purification: Crude ¹H NMR (400 MHz, CDCl₃) [symbol] 10.08 (s, 1H), 8.03-7.97 (m, 4H), 7.20-7.00 (m, 4H), 4.31 (s, 2H), 3.42 (t, J=6.0 Hz, 2H), 2.90 (t, J=6.0 Hz, 2H); ESI⁺ MS: m/z (rel intensity) 302.0 (100, [M+H]⁺).

Preparation of tert-butyl 4-(4-(3,4-dihydroisoquinolin-2(1H)-ylsulfonyl)benzyl)piperazine-1-carboxylate (3): A solution of crude 4-(3,4-dihydroisoquinolin-2(1H)-ylsulfonyl)benzaldehyde, 2, (1.95 mmol) in 1,2-dichloroethane (10 mL) was treated with 1-(Boc)piperazine (0.44 g, 2.34 mmol). The resultant mixture was warmed to 65° C. and stirred at this temperature for 2 h. The reaction was concentrated, then diluted in methanol and cooled to 0° C. Sodium borohydride was added portionwise at 0° C. The mixture was then slowly warmed to room temperature. After 1 h, a saturated aqueous solution of sodium bicarbonate (10 mL) was added. The product was extracted three times with 10 mL of dichloromethane. The combined organic layers were dried over potassium carbonate, filtered and concentrated in vacuo. The crude material was purified by silica gel chromatography. At this time 3 and 4 were not easily separable by chromatography and were carried over to the next step. Sulfonamide 3: ¹H NMR (400 MHz, CDCl₃) [symbol] 7.76 (d, J=8.0, 2H), 7.47 (d, J=8.0 Hz, 2H), 7.14-7.10 (m, 2H), 7.06-7.00 (m, 2H), 4.25 (s, 2H), 3.52 (s, 2H), 3.42-3.37 (m, 4H), 3.36 (t, J=6.0 Hz, 2H), 2.91 (t, J=6.0 Hz, 2H), 2.34 (t, J=4.8 Hz, 2H), 1.42 (s, 9H); ESI⁺ MS: m/z (rel intensity) 472.2 (90, [M+H]⁺).

Preparation of 2-(4-(piperazin-1-ylmethyl)phenylsulfonyl)-1,2,3,4-tetrahydroisoquinoline dihydrochloride salt (5): A solution of tert-butyl 4-(4-(3,4-dihydroisoquinolin-2(1H)-ylsulfonyl)benzyl)piperazine-1-carboxylate, 3, (0.13 g, 0.27 mmol) in methanol (5 mL) was treated with a large excess of thionyl chloride at room temperature. After 1 h, the solvents were removed by evaporation to obtain the dihydrochloride salt (0.05 g, 41% yield). The white solid obtained was dried under reduced pressure and submitted as 5: ¹H NMR (400 MHz, CDCl₃) [symbol] 7.91-7.80 (m, 4H), 7.12-7.05 (m, 4H), 4.17 (s, 2H), 3.50-3.20 (m, 8H), 2.83-2.80 (m, 2H), 2.46 (m, 2H); ESI⁺ MS: m/z (rel intensity) 372.1 (100, [M+H]⁺).

Preparation of 2-(4-(piperazin-1-ylmethyl)phenylsulfonyl)-1,2,3,4-tetrahydroisoquinoline (ID) and (4-(3,4-dihydroisoquinolin-2(1H)-ylsulfonyl)phenyl)methanol (4): A solution of the mixture of 3 and 4, in methanol (5 mL), was treated with a large excess of thionyl chloride at room temperature. Dihydrochloride salt 5 crashed out of solution leaving alcohol 4 in solution in methanol. The sulfonamide 5 was isolated by filtration then suspended in ethyl acetate (5 mL). An aqueous 1N solution of sodium hydroxide was added. The organic layer was separated and dried over magnesium sulfate, filtered and concentrated in vacuo to afford the free base, ID: ¹H NMR (400 MHz, CDCl₃) [symbol] 7.76 (d, J=8.0 Hz, 2H), 7.47 (d, J=8.0 Hz, 2H), 7.13-7.10 (m, 2H), 7.06-6.98 (m, 2H), 4.24 (s, 2H), 3.51 (s, 2H), 3.35 (t, J=5.6 Hz, 2H), 2.91 (t, J=6.0 Hz, 2H), 2.86 (t, J=4.8 Hz, 4H), 2.45-2.32 (m, 4H); ESI⁺ MS: m/z (rel intensity) 372.1 (100, [M+H]⁺). Alcohol 4: ¹H NMR (400 MHz, CDCl₃) [symbol] 7.81 (d, J=8.4 Hz, 2H), 7.51 (d, J=8.4 Hz, 2H), 7.14-7.10 (m, 2H), 7.08-6.98 (m, 2H), 4.77 (d, J=5.2 Hz, 2H), 4.24 (s, 2H), 3.35 (t, J=5.6 Hz, 2H), 2.91 (t, J=5.6 Hz, 2H), 1.83 (t, J=5.2 Hz, 1H); ESI⁺ MS: m/z (rel intensity) 304.1 (100, [M+H]⁺).

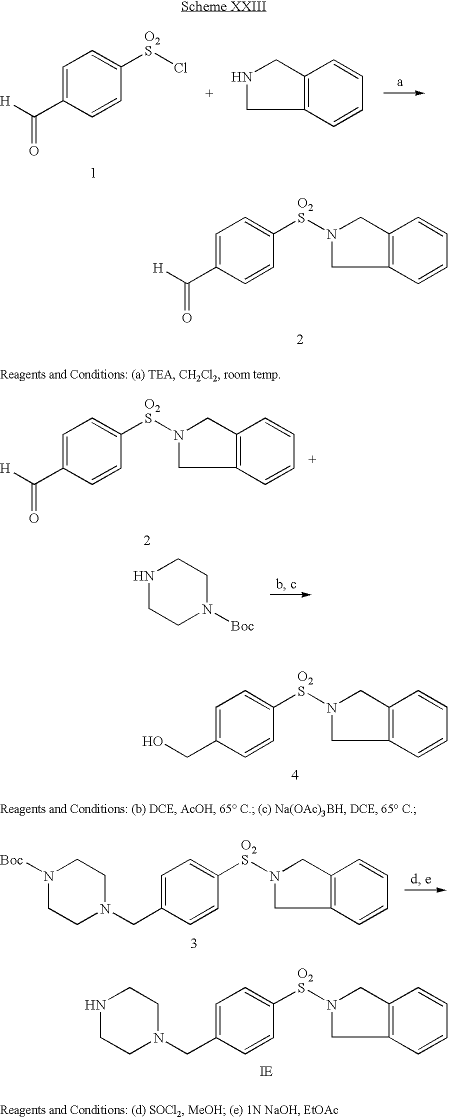

### Example 23

### 2-(4-(piperazin-1-ylmethyl)phenylsulfonyl)isoindoline (IE)

Preparation of 4-(isoindolin-2-ylsulfonyl)benzaldehyde (2): A solution of 4-formylbenzene sulfonyl chloride (1) (0.50 g, 2.44 mmol) in dichloromethane (5 mL) was treated with isoindoline (0.32 g, 2.68 mmol) and triethylamine (0.41 mL, 2.93 mmol). The resultant mixture was stirred at room temperature for 1 h. A saturated aqueous solution of sodium bicarbonate (10 mL) was added. The product was extracted three times with 10 mL of dichloromethane. The combined organic layers were dried over potassium carbonate, filtered and concentrated in vacuo. The crude material (0.67 g, 96% yield) was used in the next reaction without purification: Crude ¹H NMR (400 MHz, CDCl₃) [symbol] 10.06 (s, 1H), 8.05-8.00 (m, 4H), 7.25-7.20 (m, 2H), 7.20-7.14 (m, 2H), 4.66 (s, 4H); ESI⁺ MS: m/z (rel intensity) 288.0 (100, [M+H]⁺).

Preparation of tert-butyl 4-(4-(isoindolin-2-ylsulfonyl)benzyl)piperazine-1-carboxylate (3) and 2-(4-(piperazin-1-ylmethyl)phenylsulfonyl)isoindoline (IE): A solution of crude 4-(isoindolin-2-ylsulfonyl)benzaldehyde, 2, (0.67 g, 2.33 mmol) in 1,2-dichloroethane (10 mL) was treated with 1-(Boc)piperazine (0.48 g, 2.56 mmol) and a couple drops of acetic acid. The resultant mixture was warmed to 65° C. and stirred at this temperature for 2 h. Sodium tri(acetoxy)borohydride was added to the warm mixture that was stirred at 65° C. for 2 h. The reaction was then cooled to room temperature. A saturated aqueous solution of sodium bicarbonate (10 mL) was added. The product was extracted three times with 10 mL of dichloromethane. The combined organic layers were dried over potassium carbonate, filtered and concentrated in vacuo. The crude material was purified by silica gel chromatography (0-5% MeOH/CHCl₃). A pure fraction was isolated: ¹H NMR (400 MHz, CDCl₃) [symbol] 7.80 (d, J=8.0 Hz, 2H), 7.46 (d, J=8.0 Hz, 2H), 7.22-7.18 (m, 2H), 7.17-7.12 (m, 2H), 4.61 (s, 4H), 3.50 (s, 2H), 3.38 (t, J=4.4 Hz, 4H), 2.33 (t, J=4.4 Hz, 4H), 1.42 (s, 9H); ESI⁺ MS: m/z (rel intensity) 458.1 (100, [M+H]⁺). The impure fraction was used without further purification in the next step. A solution of the impure fraction of sulfonamide 3 in methanol (5 mL) was treated with a large excess of thionyl chloride at room temperature and stirred over the weekend. The white precipitate formed was filtered and dried under high vacuum. The dihydrochloride salt was suspended in ethyl acetate and an aqueous 1N solution of sodium hydroxide was added. The organic layer was separated and dried over magnesium sulfate, filtered and concentrated in vacuo to afford the free amine submitted as IE: ¹H NMR (400 MHz, CDCl₃) [symbol] 7.80 (d, J=8.0 Hz, 2H), 7.46 (d, J=8.0 Hz, 2H), 7.23-7.19 (m, 2H), 7.18-7.13 (m, 2H), 4.62 (s, 4H), 3.49 (s, 2H), 2.85 (t, J=4.8 Hz, 4H), 2.37 (m, 4H); ESI⁺ MS: m/z (rel intensity) 358.1 (100, [M+H]⁺).

Compounds IF, IG, 1H, FI, II, IJ, IK, IL, IM, IN, IO and IP listed below are non-limiting examples of the 1st aspect of the present invention which were prepared using methods similar to those described in Scheme II (i.e., isoindoline of step a, Scheme II was replaced with the appropriate amine derivative).

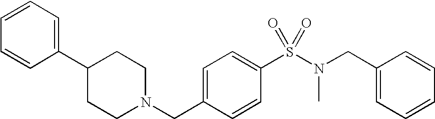

N-benzyl-N-methyl-4-((4-phenylpiperidin-1-yl)methyl)benzenesulfonamide (IF): ¹H NMR (400 MHz, CDCl₃) [symbol] 7.80 (d, J=8.0 Hz, 2H), 7.56 (d, J=8.4 Hz, 2H), 7.33-7.28 (m, 6H), 7.25-7.18 (m, 4H), 4.16 (s, 2H), 3.62 (s, 2H), 3.01-2.98 (m, 2H), 2.61 (s, 3H), 2.18-2.10 (m, 2H), 1.89-1.80 (m, 4H); ESI⁺ MS: m/z (rel intensity) 435.2 (100, [M+H]⁺).

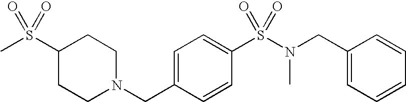

N-benzyl-N-methyl-4-((4-(methylsulfonyl)piperidin-1-yl)methyl)benzene-sulfonamide (IG): ¹H NMR (400 MHz, CDCl₃) [symbol] 7.75 (d, J=8.0 Hz, 2H), 7.47 (d, J=8.4 Hz, 2H), 7.32-7.21 (m, 5H), 4.80 (d, J=7.6 Hz, 1H), 4.11 (s, 2H), 3.53 (s, 2H), 3.32 (bs, 1H), 2.95 (s, 3H), 2.77 (bd, J=10.4 Hz, 2H), 2.57 (s, 3H), 2.15-2.10 (m, 2H), 1.96-1.94 (m, 2H), 1.66-1.52 (m, 2H); ESI⁺ MS: m/z (rel intensity) 435.2 (100, [M−H]⁺).

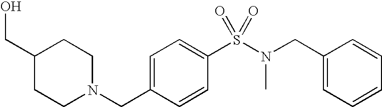

N-benzyl-4-((4-(hydroxymethyl)piperidin-1-yl)methyl)-N-methylbenzene-sulfonamide (IH): ¹H NMR (400 MHz, CDCl₃) [symbol] 7.76 (d, J=8.4 Hz, 2H), 7.51 (d, J=8.0 Hz, 2H), 7.31-7.27 (m, 5H), 4.13 (s, 2H), 3.56 (s, 2H), 3.49 (d, J=6.4 Hz, 2H), 2.90-2.87 (m, 2H), 2.58 (s, 3H), 2.04-1.98 (m, 2H), 1.74-1.70 (m, 3H), 1.53-1.47 (m, 1H), 1.35-1.28 (m, 2H); ESI⁺ MS: m/z (rel intensity) 389.2 (100, [M+H]⁺).

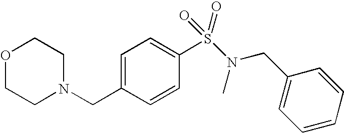

N-benzyl-N-methyl-4-(morpholinomethyl)benzenesulfonamide (II): ¹H NMR (400 MHz, CDCl₃) δ 7.77 (d, J=8.0 Hz, 2H), 7.52 (d, J=8.4 Hz, 2H), 7.32-7.27 (m, 5H), 4.13 (s, 2H), 3.71 (bs, 4H), 3.56 (s, 2H), 2.58 (s, 3H), 2.45 (bs, 4H); ESI⁺ MS: m/z (rel intensity) 361.1 (100, [M+H]⁺).

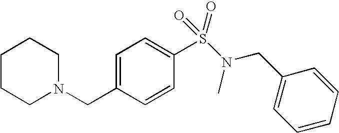

N-benzyl-N-methyl-4-(piperidin-1-ylmethyl)benzenesulfonamide (IJ): ¹H NMR (400 MHz, CDCl₃) δ 7.75 (d, J=8.8 Hz, 2H), 7.50 (d, J=8.4 Hz, 2H), 7.30-7.27 (m, 5H), 4.12 (s, 2H), 3.51 (s, 2H), 2.57 (s, 2H), 2.37 (bs, 4H), 1.58-1.54 (m, 4H), 1.44 (bs, 2H); ESI⁺ MS: m/z (rel intensity) 359.2 (100, [M+H]⁺).

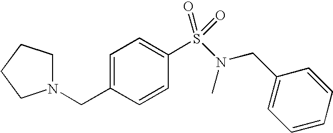

N-benzyl-N-methyl-4-(pyrrolidin-1-ylmethyl)benzenesulfonamide (IK): ¹H NMR (400 MHz, CDCl₃) δ 7.78 (d, J=8.8 Hz, 2H), 7.54 (d, J=8.0 Hz, 2H), 7.31-7.26 (m, 5H), 4.12 (s, 2H), 3.82 (s, 2H), 2.68 (bs, 4H), 2.58 (s, 3H), 1.86-1.83 (m, 4H); ESI⁺ MS: m/z (rel intensity) 345.1 (100, [M+H]⁺).

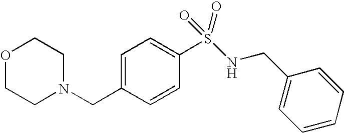

N-benzyl-4-(morpholinomethyl)benzenesulfonamide (IL): ¹H NMR (400 MHz, CDCl₃) δ 7.79 (d, J=7.6 Hz, 2H), 7.45 (d, J=7.6 Hz, 2H), 7.23-7.17 (m, 5H), 4.12 (d, J=6.0 Hz, 2H), 3.69 (bs, 4H), 3.53 (s, 2H), 2.42 (bs, 4H); ESI⁺ MS: m/z (rel intensity) 347.1 (100, [M+H]⁺).

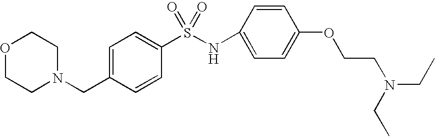

N-(4-(2-(diethylamino)ethoxy)phenyl)-4-(morpholinomethyl)benzene-sulfonamide (IM): ¹H NMR (400 MHz, CDCl₃) δ 7.62 (d, J=8.0 Hz, 2H), 7.51 (bs, NH), 7.35 (d, J=7.6 Hz, 2H), 6.94 (d, J=8.8 Hz, 2H), 6.65 (d, J=8.4 Hz, 2H), 4.02 (t, J=5.6 Hz, 2H), 3.66 (t, J=4.0 Hz, 4H), 3.47 (s, 2H), 3.00 (t, J=5.2 Hz, 2H), 2.77 (q, J=6.8 Hz, 4H), 2.37 (bs, 4H), 1.09 (t, J=7.2 Hz, 6H); ESI⁺ MS: m/z (rel intensity) 448.2 (80, [M+H]⁺).

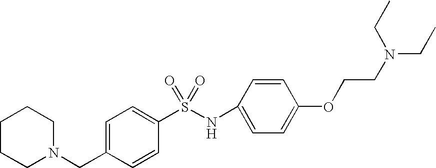

N-(4-(2-(diethylamino)ethoxy)phenyl)-4-(piperidin-1-ylmethyl)benzene-sulfonamide (IN): ¹H NMR (400 MHz, CDCl₃) δ 7.59 (d, J=8.0 Hz, 2H), 7.36 (d, J=8.0 Hz, 2H), 6.92 (d, J=8.8 Hz, 2H), 6.71 (d, J=8.8 Hz, 2H), 3.98 (t, J=6.4 Hz, 2H), 3.46 (s, 2H), 2.87 (t, J=6.4 Hz, 2H), 2.66 (q, J=7.2 Hz, 4H), 2.32 (bs, 4H), 1.56-1.51 (m, 4H), 1.40 (bs, 2H), 1.06 (t, J=6.8 Hz, 6H); ESI⁺ MS: m/z (rel intensity) 446.2 (60, [M+H]⁺).

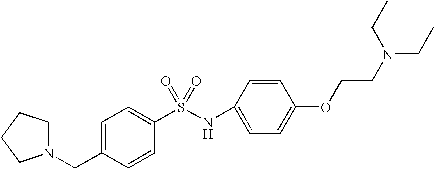

N-(4-(2-(diethylamino)ethoxy)phenyl)-4-(pyrrolidin-1-ylmethyl)benzene-sulfonamide (10): ¹H NMR (400 MHz, CDCl₃) δ 7.60 (d, J=8.0 Hz, 2H), 7.36 (d, J=8.0 Hz, 2H), 6.92 (d, J=8.8 Hz, 2H), 6.72 (d, J=8.8 Hz, 2H), 3.97 (t, J=6.0 Hz, 2H), 3.62 (s, 2H), 2.85 (t, J=6.4 Hz, 2H), 2.63 (q, J=7.2 Hz, 4H), 2.47 (bs, 4H), 1.76 (bs, 4H), 1.05 (t, J=7.6 Hz, 6H); ESI⁺ MS: m/z (rel intensity) 432.2 (60, [M+H]⁺).

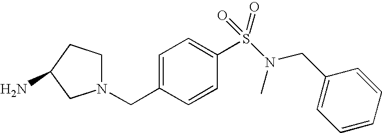

(S)-4-((3-aminopyrrolidin-1-yl)methyl)-N-benzyl-N-methylbenzene-sulfonamide (IP): ¹H NMR (400 MHz, CDCl₃) δ 7.75 (d, J=8.4 Hz, 2H), 7.50 (d, J=8.4 Hz, 2H), 7.30-7.25 (m, 5H), 4.11 (s, 2H), 3.65 (q, J=6.4 Hz, 2H), 3.53-3.49 (m, 1H), 2.74-2.67 (m, 2H), 2.56 (s, 3H), 2.45 (q, J=6.4 Hz, 1H), 2.32 (dd, J=4.0, 9.2 Hz, 1H), 2.21-2.16 (m, 1H); ESI⁺ MS: m/z (rel intensity) 360.2 (100, [M+H]⁺).

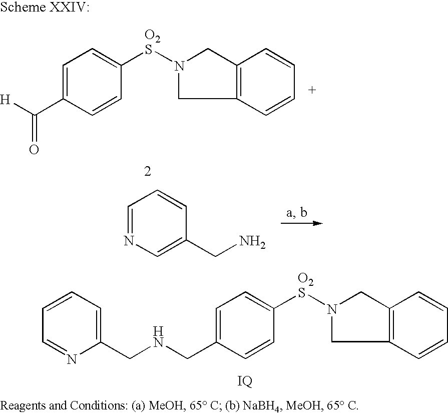

### Example 24

### N-(4-(isoindolin-2-ylsulfonyl)benzyl)-1-(pyridin-2-yl)methanamine (IQ)

Preparation of N-(4-(isoindolin-2-ylsulfonyl)benzyl)-1-(pyridin-2-yl)methanamine, (IQ): To a solution of 4-(isoindolin-2-ylsulfonyl)benzaldehyde, 2 (0.67 g, 2.33 mmol) in methanol was added 2-(amino)methylpyridine (0.27 mL, 2.56 mmol). The resulting mixture was warmed to 65° C. and stirred for 2 h. The reaction was then cooled to 0° C. and sodium borohydride (0.35 g, 9.32 mmol) was added portionwise. The reaction was slowly warmed to room temperature then a saturated aqueous solution of sodium bicarbonate (5 mL) was added. The product was extracted three times with 5 mL of dichloromethane. The combined organic layers were dried over potassium carbonate, filtered and concentrated in vacuo. The crude material was purified by silica gel chromatography (0-5% MeOH/CHCl₃) to afford 0.52 g (58% yield) of product (IQ): ¹H NMR (400 MHz, CDCl₃) [symbol] 8.52 (d, J=4.8 Hz, 1H), 7.81 (d, J=8.4 Hz, 2H), 7.60 (dt, J=2.0 Hz, 7.6 Hz, 1H), 7.50 (d, J=8.0 Hz, 2H), 7.26-7.10 (m, 6H), 4.60 (m, 4H), 3.87 (s, 2H), 3.86 (s, 2H); ESI⁺ MS: m/z (rel intensity) 380.1 (100, [M+H]⁺).

Compound IR listed below is a non-limiting example of the 1^{st} aspect of the present invention which were prepared using methods similar to those described in Scheme III by using the appropriate amine in step a.

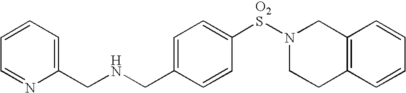

N-(4-(3,4-dihydroisoquinolin-2(1H)-ylsulfonyl)benzyl)-1-(pyridin-2-yl)methanamine (IR): ¹H NMR (400 MHz, CDCl₃) [symbol] 8.52 (d, J=4.4 Hz, 1H), 7.76 (d, J=8.0 Hz, 2H), 7.61 (dt, J=7.6 Hz, 2.0 Hz, 1H), 7.51 (d, J=8.0 Hz, 2H), 7.25 (d, J=8.4 Hz, 1H), 7.16-7.08 (m, 3H), 7.05-6.98 (m, 2H), 4.22 (s, 2H), 3.88 (bs, 4H), 3.32 (t, J=6.0 Hz, 2H), 2.89 (t, J=6.0 Hz, 2H); ESI⁺ MS: m/z (rel intensity) 394.1 (100, [M+H]⁺).

Those skilled in the art will appreciate that the other compounds listed in Table 5 can be prepared using methods similar to those described in Schemes XXII, XXIII, and XXIV, using appropriately substituted reagents.

### Fifth Aspect

In one embodiment of the compounds of formula (IE), wherein L² is —CH₂—, L is S(O)₂, and X and Y are both hydrogen, can have the following general structure (IE):

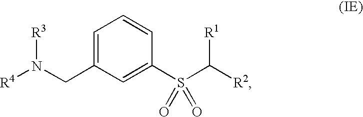

wherein —NR³R⁴, R¹, and R² are defined herein below in Table 6.

TABLE 6

| Compd. | —NR³R⁴ | R¹ | R² |
|---|---|---|---|
| LW | 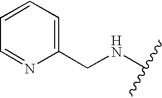 | H | benzyl |
| LX | 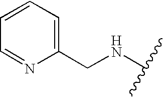 | methyl | benzyl |
| LY | 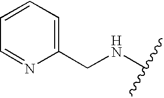 | isopropyl | benzyl |
| LZ | 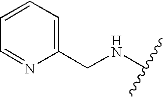 | 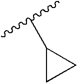 | benzyl |
| MA | 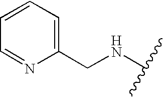 | 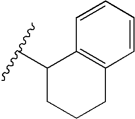 | benzyl |
| MB | 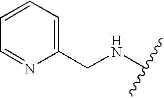 | 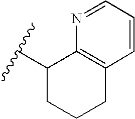 | benzyl |
| MC | 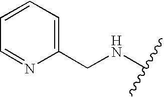 | 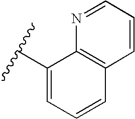 | benzyl |
| MD | 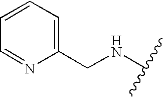 | H | 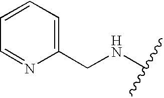 |
| ME | 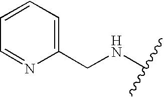 | methyl | 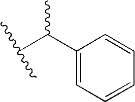 |
| MF | 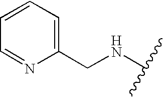 | methyl | 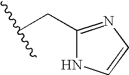 |
| MG | 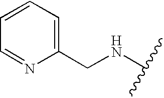 | methyl | 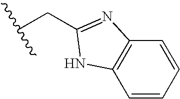 |
| MH | 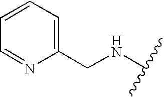 | H | 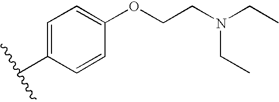 |
| MI | 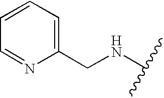 | methyl | 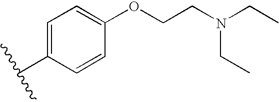 |
| MJ | 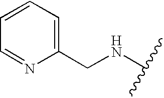 | methyl | 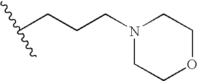 |
| MK | 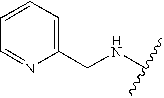 | methyl | 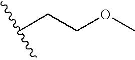 |
| ML | 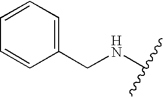 | H | benzyl |
| MM | 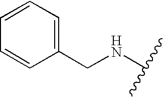 | methyl | benzyl |
| MN | 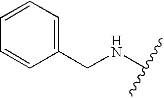 | isopropyl | benzyl |
| MO | 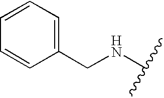 | 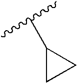 | benzyl |
| MP | 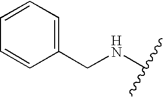 | 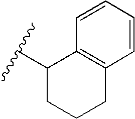 | benzyl |
| MQ | 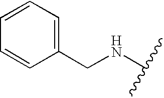 | 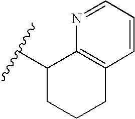 | benzyl |
| MR | 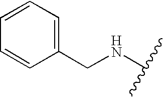 | 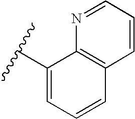 | benzyl |
| MS | 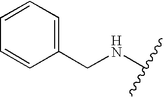 | H | 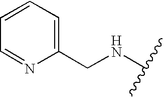 |
| MT | 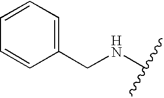 | methyl | 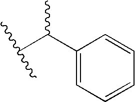 |
| MU | 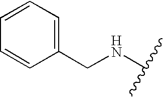 | methyl | 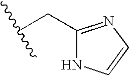 |
| MV | 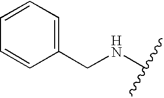 | methyl | 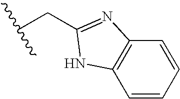 |
| MW | 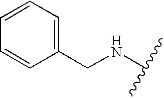 | H | 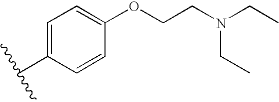 |
| MX | 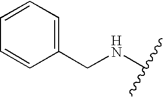 | methyl | 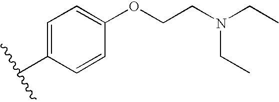 |
| MY | 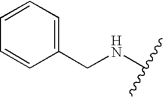 | methyl | 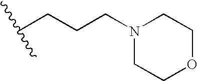 |
| MZ | 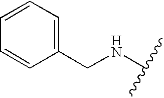 | methyl | 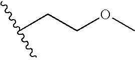 |
| NA | 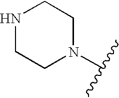 | H | benzyl |
| NB | 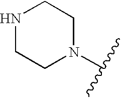 | methyl | benzyl |
| NC | 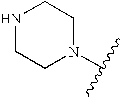 | isopropyl | benzyl |
| ND | 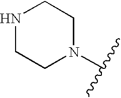 | 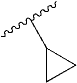 | benzyl |
| NE | 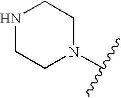 | 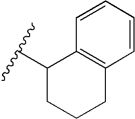 | benzyl |
| NF | 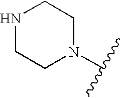 | 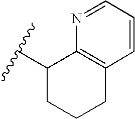 | benzyl |
| NG | 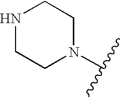 | 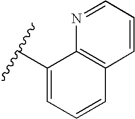 | benzyl |
| NH | 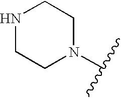 | H | 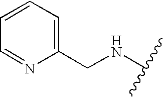 |
| NI | 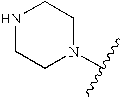 | methyl | 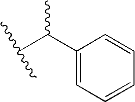 |
| NJ | 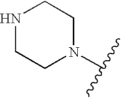 | methyl | 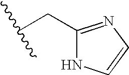 |
| NK | 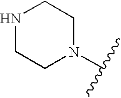 | methyl | 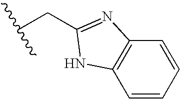 |
| NL | 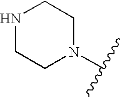 | H | 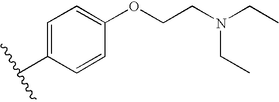 |
| NM | 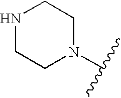 | methyl | 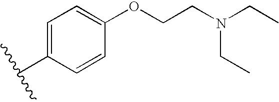 |
| NN | 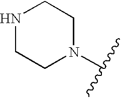 | methyl | 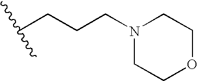 |
| NO | 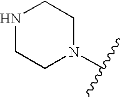 | methyl | 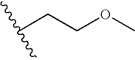 |
| NP | 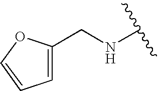 | benzyl | benzyl |
| NQ | 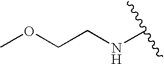 | benzyl | benzyl |
| NR | 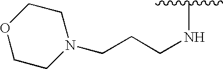 | benzyl | benzyl |
| NS | 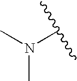 | benzyl | benzyl |
| NT | 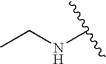 | benzyl | benzyl |
| NU | 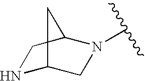 | benzyl | benzyl |
| NV | 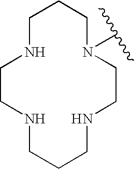 | benzyl | benzyl |
| NW | 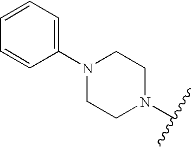 | benzyl | H |
| NX | 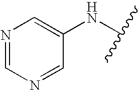 | benzyl | benzyl |
| NY | 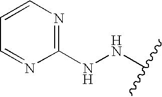 | methyl | benzyl |
| NZ | 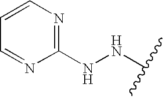 | 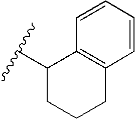 | benzyl |
| OA | 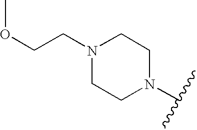 | benzyl | benzyl |
| OB | 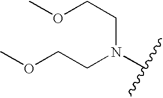 | benzyl | benzyl |
| OC | 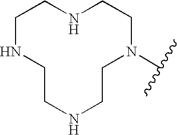 | benzyl | benzyl |
| OD | 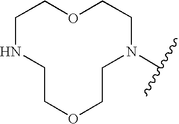 | benzyl | benzyl |
| OE | 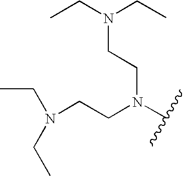 | methyl | benzyl |
| OF | 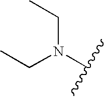 | methyl | benzyl |
| OG | 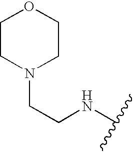 | methyl | benzyl |
| OH | 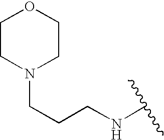 | methyl | benzyl |
| OI | 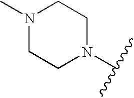 | H | 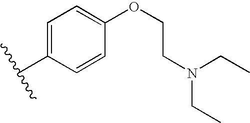 |
| OJ | 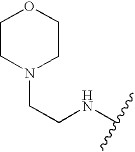 | 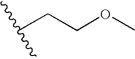 | 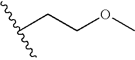 |
| OK | 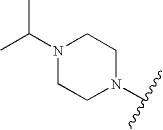 | H | 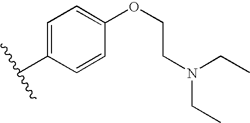 |
| OL | 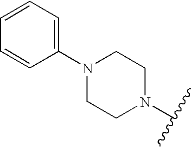 | methyl | benzyl |
| OM | 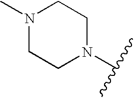 | methyl | benzyl |
| ON | 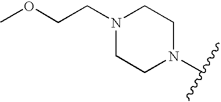 | methyl | benzyl |
| OO | 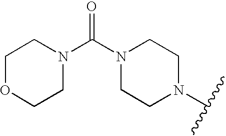 | methyl | benzyl |
| OP | 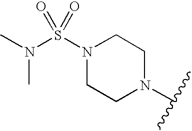 | methyl | benzyl |
| OQ | 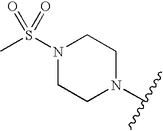 | methyl | benzyl |
| OR | 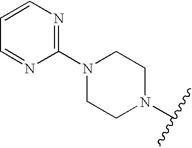 | methyl | benzyl |
| OS | 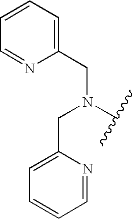 | methyl | benzyl |
| OT | 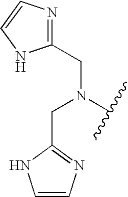 | methyl | benzyl |
| OU | 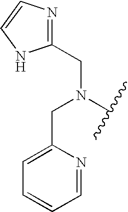 | methyl | benzyl |
| OV | 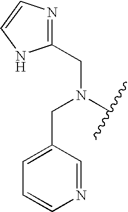 | methyl | benzyl |
| OW | 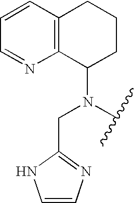 | methyl | benzyl |
| OX | 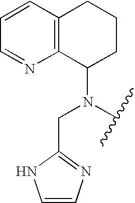 | methyl | benzyl |
| OY | 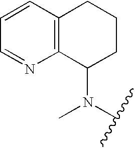 | methyl | benzyl |
| OZ | 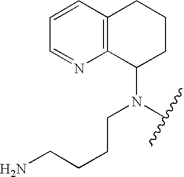 | methyl | benzyl |
| PA | 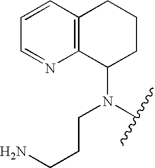 | methyl | benzyl |
| PB | 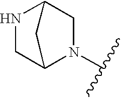 | methyl | benzyl |
| PC | 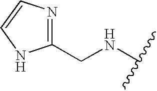 | methyl | benzyl |
| PD | 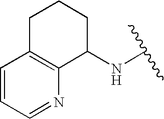 | H | benzyl |
| PE | 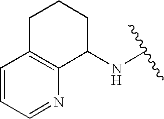 | methyl | benzyl |
| PF | 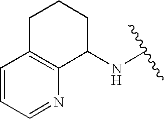 | 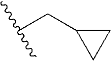 | benzyl |
| PG | 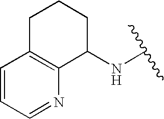 | methyl | 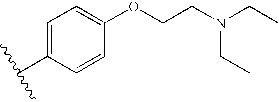 |
| PH | 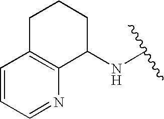 | methyl | 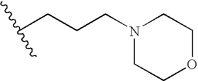 |
| PI | 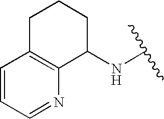 | methyl | 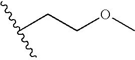 |
| PJ | 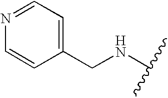 | benzyl | benzyl |
| PK | 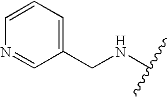 | benzyl | benzyl |
| PL | 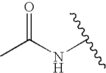 | H | 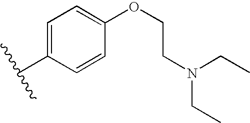 |
| PM | 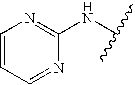 | H | 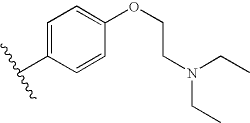 |

In another embodiment of the compounds of formula (IE), R¹ and R², taken together with the nitrogen atom to which they are both shown attached, form a substituted or unsubstituted 5- to 18-membered saturated heterocyclic ring containing at least one nitrogen atom; and/or R³ and R⁴, taken together with the nitrogen atom to which they are both shown attached, form a substituted or unsubstituted 5- to 18-membered saturated heterocyclic ring containing at least one nitrogen atom. —NR¹R² and —NR³R⁴ are exemplified in Table 7 below:

TABLE 7

| Compd. | —NR³R⁴ | —NR¹R² |
|---|---|---|
| PN | 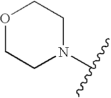 | 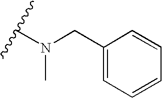 |
| PO | 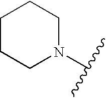 | 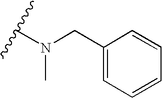 |
| PP | 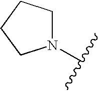 | 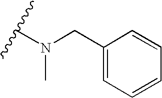 |
| PQ | 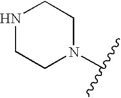 | 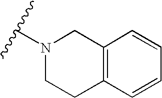 |
| PR | 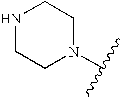 | 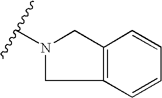 |
| PS | 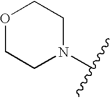 | 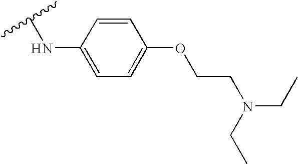 |
| PT | 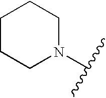 | 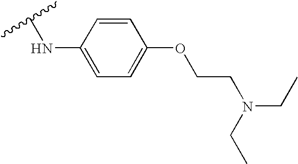 |
| PU | 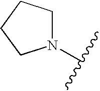 | 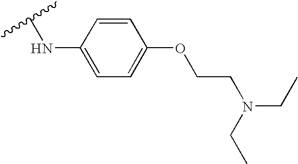 |
| PV | 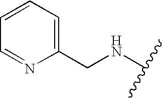 | 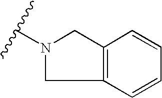 |
| PW | 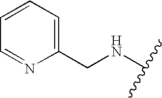 | 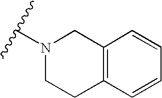 |
| PX | 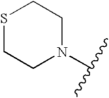 | 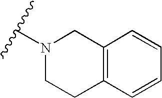 |
| PY | 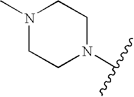 | 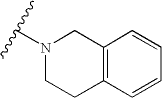 |
| PZ | 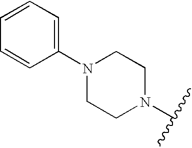 | 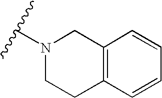 |
| QA | 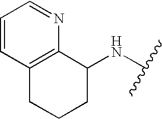 | 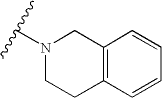 |
| QB | 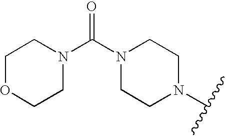 | 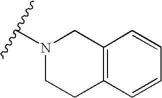 |
| QC | 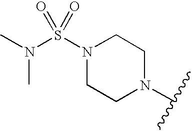 | 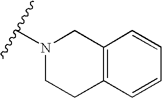 |
| QD | 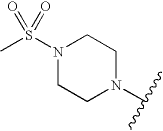 | 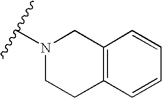 |
| QE | 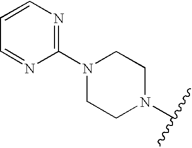 | 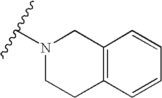 |
| QF | 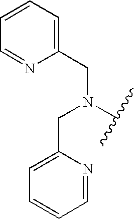 | 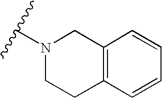 |
| QG | 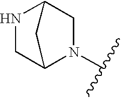 | 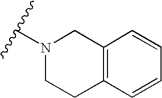 |
| QH | 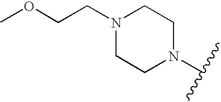 | 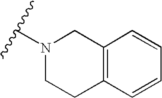 |
| QI | 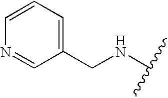 | 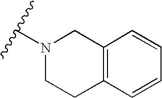 |
| QJ | 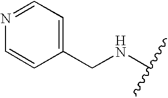 | 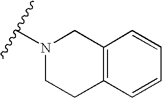 |
| QK | 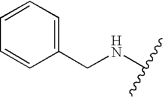 | 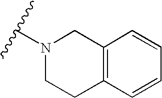 |
| QL | 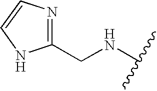 | 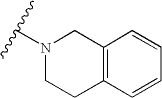 |
| QM | 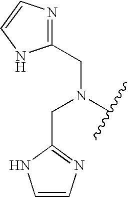 | 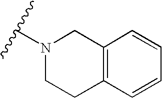 |
| QN | 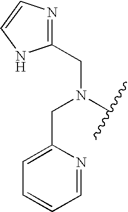 | 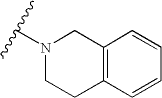 |
| QO | 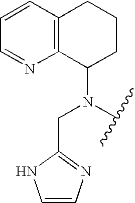 | 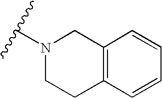 |
| QP | 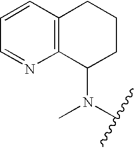 | 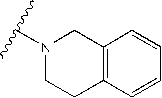 |
| QQ | 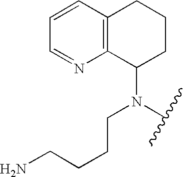 | 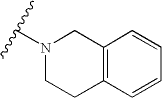 |
| QR | 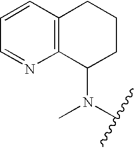 | 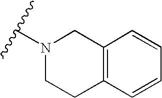 |
| QS | 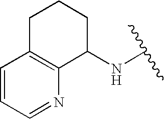 | 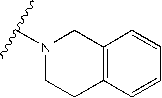 |
| QT | 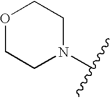 | 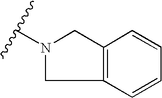 |
| QU | 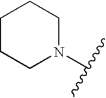 | 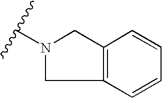 |
| QV | 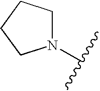 | 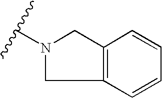 |
| QW | 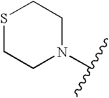 | 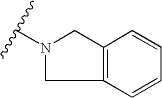 |
| QX | 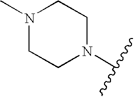 | 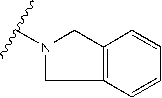 |
| QY | 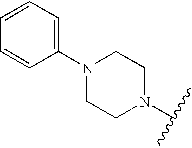 | 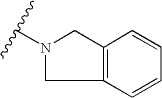 |
| QZ | 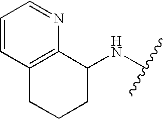 | 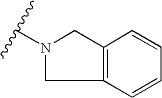 |
| RA | 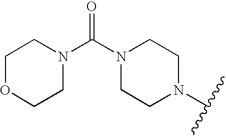 | 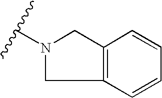 |
| RB | 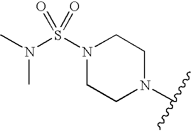 | 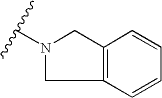 |
| RC | 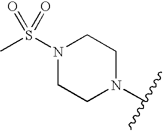 | 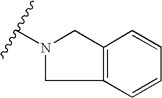 |
| RD | 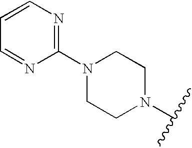 | 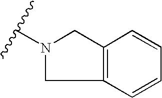 |
| RE | 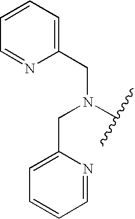 | 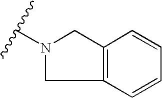 |
| RF | 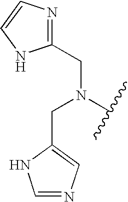 | 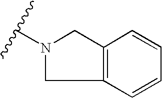 |
| RG | 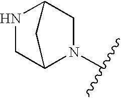 | 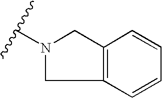 |
| RH | 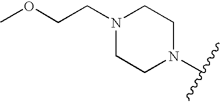 | 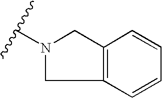 |
| RI | 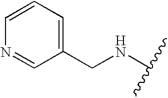 | 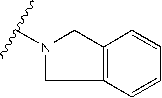 |
| RJ | 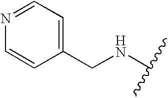 | 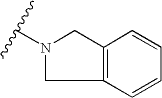 |
| RK | 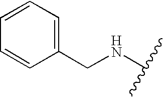 | 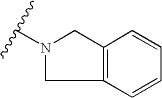 |
| RL | 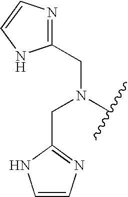 | 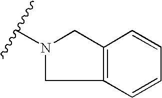 |
| RM | 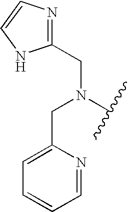 | 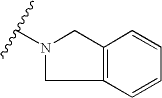 |
| RN | 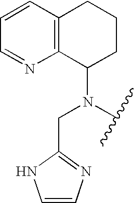 | 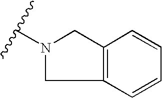 |
| RO | 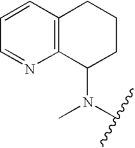 | 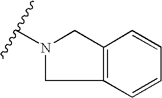 |
| RP | 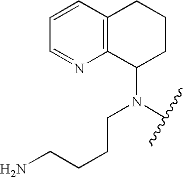 | 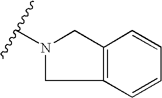 |
| RQ | 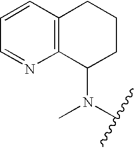 | 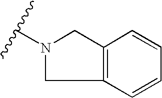 |
| RR | 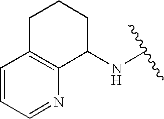 | 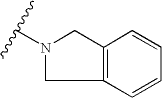 |
| RS | 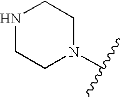 | 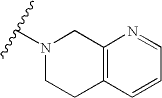 |
| RT | 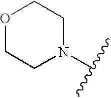 | 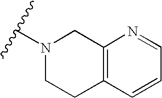 |
| RU | 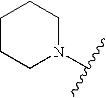 | 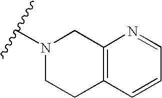 |
| RV | 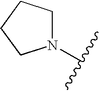 | 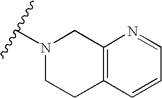 |
| RW | 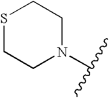 | 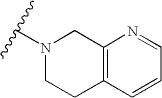 |
| RX | 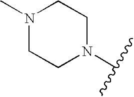 | 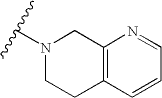 |
| RY | 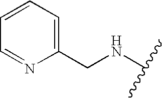 | 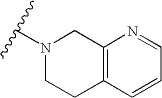 |
| RZ | 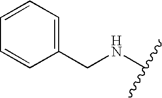 | 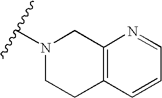 |
| SA | 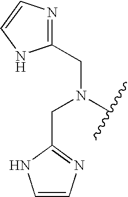 | 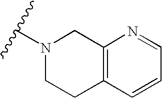 |
| SB | 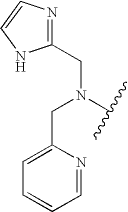 | 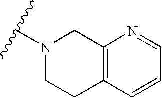 |
| SC | 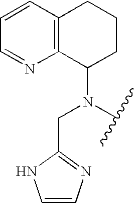 | 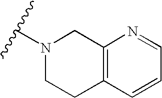 |
| SD | 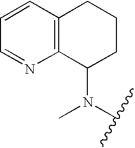 | 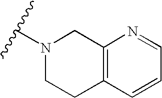 |
| SE | 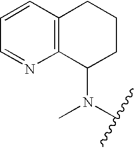 | 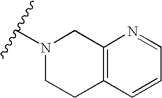 |
| SF | 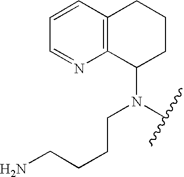 | 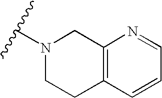 |
| SG | 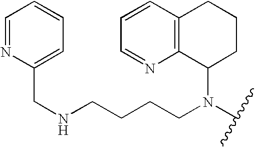 | 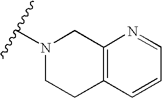 |
| SH | 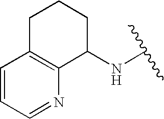 | 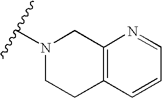 |
| SI | 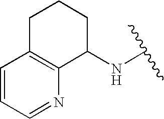 | 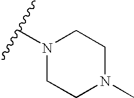 |
| SJ | 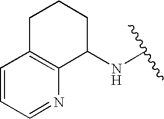 | 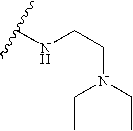 |
| SK | 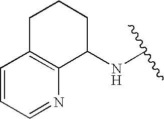 | 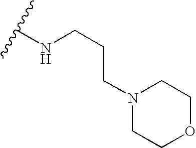 |
| SL | 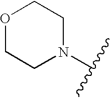 | 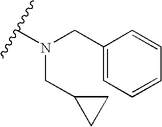 |
| SM | 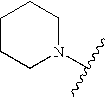 | 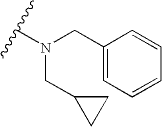 |
| SN | 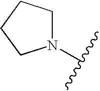 | 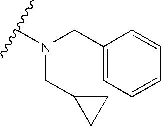 |
| SO | 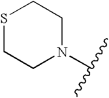 | 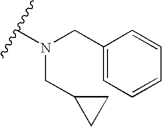 |
| SP | 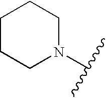 | 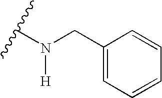 |
| SQ | 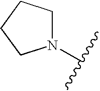 | 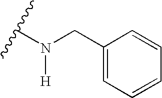 |
| SR | 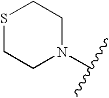 | 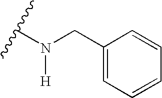 |

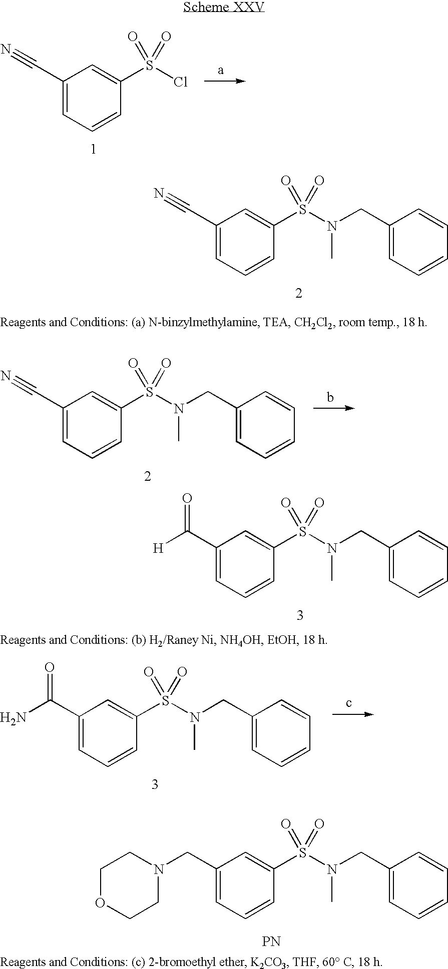

### Example 25

### N-benzyl-N-methyl-3-(morpholinomethyl)benzenesulfonamide (PN)

Preparation of N-benzyl-3-cyano-N-methylbenzenesulfonamide (2): To a solution of 3-cyanobenzene sulfonyl chloride (1) (2.0 g, 10.0 mmol) in dichloromethane (20 mL), was added triethylamine (1.52 mL, 11.0 mmol) and N-benzylmethylamine (1.40 mL, 11.0 mmol). The reaction stirred at room temperature for 4 h. The mixture was quenched with brine (5 mL), dried over magnesium sulfate, filtered and concentrated to yield (2) as a crude yellow solid: ¹H NMR (400 MHz, CDCl₃) δ 8.08-8.02 (m, 2H), 7.88-7.85 (m, 1H), 7.71-7.66 (m, 1H), 7.31-7.24 (m, 5H), 4.18 (s, 2H), 2.65 (s, 3H); ESI⁺ MS: m/z (rel intensity) 309.0 (100, [M+Na]⁺).

### Preparation of 3-(aminomethyl)-N-benzyl-N-methylbenzenesulfonamide (3)

To a solution of N-benzyl-3-cyano-N-methylbenzenesulfonamide, 2, (1.0 g, 3.49 mmol) in ethanol (40 mL) was added ammonium hydroxide (1 mL) and Raney nickel (1 mL). The reaction was stirred under H₂ atmosphere for 18 h. Upon completion, the solution was filtered through a celite pad with excessive washing with ethanol (150 mL). The liquid was concentrated in vacuo to afford (3) as a colorless viscous oil: ¹H NMR (400 MHz, CDCl₃) δ 7.77 (s, 1H), 7.70 (d, J=7.6 Hz, 1H), 7.57 (d, J=8.0 Hz, 1H), 7.51 (t, J=7.6 Hz, 1H), 7.31-7.26 (m, 5H), 4.13 (s, 2H), 3.96 (s, 2H), 2.59 (s, 3H), 1.66 (bs, NH₂); ESI⁺ MS: m/z (rel intensity) 291.1 (100, [M+H]⁺).

Preparation of N-benzyl-N-methyl-3-(morpholinomethyl)-benzenesulfonamide (PN): To a solution of 3-(aminomethyl)-N-benzyl-N-methylbenzenesulfonamide, 3, (0.30 g, 1.03 mmol) in tetrahydrofuran (5 mL) was added potassium carbonate (0.28 g, 2.07 mmol) and 2-bromoethyl ether (0.14 g, 1.14 mmol). The reaction stirred at 60° C. for 18 h. The mixture was cooled to room temperature and quenched with saturated aqueous solution of sodium bicarbonate. The product was extracted with ethyl acetate (10 mL). The combined organic layers were washed with brine, dried over magnesium sulfate and concentrated under reduced pressure. The crude material was purified by silica gel chromatography (5% MeOH/CHCl₃) to give PN (0.08 g, 20% yield): ¹H NMR (400 MHz, CDCl₃) δ 7.80 (bs, 1H), 7.71 (d, J=7.6 Hz, 1H), 7.58 (d, J=7.6 Hz, 1H), 7.50 (d, J=7.6 Hz, 1H), 7.30-7.25 (m, 5H), 4.12 (s, 2H), 3.68 (t, J=4.8 Hz, 4H), 3.55 (s, 2H), 2.58 (s, 3H), 2.43 (t, J=4.0 Hz, 4H); ESI⁺ MS: m/z (rel intensity) 361.1 (100, [M+H]⁺).

Compounds PO and PP listed below is a non-limiting example of the fifth aspect of the present invention which was prepared using methods similar to those described in Scheme XXV by using the appropriate electrophile in step c.

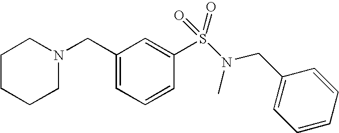

N-benzyl-N-methyl-3-(piperidin-1-ylmethyl)benzenesulfonamide (PO): ¹H NMR (400 MHz, CDCl₃) δ 7.79 (bs, 1H), 7.69 (d, J=7.6 Hz, 1H), 7.57 (d, J=7.6 Hz, 1H), 7.48 (d, J=7.6 Hz, 1H), 7.31-7.26 (m, 5H), 4.12 (s, 2H), 3.52 (s, 2H), 2.57 (s, 3H), 2.35 (bs, 4H), 1.56-1.52 (m, 4H), 1.41 (bs, 2H); ESI⁺ MS: m/z (rel intensity) 359.1 (100, [M+H]⁺).

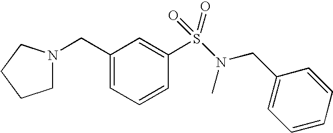

N-benzyl-N-methyl-3-(pyrrolidin-1-ylmethyl)benzenesulfonamide (PP): ¹H NMR (400 MHz, CDCl₃) δ 7.79 (s, 1H), 7.70 (d, J=8.4 Hz, 1H), 7.60 (d, J=8.0 Hz, 1H), 7.49 (d, J=8.0 Hz, 1H), 7.31-7.26 (m, 5H), 4.12 (s, 2H), 3.68 (s, 2H), 2.58 (s, 3H), 2.50 (bs, 4H), 1.79-1.76 (m, 4H); ESI⁺ MS: m/z (rel intensity) 345.2 (100, [M+H]⁺).

### Sixth Aspect

Compounds of formula (I), wherein L² is CO, L¹ is M¹-N(R⁵)-M², and X and Y are both hydrogen, can have the following general structure (IF):

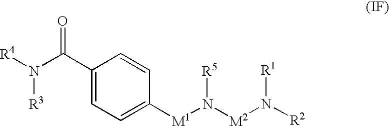

wherein M¹, M², R⁵, R¹, R², and —NR³R⁴ are defined herein below in Table 8. As shown below in Table 8, when M¹ is “-”, it denotes a covalent bond.

TABLE 8

| Cmpd. | —NR³R⁴ | M¹ | R⁵ | M² | R¹ | R² |
|---|---|---|---|---|---|---|
| SS | 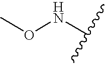 | CH₂ | 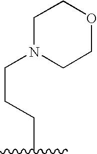 | C(O) | H | 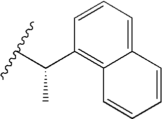 |
| ST | 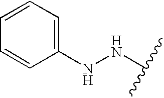 | CH₂ | 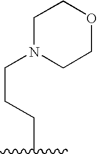 | C(O) | H | 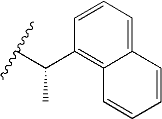 |
| SU | 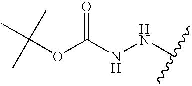 | CH₂ | 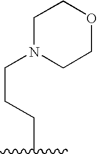 | C(O) | H | 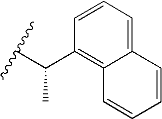 |
| SV | 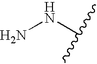 | CH₂ | 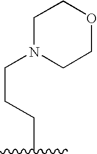 | C(O) | H | 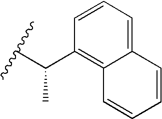 |

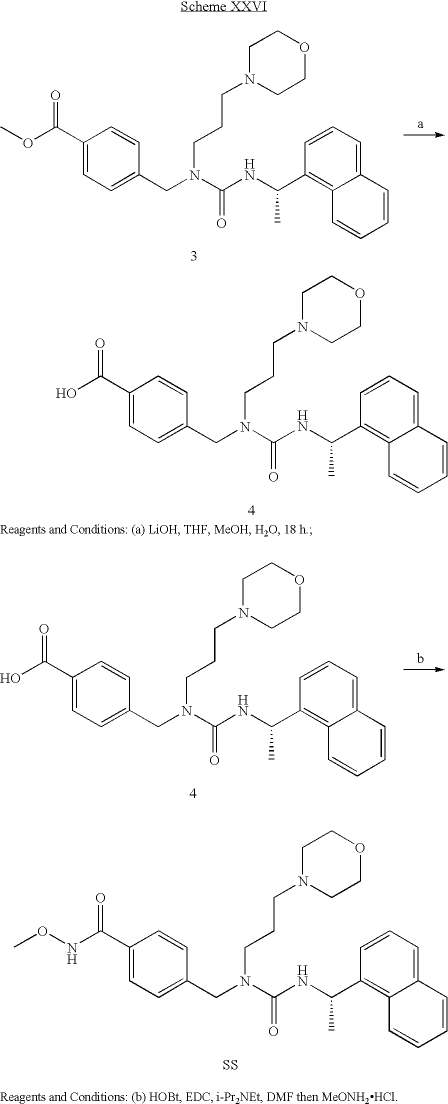

### Example 26

### (S)—N-methoxy-4-((1-(3-morpholinopropyl)-3-(1-(naphthalen-1-yl)ethyl)ureido)methyl)-benzamide (SS)

Preparation of (S)-4-((1-(3-morpholinopropyl)-3-(1-(naphthalen-1-yl)ethyl)ureido)methyl)benzoic acid (4): To a solution of (S)-methyl 4-((1-(3-morpholinopropyl)-3-(1-(naphthalen-1-yl)ethyl)ureido)methyl)benzoate, 3, (0.83 g, 1.69 mmol) in THF/MeOH/H₂O (16 mL of 10:1:5 mixture of THF/MeOH/H₂O) was added lithium hydroxide (0.48 g, 20.37 mmol). The reaction was stirred for 18 h at room temperature. The solution was poured into H₂O. The aqueous phase was extracted with EtOAc. The aqueous phase was acidified to pH-1 with 1N HCl. The aqueous phase was extracted five times with 20% i-PrOH/CHCl₃. The combined organic phases were dried (MgSO₄), filtered and concentrated in vacuo. The resulting crude residue, 716 mg of the HCl salt, was used without further purification: ¹H NMR (400 MHz, d⁶-DMSO) δ 8.13 (d, J=8.4 Hz, 1H), 7.89 (d, J=8.0 Hz, 1H), 7.85 (d, J=8.0 Hz, 2H), 7.76 (d, J=8.0 Hz, 1H), 7.55-7.41 (m, 4H), 7.26 (d, J=8.4 Hz, 2H), 7.10 (d, J=7.6 Hz, 1H), 5.67 (dq, J=7.6, 7.6 Hz, 1H), 4.59 (s, 2H), 3.86 (d, J=12.8 Hz, 1H), 3.77 (d, J=12.0 Hz, 1H), 3.67 (t, J=11.6 Hz, 1H), 3.58 (t, J=11.6 Hz, 1H), 3.36-3.20 (m, 4H), 2.96-2.80 (m, 4H), 1.83-1.78 (m, 2H), 1.46 (d, J=7.2 Hz, 3H); ESI⁺ MS: m/z (rel intensity) 476 (100, [M+H]⁺).

Preparation of (S)—N-methoxy-4-((1-(3-morpholinopropyl)-3-(1-(naphthalen-1-yl)ethyl)ureido)methyl)benzamide (SS): A solution of (S)-4-((1-(3-morpholinopropyl)-3-(1-(naphthalen-1-yl)ethyl)ureido)methyl)benzoic acid, 4, (0.16 g, 0.31 mmol) in dimethylformamide (5 mL) at 0° C. was treated with HOBt (0.05 g, 0.37 mmol), diisopropyletylamine (0.21 mL, 1.25 mmol) and EDAC (0.07 g, 0.37 mmol). After 1 h at 0° C., methoxyamine hydrochloride (0.05 g, 0.62 mmol) was added. The reaction was then slowly warmed to room temperature and stirred for 36 h. A saturated aqueous solution of sodium bicarbonate (5 mL) was added. The product was extracted three times with 5 mL of ethyl acetate. The combined organic layers were washed with brine, dried over magnesium sulfate, filtered and concentrated in vacuo. The crude material was purified by silica gel chromatography (0-10% MeOH/CH₂Cl₂) to afford 0.14 g (82% yield) of pure product SS: ¹H NMR (400 MHz, CDCl₃) [symbol] 8.13 (d, J=8.4 Hz, 1H), 7.82 (d, J=8.0 Hz, 1H), 7.76 (d, J=8.0 Hz, 1H), 7.52-7.40 (m, 4H), 7.27 (d, J=8.0 Hz, 2H), 5.90-5.80 (m, 1H), 4.59 (d, J=15.2 Hz, 1H), 4.46 (d, J=15.6 Hz, 1H), 3.86 (s, 3H), 3.35-3.25 (m, 2H), 3.20-3.00 (m, 4H), 2.20-2.05 (m, 4H), 1.94-1.85 (m, 2H), 1.67 (d, J=7.2 Hz, 3H), 1.56-1.45 (m, 2H); ESI⁺ MS: m/z (rel intensity) 505.2 (100, [M+H]⁺).

Compounds ST, SU and SV listed below are non-limiting examples of the sixth aspect of the present invention which were prepared using methods similar to those described in Scheme XXVI (i.e., methoxyamine hydrochloride of step b, Scheme XXVI was replaced with phenyl hydrazine or Boc-hydrazine). Compound SV was obtained by Boc-removal of SU using thionyl chloride in MeOH.

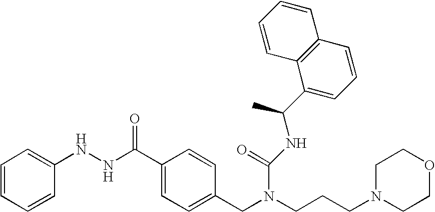

(S)-1-(3-morpholinopropyl)-3-(1-(naphthalen-1-yl)ethyl)-1-(4-(2-phenylhydrazinecarbonyl)benzyl)urea (ST): ¹H NMR (400 MHz, d⁶-DMSO) δ 8.12 (d, J=8.4 Hz, 1H), 7.90-7.76 (m, 4H), 7.53-7.42 (m, 4H), 7.28 (d, J=8.0 Hz, 2H), 7.10 (d, J=7.2 Hz, 2H), 6.82 (d, J=8.0 Hz, 1H), 6.75-6.66 (m, 2H), 5.67 (bs, 1H), 4.52 (s, 1H), 3.39 (bs, 2H), 3.32 (bs, 7H), 3.20-3.10 (m, 2H), 2.12 (bs, 4H), 1.50 (bs, 4H); ESI⁺ MS: m/z (rel intensity) 566.3 (100, [M+H]⁺).

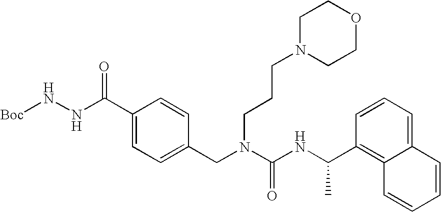

(S)-tert-butyl 2-(4-((1-(3-morpholinopropyl)-3-(1-(naphthalen-1-yl)ethyl)ureido)methyl)benzoyl)hydrazinecarboxylate (SU): ¹H NMR (400 MHz, CDCl₃) [symbol] 8.14 (d, J=8.0 Hz, 1H), 7.88-7.80 (m, 2H), 7.80-7.70 (m, 3H), 7.54-7.40 (m, 4H), 7.33 (d, J=8.4 Hz, 2H), 6.18 (bs, 1H), 5.92-5.82 (m, 1H), 4.63 (d, J=16.0 Hz, 1H), 4.48 (d, J=16.4 Hz, 1H), 3.35-3.24 (m, 2H), 3.20-3.02 (m, 4H), 2.20-2.05 (m, 4H), 1.92-1.84 (m, 2H), 1.67 (d, J=6.8 Hz, 3H), 1.60-1.49 (m, 2H), 1.48 (s, 9H); ESI⁺ MS: m/z (rel intensity) 590.3 (100, [M+H]⁺).

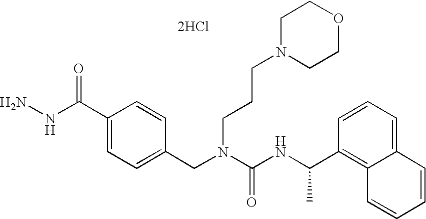

(S)-1-(4-(hydrazinecarbonyl)benzyl)-1-(3-morpholinopropyl)-3-(1-(naphthalen-1-yl)ethyl)urea (SV): ¹H NMR (400 MHz, d⁶-DMSO) [symbol] 8.13 (d, J=8.4 Hz, 1H), 7.91-7.85 (m, 3H), 7.76 (d, J=8.4 Hz, 1H), (m, 3H), 7.56-7.42 (m, 4H), 7.31 (d, J=8.4 Hz, 2H), 7.11 (d, J=8.4 Hz, 1H), 5.70-5.60 (m, 1H), 4.59 (s, 2H), 3.84-3.64 (m, 4H), 3.35-3.04 (m, 4H), 3.00-2.80 (m, 4H), 1.90-1.78 (m, 2H), 1.48 (d, J=6.4 Hz, 3H); ESI⁺ MS: m/z (rel intensity) 490.2 (100, [M+H]⁺).

### Seventh Aspect

Compounds of formula (I), wherein L² is CH₂CH₂, L¹ is M¹-N(R⁵)-M², and X and Y are both hydrogen, can have the following general structure (IG):

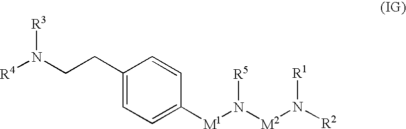

wherein M¹, M², R⁵, R¹, R², and —NR³R⁴ are defined herein below in Table 9. As shown below in Table 2, when M¹ is “-”, it denotes a covalent bond.

TABLE 9

| Cmpd. | —NR³R⁴ | M¹ | R⁵ | M² | R¹ | R² |
|---|---|---|---|---|---|---|
| SW | 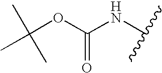 | CH2 | 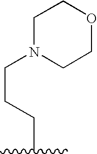 | C(O) | H | 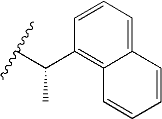 |
| SX | 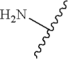 | CH2 | 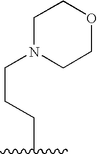 | C(O) | H | 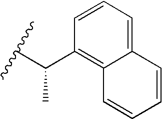 |
| SY | 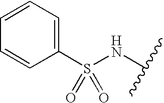 | CH2 | 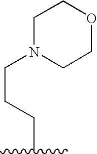 | C(O) | H | 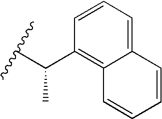 |
| SZ | 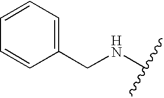 | CH2 | 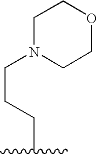 | C(O) | H | 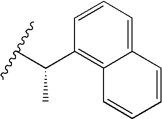 |
| TA | 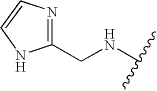 | CH2 | 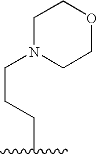 | C(O) | H | 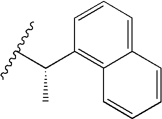 |
| TB | 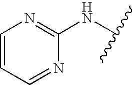 | CH2 | 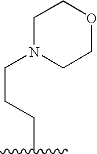 | C(O) | H | 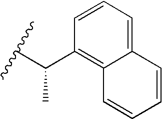 |

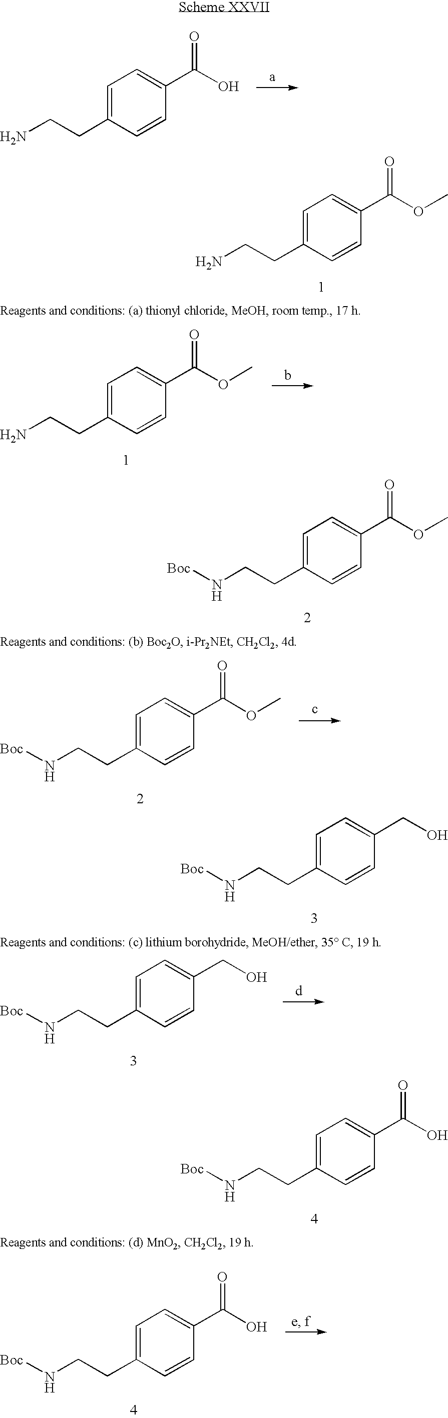

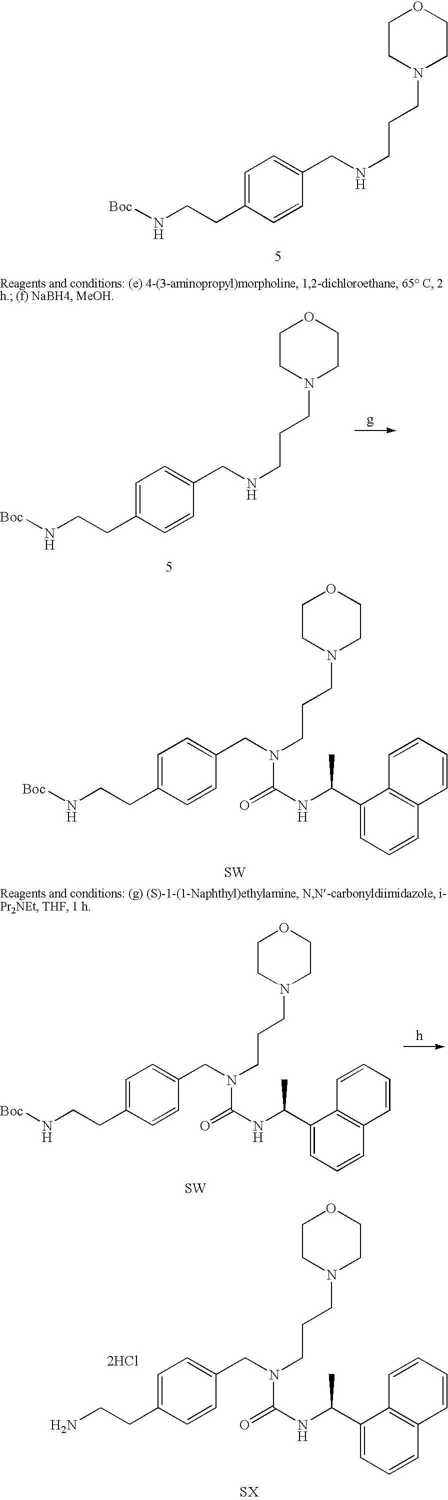

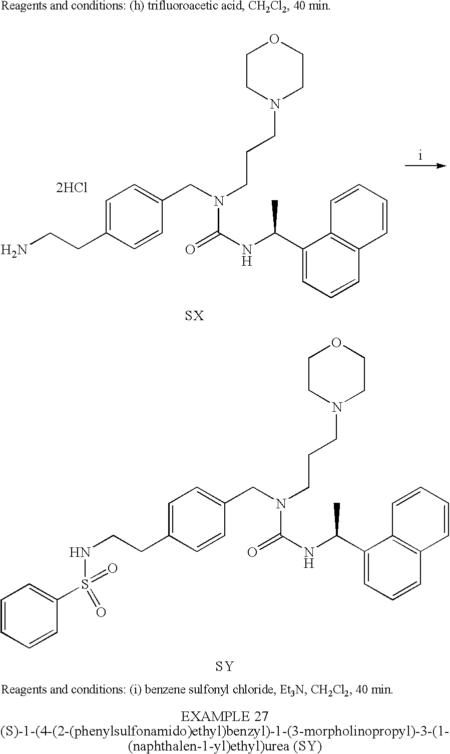

Preparation of methyl 4-(2-aminoethyl)benzoate (1): To a solution of 4-(2-aminoethyl)benzoic acid, 1, (5.0 g, 24.8 mmol) in methanol (80 mL) was added dropwise thionyl chloride (1.8 mL, 24.8 mmol). The reaction was stirred for 17 h at room temperature. The reaction mixture was concentrated in vacuo to afford the desired methyl ester as a hydrochloride salt. The resulting salt was used without further purification: ESI⁺ MS: m/z (rel intensity) 179 (100, [M+H]⁺).

Preparation of tert-butyl 4-(hydroxymethyl)phenethylearbamate (2): To a solution of methyl 4-(2-aminoethyl)benzoate hydrochloride, 1, (3.0 g, 14.0 mmol) in CH₂Cl₂ (60 mL) was added di-tert-butyldicarbonate (3.0 g, 14.0 mmol), followed by i-Pr₂NEt (4.9 mL, 28.0 mmol). The reaction was stirred for 4 days at room temperature. The solution was poured into an aqueous saturated solution of NaHCO₃. The aqueous phase was extracted with CH₂Cl₂. The organic phase was washed with an aqueous saturated solution of NH₄Cl, dried (MgSO₄), filtered and concentrated in vacuo. The resulting residue was purified over silica (0% methanol/CHCl₃ to 5% methanol/CHCl₃) to afford 2.1 g of the desired product, 2: ¹H NMR (400 MHz, d₆-DMSO) δ 7.91 (d, J=8.0 Hz, 2H), 7.37 (d, J=8.0 Hz, 2H), 6.96 (t, J=5.6 Hz, 1H), 3.87 (s, 3H), 3.20 (dd, J=13.6, 6.8 Hz, 2H), 2.80 (t, J=7.2 Hz, 2H), 1.39 (s, 9H); ESI⁺ MS: m/z (rel intensity) 302 (100, [M+Na]⁺).

Preparation of tert-butyl 4-(hydroxymethyl)phenethylcarbamate (3): To a solution of tert-butyl 4-(hydroxymethyl)phenethylcarbamate, 2, (2.0 g, 7.2 mmol) in diethyl ether (40 mL) was added methanol (1.2 mL, 28.7 mmol), followed by lithium borohydride (0.6 g, 28.7 mmol). The reaction was heated for 19 h at 35° C. The solution was poured into aqueous saturated NaHCO₃. The aqueous phase was extracted with CHCl₃. The organic phase was dried (MgSO₄), filtered and concentrated in vacuo. The resulting residue was purified over silica (0% methanol/CHCl₃ to 10% methanol/CHCl₃) to afford 1.7 g of the desired alcohol, 3: ¹H NMR (400 MHz, d₆-DMSO) δ 7.18 (d, J=7.6 Hz, 2H), 7.09 (d, J=7.6 Hz, 2H), 6.85 (t, J=5.2 Hz, 1H), 5.08 (t, J=5.6 Hz, OH), 4.40 (d, J=6.0 Hz, 2H), 3.05 (dd, J=14.4, 6.8 Hz, 2H), 2.62 (t, J=8.4 Hz, 2H), 1.33 (s, 9H); ESI⁺ MS: m/z (rel intensity) 467.2 (100, [M+Na]⁺).

Preparation of tert-butyl 4-formylphenethylcarbamate (4): To a solution of tert-butyl 4-(hydroxymethyl)phenethylcarbamate, 3, (1.7 g, 6.6 mmol) in CH₂Cl₂ (35 mL) was added manganese dioxide (3.4 g, 39.4 mmol). The reaction was stirred for 19 h at room temperature. The reaction mixture was diluted with CH₂Cl₂ and filtered through celite, which was washed thoroughly with additional CH₂Cl₂. The filtrate was concentrated in vacuo. The resulting residue was purified over silica (0% methanol/CHCl₃ to 5% methanol/CHCl₃) to afford 1.2 g of the desired aldehyde, 4: ¹H NMR (400 MHz, d₆-DMSO) δ 9.93 (s, 1H), 7.78 (d, J=7.6 Hz, 2H), 7.38 (d, J=8.0 Hz, 2H), 3.05 (dd, J=12.8, 6.4 Hz, 2H), 2.75 (t, J=7.6 Hz, 2H), 1.33 (s, 9H); ESI⁺ MS: m/z (rel intensity) 272 (100, [M+Na]⁺).

Preparation of tert-butyl 4-((3-morpholinopropylamino)methyl)-phenethylcarbamate (5): To a solution of tert-butyl 4-formylphenethylcarbamate, 4 (1.2 g, 5.0 mmol) in 1,2-dichloroethane (25 mL) was added 4-(3-aminopropyl)-morpholine (0.9 mL, 6.0 mmol). The reaction was heated for 2 h at 65° C. The mixture was concentrated in vacuo. At 0° C., the crude residue was diluted with methanol (25 mL) and sodium borohydride was added immediately. The reaction was stirred for 30 min at 0° C. The solution was poured into aqueous saturated NaHCO₃. The aqueous phase was extracted with EtOAc. The organic phase was dried (MgSO₄), filtered and concentrated in vacuo to afford 1.7 g of 5, which was used without further purification: ESI⁺ MS: m/z (rel intensity) 378 (100, [M+H]⁺).

Preparation of (S)-tert-butyl 4-((1-(3-morpholinopropyl)-3-(1-(naphthalen-1-yl)ethyl)ureido)methyl)phenethylcarbamate (SW): To a solution of tert-butyl 4-((3-morpholinopropylamino)methyl)-phenethylcarbamate, 5 (1.7 g, 4.5 mmol) in THF (25 mL) was added ⁱPr₂NEt (0.8 mL, 4.7 mmol), followed by N,N′-carbonyl diimidazole (0.8 g, 5.0 mmol). The reaction was stirred for 5 min at room temperature. A solution of (S)-1-(1-naphthyl)ethylamine (0.6 mL, 3.8 mmol) in THF (2 mL) was added to the reaction, which was then stirred for 1 h at room temperature. The mixture was poured into aqueous saturated NaHCO₃. The aqueous phase was extracted with EtOAc. The organic phase was dried (MgSO₄), filtered and concentrated in vacuo. The resulting residue was purified over silica (0% methanol/CHCl₃ to 25% methanol/CHCl₃) to afford 2.2 g of desired urea, SW: ¹H NMR (400 MHz, d₆-DMSO) δ 8.12 (d, J=8.0 Hz, 1H), 7.89 (d, J=7.6 Hz, 1H), 7.77 (d, J=7.6 Hz, 1H), 7.53-7.42 (m, 4H), 7.08 (s, 4H), 6.86-6.83 (m, 1H), 6.74 (d, J=7.2 Hz, 1H), 5.67 (dq, J=7.2, 7.2 Hz, 1H), 4.40 (s, 2H), 3.45-3.33 (m, 4H), 3.25-3.04 (m, 4H), 2.62 (t, J=8.0 Hz, 2H), 2.19-2.04 (m, 6H), 1.51-1.40 (m, 2H), 1.48 (d, J=6.8 Hz, 3H), 1.33 (s, 9H); ESI⁺ MS: m/z (rel intensity) 575 (100, [M+H]⁺).

Preparation of (S)-1-(4-(2-aminoethyl)benzyl)-1-(3-morpholinopropyl)-3-(1-(naphthalen-1-yl)ethyl)urea dihydrochloride (SX): To a solution of (S)-tert-butyl 4-((1-(3-morpholinopropyl)-3-(1-(naphthalen-1-yl)ethyl)ureido)methyl)phenethyl-carbamate, SW (1.2 g, 2.0 mmol) in dichloromethane (15 mL) was added dropwise trifluoroacetic acid (15 mL). The reaction was stirred for 40 min at room temperature. The reaction mixture was concentrated in vacuo to afford the product as a trifluoroacetate salt. The resulting salt was neutralized by addition to aqueous 1N sodium hydroxide solution. The aqueous phase was extracted with ethyl acetate, followed by 20% isopropanol/CHCl₃. The combined organic phases were dried (MgSO₄), filtered and concentrated in vacuo. After diluting the resulting residue with ether, 1N HCl (1N in ether, 2 equivalents) was added to precipitate the product. The solution was concentrated in vacuo to afford the desired product, SX, as the bishydrochloride salt, which was used without further purification: ¹H NMR (400 MHz, d₆-DMSO) δ 8.12 (d, J=8.4 Hz, 1H), 7.89 (d, J=7.6 Hz, 1H), 7.77 (d, J=7.6 Hz, 1H), 7.53-7.41 (m, 4H), 7.11-7.08 (m, 4H), 6.76 (d, J=7.6 Hz, 1H), 5.67 (dq, J=7.2, 7.2 Hz, 1H), 4.40 (s, 2H), 3.40-3.05 (m, 8H), 2.70 (t, J=6.8 Hz, 2H), 2.56 (t, J=6.8 Hz, 2H), 2.17-2.05 (m, 4H), 1.54-1.47 (m, 2H), 1.48 (d, J=6.8 Hz, 3H); ESI⁺ MS: m/z (rel intensity) 475 (100, [M+H]⁺).

Preparation of (S)-1-(4-(2-(phenylsulfonamido)ethyl)benzyl)-1-(3-morpholinopropyl)-3-(1-(naphthalen-1-yl)ethyl)urea (SY): To a solution of (S)-1-(4-(2-aminoethyl)benzyl)-1-(3-morpholinopropyl)-3-(1-(naphthalen-1-yl)ethyl)urea dihydrochloride, SW (0.12 g, 0.22 mmol) and triethylamine (0.15 mL, 1.10 mmol) in CH₂Cl₂ (3 mL) was added dropwise benzenesulfonyl chloride (0.03 mL, 0.24 mmol). The reaction was stirred for 40 min at room temperature. The mixture was poured into aqueous saturated NaHCO₃. The aqueous phase was extracted with EtOAc. The organic phase was dried (MgSO₄), filtered and concentrated in vacuo. The resulting residue was purified over silica (0% methanol/CHCl₃ to 20% methanol/CHCl₃) to afford 36 mg of desired urea, SY: ¹H NMR (400 MHz, CDCl₃) δ 8.15 (d, J=8.0 Hz, 1H), 7.84-7.75 (m, 4H), 7.58-7.52 (m, 1H), 7.50-7.40 (m, 6H), 7.16 (d, J=8.4 Hz, 2H), 6.89 (d, J=8.0 Hz, 2H), 6.06-5.96 (m, 1H), 5.68 (dq, J=6.8, 6.8 Hz, 1H), 4.49 (d, J=6.0 Hz, 2H), 4.43-4.39 (m, 1H), 3.36-3.26 (m, 2H), 3.22-3.11 (m, 6H), 2.72 (t, J=7.2 Hz, 2H), 2.21-2.10 (m, 4H), 1.97-1.89 (m, 2H), 1.66 (d, J=6.4 Hz, 3H), 1.61-1.54 (m, 2H); ESI⁺ MS: m/z (rel intensity) 615 (100, [M+H]⁺).

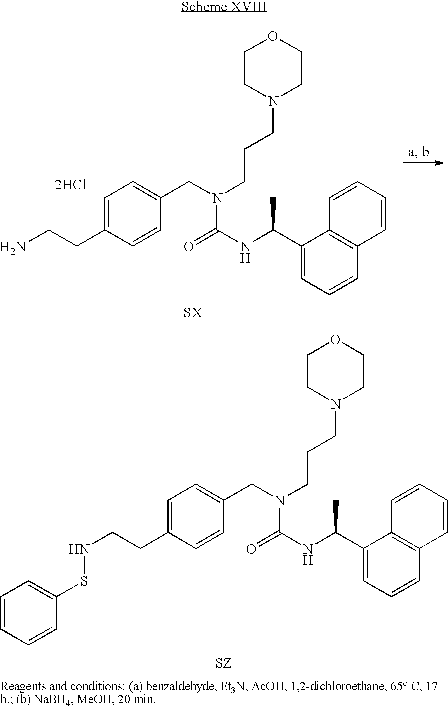

### Example 28

Preparation of (S)-1-(4-(2-(benzylamino)ethyl)benzyl)-1-(3-morpholinopropyl)-3-(1-(naphthalen-1-yl)ethyl)urea (SZ): To a solution of (S)-1-(4-(2-aminoethyl)benzyl)-1-(3-morpholinopropyl)-3-(1-(naphthalen-1-yl)ethyl)urea dihydrochloride, SX (0.23 g, 0.42 mmol) in 1,2-dichloroethane (4 mL) was added triethylamine (0.29 mL, 0.50 mmol), benzaldehyde (0.05 mL, 0.50 mmol) and acetic acid (3 drops). The reaction was heated for 19 h at 65° C. The mixture was concentrated in vacuo. At 0° C., the crude residue was diluted with methanol (25 mL) and sodium borohydride (0.03 g, 0.84 mmol) was added immediately. The reaction was stirred for 30 min at 0° C. The solution was poured into aqueous saturated NaHCO₃. The aqueous phase was extracted with EtOAc. The organic phase was dried (MgSO₄), filtered and concentrated in vacuo. The resulting residue was purified over silica (2% methanol/CHCl₃ to 25% methanol/CHCl₃) to afford 100 mg of desired urea, SZ: ¹H NMR (400 MHz, d⁶-DMSO) δ 9.46 (bs, 2H), 8.14 (d, J=7.6 Hz, 1H), 7.89 (d, J=8.0 Hz, 1H), 7.75 (d, J=8.0 Hz, 1H), 7.55-7.39 (m, 9H), 7.13 (s, 4H), 7.06-7.01 (m, 1H), 5.67 (dq, J=7.2, 7.2 Hz, 1H), 4.47 (s, 2H), 4.13 (s, 2H), 3.90-3.58 (m, 4H), 3.30-2.80 (m, 12H), 1.86-1.76 (m, 2H), 1.47 (d, J=7.2 Hz, 3H); ESI⁺ MS: m/z (rel intensity) 615 (100, [M+H]⁺).

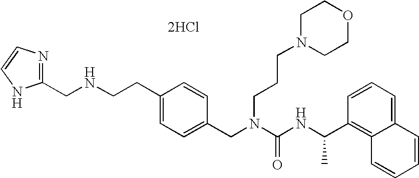

(S)-1-(4-(2-((1H-imidazol-2-yl)methylamino)ethyl)benzyl)-1-(3-morpholinopropyl)-3-(1-(naphthalen-1-yl)ethyl)urea dihydrochloride salt (TA): ¹H NMR (400 MHz, d⁶-DMSO) δ 8.13 (d, J=8.0 Hz, 1H), 7.89 (d, J=7.2 Hz, 1H), 7.76 (d, J=7.6 Hz, 1H), 7.58-7.40 (m, 4H), 7.36-7.26 (m, 1H), 7.20-7.11 (m, 3H), 7.08-7.02 (m, 2H), 5.72-5.62 (m, 1H), 4.47 (bs, 2H), 4.29 (bs, 2H), 3.90-2.80 (m, 16H), 1.88-1.72 (m, 2H), 1.47 (d, J=6.8 Hz, 3H); ESI⁺ MS: m/z (rel intensity) 555.2 (100, [M+H]⁺).

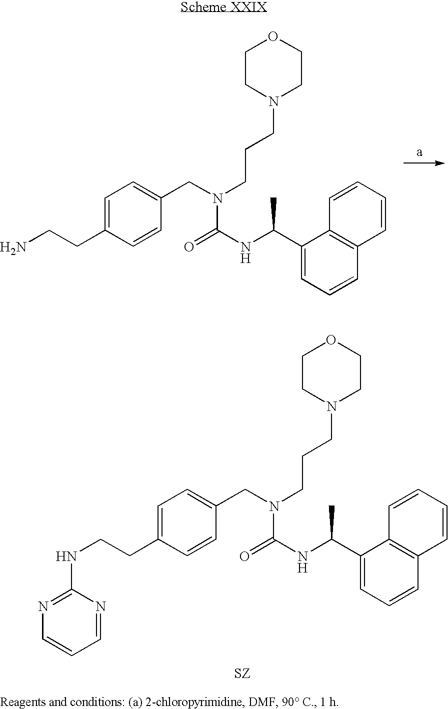

### Example 29

Preparation of (S)-1-(4-(2-(pyrimidin-2-ylamino)ethyl)benzyl)-1-(3-morpholinopropyl)-3-(1-(naphthalen-1-yl)ethyl)urea (TB): To a solution of (S)-1-(4-(2-aminoethyl)benzyl)-1-(3-morpholinopropyl)-3-(1-(naphthalen-1-yl)ethyl)urea, SX (0.21 g, 0.44 mmol) in DMF (4 mL) was added 2-chloropyrimidine (0.05 g, 0.44 mmol). The reaction was heated for 1 h at 85° C. before adding triethylamine (0.06 mL, 0.44 mmol) to the mixture. The reaction was heated for an additional 3 days at 85° C. The solution was poured into aqueous saturated NaHCO₃. The aqueous phase was extracted with EtOAc. The organic phase was washed twice with brine, dried (MgSO₄), filtered and concentrated in vacuo. The resulting residue was purified over silica (2% methanol/CHCl₃ to 20% methanol/CHCl₃) to afford 58 mg of desired urea, TB: ¹H NMR (400 MHz, d⁶-DMSO) δ 8.22 (m, 1H), 8.12 (d, J=8.4 Hz, 1H), 7.89 (d, J=7.6 Hz, 1H), 7.76 (d, J=8.4 Hz, 1H), 7.56-7.40 (m, 4H), 7.15-7.07 (m, 4H), 6.75 (d, J=7.6 Hz, 2H), 6.51 (t, J=7.2 Hz, 1H), 5.68 (m, 1H), 4.40 (s, 2H), 3.42-3.35 (m, 6H), 3.40-3.05 (m, 4H), 2.80-2.72 (m, 2H), 2.37-2.05 (m, 4H), 1.55-1.39 (m, 2H), 1.47 (d, J=6.4 Hz, 1H); ESI⁺ MS: m/z (rel intensity) 565 (100, [M+H]⁺).

### Assays

Small molecule chemokine receptor modulation, agonism or antagonism, can be mediated by direct binding to the receptor affecting the signaling and chemotatic effects of the natural ligand for its receptor. In addition modulation can be obtained by interaction of the small molecule with effectors of the particular chemokine receptor pathway. For example, modulation of CXCR4 homodimerization (Rodriguez-Frade, et al., J. Cell. Biol. 1999; Mellado et al., Annual Review of Immunology 2001; Toth et al., J. Pharm. and Exp. Ther. 2004; Wang et al., Mol. Cancer. Ther. 2006), Heterodimerization with CCR2 (Percherancier, et al. JBC 2005, Sohy et al. JBC 2007) or CCR5 (Babcock, et al., JBC 2003) or CXCR7 (Sierro et al., PNAS 2007) or delta opioid receptor (DOR) (Pello et al European J of 1 mm. 2008, Hereld and Jin European J. of 1 mm. 2008), T cell receptor (Kumar et al., Immunity 2006). Modulation of the SDF-1/CXCR4 pathway can also be accomplished by modulation of GPR54/KISS receptor (Navenot et al., Cancer Res. 2005), cannabanoid receptor 2 (CB2R) (Coopman et al., International Immunopharmacology 2007), ZAP-70 tyrosine kinase (Ottoson et al., J. Immunology 2001) or sphingosine 1-phosphate receptors (Yopp et al., J. Immunology 2005).

### Assay 1: Test Compound Activity Against HIV Strains

A selected set of compounds are tested for their ability to inhibit the cellular entry of T-tropic HIV. The assay for this inhibition is carried out on a contractural basis at Monogram Biosciences, Inc. using their well established Phenoscreen™ assay. Briefly, HIV strains of interest are tagged with a luciferase indicator gene to create an appropriate test vector. The test vector is amplified through transfection and the resulting virus is incubated in the presence of target host cells where intracellular florescence activity then becomes a measure of infection. Amplified virus is exposed to target host cells in the presence of a range of test drug concentrations to determine IC₅₀ measurements of entry inhibition. A modification of this test is further reapplied as a novel drug assay used in partnership with a number of pharmaceutical companies to test the effectiveness of novel entry inhibitors that target specific chemokines. It can used to detect activity against T-tropic, M-tropic, and dual-tropic viruses and Monogram Biosciences has a large bank of over 10,000 different virus strains to ultimately assess the range of applicability of our chemokine modulators. Certain compounds are tested to establish efficacy in a number of viral strains.

The compounds of the invention generally have an IC₅₀ value for viral entry inhibition in the one of the above HIV viral strains of interest of less than or equal to 100 μM. For example, compounds DE, DH, DX, DZ and EB have IC₅₀ values of less than or equal to 10 μM.

### Assay 2: Screening for CXCR7 Activity

CXCR7 modulation activity was accessed using PathHunter™ β-Arrestin GPCR Assay Pharmacology from DiscoveRx using the protocol recommended by the manufacture for their CXCR7β-Arrestin cell line. The compounds of the invention generally have an IC₅₀ value below 100 micromolar for CXCR7 modulation activity using this assay.

### Assay 3: Screening by Competition Assay Using Radiolabeled SDF-1

For radioligand binding competition test of CXCR4 or CXCR7, the following components are mixed in the wells of a 96 well plate (Master Block, Greiner, 786201) up to 100 μl assay buffer (50 mM HEPES; 5 mM MgCl₂; 1 mM CaCl₂, 250 mM Sucrose, 100 mM NaCl and 0.5% BSA), compounds to be tested or 200-fold excess of cold ligand for non specific binding determination (SDF1-αR&D, 350-NS), radioligand [125I]-SDF-1α (PKI NEX346, 2200 Ci/mmol, diluted in assay buffer to give 0.03 nM) and 1 μg membrane extracts. The plate is incubated during 30 min at 37° C. in a water bath, filtered over GF/B filters (presoaked in 0.5% PEI for 1 h at room temperature) with a Filtermate Harvester (Perkin Elmer), and washed 6 times with 0.5 ml of ice cold filtration buffer (50 mM HEPES; 5 mM MgCl2; 1 mM CaCl2, 250 mM Sucrose, 0.5 M NaCl and 0.5% BSA). Following addition of 50 μl of Microscint 20 (Packard), and incubation during 15 min. on an orbital shaker, the plates are counted with a TopCount™ for 1 min/well. The compounds of the invention generally have an IC₅₀ value below 100 micromolar for competitive binding versus CXCR4 or CXCR7 activity using this assay.

Various references have been cited herein, each of which is incorporated herein by reference in its entirety for all purposes.

We claim:

1. A compound of formula (I), or a pharmaceutically acceptable salt, solvate, prodrug, tautomer, or ester thereof: 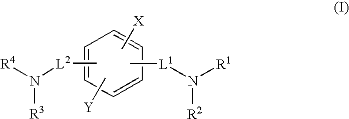
wherein
L¹ is —C(O)—, —S(O)—, —S(O)₂—, —N(R⁵)—C(O)—, —N(R⁵)—S(O)—, —N(R)—S(O)₂—, -alkylene-N(R⁵)—C(O)—, -alkylene-N(R⁵)—S(O)—, or -alkylene-N(R⁵)—S(O)₂—;
L² is alkylene, —C(O)—, —S(O)—, —S(O)₂—, or a covalent bond;
R¹, R², R³, R⁴ and R⁵ are each independently hydrogen, substituted or unsubstituted alkyl, substituted or unsubstituted alkenyl, substituted or unsubstituted alkynyl, alkoxy, alkoxyalkyl, alkoxyacyl, haloalkyl, cyanoalkyl, hydroxyalkyl, thioalkyl, alkylthioalkyl, substituted or unsubstituted aminoalkyl, substituted or unsubstituted aminoacylalkyl, substituted or unsubstituted amino, substituted or unsubstituted arylamino, substituted or unsubstituted heteroarylamino, substituted or unsubstituted arylacyl, substituted or unsubstituted heteroarylacyl, substituted or unsubstituted aminoacyl, substituted or unsubstituted arylalkyl, substituted or unsubstituted heteroarylalkyl, substituted or unsubstituted heterocyclylalkyl, substituted or unsubstituted aryl, substituted or unsubstituted heteroaryl, substituted or unsubstituted cycloalkyl, substituted or unsubstituted cycloalkylalkyl, substituted or unsubstituted heterocyclyl, or —S(O)₂—R^{z}, wherein R^{z} is selected from the group consisting of substituted or unsubstituted alkyl, substituted or unsubstituted arylalkyl, substituted or unsubstituted heteroarylalkyl, substituted or unsubstituted aryl, or substituted or unsubstituted heteroaryl; or
R¹ and R², taken together with the nitrogen atom to which they are both shown attached, form a substituted or unsubstituted 5- to 18-membered saturated heterocyclic ring containing at least one nitrogen atom; or
R³ and R⁴, taken together with the nitrogen atom to which they are both shown attached, form a substituted or unsubstituted 5- to 18-membered saturated heterocyclic ring containing at least one nitrogen atom;
X and Y are independently hydrogen, halogen, —CN, —OR^{x}, —N(R^{x}R^{y}), —SR^{x}, acyl, alkyl, alkoxyalkyl, haloalkyl, cyanoalkyl, hydroxyalkyl, aminoalkyl, thioalkyl, N-alkylaminoalkyl, N,N-dialkylaminoalkyl, alkylthioalkyl, —S(O)—R^{x}, —S(O)₂—R^{x}, —S(O)₂—N(R^{x}R^{y}), N-acylamino, —C(O)—R^{x}, —C(O)₂—R^{x}, and —C(O)₂—N(R^{x}R^{y}); wherein R^{x} and R^{y} are independently hydrogen, substituted or unsubstituted alkyl, substituted or unsubstituted arylalkyl, substituted or unsubstituted heteroarylalkyl, substituted or unsubstituted aryl, or substituted or unsubstituted heteroaryl;
with the following provisos:
(i) R¹ and R² are not both hydrogen;
(ii) when R³ and R⁴ are both hydrogen; then neither R¹ nor R² is hydrogen;
(iii) L¹ and L² are not both —C(O)—;
(iv) when L² is a covalent bond, then L¹ is —C(O)—, —S(O)—, or —S(O)₂—; and
(v) at least one of R¹, R², R³ and R⁴ is substituted or unsubstituted arylalkyl, substituted or unsubstituted heteroarylalkyl, substituted or unsubstituted aryl, or substituted or unsubstituted heteroaryl.

2. The compound of claim 1 having the formula (IA):

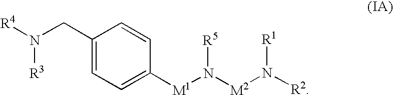

3. The compound of claim 2, wherein:
M¹ is alkylene;
M² is —C(O)— or —S(O)—, or —S(O)₂—; and
R⁵ is selected from the group consisting of H, substituted or unsubstituted alkyl, substituted or unsubstituted aminoalkyl, substituted or unsubstituted arylalkyl, substituted or unsubstituted heterocyclyl, and substituted or unsubstituted heteroarylalkyl.

4. The compound of claim 1 having the formula (IB):

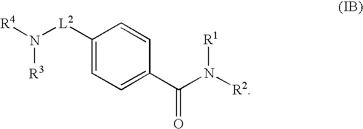

5. The compound of claim 4, wherein:
R¹ is selected from the group consisting of H, substituted or unsubstituted arylalkyl, substituted or unsubstituted heteroarylalkyl, alkyl, and substituted or unsubstituted aminoalkyl;
R² is selected from the group consisting of substituted or unsubstituted arylalkyl, and substituted or unsubstituted heteroarylalkyl;
R³ and R⁴ are each independently selected from the group consisting of H, substituted or unsubstituted arylalkyl, substituted or unsubstituted heteroarylalkyl, alkoxyalkyl, hydroxyalkyl, substituted or unsubstituted aryl, substituted or unsubstituted heteroaryl, substituted or unsubstituted aminoalkyl, substituted or unsubstituted aminoacylalkyl, substituted or unsubstituted aminoalkyl, and substituted or unsubstituted heterocyclylalkyl; or
R³ and R⁴, together with the nitrogen atom to which they are both shown attached, form a substituted or unsubstituted 5- to 18-membered saturated heterocyclic ring containing at least two nitrogen atoms; and
L² is alkylene.

6. The compound of claim 1, having the formula (IC):

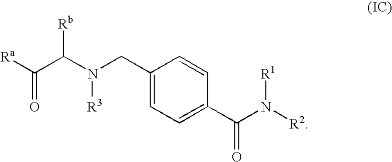

7. The compound of claim 6, wherein:
R^{a} is selected from the group consisting of substituted or unsubstituted amino and substituted or unsubstituted heterocyclyl; and
R^{b} is selected from the group consisting of H, substituted or unsubstituted arylalkyl, substituted or unsubstituted heteroarylalkyl, and aminoalkyl.

8. The compound of claim 1 having the structure ID:

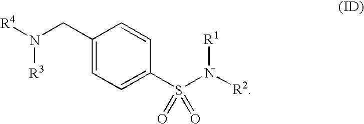

9. The compound of claim 8, wherein:
R¹ is selected from the group consisting of H, substituted or unsubstituted alkyl, alkoxyalkyl, substituted or unsubstituted arylalkyl, substituted or unsubstituted heteroarylalkyl, substituted or unsubstituted cycloalkyl, substituted or unsubstituted aryl, substituted or unsubstituted heteroaryl, and substituted or unsubstituted aminoalkyl;
R² is selected from the group consisting of H, substituted or unsubstituted alkyl, alkoxyalkyl, substituted or unsubstituted aminoalkyl, substituted or unsubstituted arylalkyl, substituted or unsubstituted heteroarylalkyl, substituted or unsubstituted aryl, substituted or unsubstituted heteroaryl, and substituted or unsubstituted cycloalkyl; or
R¹ and R², taken together with the nitrogen atom to which they are both shown attached, form a substituted or unsubstituted 5- to 18-membered saturated heterocyclic ring containing at least one nitrogen atom;
R³ and R⁴ are each independently selected from the group consisting of H, substituted or unsubstituted arylalkyl, substituted or unsubstituted heteroarylalkyl, alkoxyalkyl, hydroxyalkyl, alkyl, substituted or unsubstituted aminoacyl, substituted or unsubstituted aminoacylalkyl, substituted or unsubstituted aminoalkyl, substituted or unsubstituted cycloalkyl, substituted or unsubstituted aryl, substituted or unsubstituted heteroaryl, substituted or unsubstituted arylacyl, substituted or unsubstituted heteroarylacyl, and heterocyclylalkyl; or
R³ and R⁴, taken together with the nitrogen atom to which they are both shown attached, form a substituted or unsubstituted 5- to 18-membered saturated heterocyclic ring containing at least one nitrogen atom.

10. The compound of claim 1, having the structure (IE):

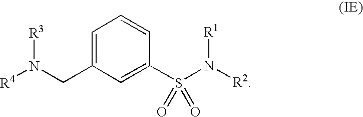

11. The compound of claim 10, wherein:
R¹ and R² are each independently selected from the group consisting of H, substituted or unsubstituted alkyl, substituted or unsubstituted arylalkyl, substituted or unsubstituted heteroarylalkyl, substituted or unsubstituted cycloalkyl, substituted or unsubstituted aryl, substituted or unsubstituted heteroaryl, and substituted or unsubstituted aminoalkyl; or
R¹ and R², taken together with the nitrogen atom to which they are both shown attached, form a substituted or unsubstituted 5- to 18-membered saturated heterocyclic ring containing at least one nitrogen atom; and
R³ and R⁴ are each independently selected from the group consisting of H, acyl, substituted or unsubstituted arylalkyl, substituted or unsubstituted heteroarylalkyl, substituted or unsubstituted heteroaryl, substituted or unsubstituted alkyl, amino, substituted or unsubstituted aminoalkyl, substituted or unsubstituted cycloalkyl; or
R³ and R⁴, taken together with the nitrogen atom to which they are both shown attached, form a substituted or unsubstituted 5- to 18-membered saturated heterocyclic ring containing at least one nitrogen atom.

12. The compound of claim 1, having the structure (IF):

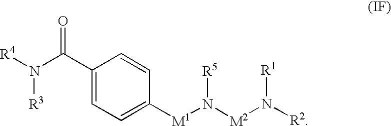

13. The compound of claim 12, wherein:
M¹ is alkylene;
M² is C(O);
R¹ and R² are each independently selected from the group consisting of H, substituted or unsubstituted alkyl, substituted or unsubstituted arylalkyl, and substituted or unsubstituted heteroarylalkyl;
R⁵ is selected from the group consisting of substituted or unsubstituted alkyl, substituted or unsubstituted aminoalkyl, substituted or unsubstituted cycloalkyl, substituted or unsubstituted heterocycloalkyl; and
R³ and R⁴ are each independently selected from the group consisting of H, alkoxy, and substituted or unsubstituted amino.

14. The compound of claim 1, having the structure (IG):

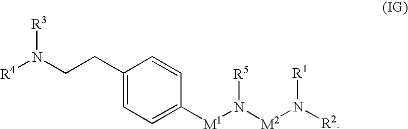

15. The compound of claim 14, wherein:
M¹ is alkylene;
M² is C(O);
R¹ and R² are each independently selected from the group consisting of H, substituted or unsubstituted alkyl, substituted or unsubstituted arylalkyl, and substituted or unsubstituted heteroarylalkyl;
R⁵ is selected from the group consisting of substituted or unsubstituted alkyl, substituted or unsubstituted aminoalkyl, substituted or unsubstituted cycloalkyl, substituted or unsubstituted heterocycloalkyl; and
R³ and R⁴ are each independently selected from the group consisting of H, alkoxycarbonyl, substituted or unsubstituted aryl-S(O)₂—, substituted or unsubstituted arylalkyl, substituted or unsubstituted heteroarylalkyl, substituted or unsubstituted aryl, and substituted or unsubstituted heteroaryl.

16. The compound of claim 1, which is selected from the group consisting of 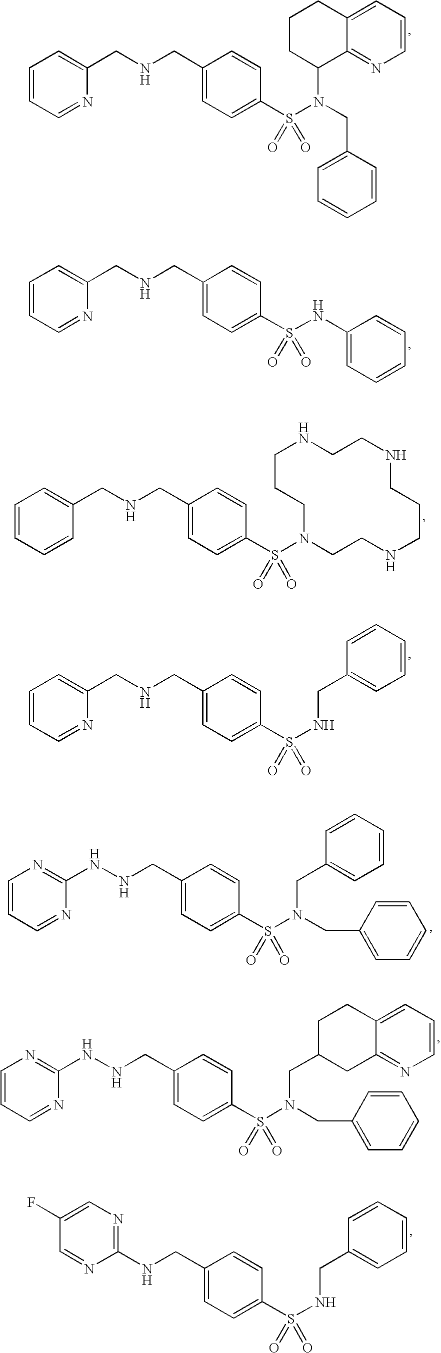 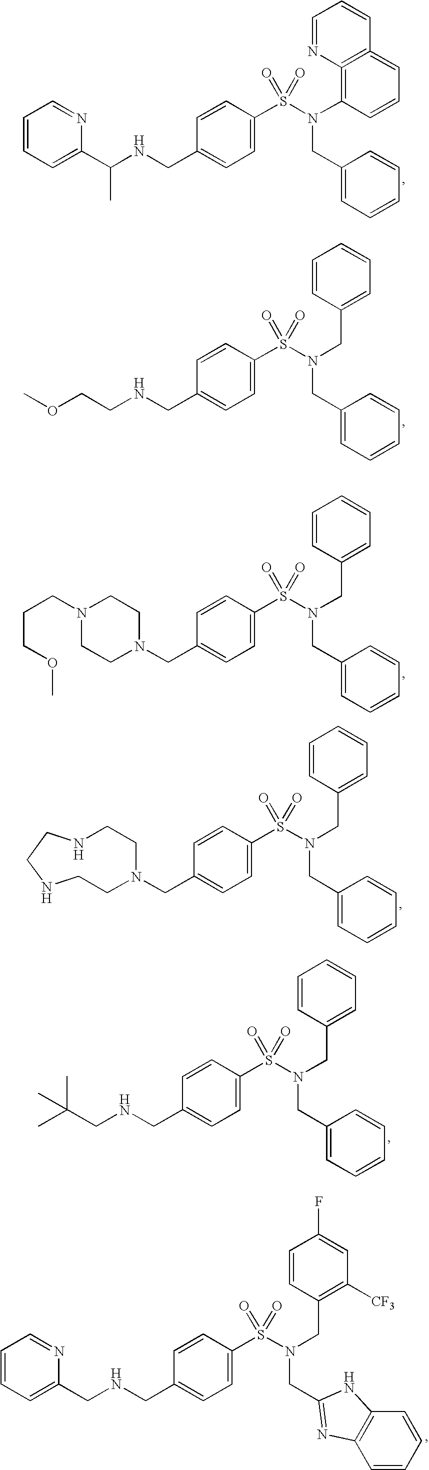 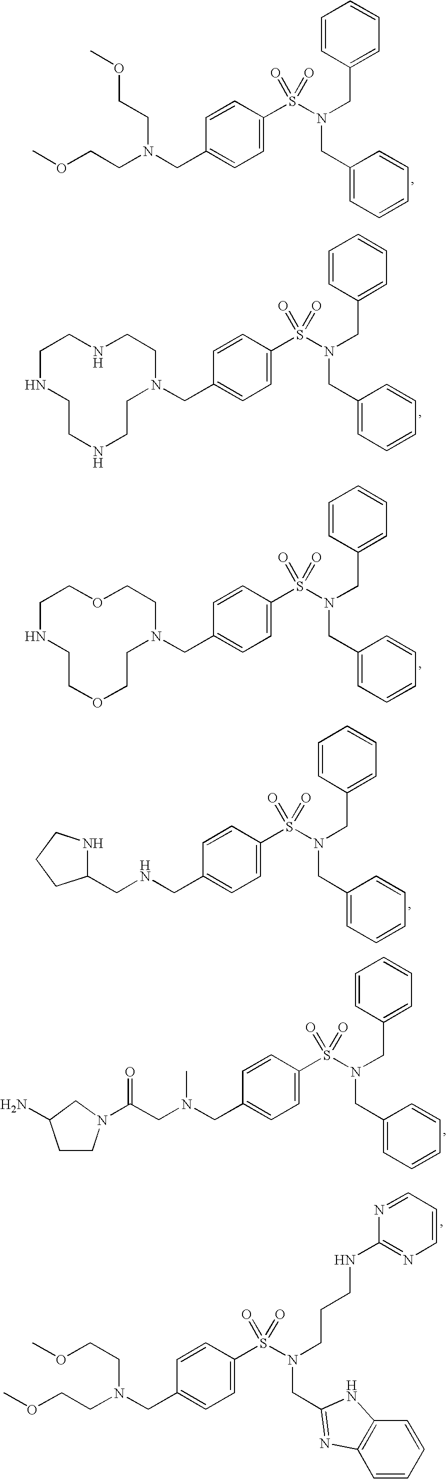 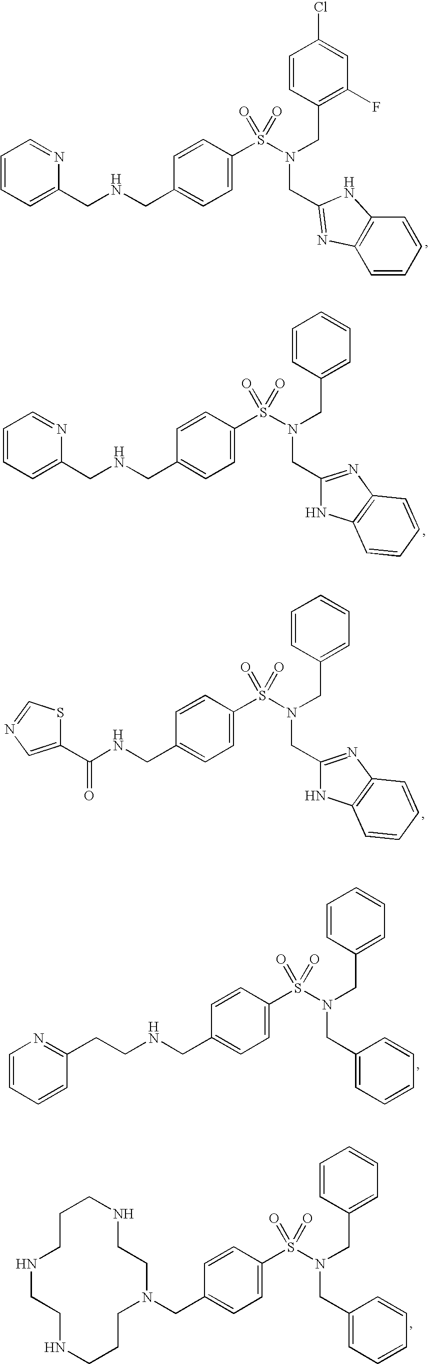 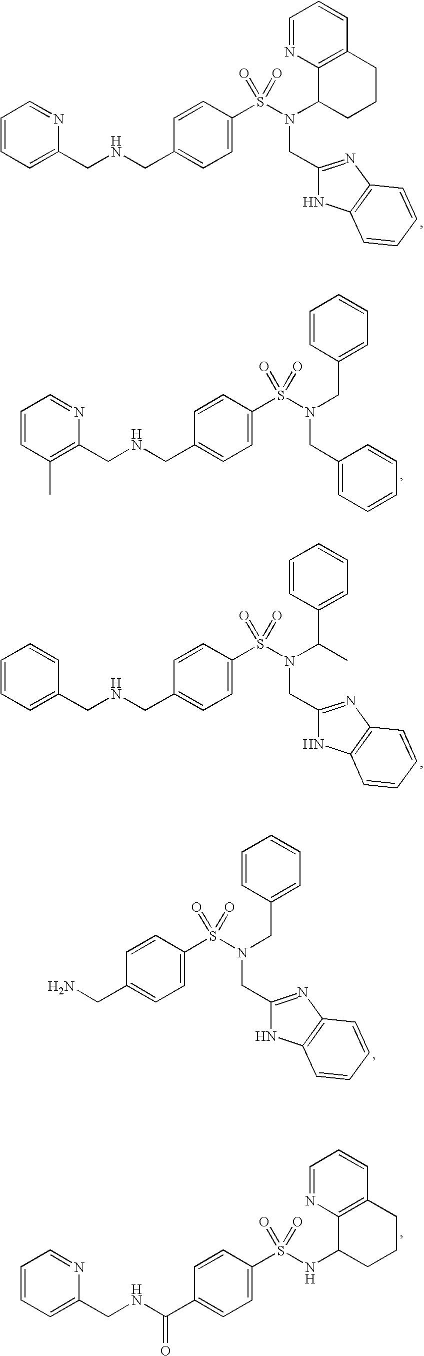 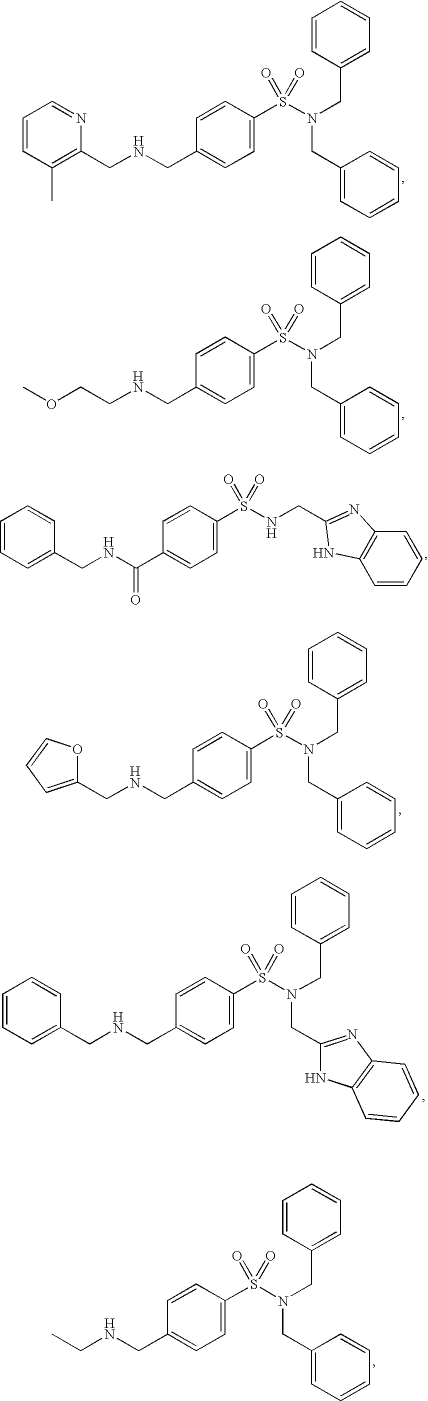 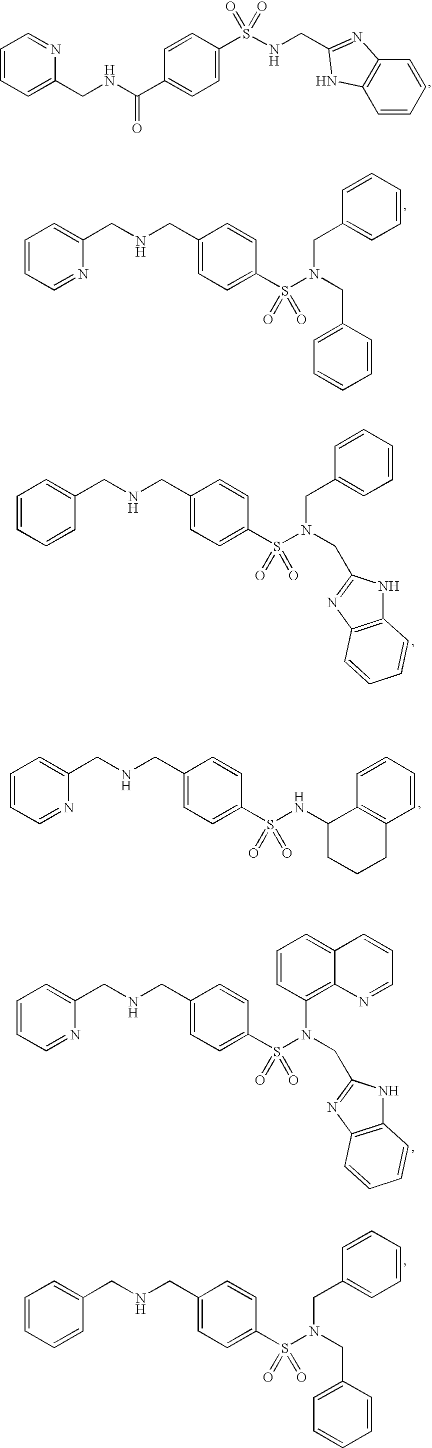 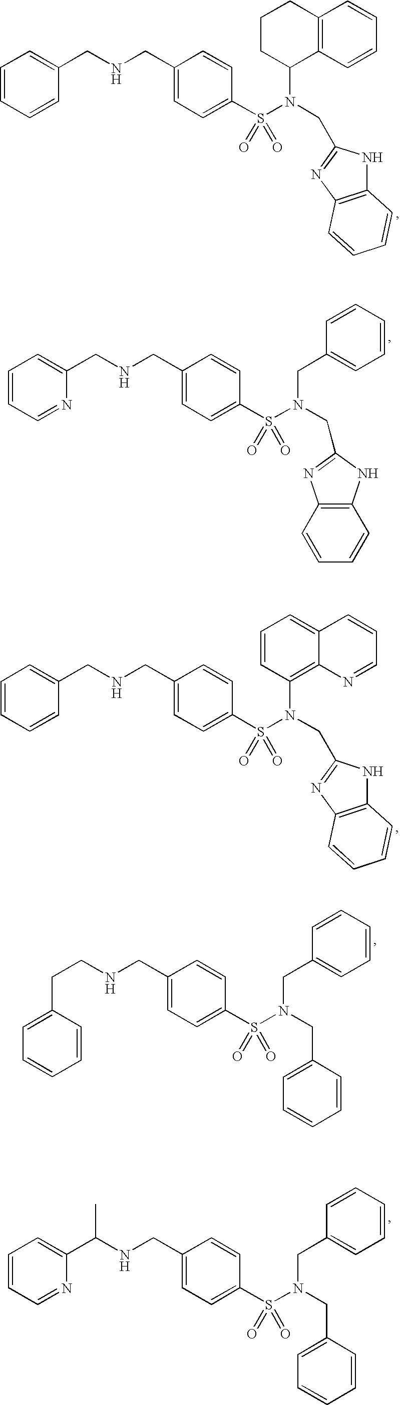 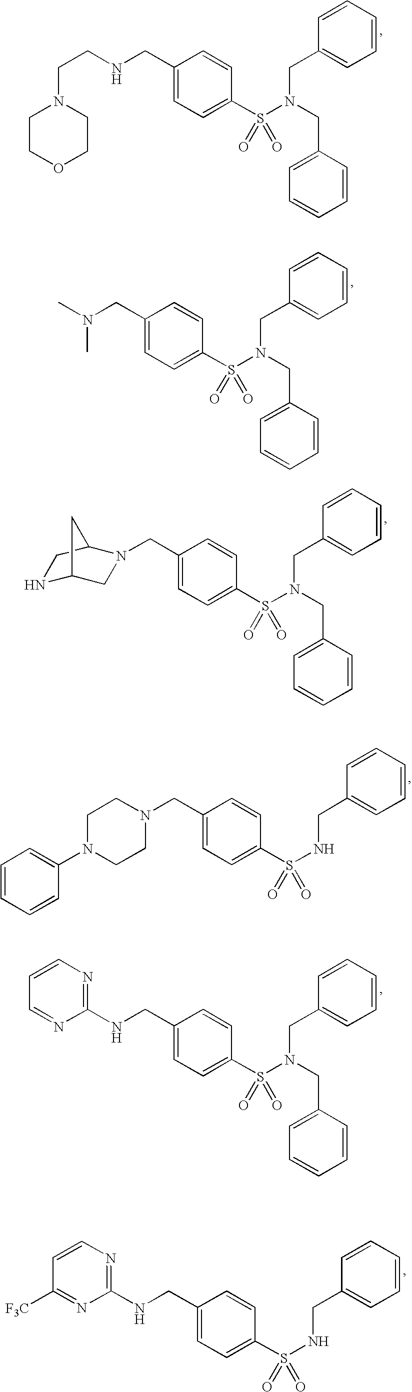 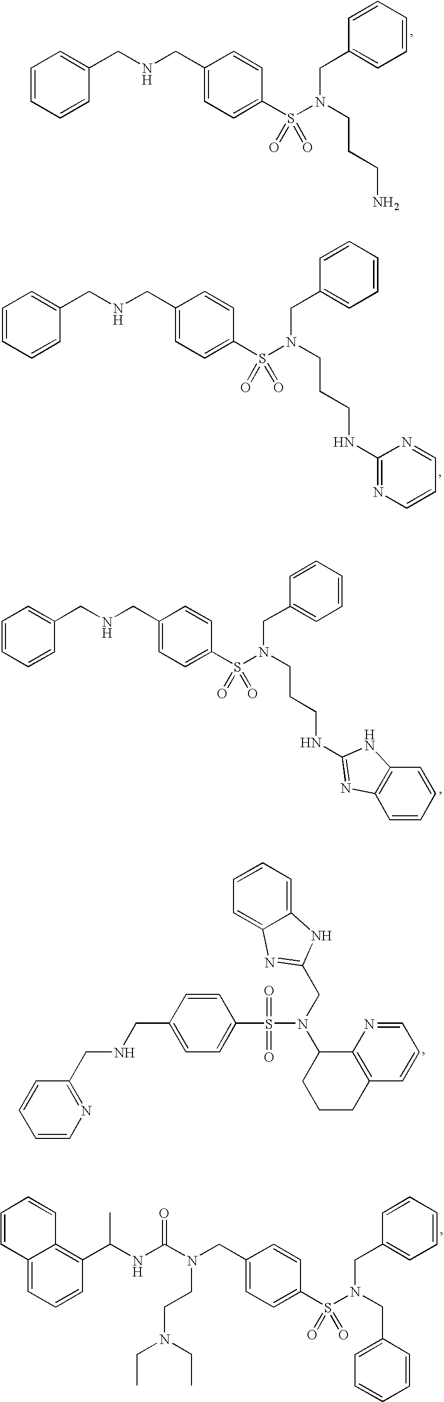 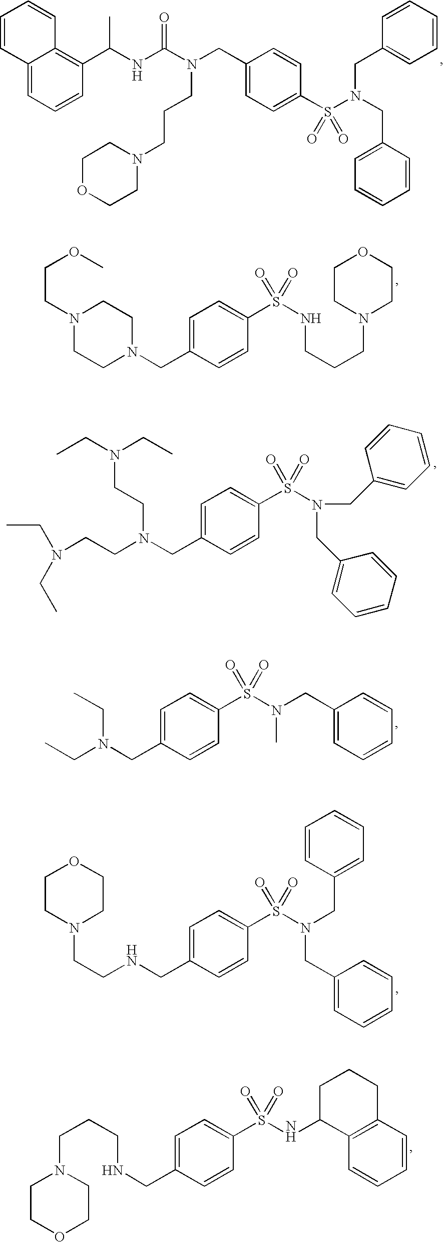 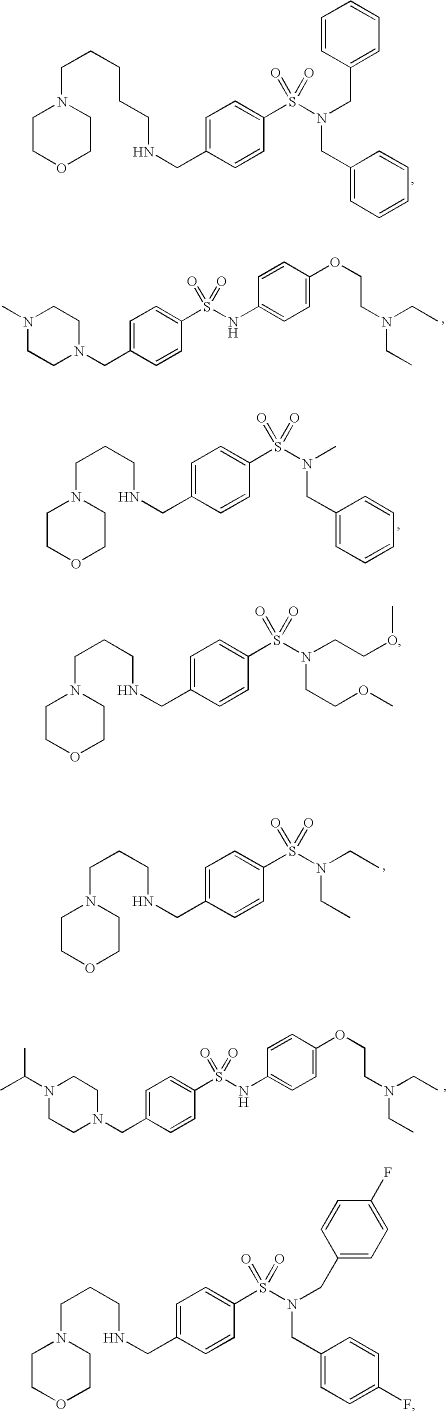 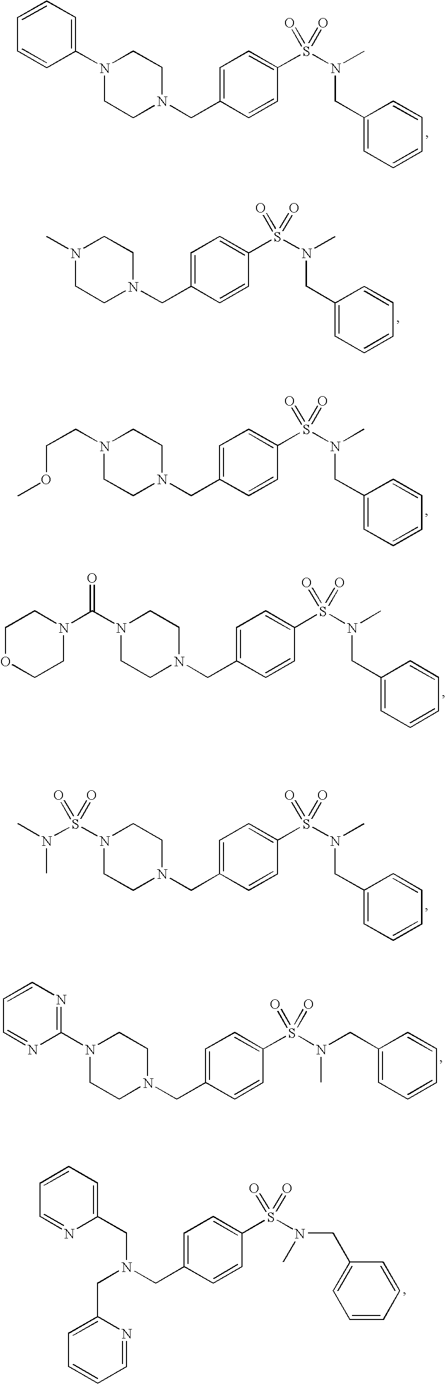 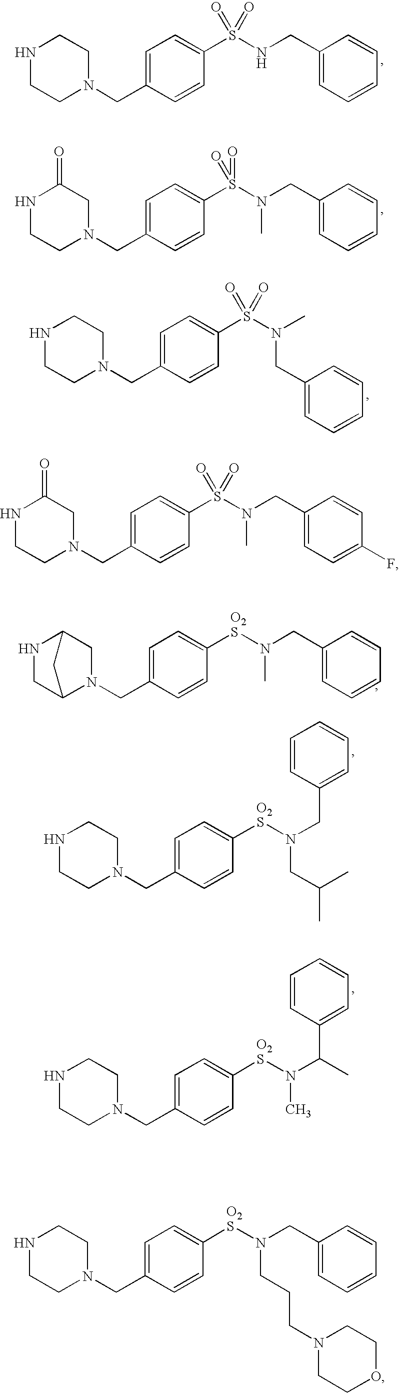 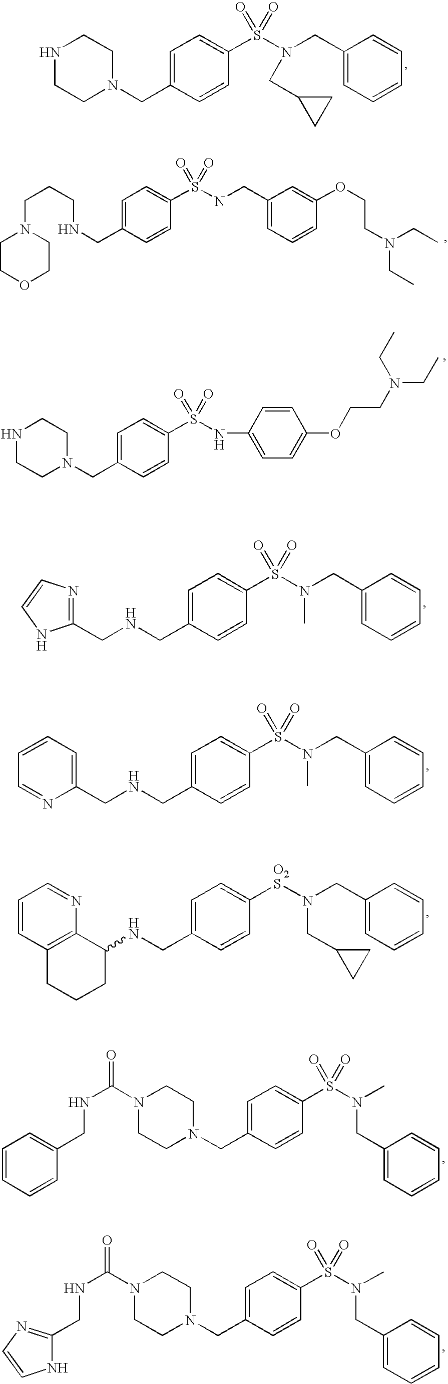 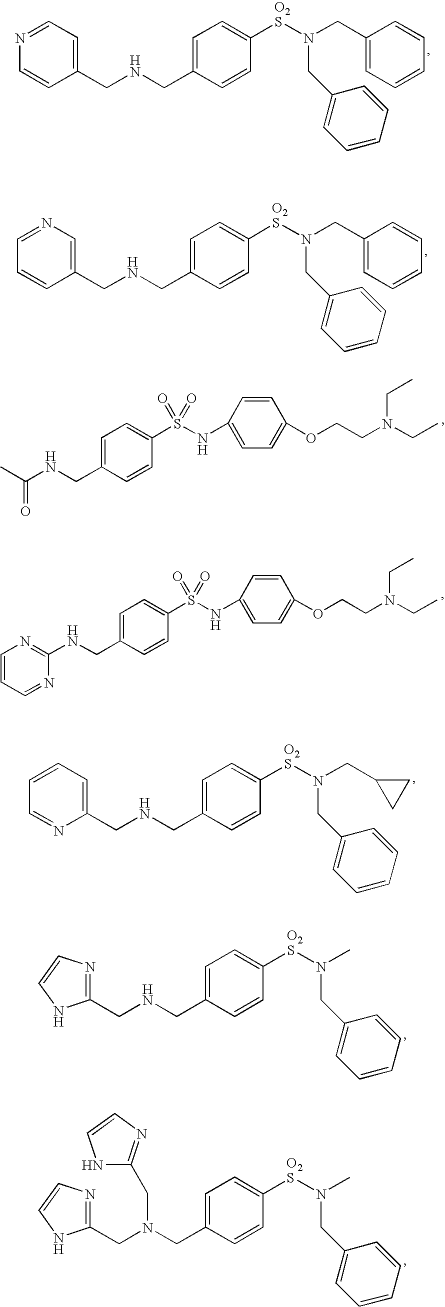 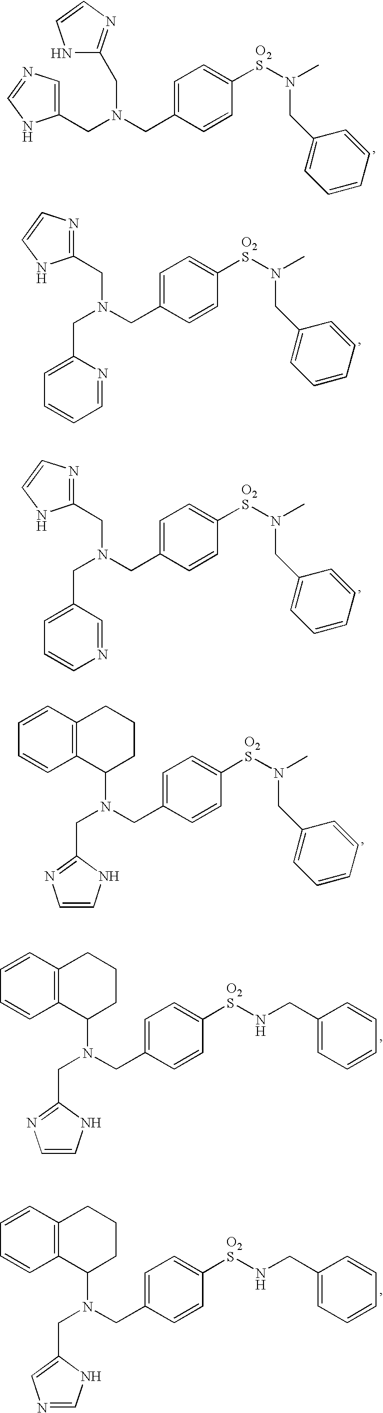 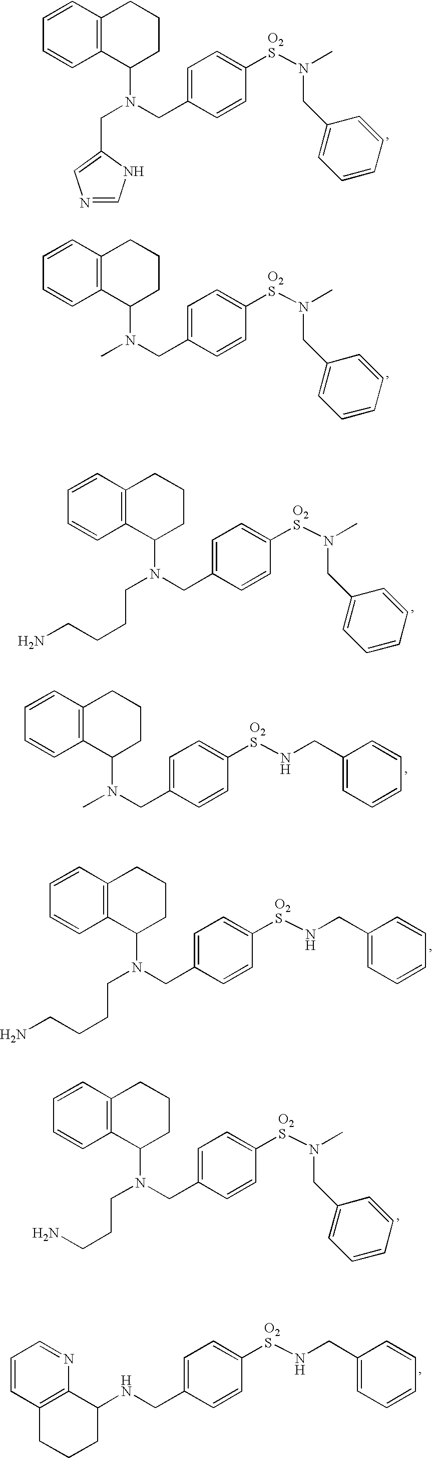 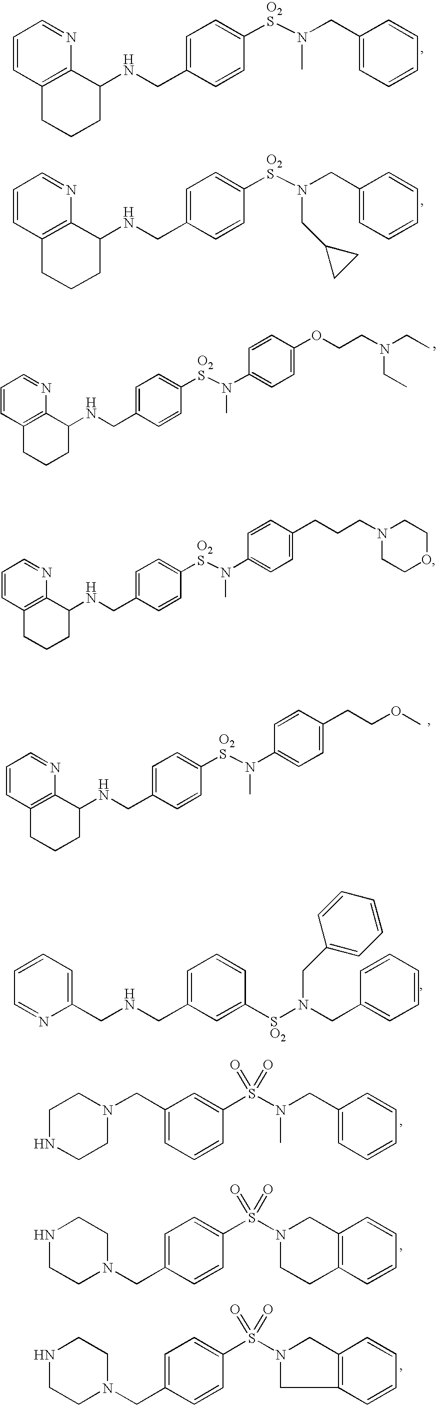 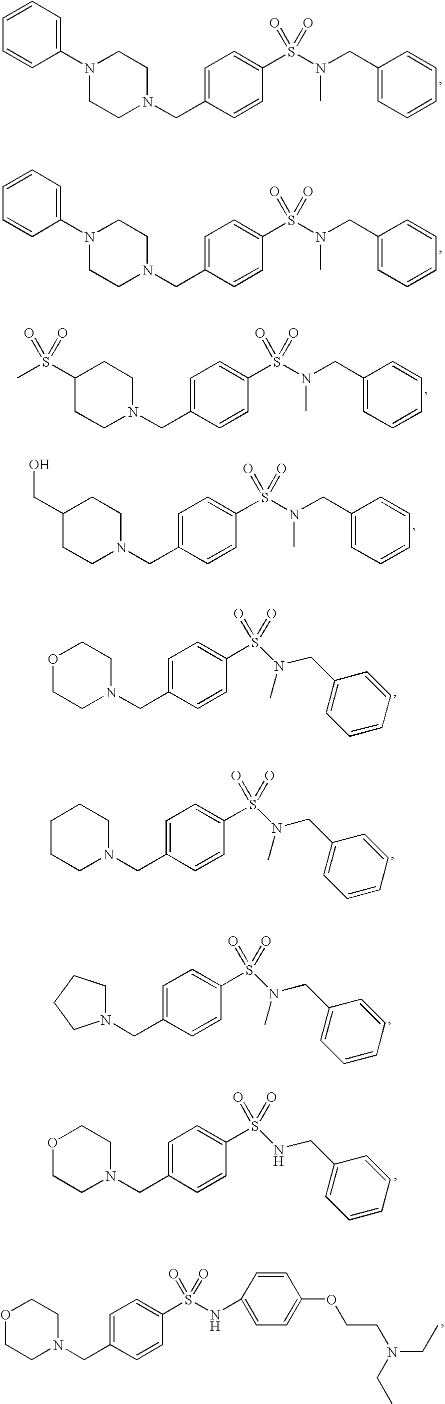 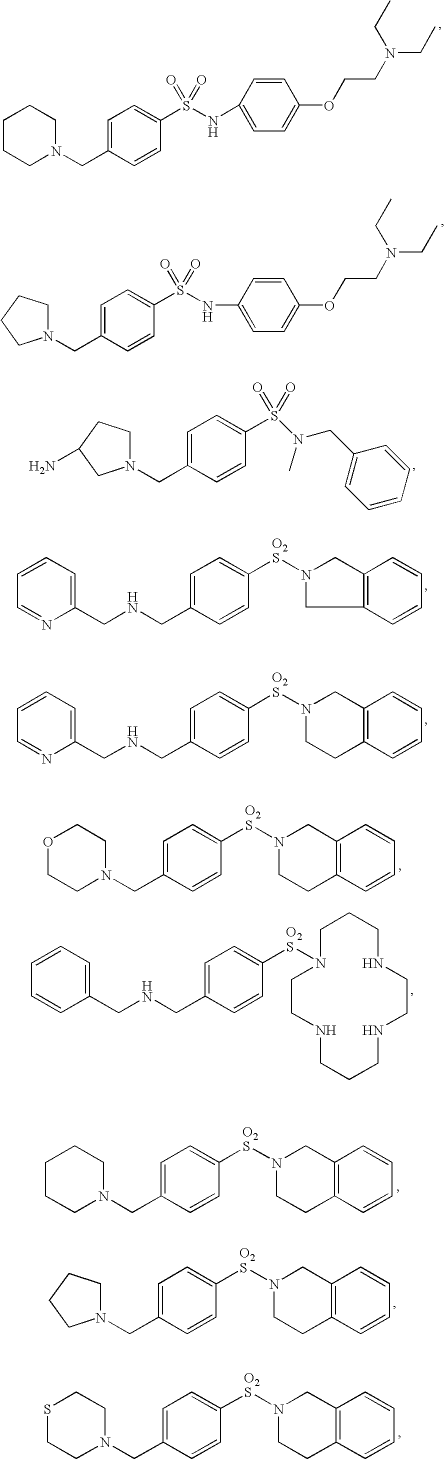 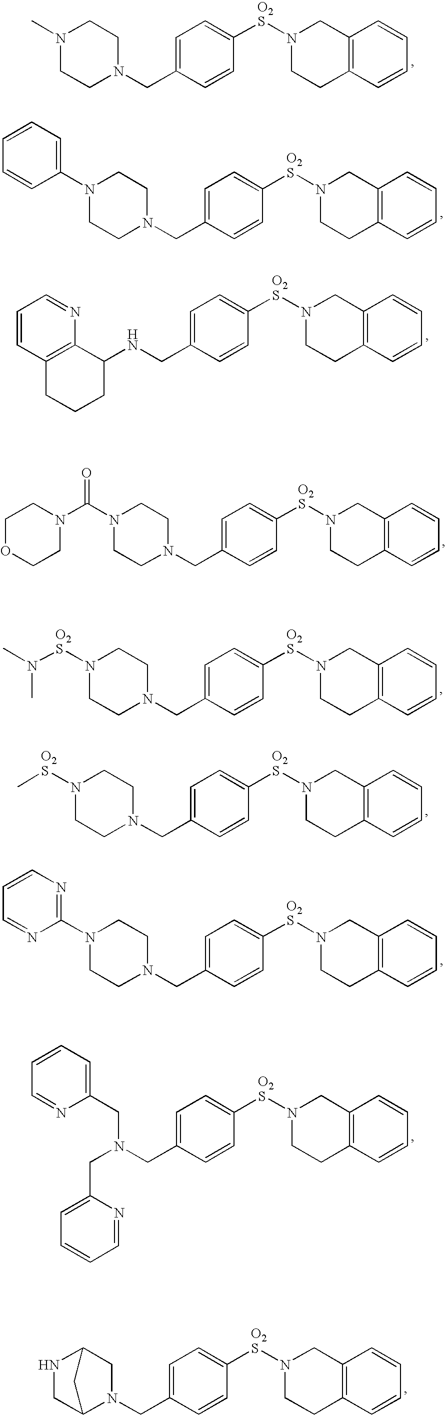 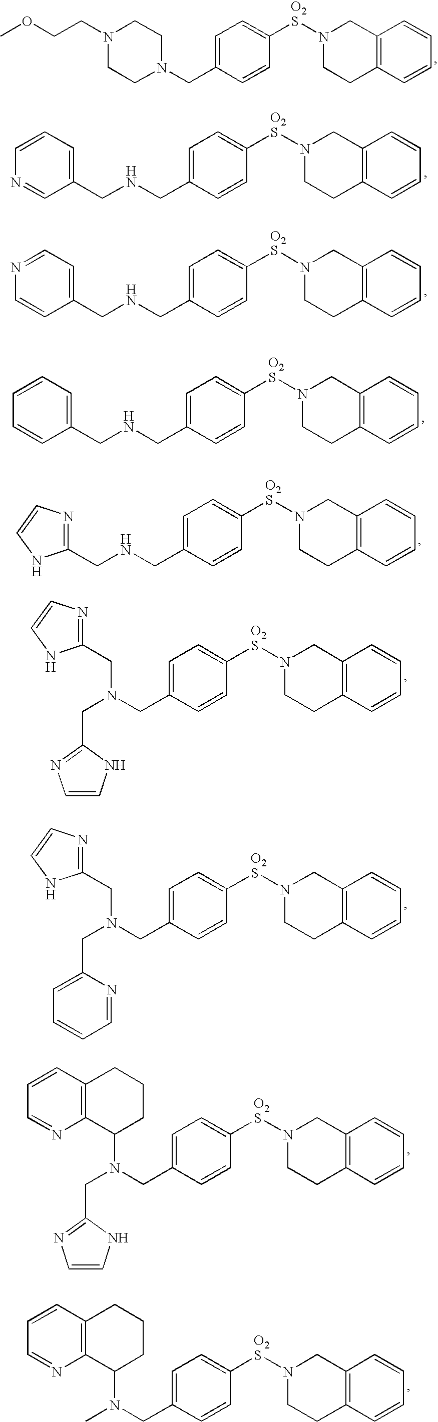 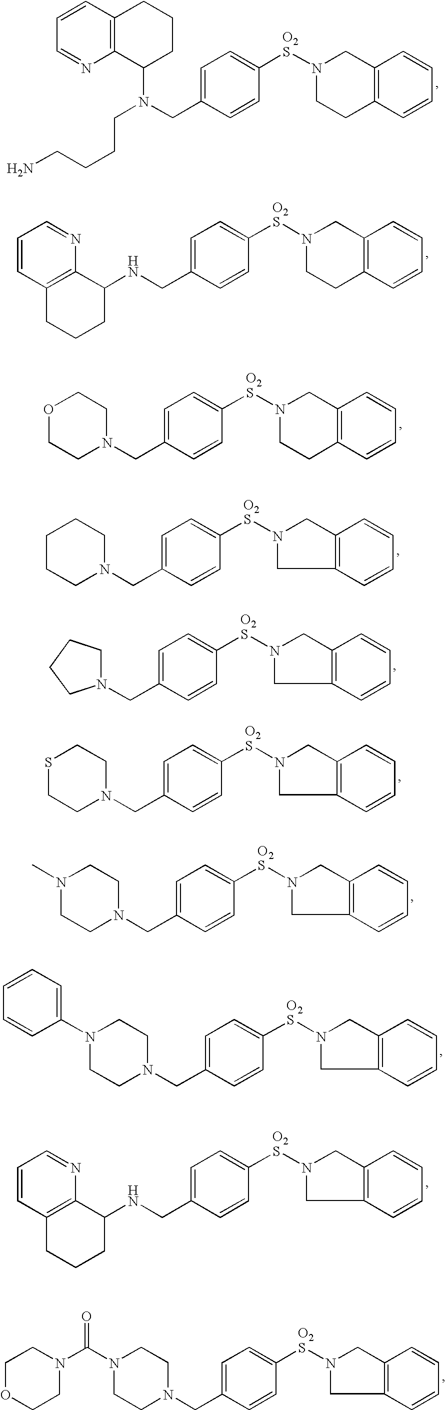 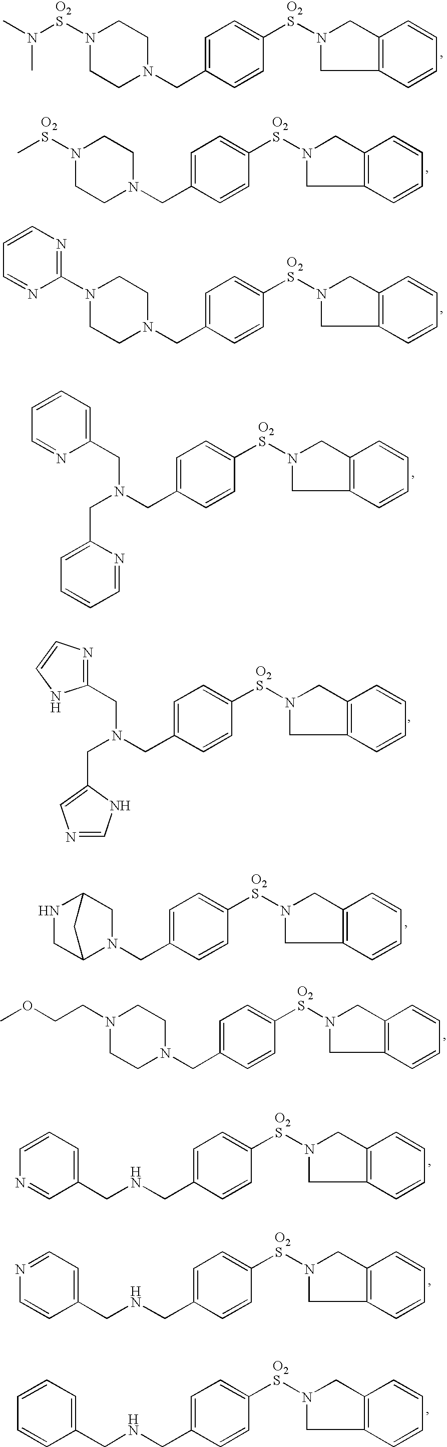 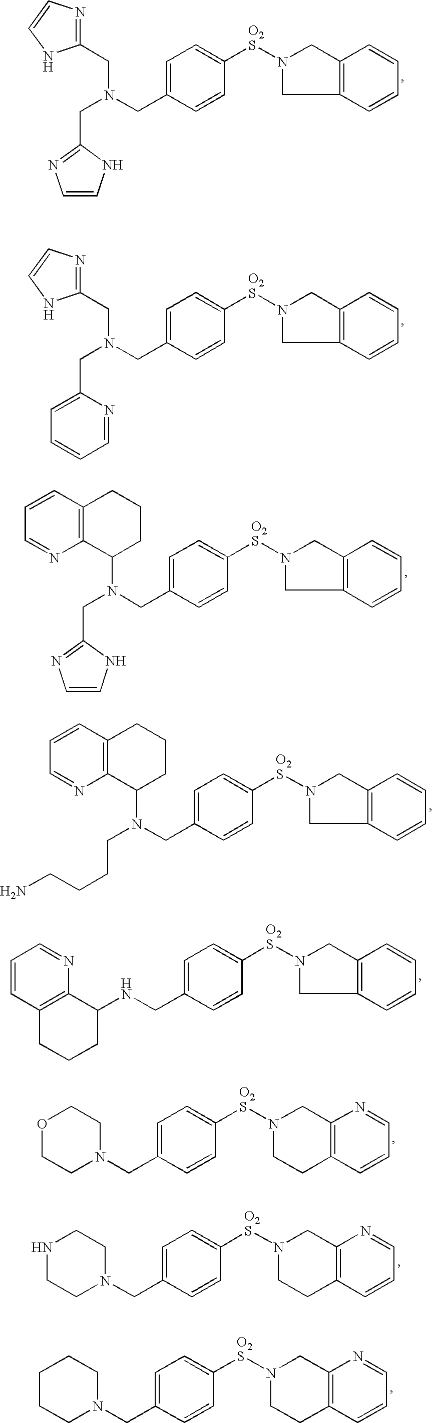 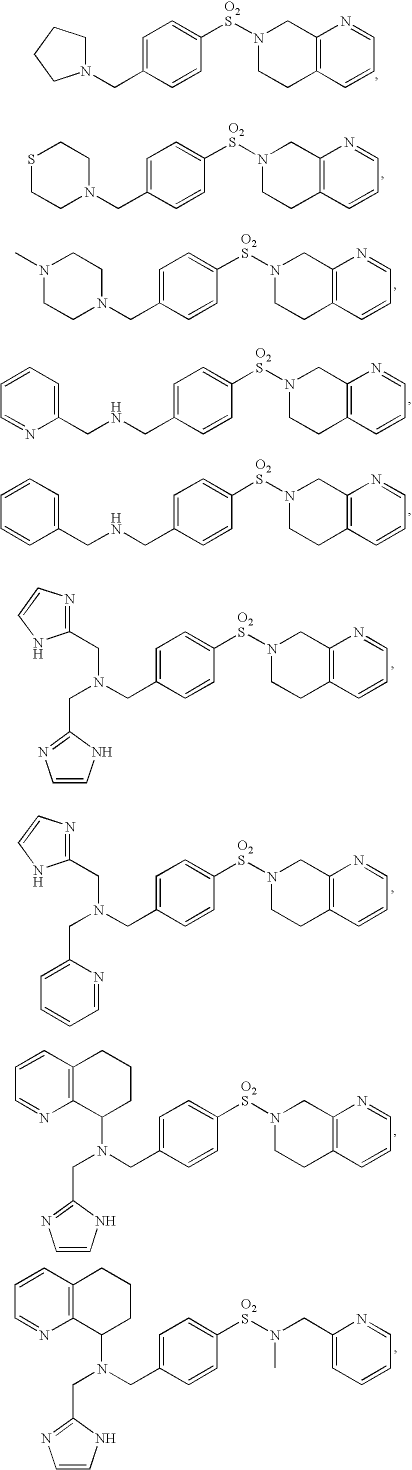 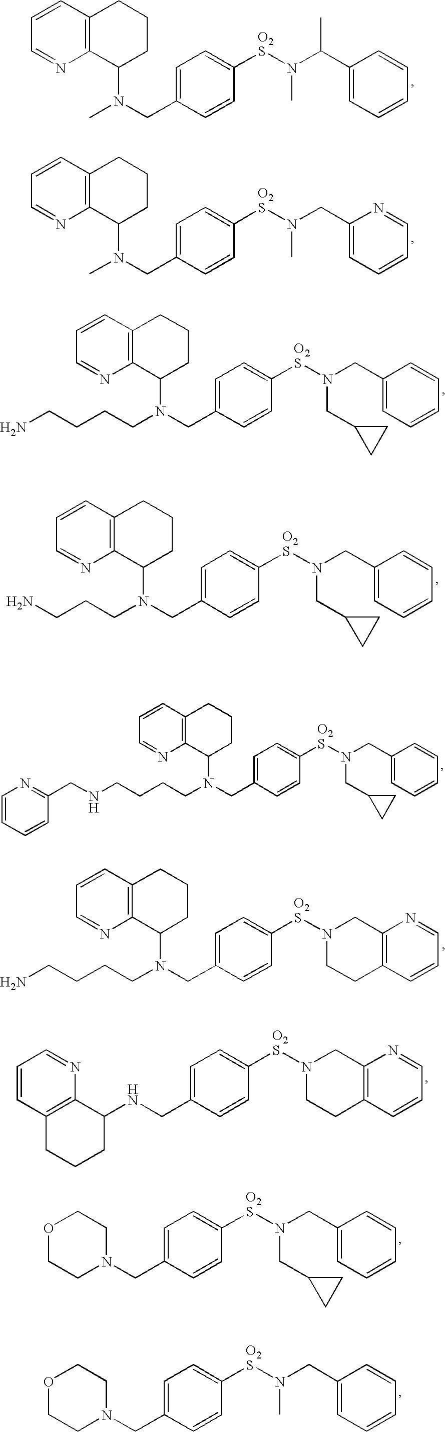 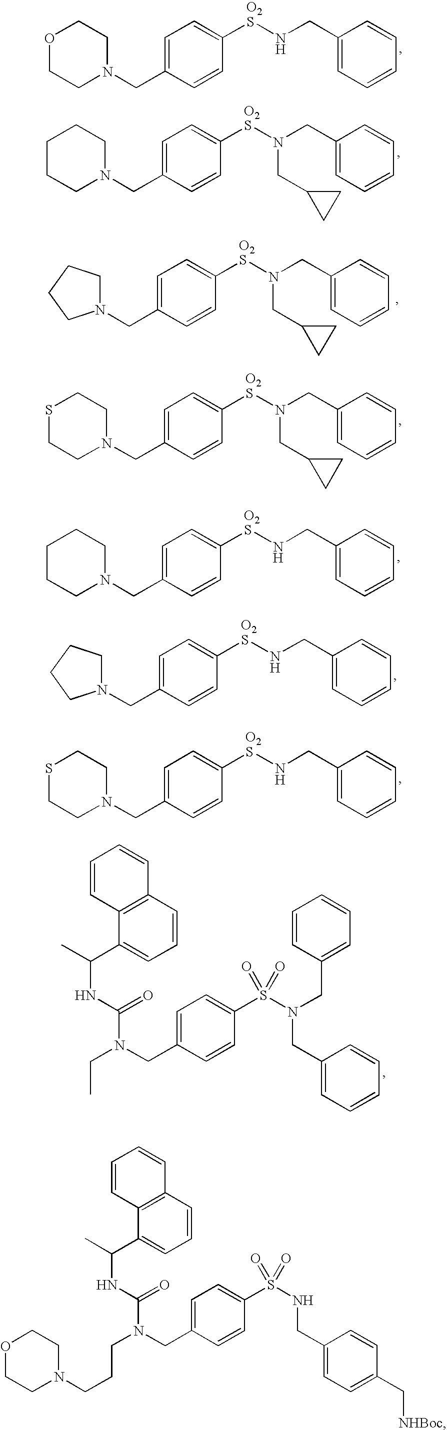 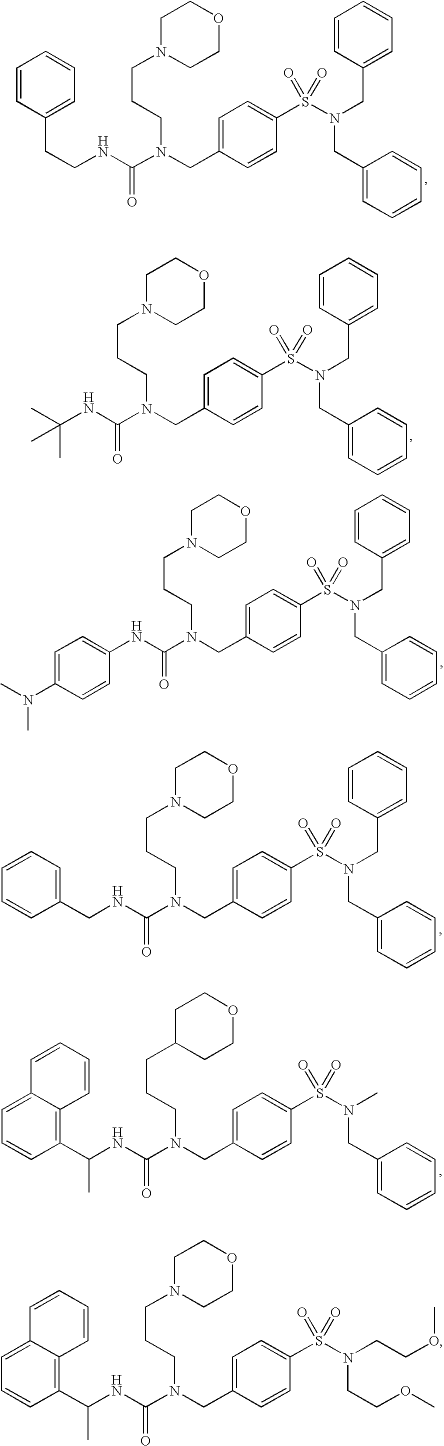 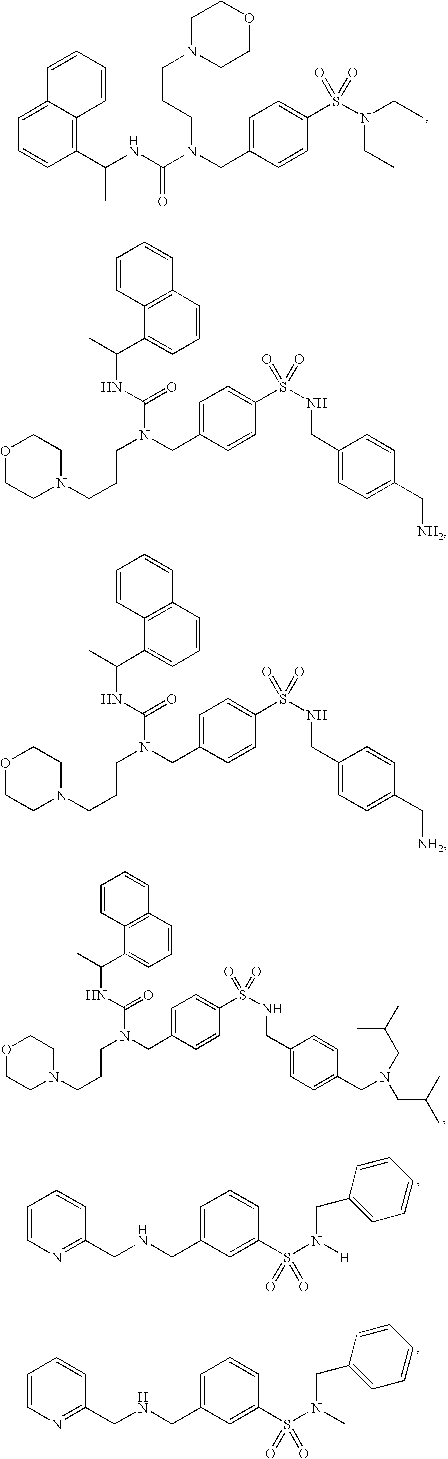 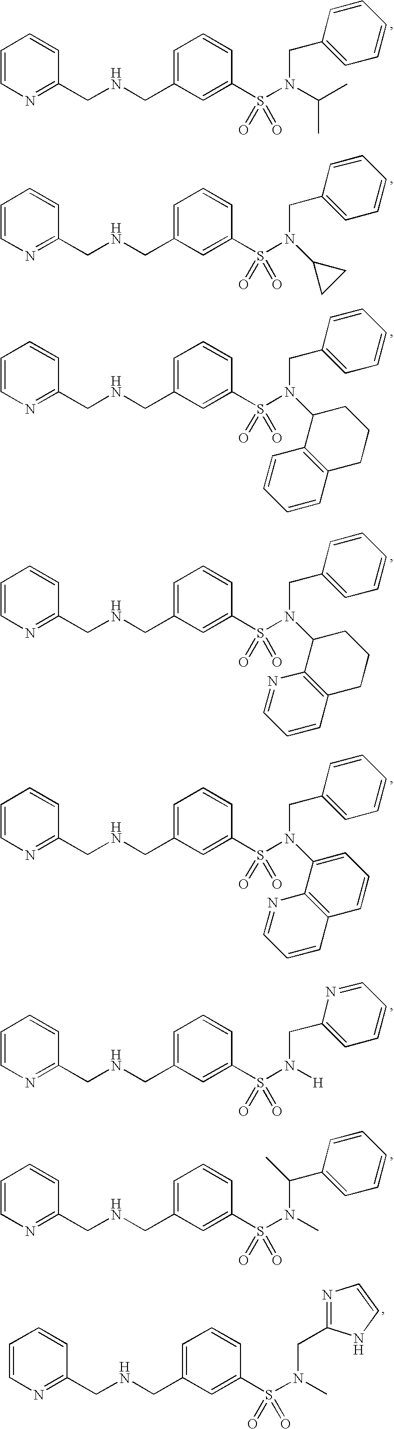 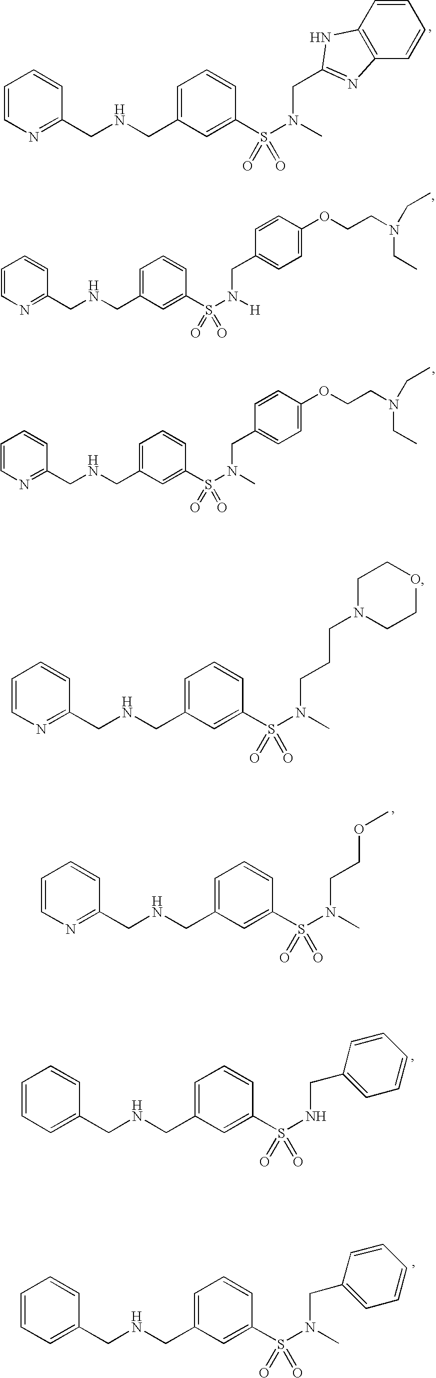 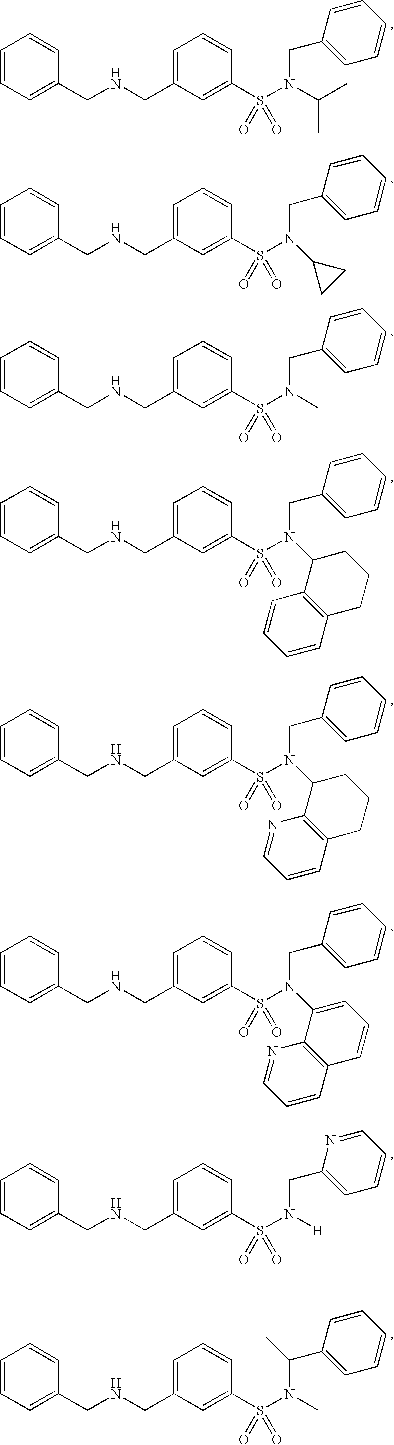 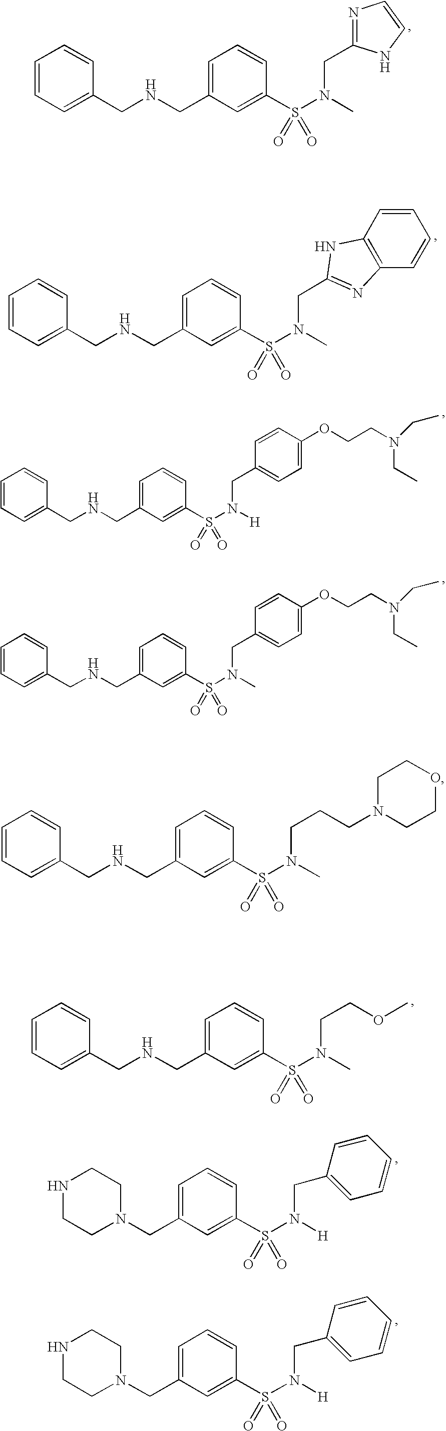 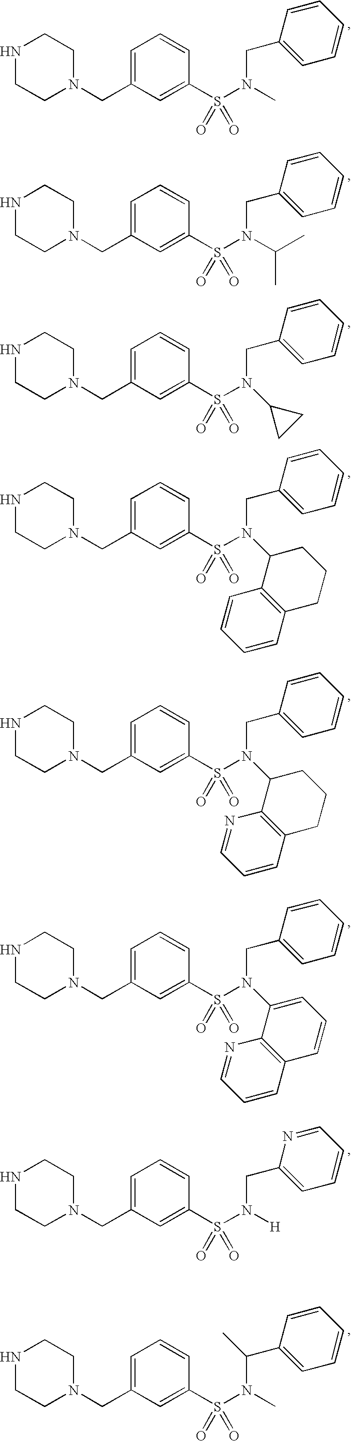 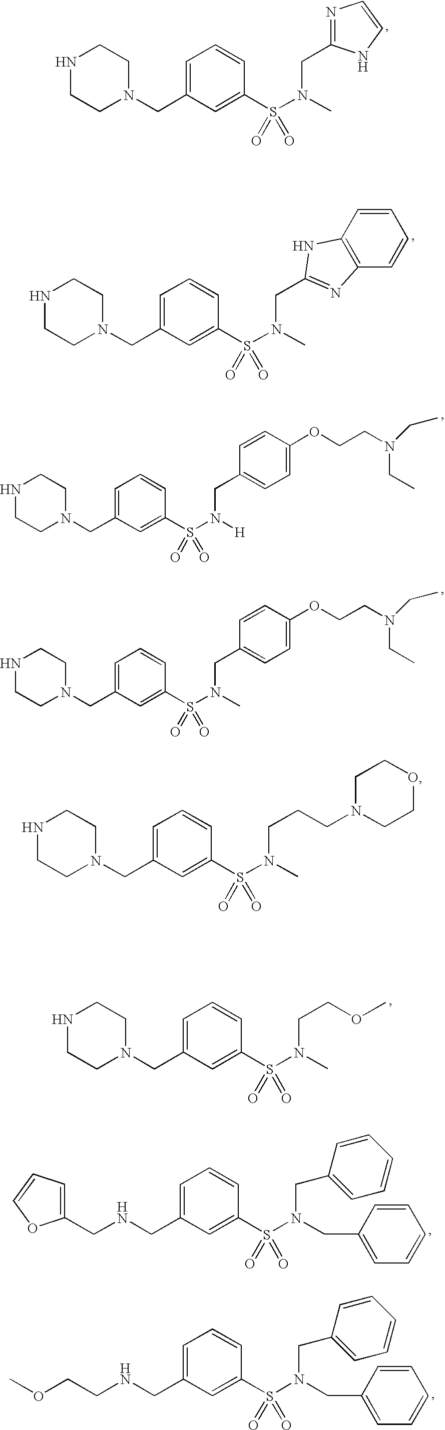 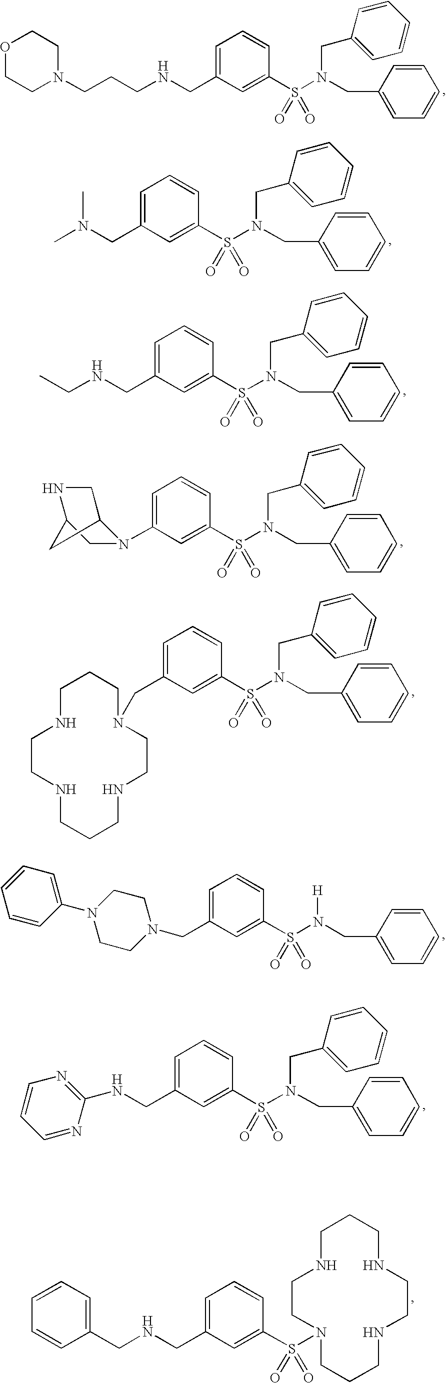 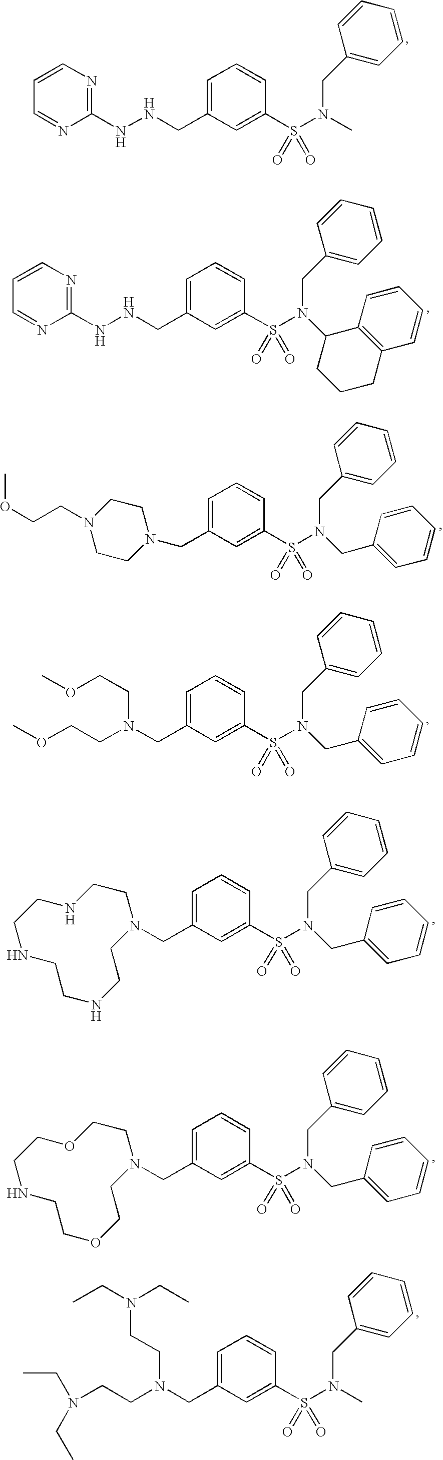 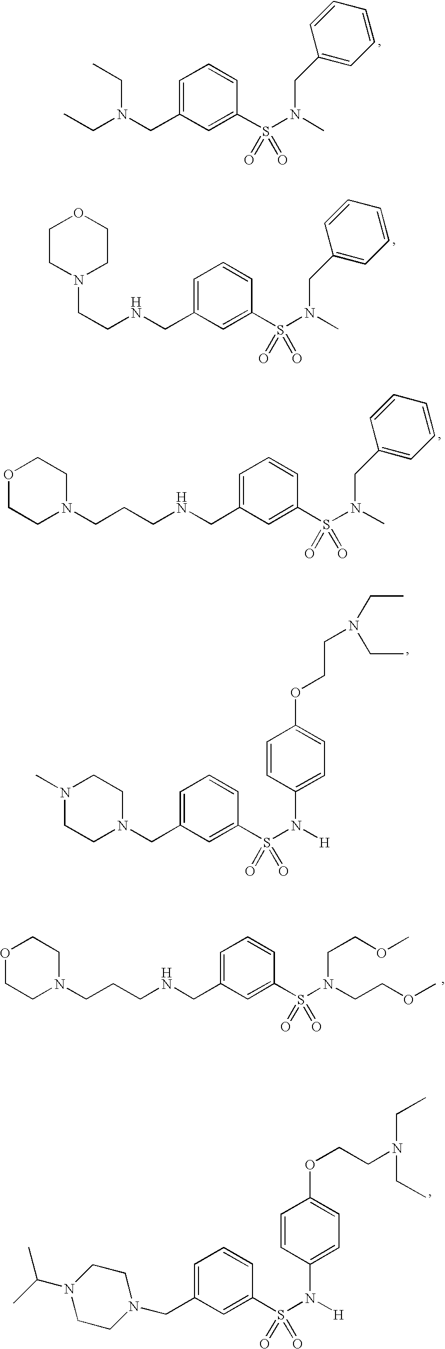 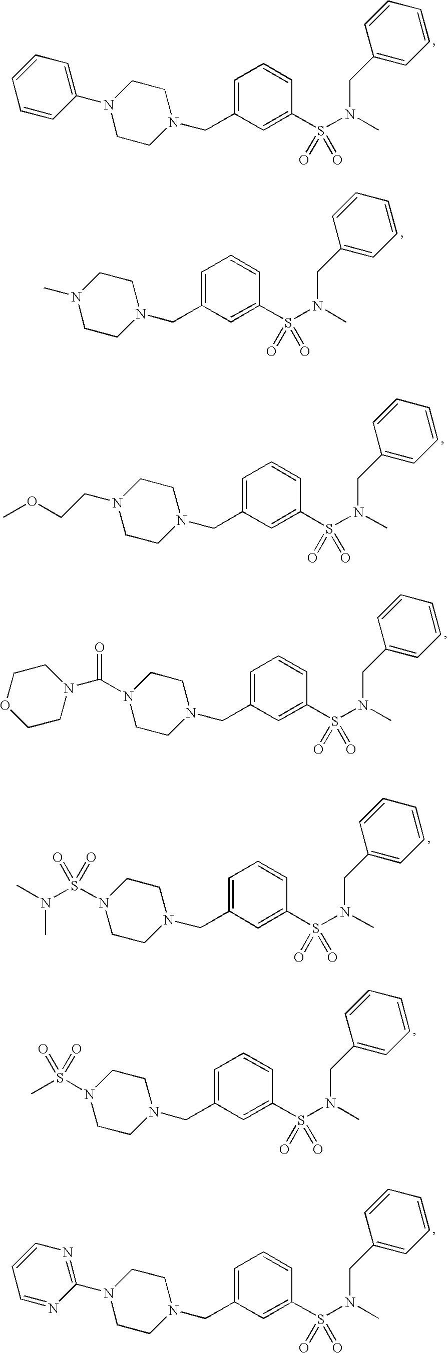 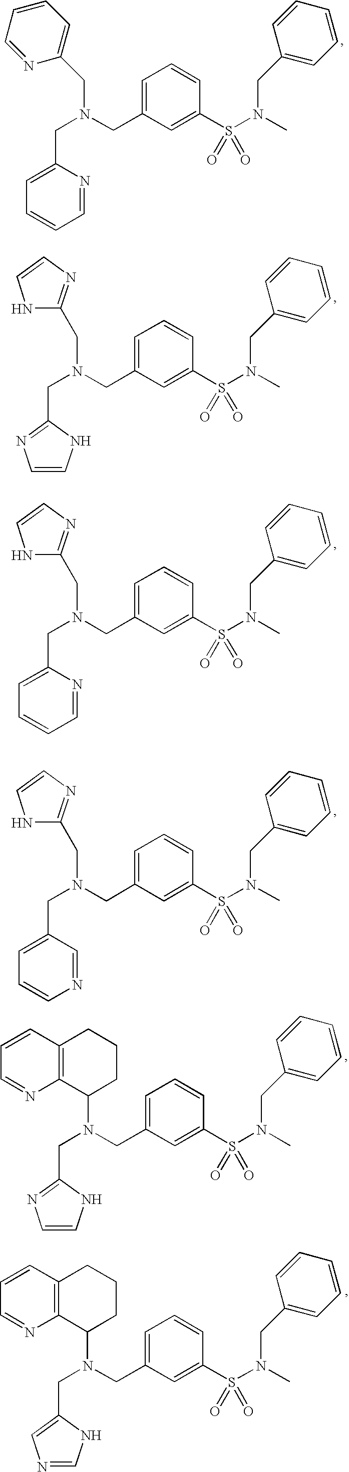 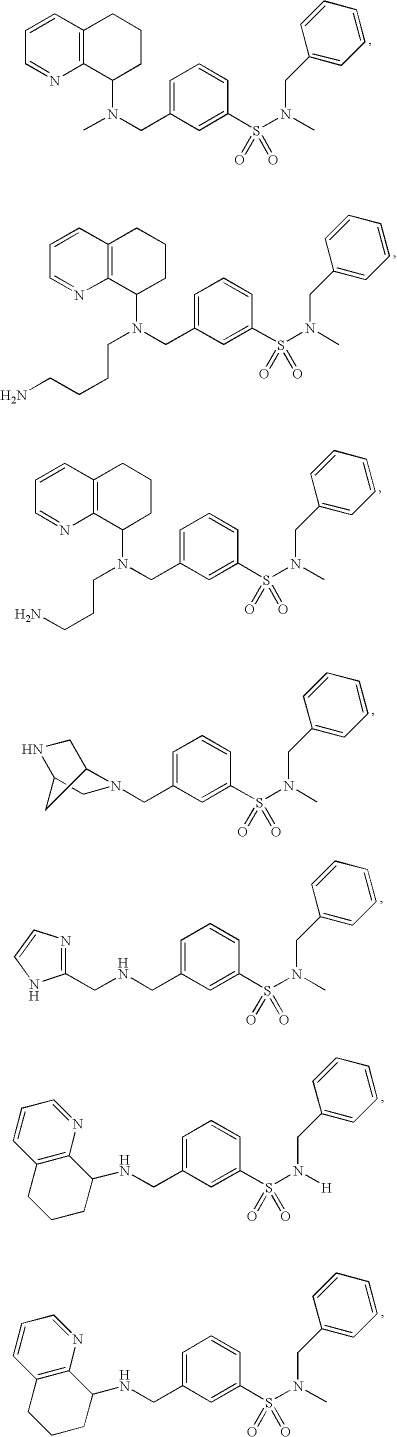 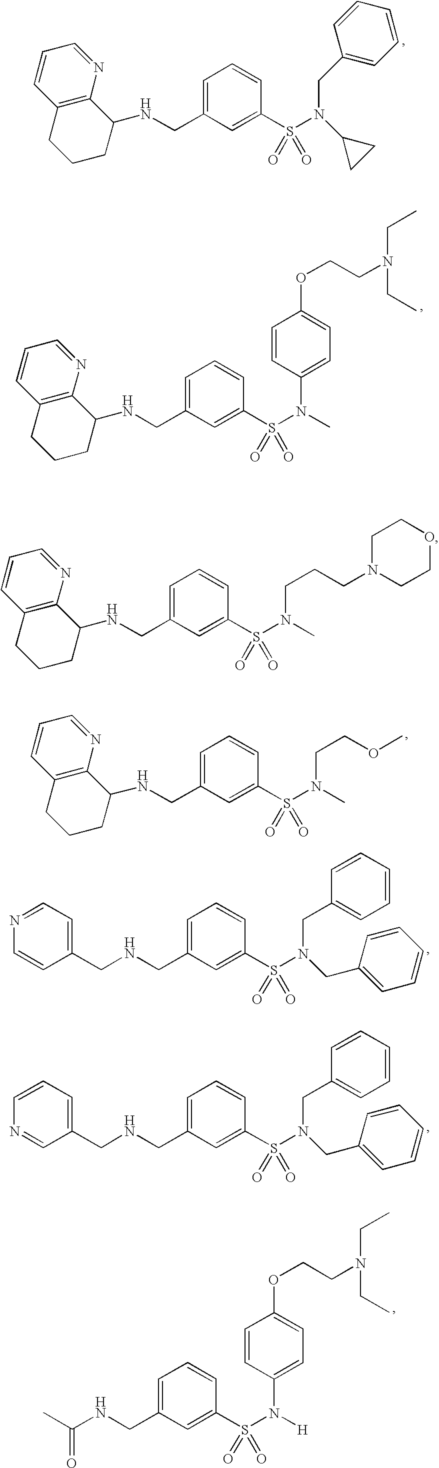 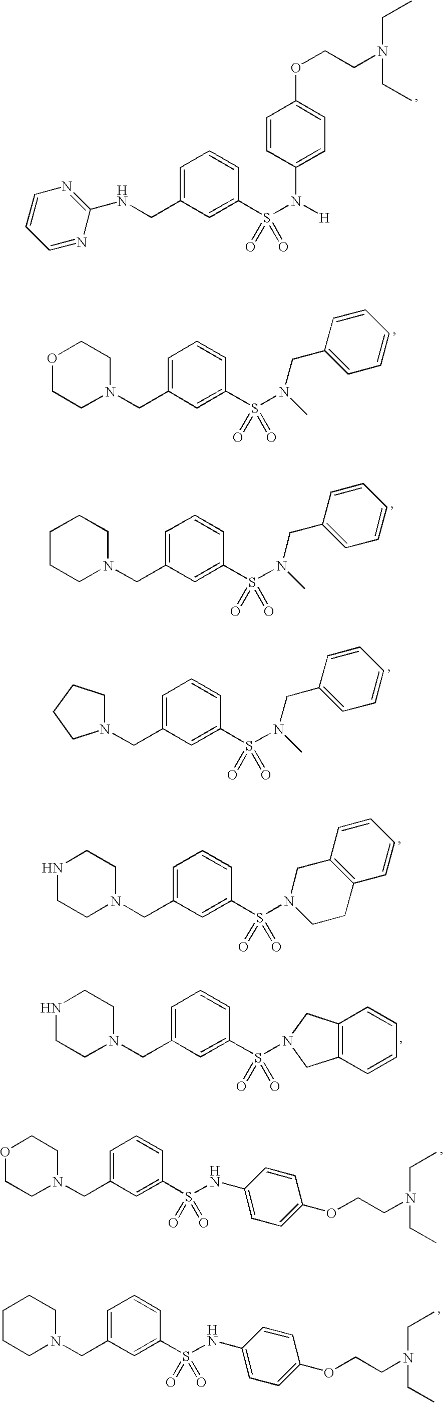 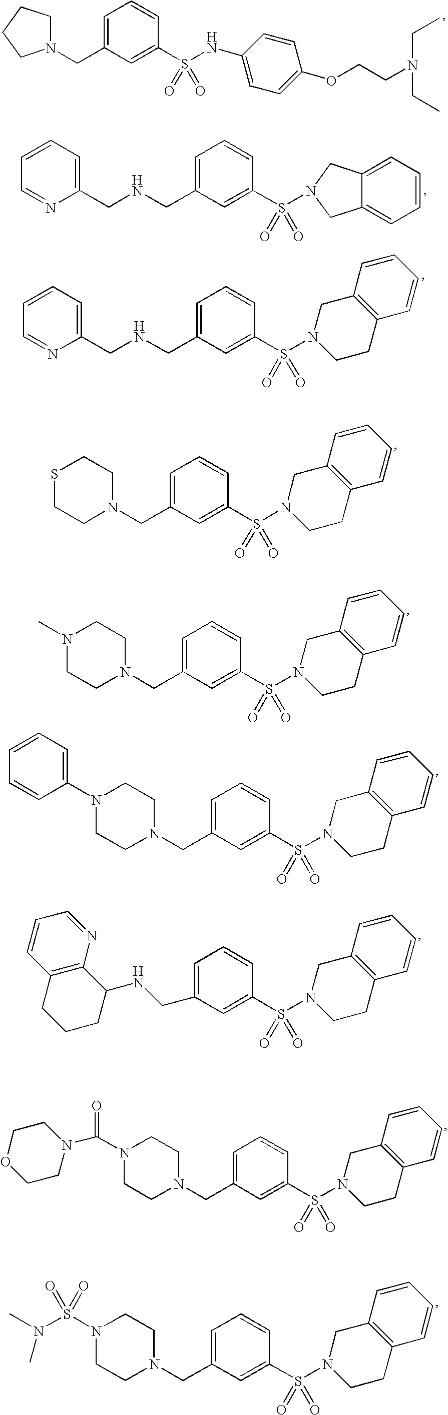 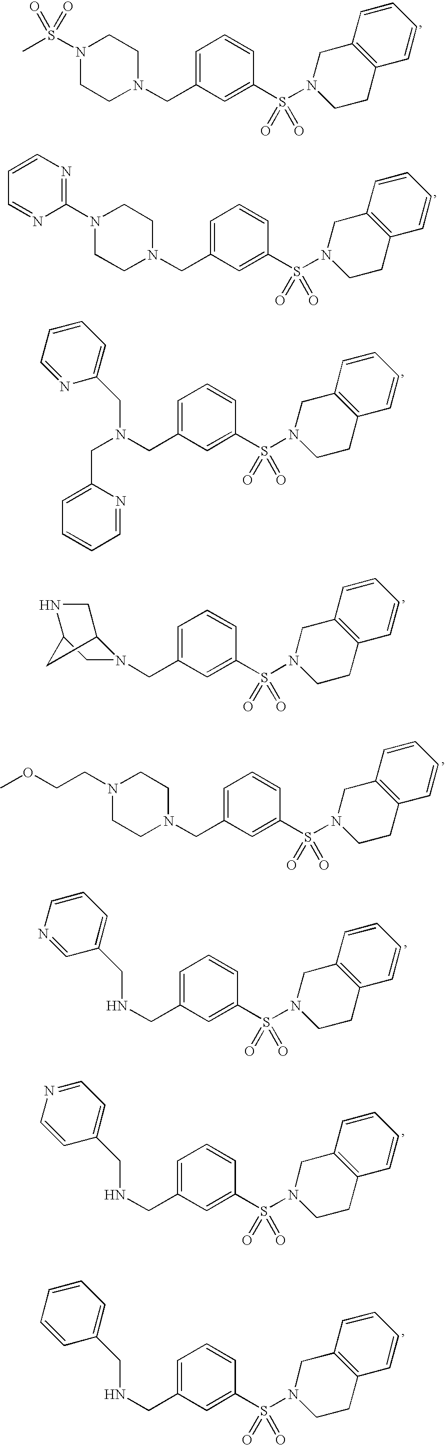 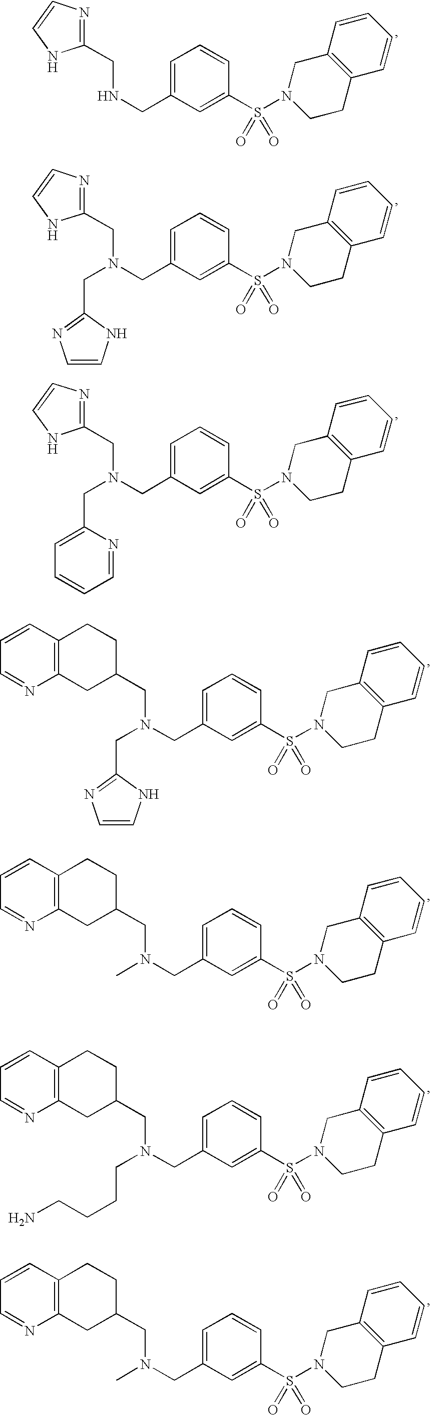 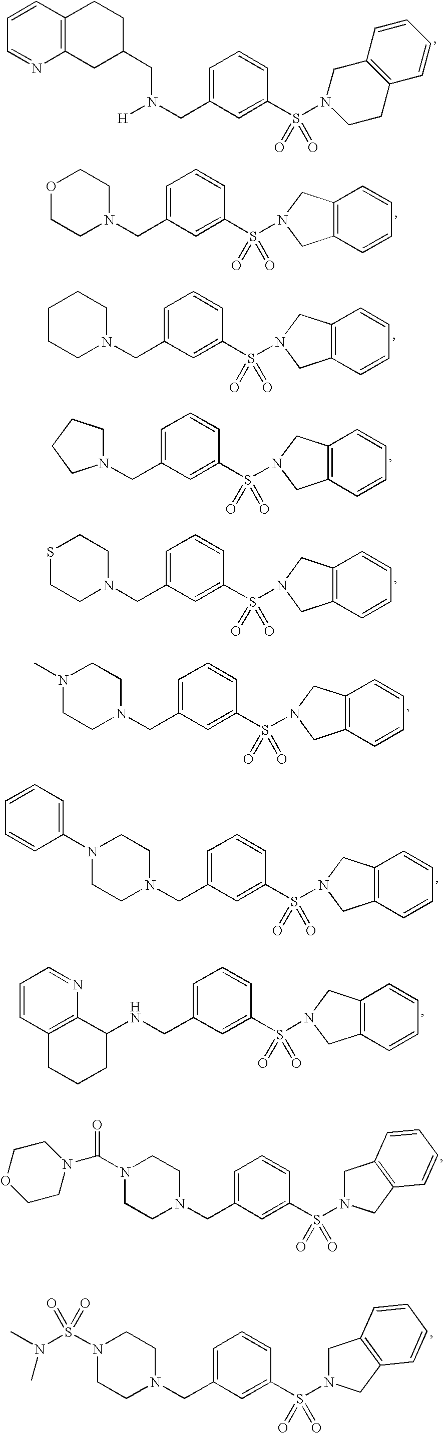 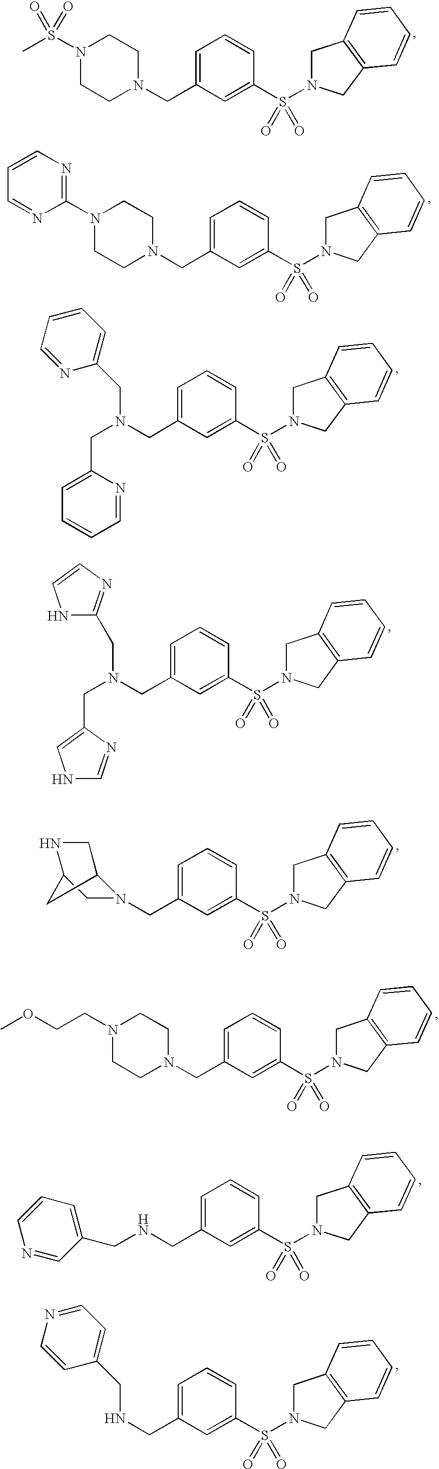 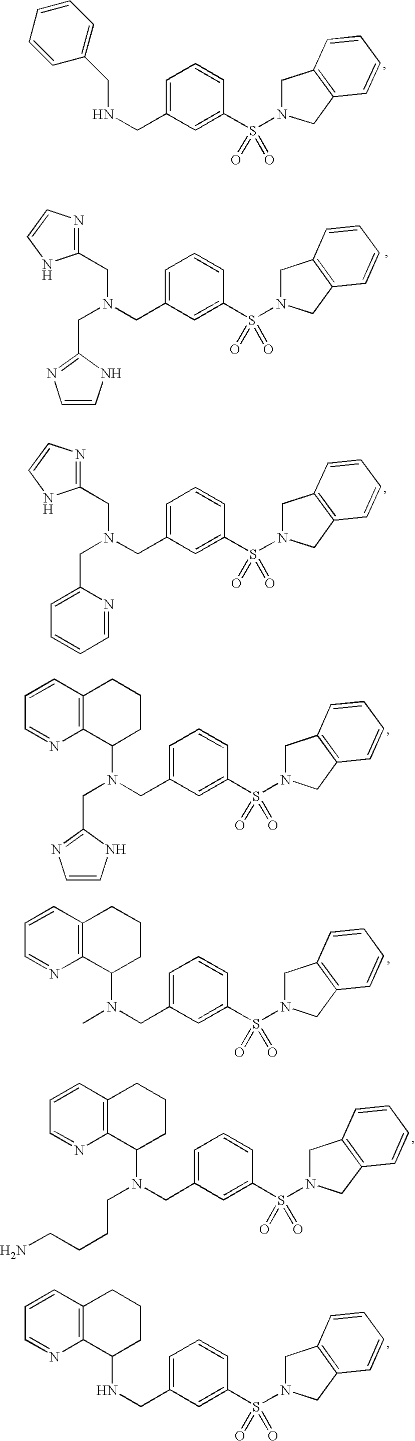 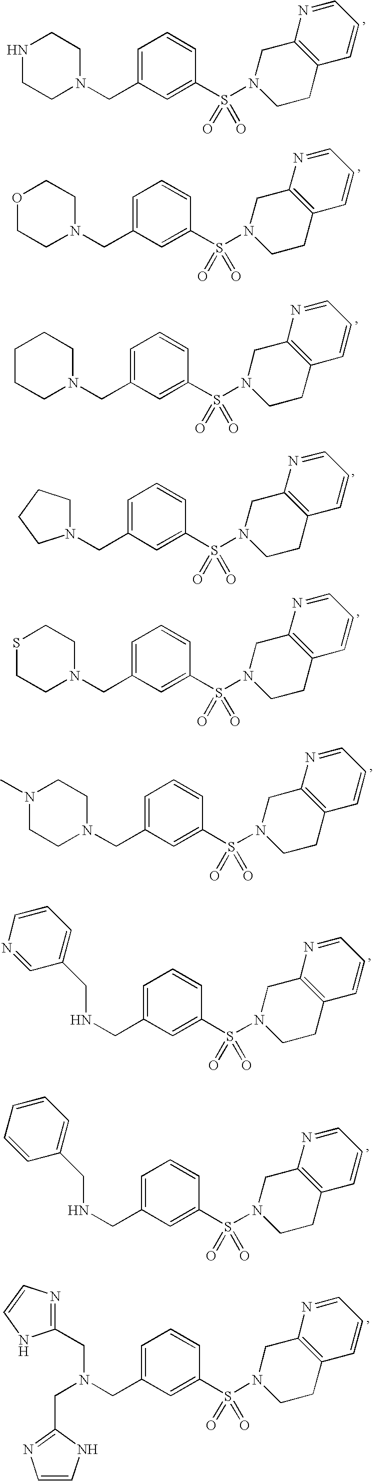 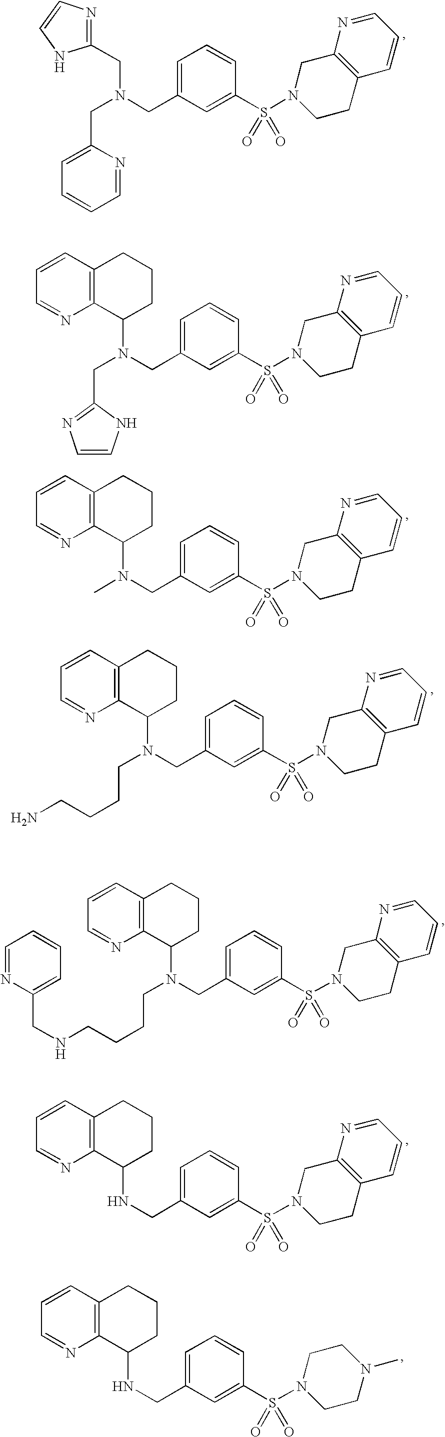 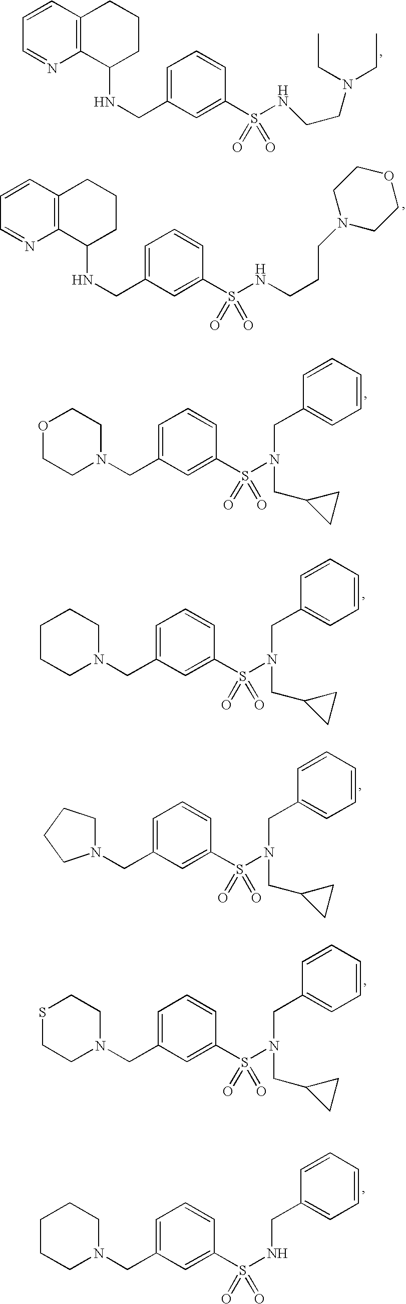 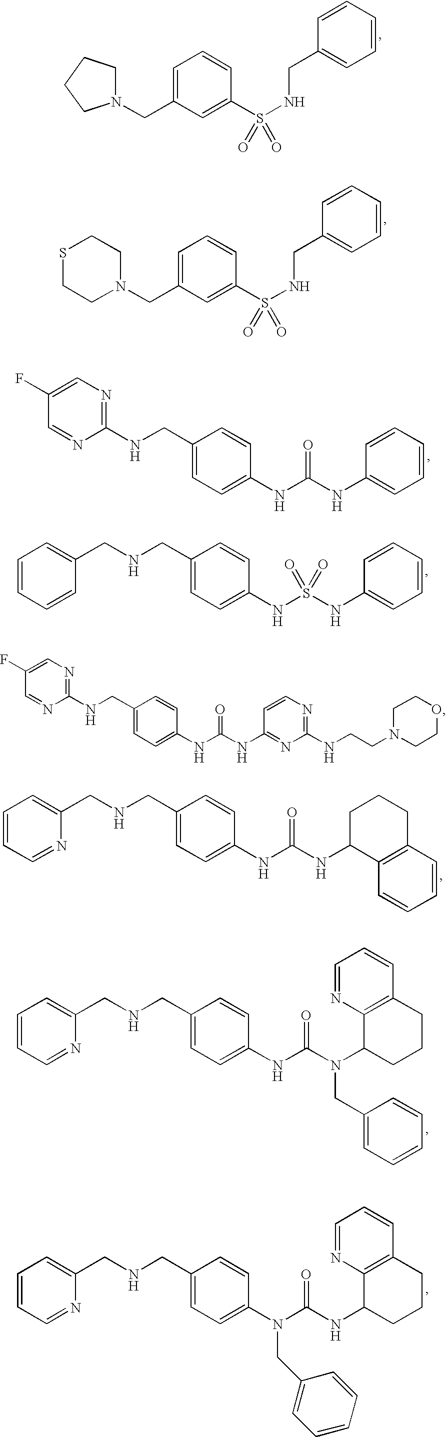 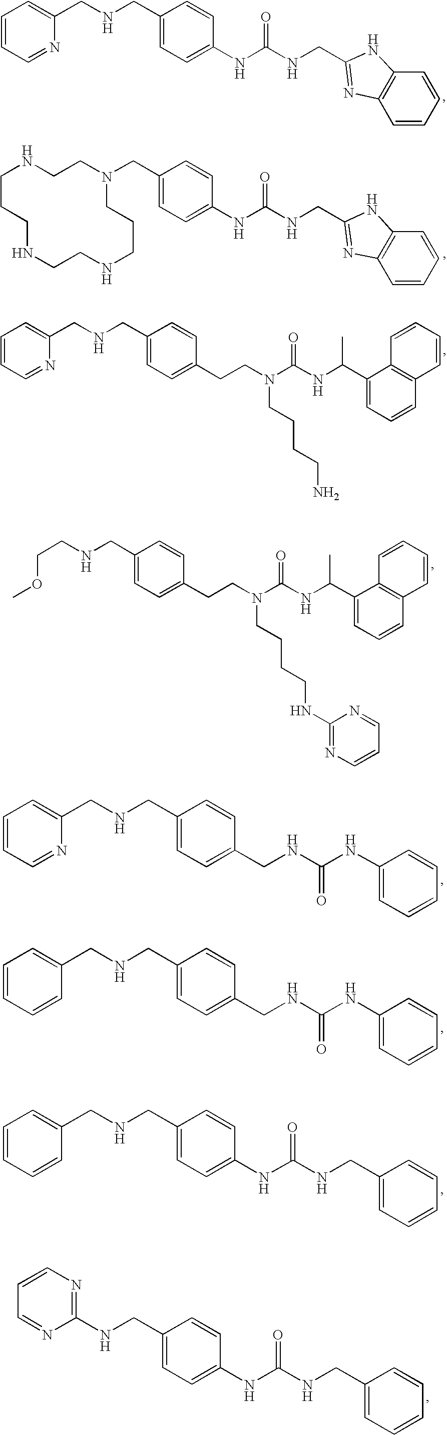 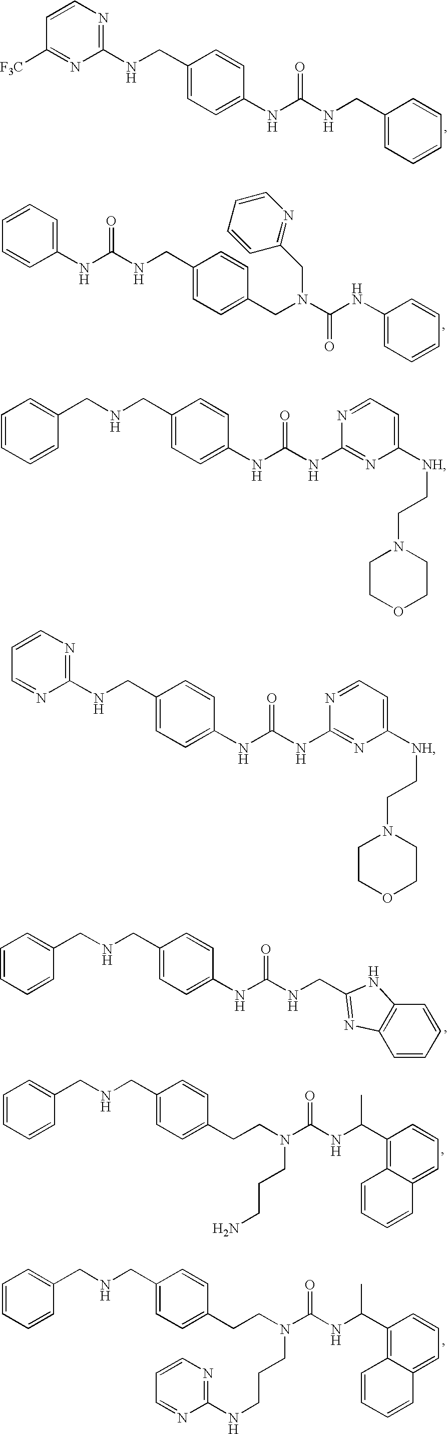 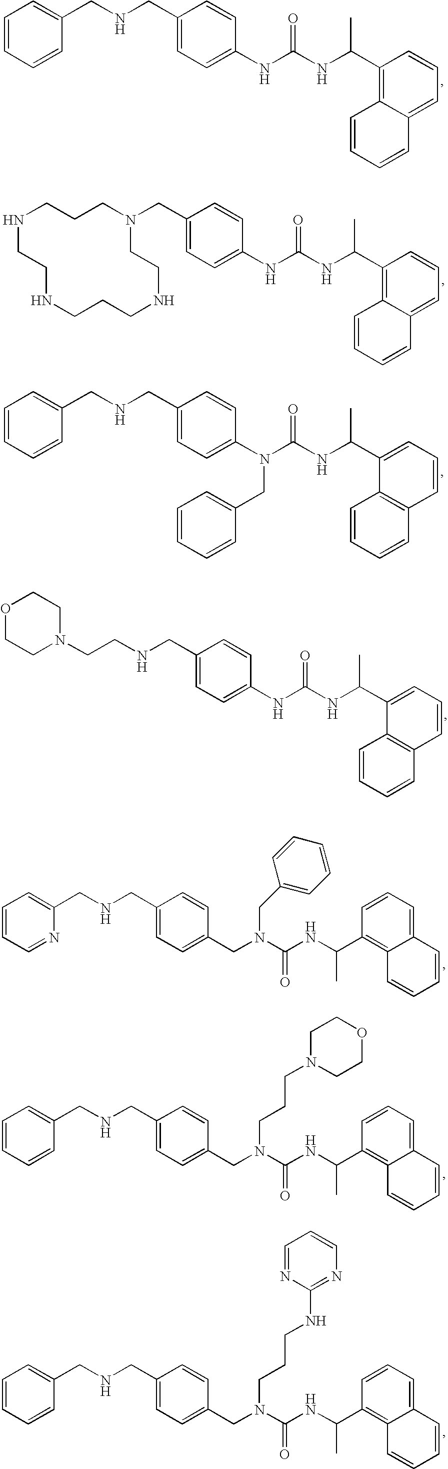 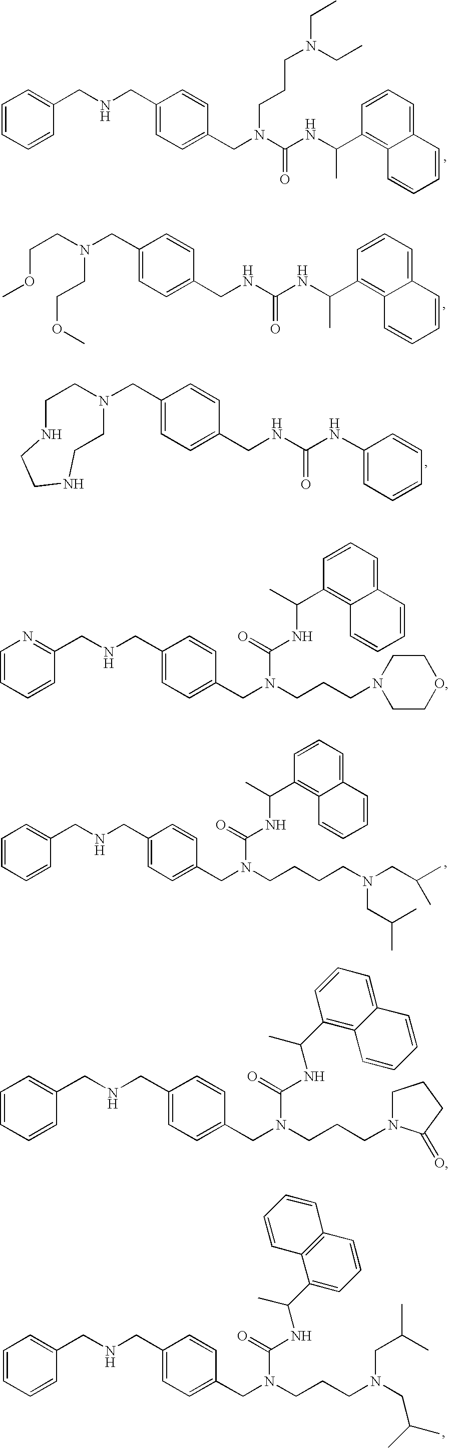 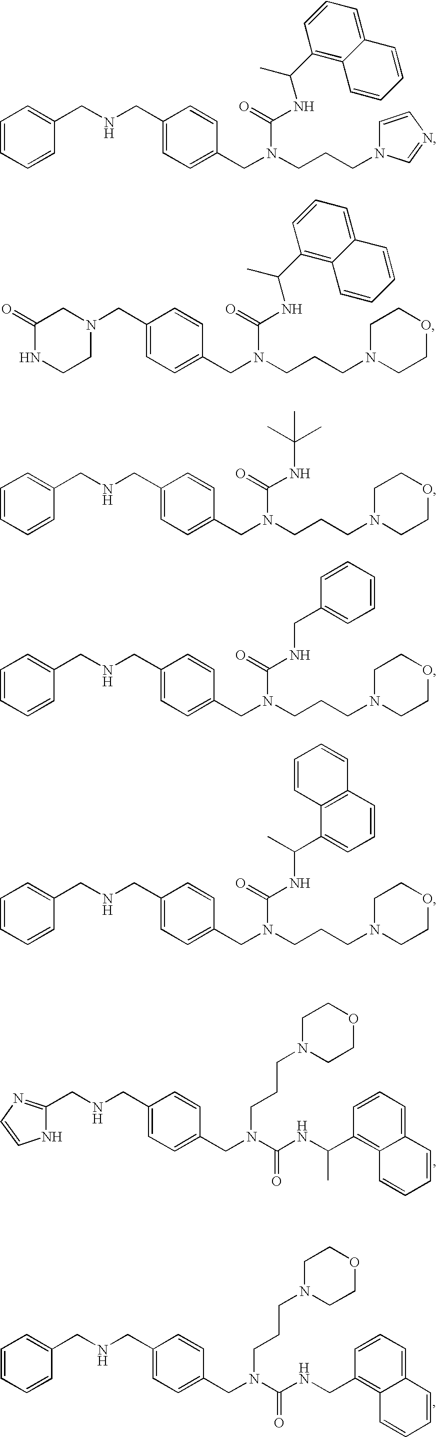 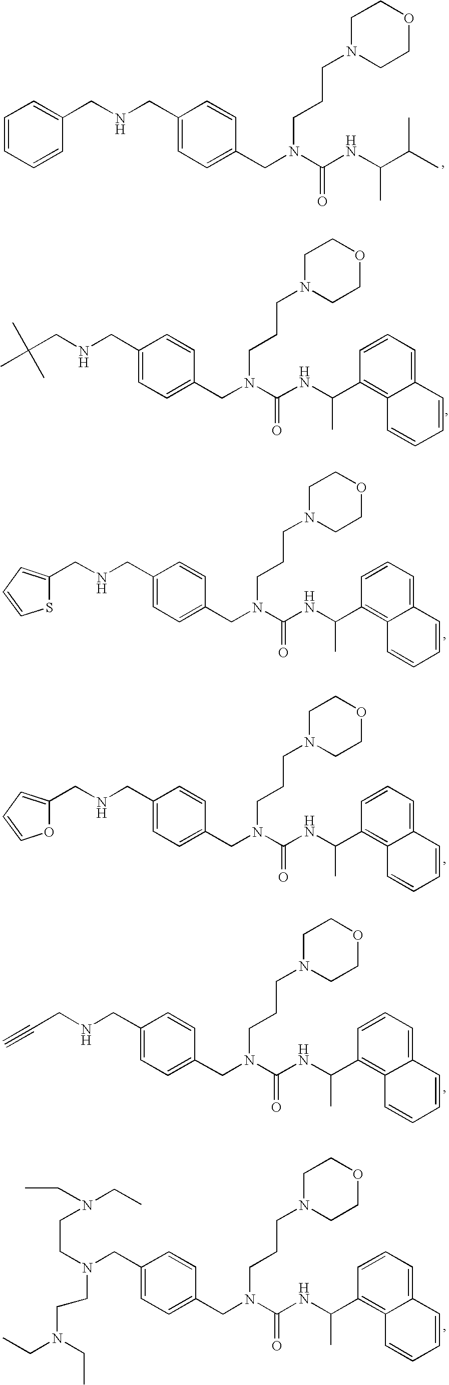 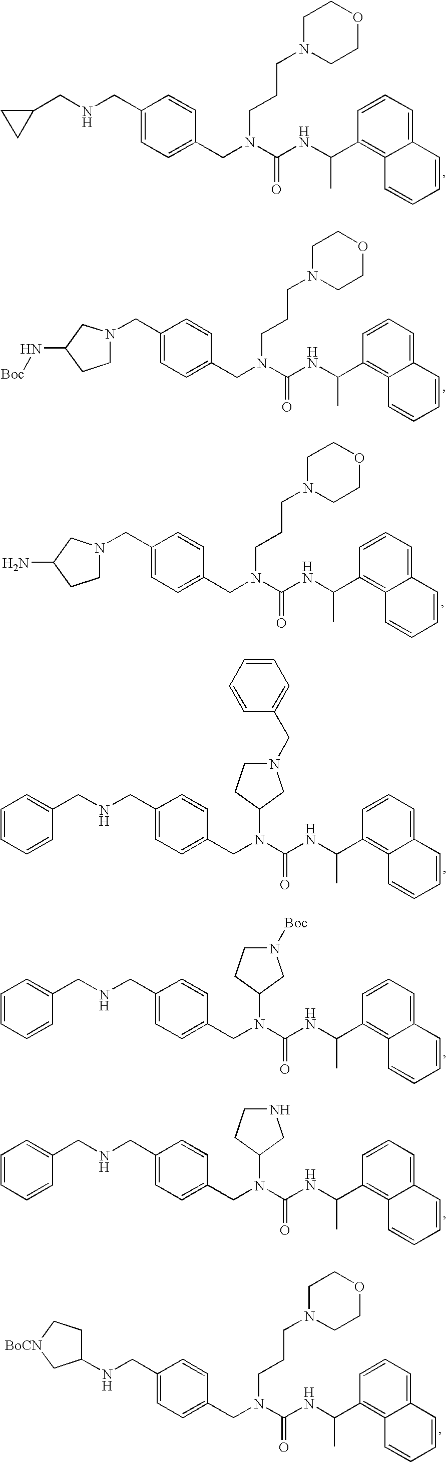 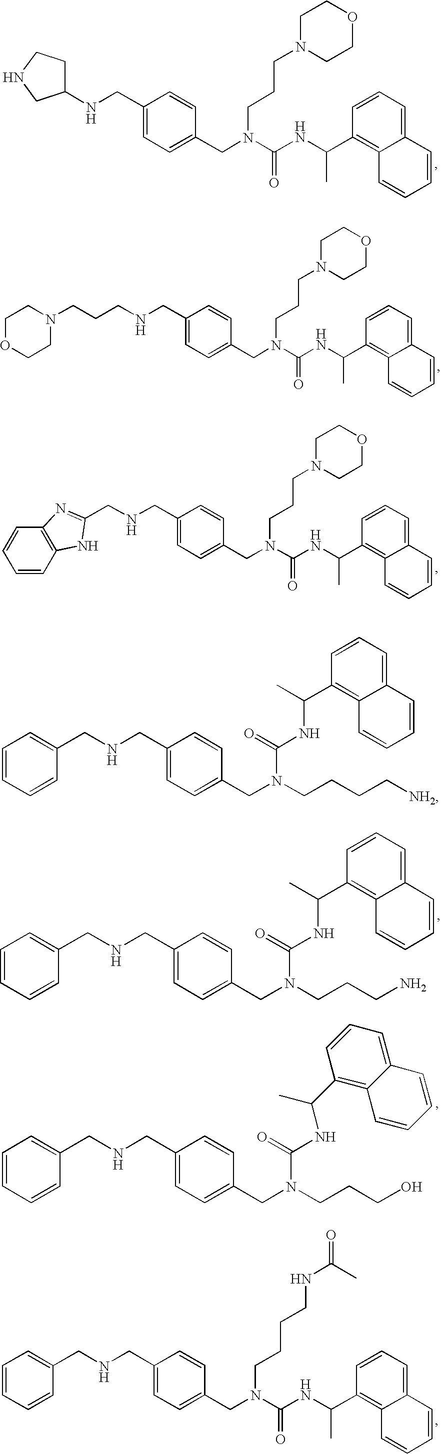 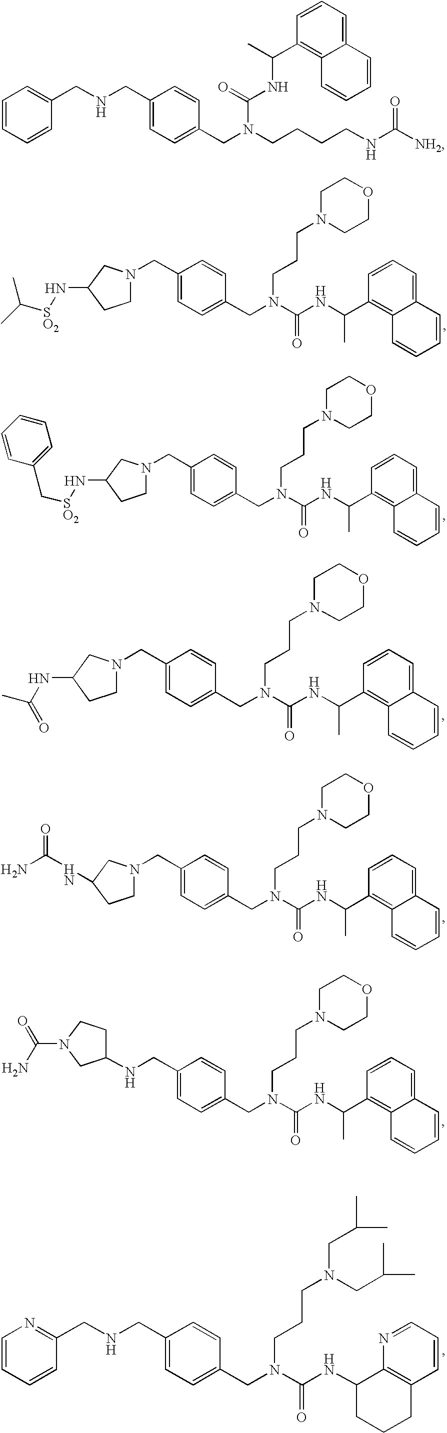 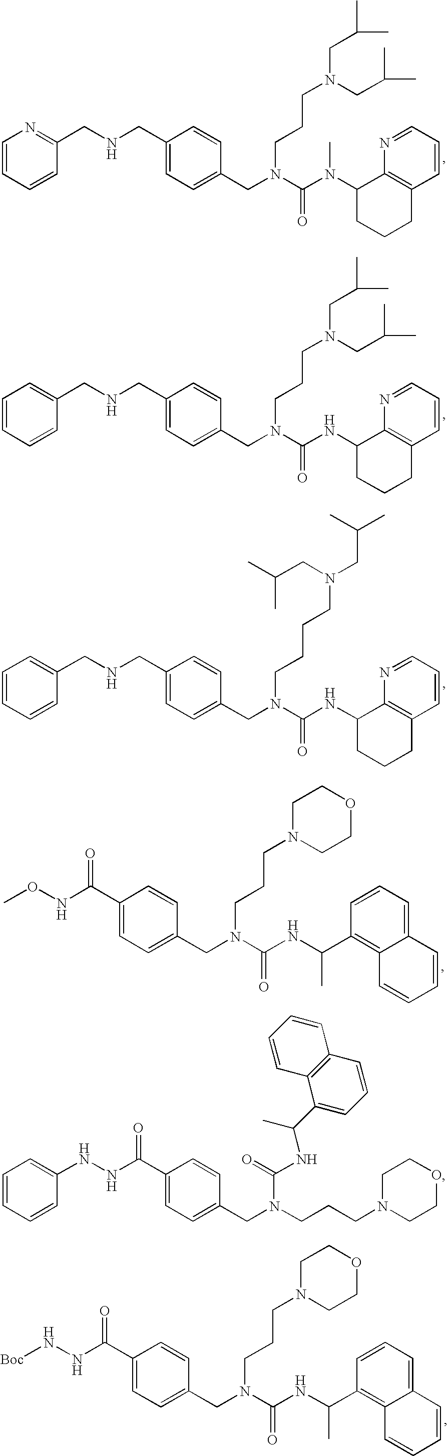 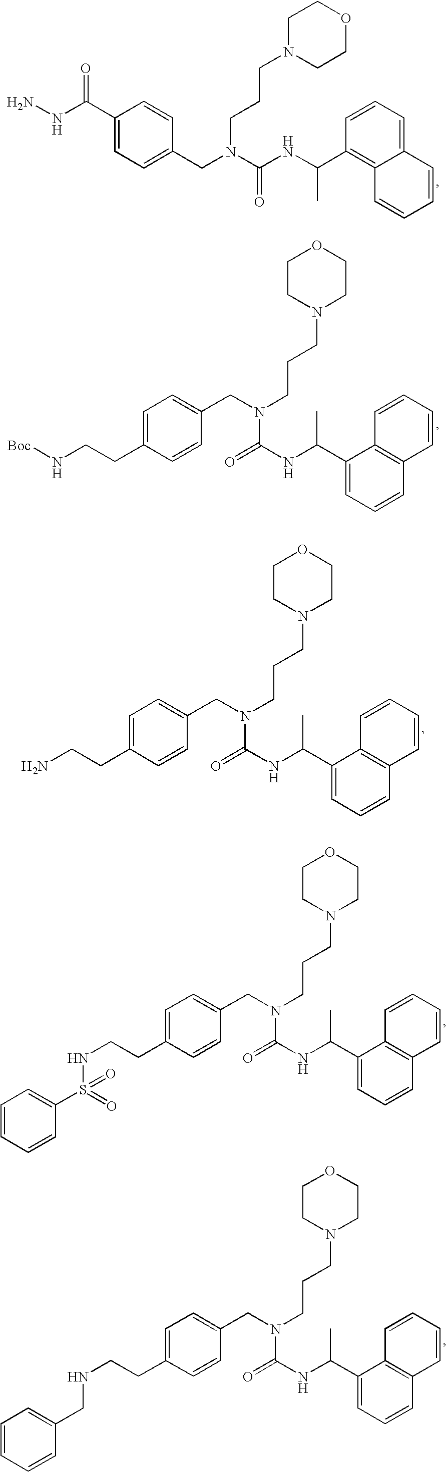 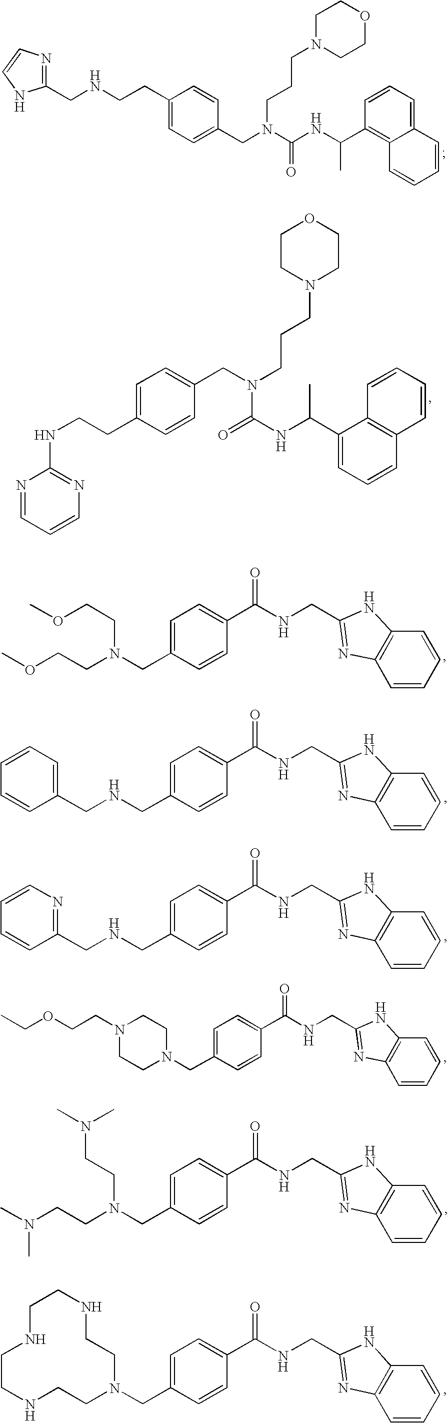 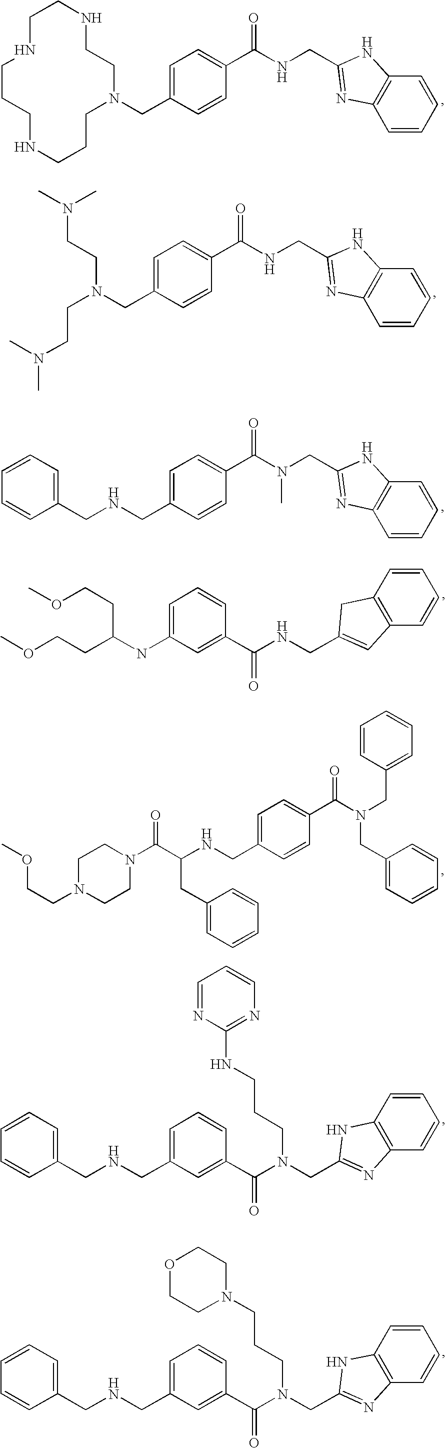 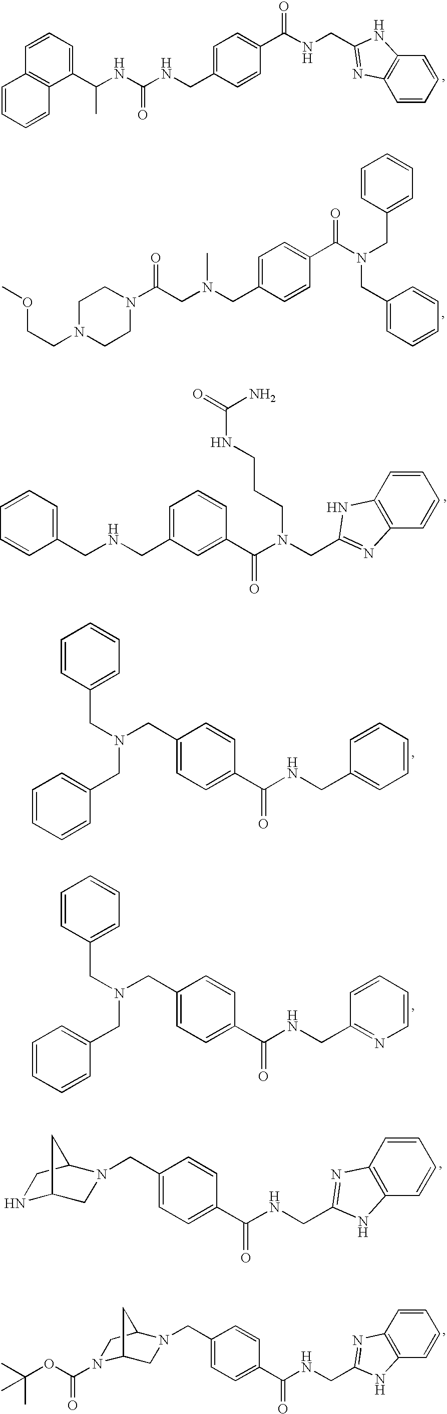 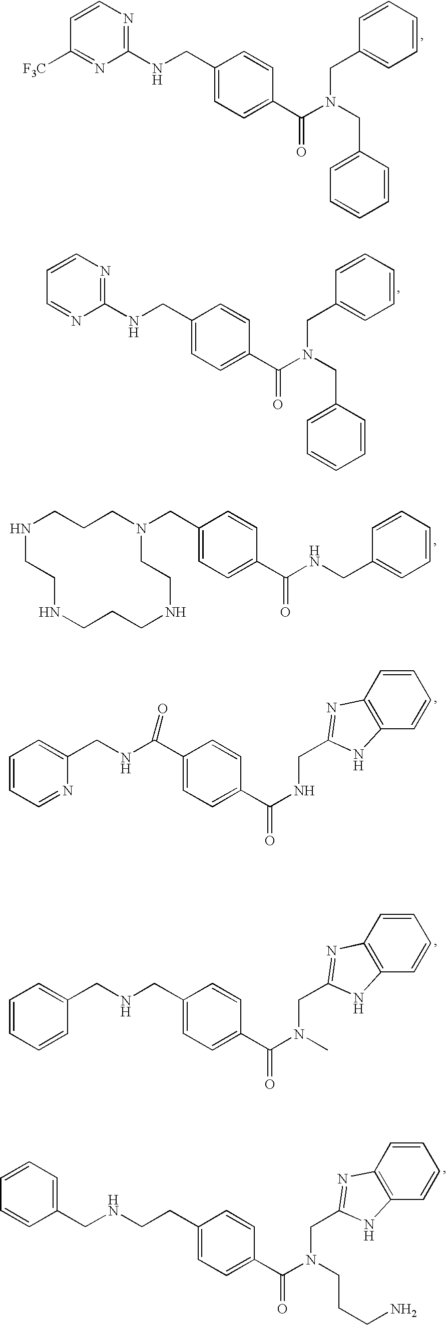 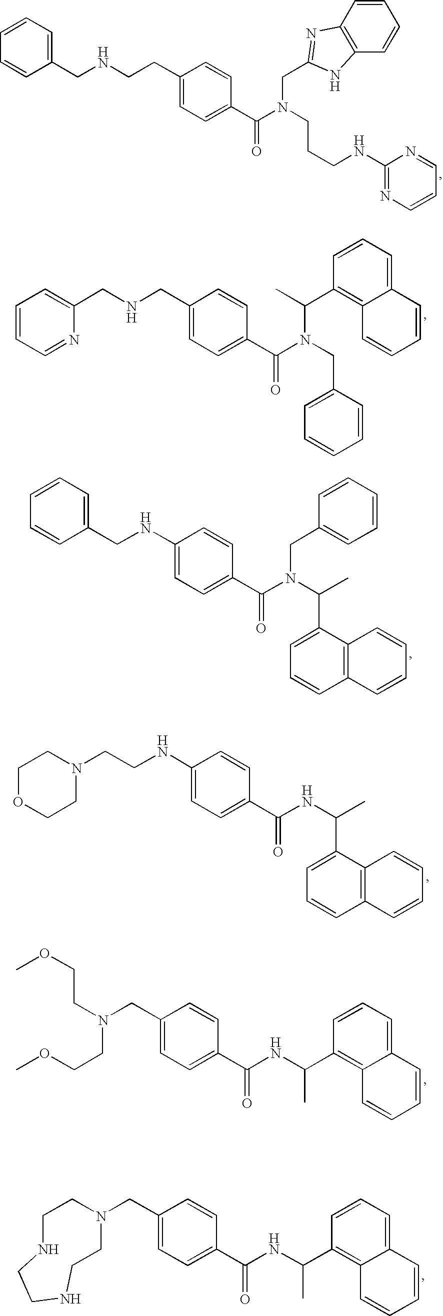 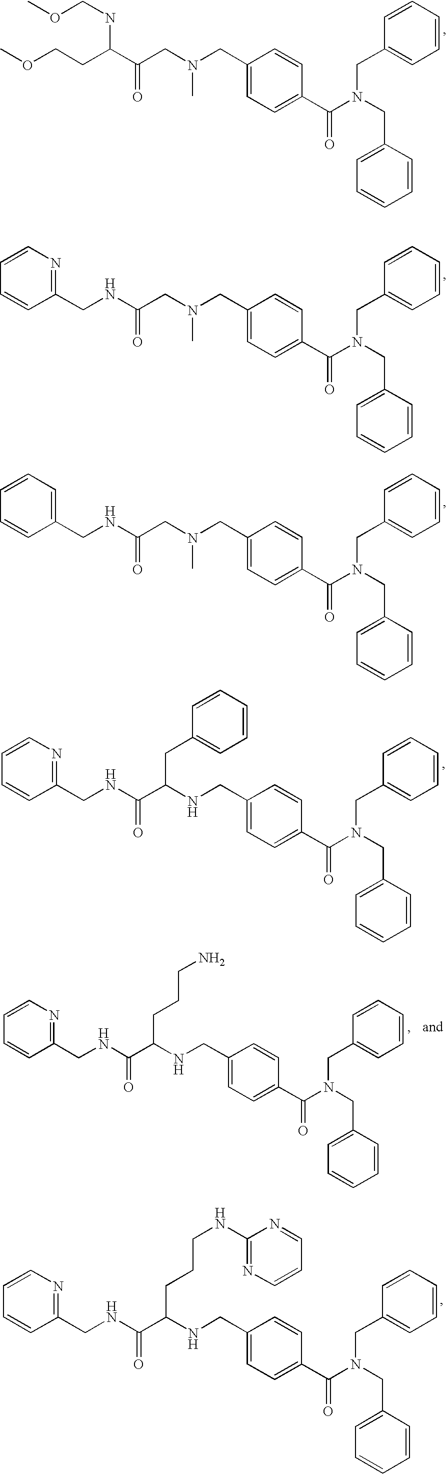
or a pharmaceutically acceptable salt, solvate, prodrug, tautomer, or ester thereof.

17. A pharmaceutical composition comprising a compound of claim 1, or a pharmaceutically acceptable salt, solvate, prodrug, tautomer, or ester thereof, and a pharmaceutically acceptable excipient.

18. The pharmaceutical composition of claim 17, further comprising at least one additional pharmaceutically active compound.

19. The pharmaceutical composition of claim 18, wherein the at least one additional pharmaceutically active compound is selected from the group consisting of amprenavir, lamivudine, zidovudine, indinavir, IDV, MK-639, FTC, emtricitabine, lamivudine, 3TC, abacavir, lamivudine, saquinavir, enfuvirtide, T-20, zalcitabine, ddC, dideoxycytidine, saquinavir, SQV, lopinavir, ritonavir, Fosamprenavir Calcium, ABT-538, delavirdine, DLV, AZT, azidothymidine, ZDV, atazanavir sulfate, efavirenz, tenofovir disoproxil, didanosine, ddI, dideoxyinosine, nelfinavir, NFV, nevirapine, BI-RG-587, tenofovir disoproxil fumarate, stavudine, d4T, abacavir, GW5634, (+)Calanolide A, Capravirine, MIV-150, TMC125, RO033-4649, TMC114, Tipranavir, GW640385, Elvucitabine, Alovudine, MIV-210, Racivir, SPD754, Reverset, FP21399, AMD070, GW873140, BMS-488043, PRO542, TAK-220, TNX-355, UK-427,857, AMD070, BMS-488043, FP21399, GW873140, PRO542, Schering SCH 417690, TAK-220, TNX-355 UK-427,857; Integrase Inhibitors;
Maturation Inhibitors, PA457; Zinc Finger Inhibitors, azodicarbonamide; Antisense Drugs, HGTV43, GEM92; Immune Stimulators, Ampligen, IL-2 (Proleukin), Bay 50-4798, Multikine, IR103; Vaccine-Like Treatment, HRG214, DermaVir, VIR201; and pharmaceutically acceptable salts, solvates, and esters thereof.

20. The pharmaceutical composition of claim 19, wherein the at least one additional pharmaceutically active compound is selected from the group consisting of 13-cis-Retinoic Acid, 2-Amino-6-Mercaptopurine, 2-CdA, 2-Chlorodeoxyadenosine, 5-fluorouracil, 5-FU, 6-TG, 6-Thioguanine, 6-Mercaptopurine, 6-MP, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Ara-C, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethason Acetate, dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia-L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxyrnesterone, Flutamide, Folinic Acid, FUDR, Fulvestrant, G-CSF, Gefitinib, Gemcitabine, Gemtuzumab ozogamicin, Gemzar, Gleevec, Gliadel wafer, Glivec, GM-CSF, Goserelin, granulocyte colony stimulating factor, Granulocyte macrophage colony stimulating factor, Halotestin, Herceptin, Hexadrol, Hexylen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL-2, IL-11, Imatinib mesylate, Imidazole Carboxamide, Interferon alfa, Interferon Alfa-2b (PEG conjugate), Interleukin-2, Interleukin-11, Intron A (interferon alfaL2b), Iressa, Irinotecan, Isotretinoin, Kidrolase, Lanacort, L-asparaginase, LCR, Letrozole, Leucovorin, Leukeran, Leukine, Leuprolide, Leurocristine, Leustatin, Liposomal Ara-C, Liquid Pred, Lomustine, L-PAM, L-Sarcolysin, Lupron, Lupron Depot, Matulane, Maxidex, Mechlorethamine, Mechlorethamine hydrochloride, Medralone, Medrol, Megace, Megestrol, Megestrol Acetate, Melphalan, Mercaptopurine, Mesna, Mesnex, Methotrexate, Methotrexate Sodium, Methylprednisolone, Meticorten, Mitomycin, Mitomycin-C, Mitoxantrone, M-Prednisol, MTC, MTX, Mylocel, Mylotarg, Navelbine, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon α-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinorelbine, Vinorelbine tartrate, VLB, VM-26, VP-16, Vumon, Xeloda, Zanosar, Zevalin, Zinecard, Zoladex, Zoledronic acid, Zometa, and pharmaceutically acceptable salts, solvates, and esters thereof.

21. A method of treating a disorder, symptom or disease in a patient in need of such treatment, comprising administering to the patient an effective amount of at least one compound of claim 1.

22. The method of claim 21, wherein said disorder, symptom or disease is a disorder, symptom or disease that is modulated by chemokine receptor activity or signaling.

23. The method of claim 22 wherein said treating is treatment or prophylaxis and the disorder, symptom or disease that is modulated by chemokine receptor activity or signaling is human immunodeficiency virus infections, flavivirus infections, pestivirus infections or cancer.

24. The method of claim 23, wherein the disorder, symptom or disease that is modulated by chemokine receptor activity or signaling is a cancer selected from the group consisting of bladder cancer, breast cancer, colorectal cancer, endometrial cancer, head & neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, ependymoma, Ewing's sarcoma family of tumors, germ cell tumor, extracranial cancer, Hodgkin's disease, leukemia, acute lymphoblastic leukemia, acute myeloid leukemia, liver cancer, medulloblastoma, neuroblastoma, brain tumors generally, non-Hodgkin's lymphoma, ostessarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, and small-cell lung cancer.

25. The method of claim 24, further comprising administering at least one additional pharmaceutically active compound is selected from the group consisting of amprenavir, lamivudine, zidovudine, indinavir, IDV, MK-639, FTC, emtricitabine, lamivudine, 3TC, abacavir, lamivudine, saquinavir, enfuvirtide, T-20, zalcitabine, ddC, dideoxycytidine, saquinavir, SQV, lopinavir, ritonavir, Fosamprenavir Calcium, ABT-538, delavirdine, DLV, AZT, azidothymidine, ZDV, atazanavir sulfate, efavirenz, tenofovir disoproxil, didanosine, ddI, dideoxyinosine, nelfinavir, NFV, nevirapine, BI-RG-587, tenofovir disoproxil fumarate, stavudine, d4T, abacavir, GW5634, (+)Calanolide A, Capravirine, MIV-150, TMC125, R0033-4649, TMC114, Tipranavir, GW640385, Elvucitabine, Alovudine, MIV-210, Racivir, SPD754, Reverset, FP21399, AMD070, GW873140, BMS-488043, PRO542, TAK-220, TNX-355, UK-427,857, AMD070, BMS-488043, FP21399, GW873140, PRO542, Schering SCH 417690, TAK-220, TNX-355 UK-427,857; Integrase Inhibitors; Maturation Inhibitors, PA457; Zinc Finger Inhibitors, azodicarbonamide; Antisense Drugs, HGTV43, GEM92; Immune Stimulators, Ampligen, IL-2 (Proleukin), Bay 50-4798, Multikine, IR103; Vaccine-Like Treatment, HRG214, DermaVir, VIR201; and pharmaceutically acceptable salts, solvates, and esters thereof

26. The method of claim 25, further comprising administering at least one additional pharmaceutically active compound is selected from the group consisting of 13-cis-Retinoic Acid, 2-Amino-6-Mercaptopurine, 2-CdA, 2-Chlorodeoxyadenosine, 5-fluorouracil, 5-FU, 6-TG, 6-Thioguanine, 6-Mercaptopurine, 6-MP, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Ara-C, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethason Acetate, dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia-L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxyrnesterone, Flutamide, Folinic Acid, FUDR, Fulvestrant, G-CSF, Gefitinib, Gemcitabine, Gemtuzumab ozogamicin, Gemzar, Gleevec, Gliadel wafer, Glivec, GM-CSF, Goserelin, granulocyte colony stimulating factor, Granulocyte macrophage colony stimulating factor, Halotestin, Herceptin, Hexadrol, Hexylen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL-2, IL-11, Imatinib mesylate, Imidazole Carboxamide, Interferon alfa, Interferon Alfa-2b (PEG conjugate), Interleukin-2, Interleukin-11, Intron A (interferon alfaL2b), Iressa, Irinotecan, Isotretinoin, Kidrolase, Lanacort, L-asparaginase, LCR, Letrozole, Leucovorin, Leukeran, Leukine, Leuprolide, Leurocristine, Leustatin, Liposomal Ara-C, Liquid Pred, Lomustine, L-PAM, L-Sarcolysin, Lupron, Lupron Depot, Matulane, Maxidex, Mechlorethamine, Mechlorethamine hydrochloride, Medralone, Medrol, Megace, Megestrol, Megestrol Acetate, Melphalan, Mercaptopurine, Mesna, Mesnex, Methotrexate, Methotrexate Sodium, Methylprednisolone, Meticorten, Mitomycin, Mitomycin-C, Mitoxantrone, M-Prednisol, MTC, MTX, Mylocel, Mylotarg, Navelbine, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon α-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinorelbine, Vinorelbine tartrate, VLB, VM-26, VP-16, Vumon, Xeloda, Zanosar, Zevalin, Zinecard, Zoladex, Zoledronic acid, Zometa, and pharmaceutically acceptable salts, solvates, and esters thereof.
